(19) United States
(12) Patent Application Publication
(10) Pub. No.: US 2025/0145609 A1
(43) Pub. Date: May 8, 2025

(54) 3-SUBSTITUTED PYRIDAZINE COMPOUNDS AS SMARCA2 AND/OR SMARCA4 DEGRADERS

(71) Applicant: Aurigene Oncology Limited, Bangalore (IN)

(72) Inventors: Chandrasekhar ABBINENI, Hyderabad (IN); Susanta SAMAJDAR, Bangalore (IN); Sanjita SASMAL, Hyderabad (IN); Bilash KUILA, Howrah (IN); Subhendu MUKHERJEE, HOOGHLY (IN); Suraj TATYASAHEB GORE, Maharashtra (IN)

(21) Appl. No.: 18/835,518

(22) PCT Filed: Feb. 9, 2023
(86) PCT No.: PCT/IB2023/051152

§ 371 Date: Aug. 2, 2024

(30) Foreign Application Priority Data
Feb. 9, 2022 (IN) 202241006964

(51) Int. Cl. C07D 417/14; A61K 31/501; A61P 35/00

(52) U.S. Cl. CPC C07D 417/14; A61K 31/501; A61P 35/00

(57) ABSTRACT

The present invention provides 3-substituted pyridazine compound of formula (I), which are therapeutically useful as SMARCA2 and/or SMARCA4 degraders. These compounds are useful in the treatment and/or prevention of diseases or disorders dependent upon SMARCA2 and/or SMARCA4 in a subject. The present invention also provides preparation of the compounds and pharmaceutical compositions comprising a compound of formula (I) or a pharmaceutically acceptable salt or a stereoisomer or a tautomer thereof.

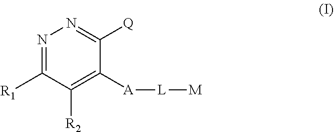

### RELATED APPLICATION

This application claims priority of Indian Provisional Application No. 202241006964, filed on 9 Feb. 2022; the specification of which is hereby incorporated by reference in its entirety.

### FIELD OF THE INVENTION

The present invention relates to 3-substituted pyridazine compounds and a pharmaceutically acceptable salt or a stereoisomer or a tautomer thereof which are useful as SMARCA2 and/or SMARCA4 degraders and, for the treatment of diseases or disorders dependent on SMARCA2 and/or SMARCA4. The present invention also relates to a method of preparation of the said pyridazine compounds and pharmaceutical compositions comprising the said compounds.

### BACKGROUND OF THE INVENTION

One of the most significant findings from the cancer genome profiling is the discovery of frequent mutations in various subunits of the mammalian SWI/SNF (SWItch/Sucrose Non-Fermentable) chromatin remodelling complex. Approximately 20% of human cancers are associated with somatic mutations in subunits of the SWI/SNF complex, a chromatin remodelling complex that influences gene regulation by disrupting histone-DNA contacts (PNAS Feb. 25, 2014. 111 (8) 3128-3133).

SWI/SNF complexes contain either of two closely related and evolutionarily conserved catalytic ATPase subunits: Brahma (BRM/SMARCA2) or Brahma-related gene 1 (BRG1/SMARCA4). They share approximately 75% identity at the protein level. Although BRG1—and BRM-containing complexes show some redundancy, they may function distinctively. In human cancer, BRG1 seems to be one of the most frequently mutated subunit genes, whereas the BRM gene is rarely mutated. BRG1/SMARCA4 mutations occurring in ˜10-15% of lung adenocarcinomas. BRM/SMARCA2 is essential for the growth of tumour cells that harbor loss of function mutations in BRG1/SMARCA4. Depletion of BRM in BRG1-deficient cancer cells leads to a cell cycle arrest, induction of senescence and increased levels of global H3K9me31 (PNAS Feb. 25, 2014. 111 (8) 3128-3133).

In some tumour types, mutations within the SWI/SNF complex lead to context specific vulnerabilities such as the requirement of SMARCA2 for survival of tumour cells lacking SMARCA4. This finding of SMARCA2/4 synthetic lethal relationship translates in vivo which emphasizes SMARCA2 as a promising therapeutic target for the treatment SMARCA4-deficient cancers. Moreover, the SMARCA4-deficient patient population generally lacks targetable oncogenes (such as mutant EGFR or ALK translocations), which further emphasizes the potential of developing SMARCA2 inhibitors. Characterization of SMARCA4 function in tumours with high SMARCA4 levels, shows effects on signalling pathways that result in increased proliferation and survival. SMARCA4 knockdown in tumours that show elevated levels known to inhibit proliferation and other cancer cell properties. Studies have also shown that SMARCA4 knock down/modulation increases sensitivity to known chemotherapeutic agents, thereby indicating that SMARCA4 targeting could also be an adjuvant therapy to existing chemotherapeutic approaches (PNAS Feb. 25, 2014. 111 (8) 3128-3133; J Pathol. 2016 February; 238(3): 389-400).

Contrary to the point that genetic silencing of SMARCA2 leading to potent anti-proliferative activity in SMARCA4-deficient cancer cell lines, pharmacological studies of PFI-3, a selective cell permeable SMARCA2/4 bromodomain inhibitor which is capable of binding to SMARCA2 and SMARCA4 bromodomain, fail to display an antiproliferative phenotype. This indicates that bromodomain function of SMARCA2/4 is dispensable for tumor cell proliferation, while the catalytic ATPase activity is essential (Cancer Res. 2015 Sep. 15; 75(18): 3865-3878). In order to mimic the phenotype achieved by genetic silencing, approaches that lead to reduction or complete elimination of SMARCA2/4 may be needed.

The ubiquitin-proteasome system (UPS) is a major pathway that regulates the levels of intracellular proteins and provides a fine balance between protein synthesis and degradation required for normal maintenance of cellular functions, such as proliferation, differentiation and cell death. Ubiquitination is a post-translational modification, where a small protein, ubiquitin, is covalently attached to lysine residues on a substrate protein which is carried out sequentially by a cascade of enzymatic reactions involving an intimate collaboration between E1 activating, E2 conjugating and E3 ligating enzymes and subsequent degradation of the tagged proteins (J. Biosci. 31(1), March 2006, 137-155; Expert Opin Ther Targets. 2013 September; 17(9): 1091-1108 and Cell Research (2016) 26:484-498).

Proteolysis targeting chimeras (PROTACs) are the heterobifunctional molecules contain a ligand for a target protein of interest connected via a linker to a ligand for an E3 ubiquitin ligase. Upon such bi-functional molecule-mediated heterodimerization of the two bound proteins, the target protein is ubiquitinated and degraded by the proteasome in cells. Many such bi-functional molecules have been developed to recruit E3 ubiquitin ligases to a variety of substrates using high-affinity ligands for the protein of interest. Proteins effectively degraded using these approaches include RIPK2 and ERRα, BRD4, BRD9, BCR/Abl and Abl and Erα. (Cell Chemical Biology 25, 1-10, Jan. 18, 2018). E3 ubiquitin ligases (of which over 600 are known in humans) confer substrate specificity for ubiquitination and are more attractive therapeutic targets than general proteasome inhibitors due to their specificity for certain protein substrates (Cancer Res. 2017 May 1; 77(9):2476-2487).

Small molecule ligands targeting the bromodomains of SMARCA2 and SMARCA4 have been reported in the literature (Journal of Medicinal Chemistry 2016, 59, 4800-4811; Hoffman et al., PNAS, 2014b, 777, 3128-3133; Sutherell et al., 2016, Journal of Medicinal Chemistry 59, 5095-5101 and WO2016138114). Although cells lacking SMARCA4 activity are vulnerable to the loss of SMARCA2 (2014a, PNAS 777, 3128-3133). SMARCA2/4 inhibitors have failed to phenocopy these anti-proliferative effects. In agreement with this, re-expression of SMARCA2 variants in cells, where the endogenous protein had been suppressed, showed that an intact bromodomain is not required to maintain proliferation (Cancer Res. 2015, Sep. 15; 75(18): pp. 3865-3878). SMARCA2/4 Bromo domain inhibitors are thus precluded from use for the treatment of SMARCA4 mutant cancers but could provide attractive ligands for PROTAC conjugation.

It is therefore reasoned that a PROTAC targeting the non-functional bromodomain of SMARCA2 and/or SMARCA4 should offer an opportunity to exploit the vulnerability of SMARCA2 in SMARCA4 mutated cancer cells for therapeutic purposes. The principle of conjugation of a suitable SMARCA ligand with an E3 ligase binder has been described in WO2016/105518, WO2016138114, WO2017007612, WO2017011371, WO2019195201, WO2019207538, WO2019213005, WO2020010227, WO2020078933, WO2020251972, WO2020251969, WO2020251971, WO2020251974, WO2021083949, WO2021133920, WO2021133917, WO2021142247 and WO2021207291. However, none of the publications demonstrate any concrete example for degradation of SMARCA proteins.

### SUMMARY OF THE INVENTION

Provided herein 3-substituted pyridazine compounds and pharmaceutical compositions thereof that are useful as SMARCA2 and/or SMARCA4 degraders and for treating diseases or conditions or disorders that are dependent upon or mediated by SMARCA2 and/or SMARCA4.

In one aspect, the present invention provides compound of formula (I):

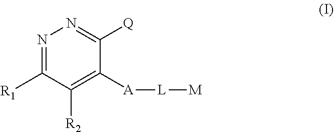

or a pharmaceutically acceptable salt or a stereoisomer or a tautomer thereof;
wherein,
   A represents 5- to 6-membered heteroarylenyl or 6-membered arylenyl; wherein the arylenyl and heteroarylenyl are unsubstituted or substituted with 1, 2 or 3 Rₐ;
   Rₐ, at each occurrence, independently represents hydroxy, hydroxyalkyl, halogen, alkoxy, alkyl, haloalkyl or cyano;
   R₁ is halogen, alkyl, haloalkyl, alkoxy, hydroxy, hydroxyalkyl, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano;
   R₂ is hydrogen, hydroxy, hydroxyalkyl, halogen, alkoxy, alkyl, amino, aminoalkyl, haloalkyl or cyano;
   Q is amino, aminoalkyl, hydroxy, hydroxyalkyl, halogen, alkoxy, alkyl, haloalkyl or cyano;
   L is a bond, —N(Rₓ)—(CRₓR_{y})p-O—, —O—(CRₓR_{y})p-, —O—(CRₓR_{y})p-O—, —C≡C—(CRₓR_{y})p-, -heterocycloalkylenyl-heterocycloalkylenyl-, -heterocycloalkylenyl-O—, -heterocycloalkylenyl-C≡C—, —C≡C-heterocycloalkylenyl-, —O-heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p₁-heterocycloalkylenyl- or -heterocycloalkylenyl-O-heterocycloalkylenyl-; wherein the cycloalkylenyl, heteroarylenyl and heterocycloalkylenyl are unsubstituted or substituted with 1, 2 or 3 R_{d}; and the left side of the L group is attached to A and the right side of L group is attached to M;
   R_{d}, at each occurrence, is independently selected from hydroxy, halogen, alkyl and alkoxy; or any two R_{d} groups attached with the same C atom together form an oxo group;
   Rₓ and R_{y}, at each occurrence, are independently selected from hydrogen and alkyl;
   M is M-1 and M-2:

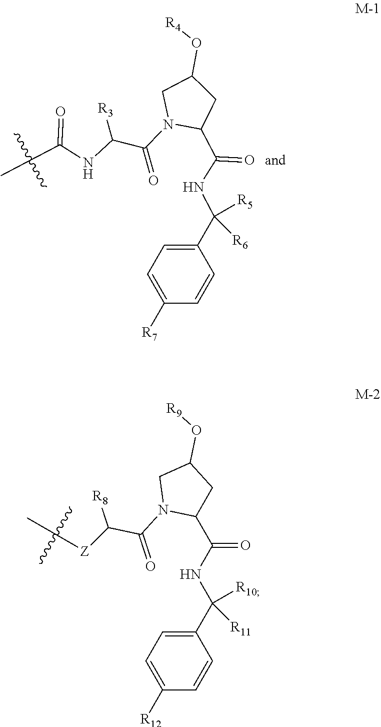

wherein
Z is 5- to 6-membered heteroarylenyl or 5- to 6-membered heterocycloalkylenyl, wherein the heteroarylenyl and heterocycloalkylenyl groups are unsubstituted or substituted with oxo, hydroxy, halogen, alkyl or alkoxy;
R₃ and R₈ independently represents alkyl, haloalkyl or hydroxyalkyl;
R₄ and R₉ independently represents hydrogen, alkyl, haloalkyl or hydroxyalkyl;
R₅, R₆, R₁₀ and R₁₁ are independently selected from hydrogen, alkyl, halogen, heteroalkyl, haloalkyl, hydroxyalkyl and acyl;
R₇ and R₁₂ independently represents thiazolyl which is unsubstituted or substituted with alkyl, hydroxy, amino or haloalkyl;
‘p’ is an integer selected from 0, 1, 2, 3, 4, 5 and 6; and
‘p₁’ is an integer selected from 1, 2, 3 and 4.

In another aspect, the present invention provides a pharmaceutical composition comprising a compound of formula (I) or a pharmaceutically acceptable salt or a stereoisomer or a tautomer thereof and at least one pharmaceutically acceptable excipient (such as a pharmaceutically acceptable carrier or diluent).

In yet another aspect, the present invention relates to the preparation of compound of formula (I).

In another aspect, the present invention provides a pharmaceutical composition comprising a compound of formula (I) or a pharmaceutically acceptable salt or a stereoisomer or a tautomer thereof for treating diseases or conditions or disorders that are dependent upon or mediated by SMARCA2 and/or SMARCA4.

In another aspect, the present invention provides a pharmaceutical composition comprising a compound of formula (I) or a pharmaceutically acceptable salt or a stereoisomer or a tautomer thereof for treating diseases or conditions or disorders that have altered SMARCA2 and/or SMARCA4 including mutations and overexpression.

In another aspect, the present invention provides a pharmaceutical composition comprising a compound of formula (I) or a pharmaceutically acceptable salt or a stereoisomer or a tautomer thereof for treating diseases or conditions or disorders wherein degradation of SMARCA2 and/or SMARCA4 proteins provide a benefit, e.g., cancer.

In another aspect, the present invention provides methods of treating a condition or a disease or a disorder by administering a therapeutically effective amount of a compound of formula (I) or a pharmaceutically acceptable salt or a stereoisomer or a tautomer thereof to an individual, e.g., a human, in need thereof. The conditions or diseases or disorders of interest is treatable by degradation of SMARCA2 and/or SMARCA4, for example, a cancer, a chronic autoimmune disorder, an inflammatory condition, a proliferative disorder, sepsis or a viral infection.

In another aspect, the present invention provides a composition of a compound of formula (I) or a pharmaceutically acceptable salt or a stereoisomer or a tautomer thereof for treating diseases or conditions or disorders that are dependent upon altered activity of SWI/SNF complex with or without chromatic remodeling activities.

In another aspect, the present invention provides a use of a compound of formula (I) or a pharmaceutically acceptable salt or a stereoisomer or a tautomer thereof for the manufacture of a medicament for treating a disease or a condition or a disorder of interest, e.g., cancer.

### DETAILED DESCRIPTION OF THE INVENTION

The present invention provides 3-substituted pyridazine compounds, referred as a compound of formula (I), which are useful as SMARCA2 and/or SMARCA4 degraders and for the treatment of conditions dependent on or mediated by SMARCA2 and/or SMARCA4. The present invention further provides pharmaceutical compositions comprising the said compound or a stereoisomer or a tautomer thereof as therapeutic agents.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In one embodiment, the present invention provides compound of formula (I),

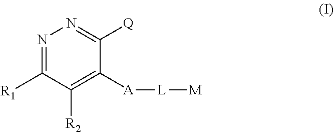

or a pharmaceutically acceptable salt or a stereoisomer or a tautomer thereof;
wherein,
   A represents 5- to 6-membered heteroarylenyl or 6-membered arylenyl; wherein the arylenyl and heteroarylenyl are unsubstituted or substituted with 1, 2 or 3 Rₐ;
   Rₐ, at each occurrence, independently represents hydroxy, hydroxyalkyl, halogen, alkoxy, alkyl, haloalkyl or cyano;
   R₁ is halogen, alkyl, haloalkyl, alkoxy, hydroxy, hydroxyalkyl, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano;
   R₂ is hydrogen, hydroxy, hydroxyalkyl, halogen, alkoxy, alkyl, amino, aminoalkyl, haloalkyl or cyano;
   Q is amino, aminoalkyl, hydroxy, hydroxyalkyl, halogen, alkoxy, alkyl, haloalkyl or cyano;
   L is a bond, —N(Rₓ)—(CRₓR_{y})p-O—, —O—(CRₓR_{y})p-, —O—(CRₓR_{y})p-O—, —C≡C—(CRₓR_{y})p-, -heterocycloalkylenyl-heterocycloalkylenyl-, -heterocycloalkylenyl-O—, -heterocycloalkylenyl-C≡C—, —C≡C-heterocycloalkylenyl-, —O-heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p₁-heterocycloalkylenyl- or -heterocycloalkylenyl-O-heterocycloalkylenyl-; wherein the cycloalkylenyl, heteroarylenyl and heterocycloalkylenyl are unsubstituted or substituted with 1, 2 or 3 R_{d}; and the left side of L group is attached to A and right side of L group is attached to M;
   R_{d}, at each occurrence, is independently selected from hydroxy, halogen, alkyl and alkoxy; or any two R_{d} groups attached with the same C atom together form an oxo group;
   Rₓ and R_{y}, at each occurrence, are independently selected from hydrogen and alkyl;
   M is selected from M-1 and M-2:

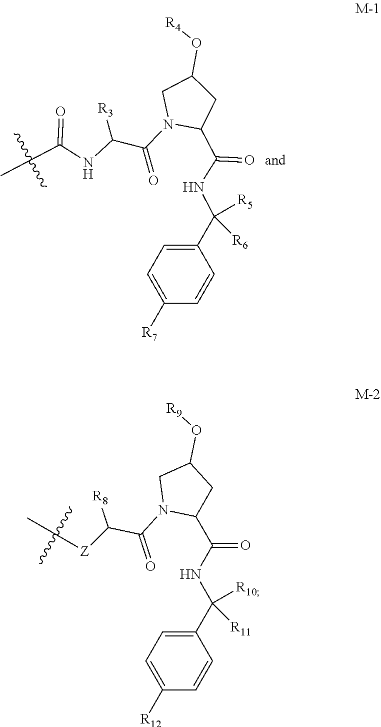

wherein
Z is 5- to 6-membered heteroarylenyl or 5- to 6-membered heterocycloalkylenyl, wherein the heteroarylenyl and heterocycloalkylenyl groups are unsubstituted or substituted with oxo, hydroxy, halogen, alkyl or alkoxy;
R₃ and R₈ independently represents alkyl, haloalkyl or hydroxyalkyl;
R₄ and R₉ independently represents hydrogen, alkyl, haloalkyl or hydroxyalkyl;
R₅, R₆, R₁₀ and R₁₁ are independently selected from hydrogen, alkyl, halogen, heteroalkyl, haloalkyl, hydroxyalkyl and acyl;
R₇ and R₁₂ independently represents thiazolyl which is unsubstituted or substituted with alkyl, hydroxy, amino or haloalkyl;
‘p’ is an integer selected from 0, 1, 2, 3, 4, 5 and 6; and
‘p₁’ is an integer selected from 1, 2, 3 and 4.

In one embodiment,
A represents 5- to 6-membered heteroarylenyl or 6-membered arylenyl; wherein the arylenyl and heteroarylenyl are unsubstituted or substituted with 1, 2 or 3 Rₐ;
Rₐ, at each occurrence, independently represents hydroxy, hydroxyalkyl, halogen, alkoxy, alkyl, haloalkyl or cyano;
R₁ is halogen, alkyl, haloalkyl, alkoxy, hydroxy, hydroxyalkyl, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano;
R₂ is hydrogen, hydroxy, hydroxyalkyl, halogen, alkoxy, alkyl, amino, aminoalkyl, haloalkyl or cyano; Q is amino, aminoalkyl, hydroxy, hydroxyalkyl, halogen, alkoxy, alkyl, haloalkyl or cyano;
L is a bond, —N(Rₓ)—(CRₓR_{y})p-O—, —O—(CRₓR_{y})p-, —O—(CRₓR_{y})p-O—, —C≡C—(CRₓR_{y})p-, -heterocycloalkylenyl-heterocycloalkylenyl-, -heterocycloalkylenyl-O—, -heterocycloalkylenyl-C≡C—, —C≡C-heterocycloalkylenyl-, —O-heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p-heterocycloalkylenyl- or -heterocycloalkylenyl-O-heterocycloalkylenyl-; wherein the cycloalkylenyl, heteroarylenyl and heterocycloalkylenyl are unsubstituted or substituted with 1, 2 or 3 R_{d}; and the left side of the L group is attached to A and the right side of L group is attached to M;
R_{d}, at each occurrence, is independently selected from hydroxy, halogen, alkyl and alkoxy; or any two R_{d} groups attached with the same C atom together form an oxo group;
Rₓ and R_{y}, at each occurrence, are independently selected from hydrogen and alkyl;
M is M-1 and M-2:

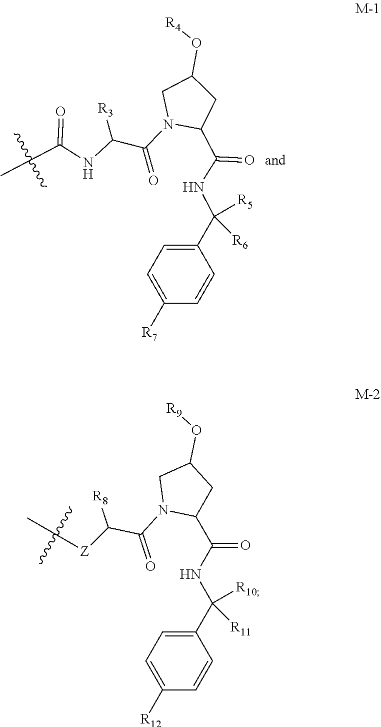

wherein
Z is 5- to 6-membered heteroarylenyl or 5- to 6-membered heterocycloalkylenyl, wherein the heteroarylenyl and heterocycloalkylenyl groups are unsubstituted or substituted with oxo, hydroxy, halogen, alkyl or alkoxy;
R₃ and R₈ independently represents alkyl, haloalkyl or hydroxyalkyl;
R₄ and R₉ independently represents hydrogen, alkyl, haloalkyl or hydroxyalkyl;
R₅, R₆, R₁₀ and R₁₁ are independently selected from hydrogen, alkyl, halogen, heteroalkyl, haloalkyl, hydroxyalkyl and acyl;
R₇ and R₁₂ independently represents thiazolyl which is unsubstituted or substituted with alkyl, hydroxy, amino or haloalkyl; and
‘p’ is an integer selected from 0, 1, 2, 3, 4, 5 and 6.

In one embodiment, R₁ is halogen, alkyl, haloalkyl, hydroxy, alkoxy, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano.

In one embodiment, R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, methoxy, fluoro, chloro, difluoromethyl and trifluoromethyl.

In one embodiment, R₂ is hydrogen, hydroxy, halogen, alkoxy, alkyl or haloalkyl.

In one embodiment, R₂ is hydrogen.

In one embodiment, Q is amino, aminoalkyl, hydroxy, hydroxyalkyl, halogen, alkoxy or alkyl.

In one embodiment, Q is amino or alkoxy.

In one embodiment, Q is —NH₂, —O—CH₃ or —O—CH₂CH₃.

In one embodiment, A represents phenylenyl which is unsubsituted or substituted with 1 or 2 Rₐ; wherein Rₐ, at each occurrence, independently represents hydroxy, halogen, alkoxy, alkyl or haloalkyl.

In one embodiment, A represents phenylenyl which is unsubsituted or substituted with 1 or 2 substituents selected from hydrogen and halogen.

In one embodiment, A represents phenylenyl which is unsubsituted or substituted with hydrogen or fluoro.

In one embodiment, A represents 5- to 6-membered heteroarylenyl which is unsubsituted or substituted with 1 or 2 Ra.

In one embodiment, A represents phenylenyl, furanylenyl, thienylenyl, pyrrolylenyl, pyrazolylenyl, imidazolylenyl, oxazolylenyl, isoxazolylenyl, thiazolylenyl, isothiazolylenyl, 1H-tetrazolylenyl, oxadiazolylenyl, triazolylenyl, pyridylenyl, pyrimidinylenyl, pyrazinylenyl, pyridazinylenyl, 1,2,3-triazinylenyl, 1,2,4-triazinylenyl or 1,3,5-triazinylenyl; wherein each group is unsubstituted or substituted with 1 or 2 substituent(s) independently selected from hydrogen, hydroxy, hydroxyalkyl, halogen, alkoxy, alkyl, haloalkyl and cyano.

In one embodiment, A represents pyridylenyl, pyrimidinylenyl or pyrazinylenyl, wherein the pyridylenyl, pyrimidinylenyl and pyrazinylenyl groups are unsubstituted or substituted with 1 or 2 substituents selected from hydrogen, hydroxy, hydroxyalkyl, halogen, alkoxy, alkyl, haloalkyl and cyano.

In one embodiment, L is a bond. In one embodiment, L is a bond and M is attached to A.

In one embodiment, L is —N(Rₓ)—(CRₓR_{y})p-O—, —O—(CRₓR_{y})p-, —O—(CRₓR_{y})p-O— or —C≡C—(CRₓR_{y})p-. In one embodiment, Rₓ and R_{y}, at each occurrence, are independently selected from hydrogen and alkyl.

In one embodiment, L is —N(CH₃)—CH₂CH₂—O—, —N(CH₃)—CH₂CH₂CH₂—O—, —N(CH₃)—CH₂CH₂CH₂CH₂—O—, —NH—CH₂CH₂—O—, —NH—CH₂CH₂CH₂—O—, —O—CH₂—, —O—CH₂—CH₂—, —O—CH₂—CH₂—CH₂—, —O—CH₂—CH₂—O—, —O—CH₂CH₂CH₂CH₂—, —C≡C—CH₂—, —C≡C—CH₂CH₂— or —C≡C—CH₂CH₂CH₂—.

In one embodiment, L is —N(Rₓ)—(CRₓR_{y})p-O—. In one embodiment, L is —N(CH₃)—CH₂—CH₂—O—, —N(CH₃)—CH₂CH₂CH₂—O—, —N(CH₃)—CH₂CH₂CH₂CH₂—O—, —NH—CH₂—CH₂—O— or —NH—CH₂CH₂CH₂—O—.

In one embodiment, L is —O—(CRₓR_{y})p-. In one embodiment, L is —O—CH₂—, —O—CH₂—CH₂—, —O—CH₂—CH₂—CH₂— or —O—CH₂—CH₂—CH₂—CH₂—.

In one embodiment, L is —O—(CRₓR_{y})p-O—. In one embodiment, L is —O—CH₂—O— or —O—CH₂CH₂—O—.

In one embodiment, L is —C≡C—(CRₓR_{y})p-. In one embodiment, L is —C≡C—(CH₂)₁₋₄—. In one embodiment, L is —C≡C—CH₂—, —C≡C—CH₂CH₂— or —C≡C—CH₂CH₂CH₂—.

In one embodiment, L is a bond, -heterocycloalkylenyl-heterocycloalkylenyl-, -heterocycloalkylenyl-O—, -heterocycloalkylenyl-C≡C—, —C≡C-heterocycloalkylenyl-, —O— heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p₁-heterocycloalkylenyl- or -heterocycloalkylenyl-O-heterocycloalkylenyl-; wherein the heterocycloalkylenyl, cycloalkylenyl or heteroarylenyl are unsubstituted or substituted with 1, 2 or 3 R_{d}.

In one embodiment, heterocycloalkylenyl is azetidinylenyl, pyrrolidinylenyl, piperidinylenyl, piperazinylenyl, tetrahydropyranyl, tetrahydropyridazinylenyl, morpholinylenyl, thiomorpholinylenyl, 1,4-dioxanylenyl, dioxidothiomorpholinylenyl, oxapiperazinylenyl, oxapiperidinylenyl, tetrahydropyranylenyl, dihydropyranylenyl or dihydropyrimidinylenyl; wherein each group is unsubstituted or substituted with 1 or 2 R_{d}.

In one embodiment, cycloalkylenyl is cyclopropylenyl, cyclobutylenyl, cyclopentylenyl, cyclohexylenyl or cycloheptylenyl. In one embodiment, L is cyclohexylenyl.

In one embodiment, heteroarylenyl is, furanylenyl, thienylenyl, pyrrolylenyl, pyrazolylenyl, imidazolylenyl, oxazolylenyl, isoxazolylenyl, thiazolylenyl, isothiazolylenyl, 1H-tetrazolylenyl, oxadiazolylenyl, triazolylenyl, pyridylenyl, pyrimidinylenyl, pyrazinylenyl, pyridazinylenyl, 1,2,3-triazinylenyl, 1,2,4-triazinylenyl or 1,3,5-triazinylenyl.

In one embodiment, L is piperidinylenyl, piperazinylenyl, oxapiperazinylenyl, oxapiperidinylenyl, dihydropyrimidinylenyl, cyclohexylenyl, bipiperidinylenyl, 1-(piperidin-1-ylmethyl)piperazine, 4,4′-oxydipiperidinylenyl, 4-ethynylpiperidinylenyl or 1-(azetidin-3-yl)piperazinylenyl; wherein each group is unsubstituted or substituted with 1, 2 or 3 R_{d}.

In one embodiment, L is —N(CH₃)—CH₂CH₂—O—, —NH—CH₂CH₂—O—, —O—CH₂—, —O—CH₂—CH₂—O—, —O—CH₂CH₂CH₂CH₂—, —C≡C—CH₂—, —C≡C—CH₂CH₂—, —C≡C—CH₂CH₂CH₂\.

In one embodiment, L is —N(CH₃)—CH₂CH₂—O—, —N(CH₃)—CH₂CH₂CH₂—O—, —N(CH₃)—CH₂CH₂CH₂CH₂—O—, —NH—CH₂CH₂—O—, —NH—CH₂CH₂CH₂—O—, —O—CH₂—, —O—CH₂—CH₂—, —O—CH₂—CH₂—CH₂—, —O—CH₂—CH₂—O—, —O—CH₂CH₂CH₂CH₂—, —C≡C—CH₂—, —C≡C—CH₂CH₂— or —C≡C—CH₂CH₂CH₂—,

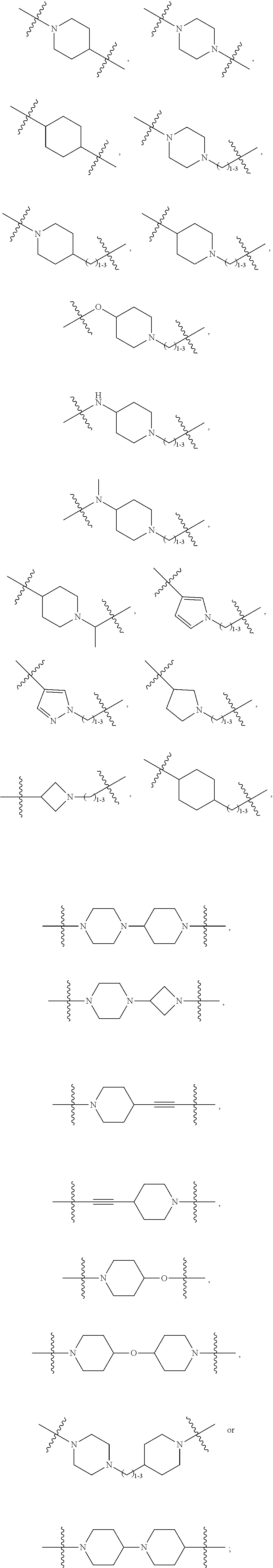

wherein each ring is unsubstituted or substituted with 1 or 2 R_{d}.

In one embodiment, R_{d} is alkyl, hydroxy, halogen or alkoxy; or any two R_{d} groups attached with the same C atom together form an oxo group; In one embodiment, the left side of L group is attached to A of compound of formula (I) and right side of L group is attached to M of compound of formula (I).

In one embodiment, p represents 0, 1, 2, 3, 4, 5 or 6.

In one embodiment, M is selected from M-1 and M-2;

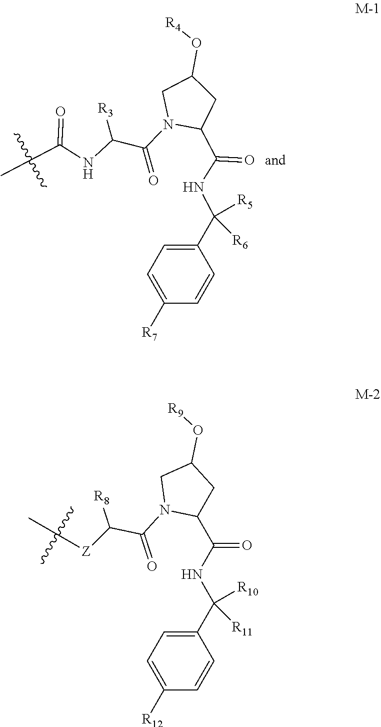

In one embodiment, M is M-1; In one embodiment, M is M-2; wherein R₃, R₄, R₅, R₆, R₇, R₈, R₉, R₁₀, R₁₁ and R₁₂ are as defined in compound of formula (I).

In one embodiment, M is M-1; wherein R₃ represents (C₁-C₄) alkyl; R₄ represents hydrogen or alkyl; R₅ and R₆ are independently selected from hydrogen, alkyl and haloalkyl; and R₇ is thiazolyl which is unsubstituted or substituted with alkyl, hydroxy, amino or haloalkyl.

In one embodiment, M is M-1;

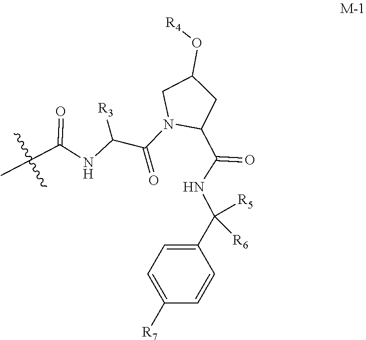

wherein
   R₃ represents alkyl, haloalkyl or hydroxyalkyl;
   R₄ represents hydrogen, alkyl, haloalkyl or hydroxyalkyl;
   R₅ and R₆ are independently selected from hydrogen, alkyl, halogen, haloalkyl and hydroxyalkyl; and
   R₇ represents thiazolyl which is unsubstituted or substituted with alkyl or haloalkyl.

In one embodiment, M-1 is selected from M-1A and M-1B:

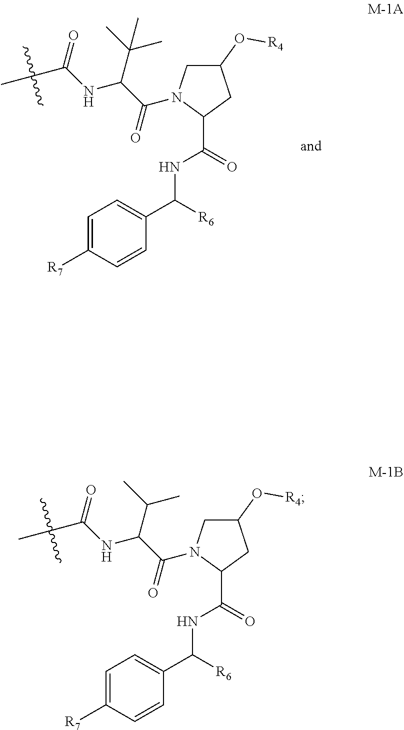

wherein
R₄ is hydrogen or alkyl;
R₆ is hydrogen, alkyl or haloalkyl; and
R₇ represents thiazolyl which is unsubstituted or substituted with alkyl or haloalkyl

In one embodiment, M-1A is represented by the formula,

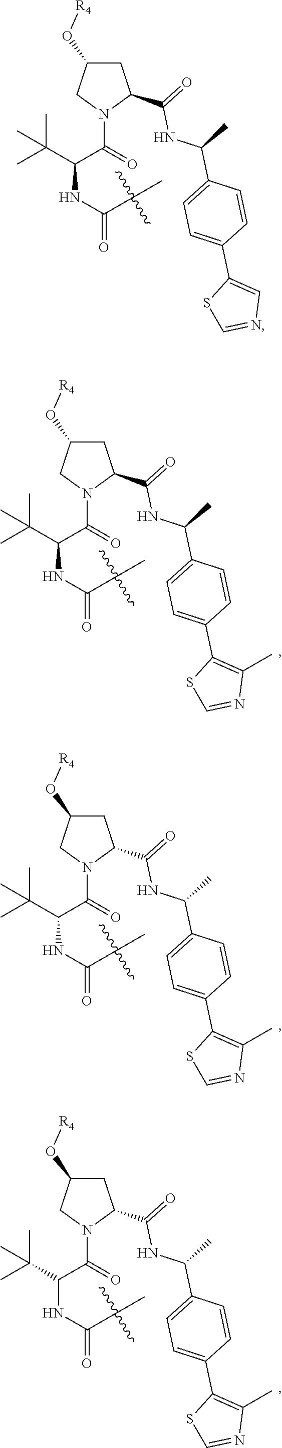

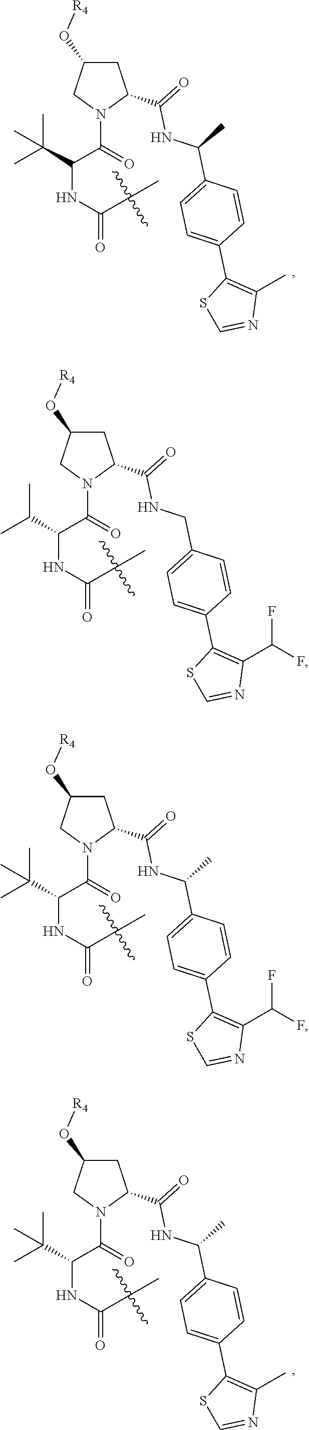

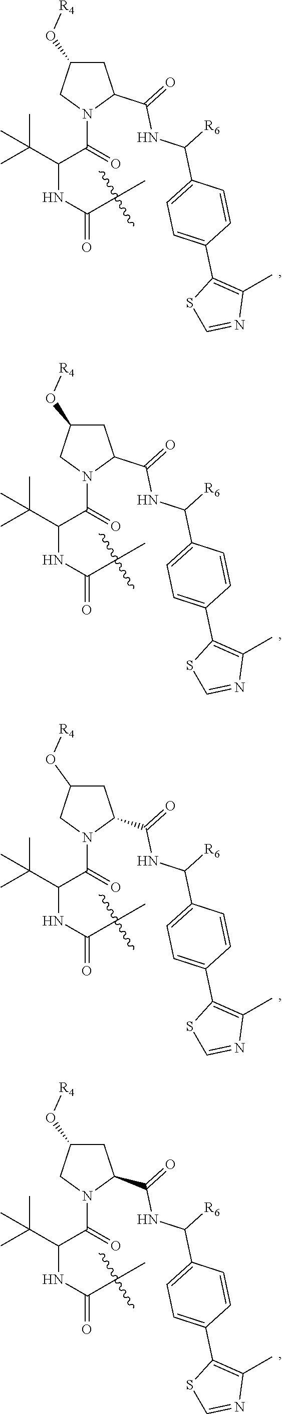

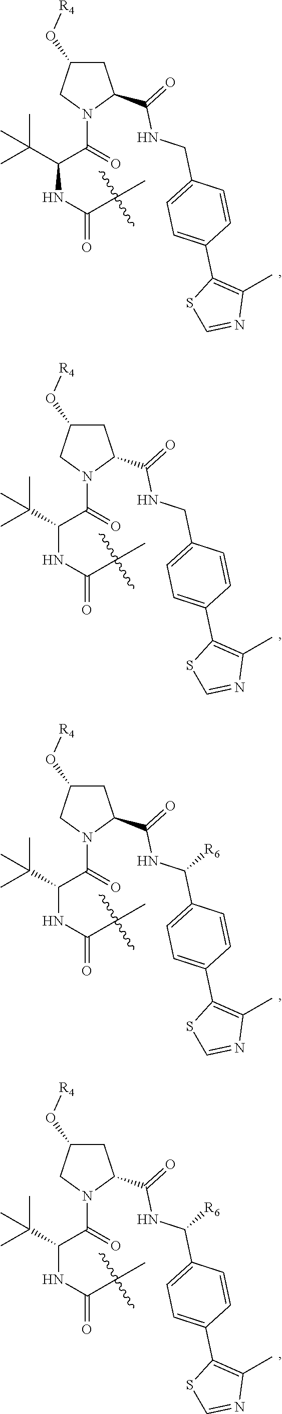

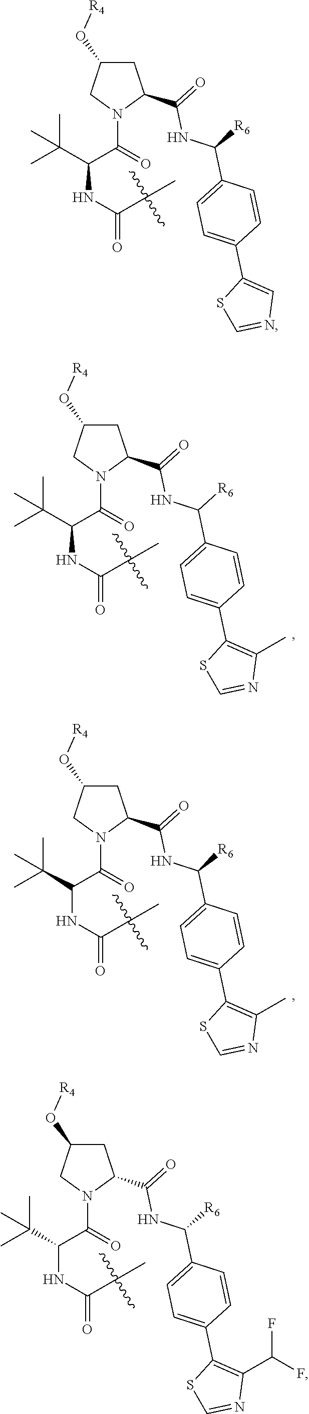

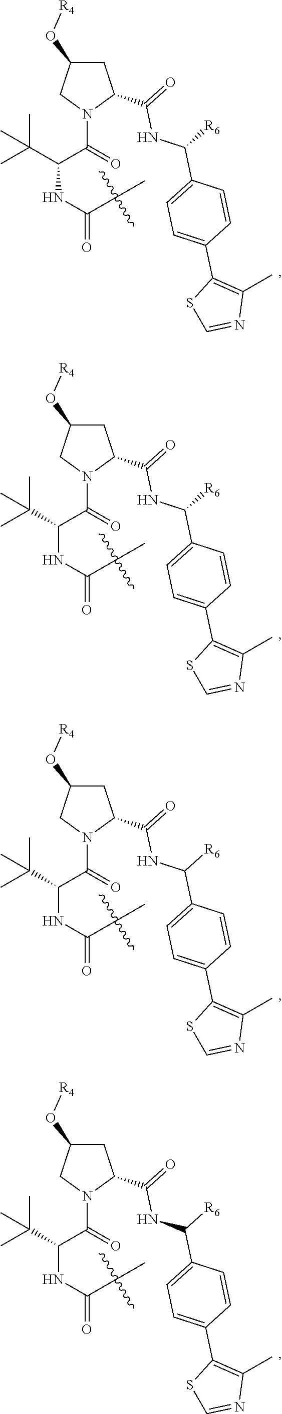

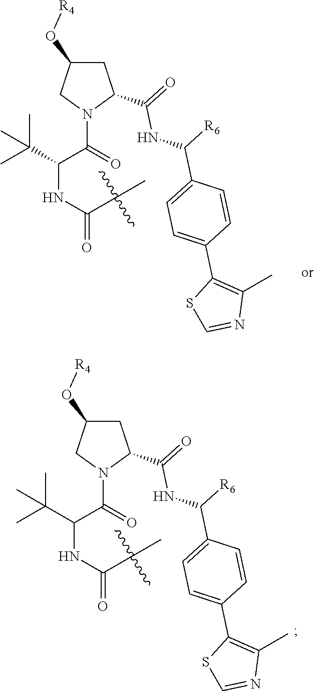

wherein
   R₄ is selected from hydrogen and alkyl; and
   R₆ is selected from hydrogen, alkyl and haloalkyl.

In one embodiment, M-1B is,

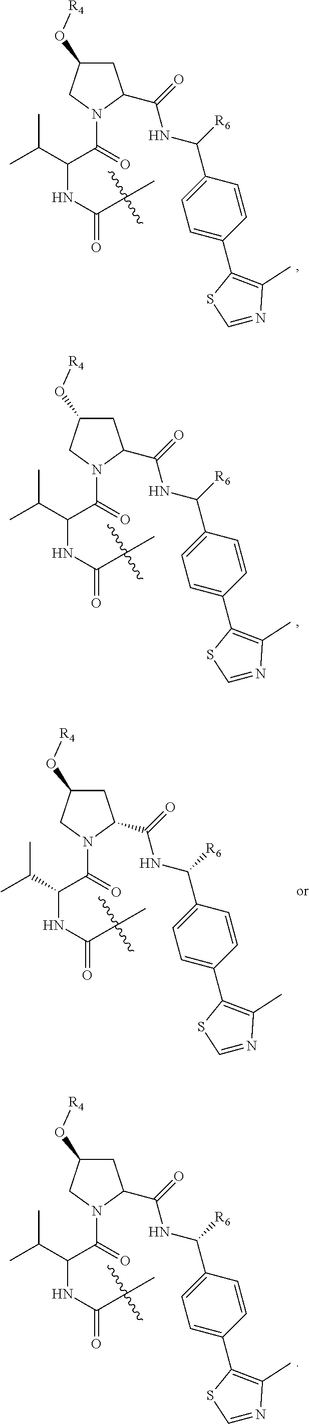

wherein
R₄ is selected from hydrogen and alkyl; and
R₆ is selected from hydrogen, alkyl and haloalkyl.

In certain embodiment, M-1 represented by the formula:

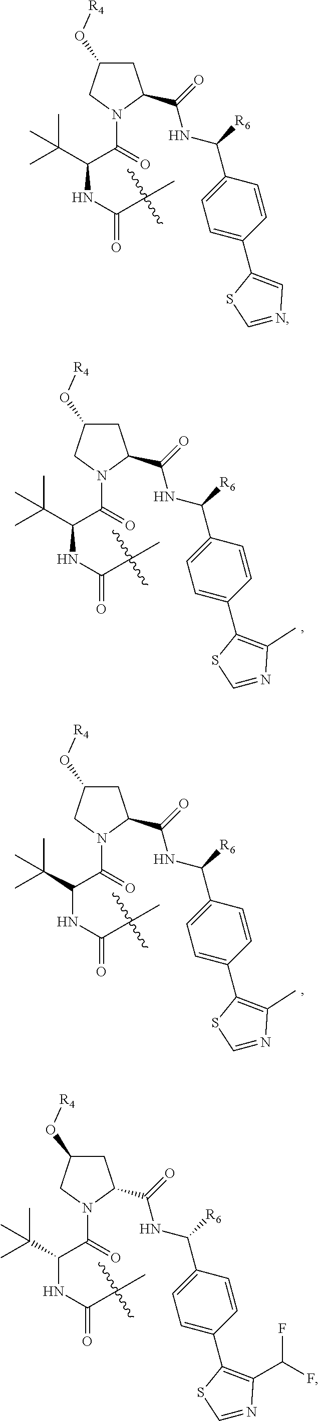

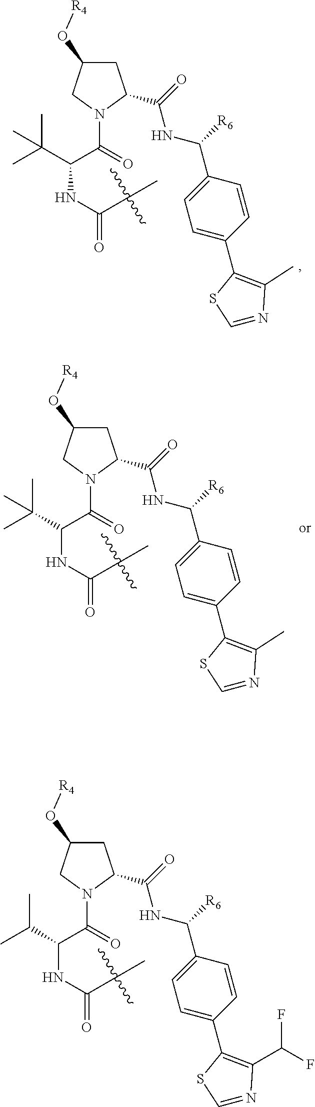

wherein R₄ and R₆ are same as defined in compound of formula (I).

In certain embodiment, M-1A is,

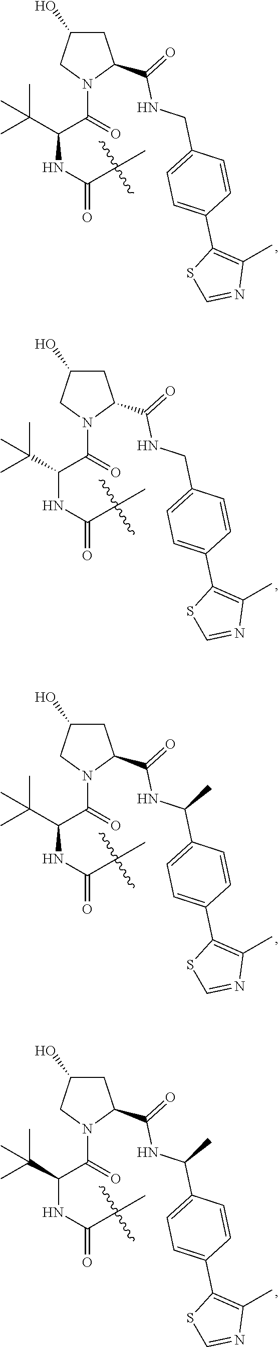

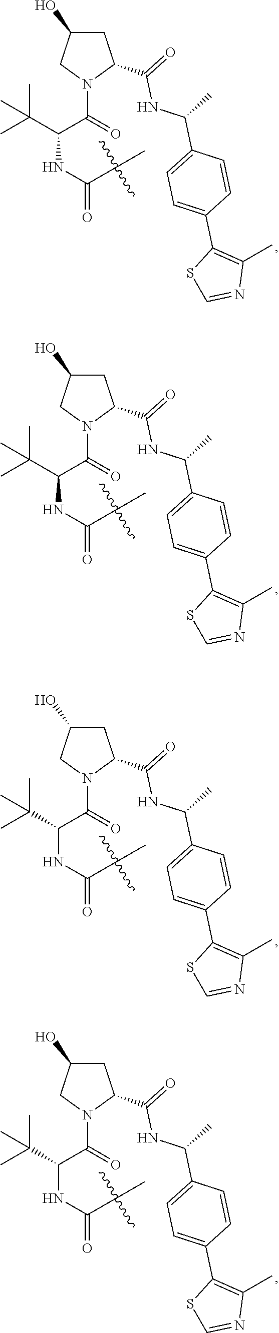

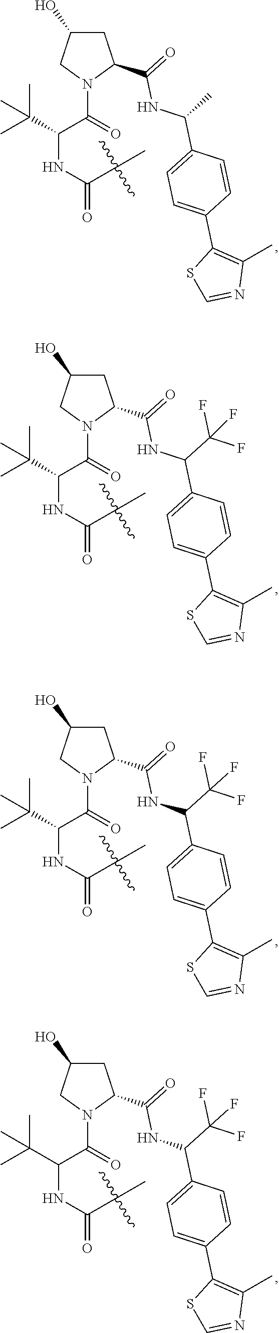

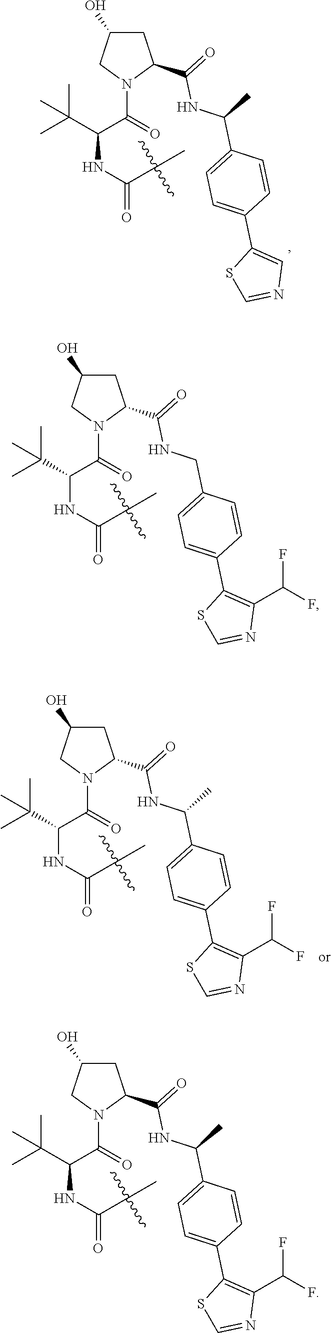 [SMILES: *C(=O)N[C@H](C(=O)N1C[C@H](O)C[C@H]1C(=O)N[C@@H](C)c1ccc(-c2cncs2)cc1)C(C)(C)C ; *C(=O)N[C@@H](C(=O)N1C[C@@H](O)C[C@@H]1C(=O)NCc1ccc(-c2scnc2C(F)F)cc1)C(C)(C)C ; *C(=O)N[C@@H](C(=O)N1C[C@@H](O)C[C@@H]1C(=O)N[C@H](C)c1ccc(-c2scnc2C(F)F)cc1)C(C)(C)C ; *C(=O)N[C@H](C(=O)N1C[C@H](O)C[C@H]1C(=O)N[C@@H](C)c1ccc(-c2scnc2C(F)F)cc1)C(C)(C)C]

In certain embodiment, M-1 is

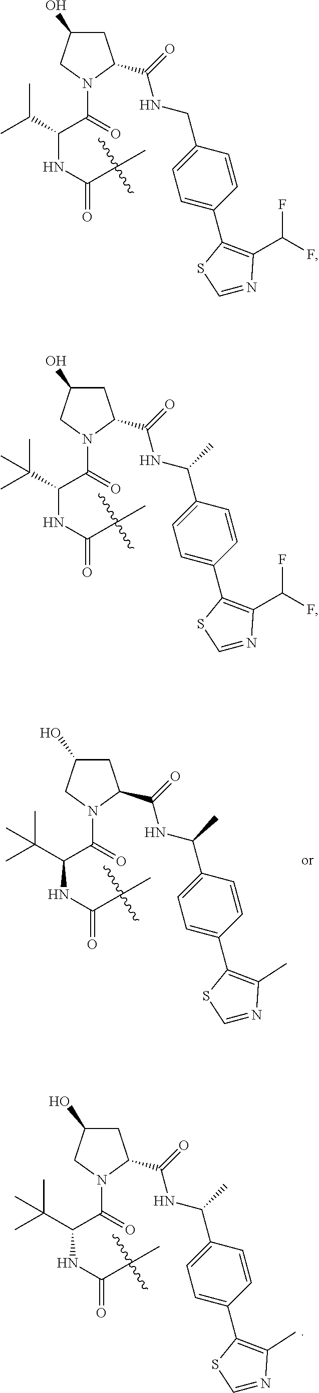

In one embodiment, M is M-2; wherein R₈ represents (C₁-C₄) alkyl; R₉ represents hydrogen or alkyl; R₁₀ and R₁₁ are independently selected from hydrogen, alkyl and haloalkyl; and R₁₂ is thiazolyl which is unsubstituted or substituted with alkyl, hydroxy, amino or haloalkyl.

In one embodiment, M is M-2:

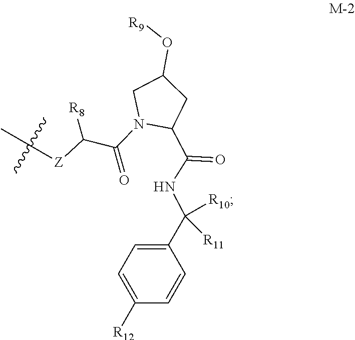

wherein
Z is oxazolylenyl, pyrazolylenyl, isoxazolylenyl or piperidinylenyl;
R₈ represents alkyl, heteroalkyl, haloalkyl or hydroxyalkyl;
R₉ represents hydrogen, alkyl, haloalkyl or hydroxyalkyl; and
R₁₀ and R₁₁ are independently selected from hydrogen, alkyl, halogen, heteroalkyl, haloalkyl and hydroxyalkyl; and
R₁₂ represents thiazolyl which is unsubstituted or substituted with alkyl or haloalkyl.

In one embodiment, M-2 is selected from M-2A and M-2B:

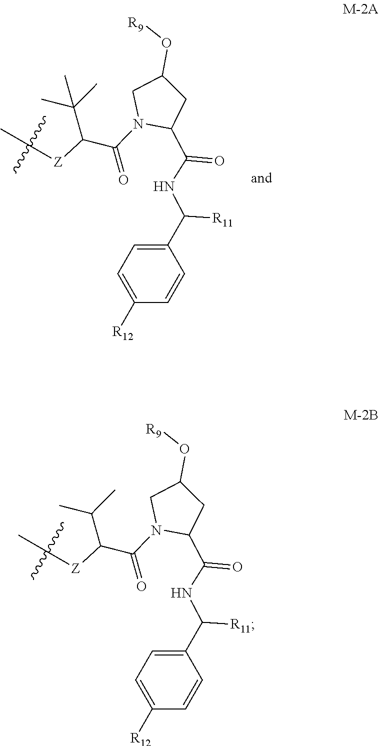

wherein
   Z represents isoxazolylenyl, oxazolylenyl, pyrazolylenyl or piperidinylenyl;
   R₉ is hydrogen or alkyl;
   Ru is hydrogen, alkyl or haloalkyl; and
   R₁₂ represents thiazolyl which is unsubstituted or substituted with alkyl or haloalkyl

In one embodiment, Z represents isoxazolylenyl, oxazolylenyl or pyrrazolylenyl;

In certain embodiment, M-2 is

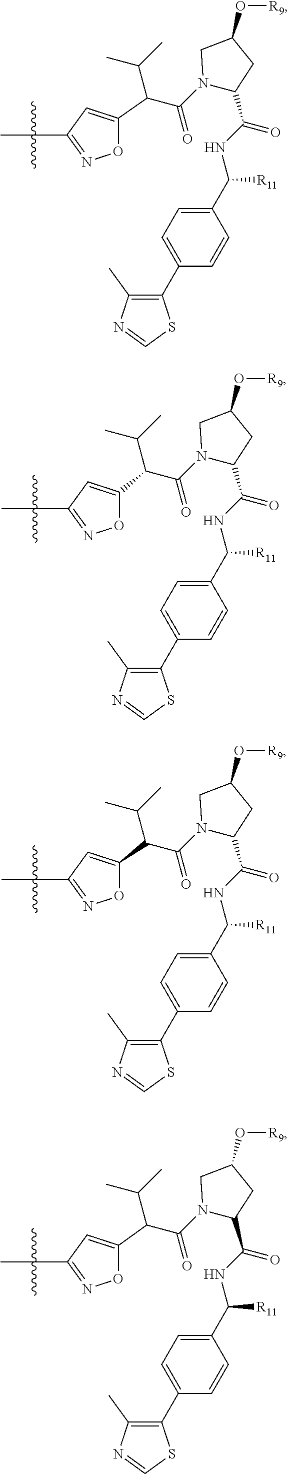

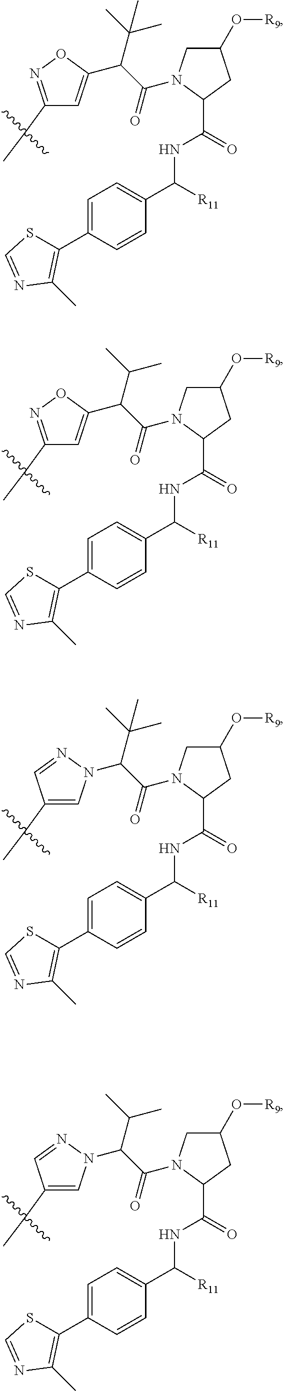

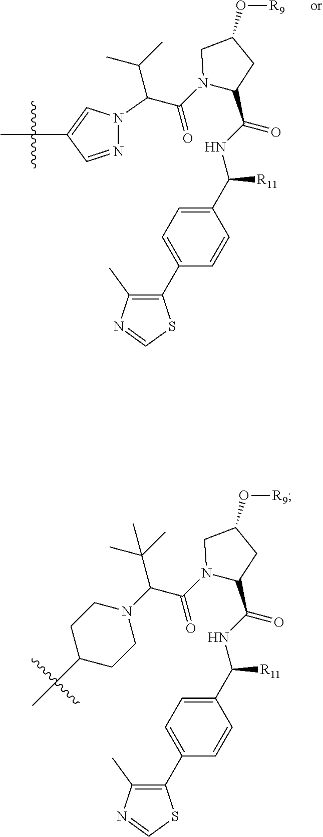

wherein
R₉ represents hydrogen, alkyl, haloalkyl or hydroxyalkyl;
R₁₁ is selected from hydrogen, alkyl, halogen, heteroalkyl, haloalkyl and hydroxyalkyl.

In one embodiment, M-2 is represented by formula:

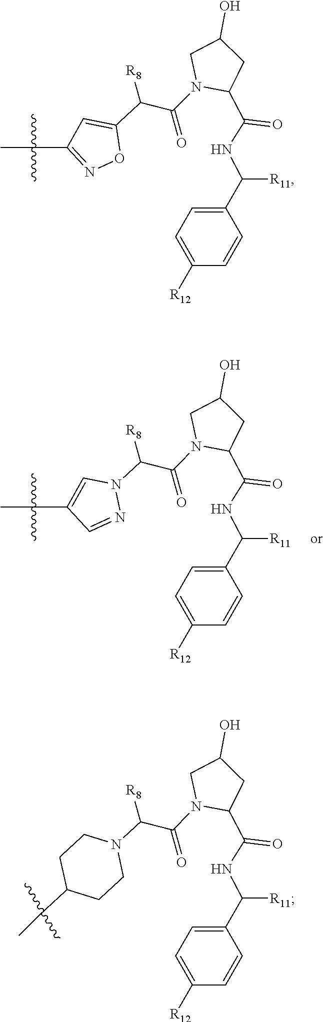

wherein, R₈ represents alkyl;
R₁₁ represents hydrogen or alkyl; and
R₁₂ represents thiazolyl which is unsubstituted or substituted with alkyl or haloalkyl.

In certain embodiment, M is

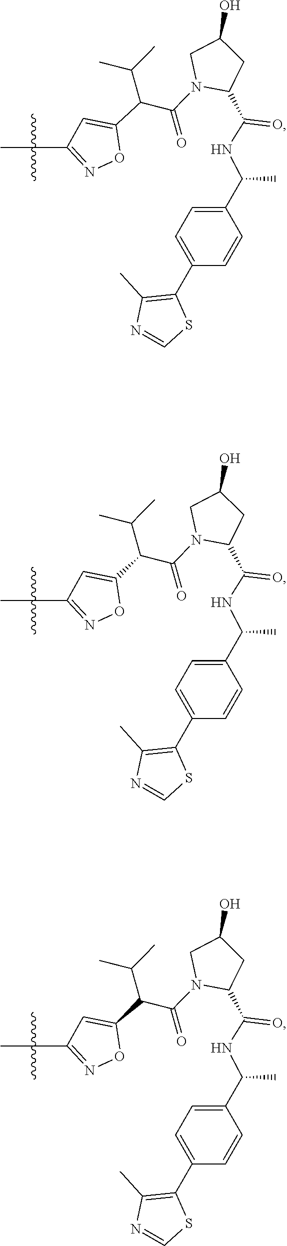

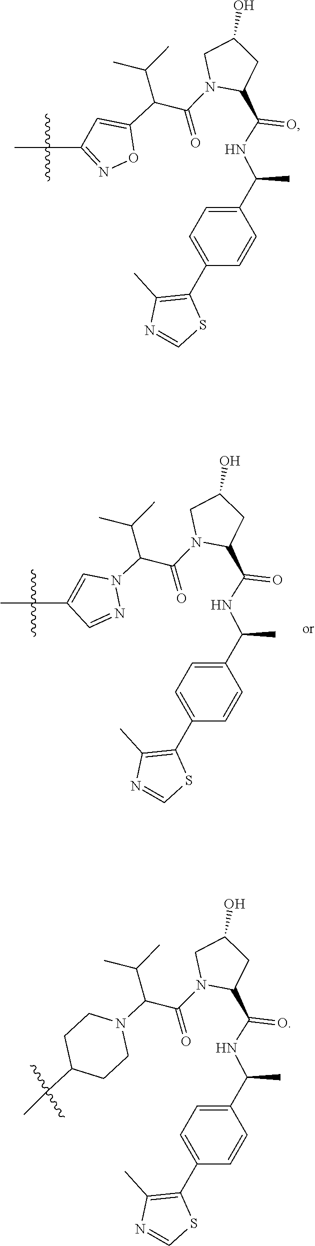

In certain embodiment, if L is a bond, M is attached to A of compound of formula (I).
In one embodiment, ‘p’ is selected from 0, 1, 2, and 3.
In one embodiment, ‘p’ is 0.
In one embodiment, ‘p’ is 1 or 2.
In one embodiment, ‘p₁’ is selected from 1, 2 and 3.
In one embodiment, ‘p₁’ is 1 or 2.

In one embodiment, the present invention provides compound of formula (IA) or a pharmaceutically acceptable salt or a stereoisomer or a tautomer thereof:

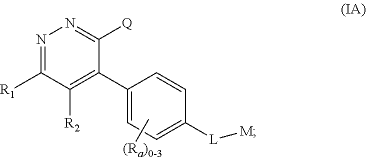

wherein R₁, R₂, Q, Rₐ, L and M are as defined in compound of formula (I).

In one embodiment of compound of formula (IA), R₁ is halogen, alkyl, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen and haloalkyl.

In one embodiment of compound of formula (IA), R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, alkoxy, halogen, alkyl, haloalkyl and amino.

In one embodiment of compound of formula (IA), R₂ is hydrogen, hydroxy, halogen, alkoxy, alkyl or haloalkyl.

In one embodiment of compound of formula (IA), R₂ is hydrogen.

In one embodiment of compound of formula (IA), Q is selected from amino and alkoxy.

In one embodiment of compound of formula (IA), Rₐ, at each occurrence, independently represents hydroxy, hydroxyalkyl, halogen, alkoxy, alkyl, haloalkyl or cyano.

In one embodiment of compound of formula (IA), Rₐ, at each occurrence, independently represents halogen.

In one embodiment of compound of formula (IA), L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —C≡C—CH₂CH₂—, —C≡C—CH₂CH₂CH₂—

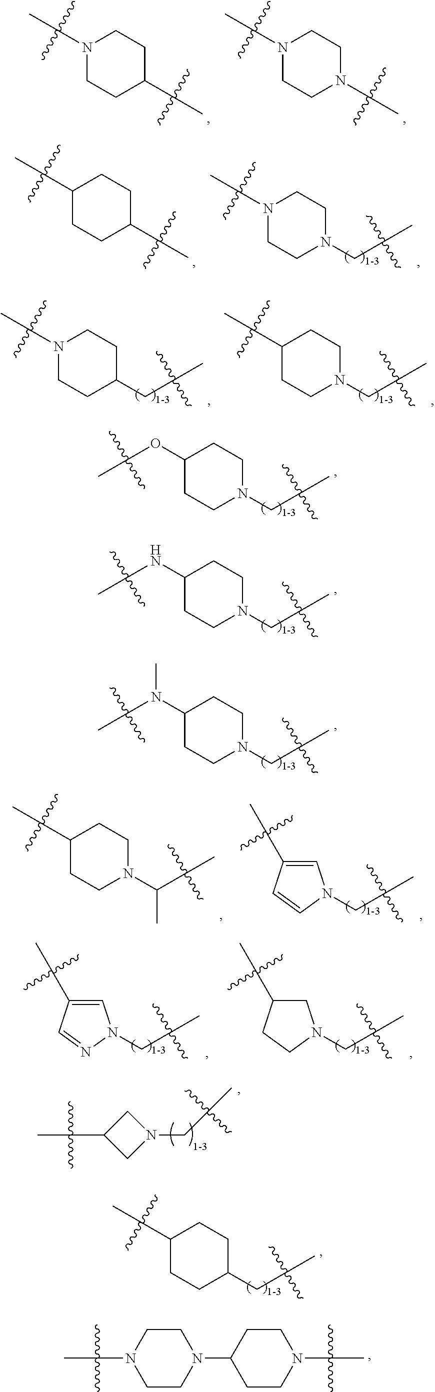

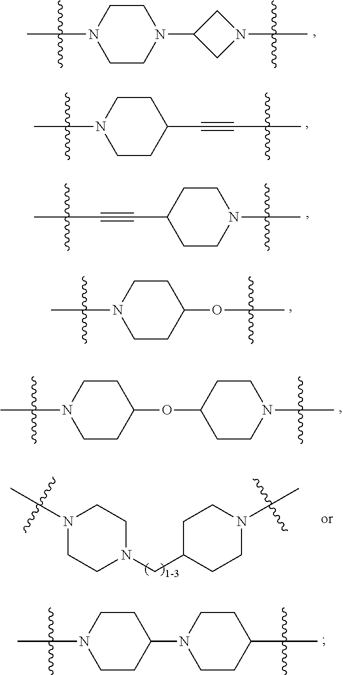

wherein each ring is unsubstituted or substituted with 1 or 2 R_{d}.

In one embodiment of compound of formula (IA),
Z is oxazolylenyl, pyrazolylenyl, isoxazolylenyl or piperidinylenyl;
R₃ and R₈ independently represents alkyl;
R₄ and R₉ independently represents hydrogen or alkyl;
R₅, R₆, R₁₀ and R₁₁ are independently selected from hydrogen and alkyl; and
R₇ and R₁₂ independently represents thiazolyl which is unsubstituted or substituted with alkyl or haloalkyl.

In one embodiment of compound of formula (IA),
Rₐ, at each occurrence, independently represents halogen;
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen and haloalkyl;
R₂ is hydrogen;
Q is amino or alkoxy;
L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —C≡C—CH₂CH₂—, —C≡C—CH₂CH₂CH₂—,

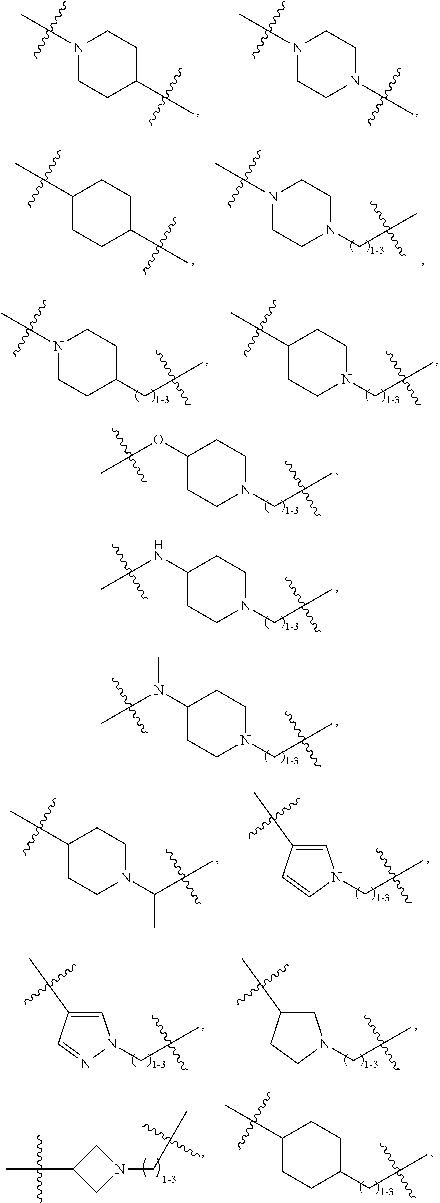

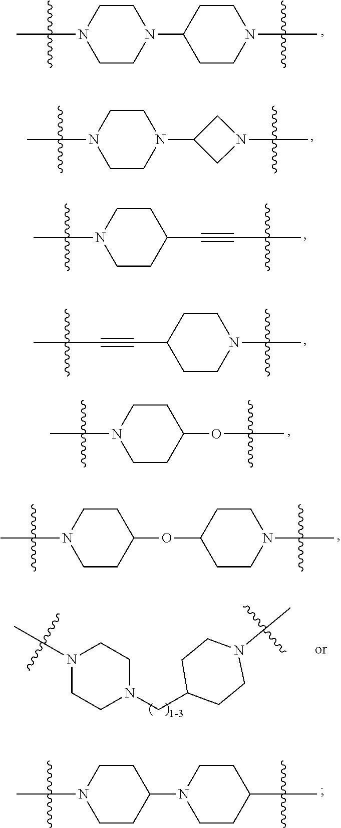

wherein each ring is unsubstituted or substituted with 1 or 2 R_{d}.
R_{d}, at each occurrence, is independently selected from alkyl, hydroxy and halogen; or any two R_{d} groups attached with the same C atom together form an oxo group;
M is selected from M-1 and M-2;
Z is oxazolylenyl, pyrazolylenyl, isoxazolylenyl or piperidinylenyl;
R₃ and R₈ independently represents alkyl;
R₄ and R₉ independently represents hydrogen;
R₅, R₆, R₁₀ and R₁₁ are independently selected from hydrogen and alkyl; and R₇ and R₁₂ independently represents thiazolyl which is unsubstituted or substituted with alkyl or haloalkyl.

In one embodiment, the present invention provides compound of formula (IB) or a pharmaceutically acceptable salt or a stereoisomer or a tautomer thereof:

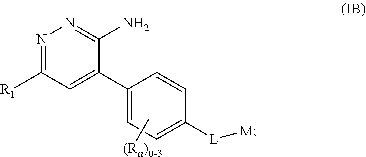

wherein R₁, Rₐ, L and M are as defined in compound of formula (I).

In one embodiment of compound of formula (IB), Rₐ at each occurrence, independently represents halogen.

In one embodiment of compound of formula (IB), R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen, alkyl, haloalkyl and amino.

In one embodiment of compound of formula (IA), R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, alkoxy, halogen, alkyl, haloalkyl and amino.

In one embodiment of compound of formula (IB), L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —

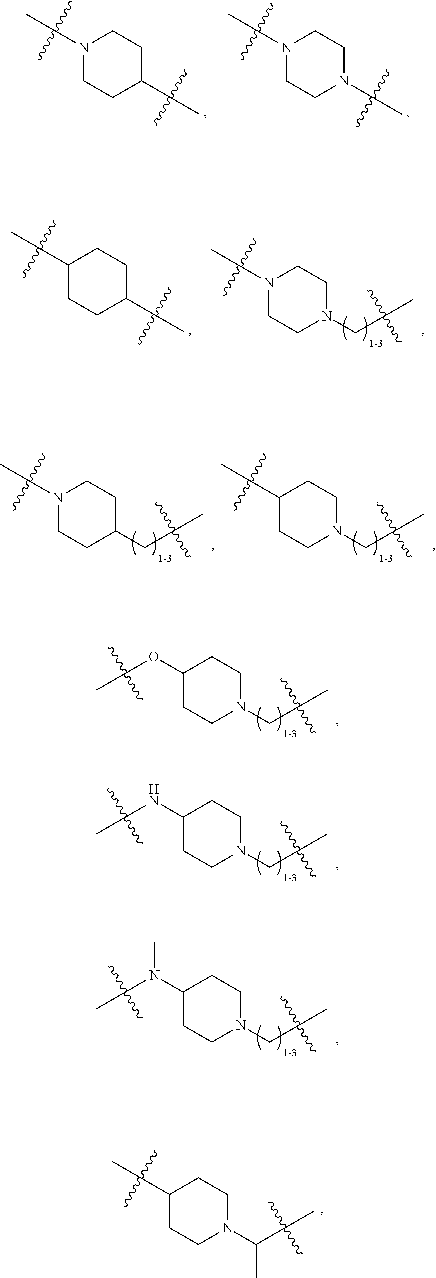

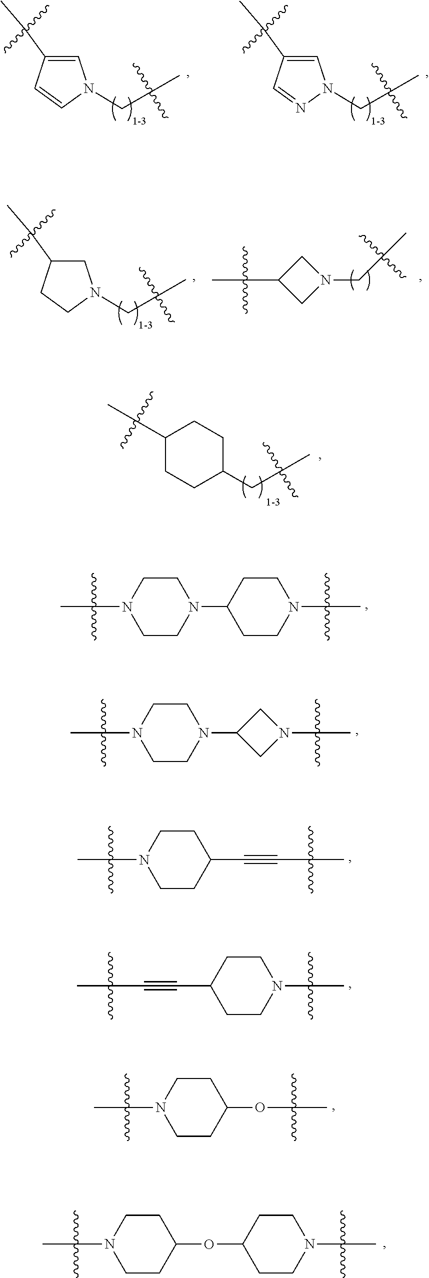

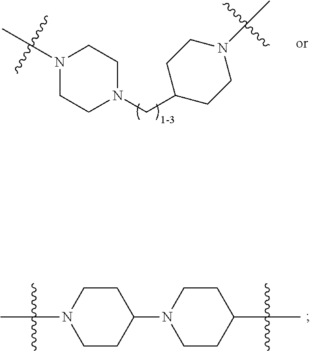

wherein each ring is unsubstituted or substituted with 1 or 2 R_{d}.

In one embodiment of compound of formula (IB), M is selected from M-1 and M-2; wherein
Z is oxazolylenyl, pyrazolylenyl, isoxazolylenyl or piperidinylenyl;
R₃ and R₈ independently represents alkyl;
R₄ and R₉ independently represents hydrogen or alkyl;
R₅, R₆, R₁₀ and R₁₁ are independently selected from hydrogen and alkyl; and
R₇ and R₁₂ independently represents thiazolyl which is unsubstituted or substituted with alkyl or haloalkyl.

In one embodiment of compound of formula (IB),
Rₐ, at each occurrence, independently represents halogen;
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —C≡C—CH₂CH₂—, —C≡C—CH₂CH₂CH₂—,

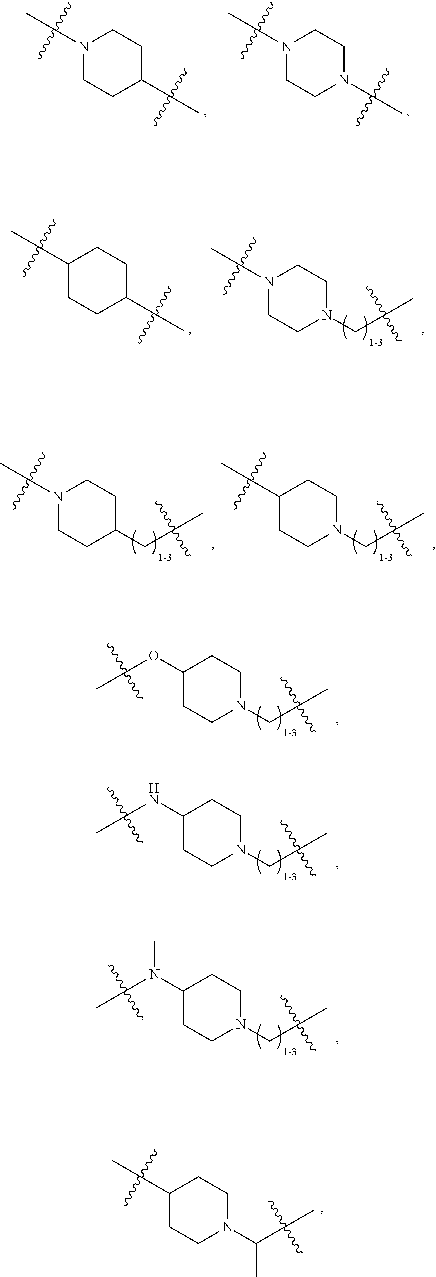

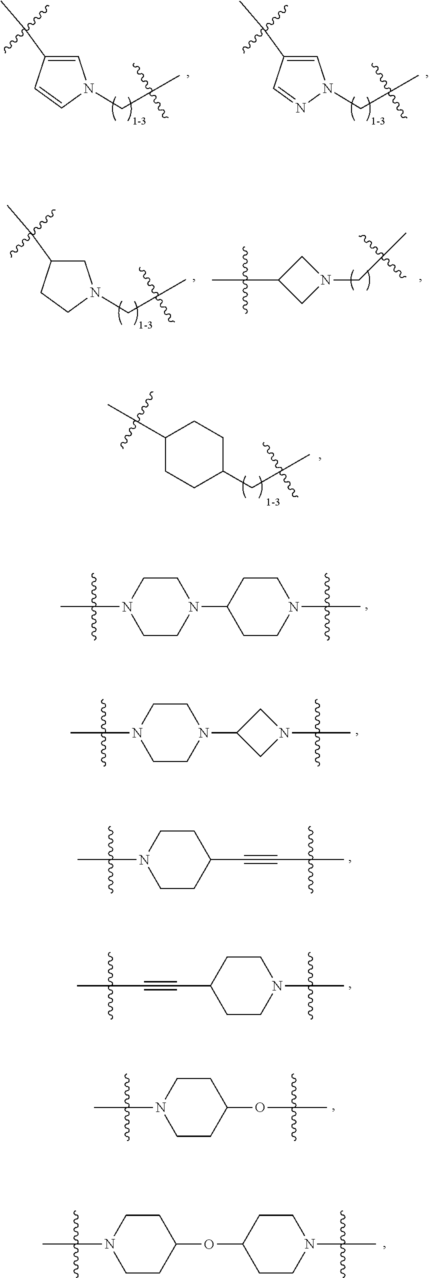

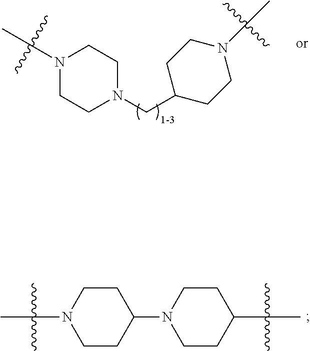

wherein each ring is unsubstituted or substituted with 1 or 2 R_{d};
R_{d}, at each occurrence, is independently selected from alkyl, hydroxy and halogen; or any two R_{d} groups attached with the same C atom together form an oxo group;
M is selected from M-1 and M-2;
Z is oxazolylenyl, pyrazolylenyl, isoxazolylenyl or piperidinylenyl;
R₃ and R₈ independently represents alkyl;
R₄ and R₉ independently represents hydrogen or alkyl;
R₅, R₆, R₁₀ and R₁₁ are independently selected from hydrogen and alkyl; and
R₇ and R₁₂ independently represents thiazolyl which is unsubstituted or substituted with alkyl or haloalkyl.

In one embodiment, the present invention provides compound of formula (IC) or a pharmaceutically acceptable salt or a stereoisomer or a tautomer thereof:

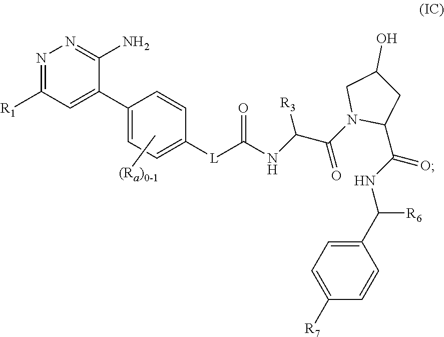

wherein Rₐ, R₁, L, R₃, R₆ and R₇ are as defined in compound of formula (I).

In one embodiment of compound of formula (IC), R₁ is halogen, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl.

In one embodiment of compound of formula (IA), R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, alkoxy, halogen, alkyl, haloalkyl and amino.

In one embodiment of compound of formula (IC), Rₐ represents hydroxy, hydroxyalkyl, halogen, alkoxy, alkyl or haloalkyl.

In one embodiment of compound of formula (IC), Rₐ represents halogen.

L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —C≡C—CH₂CH₂—, —C≡C—CH₂CH₂CH₂—,

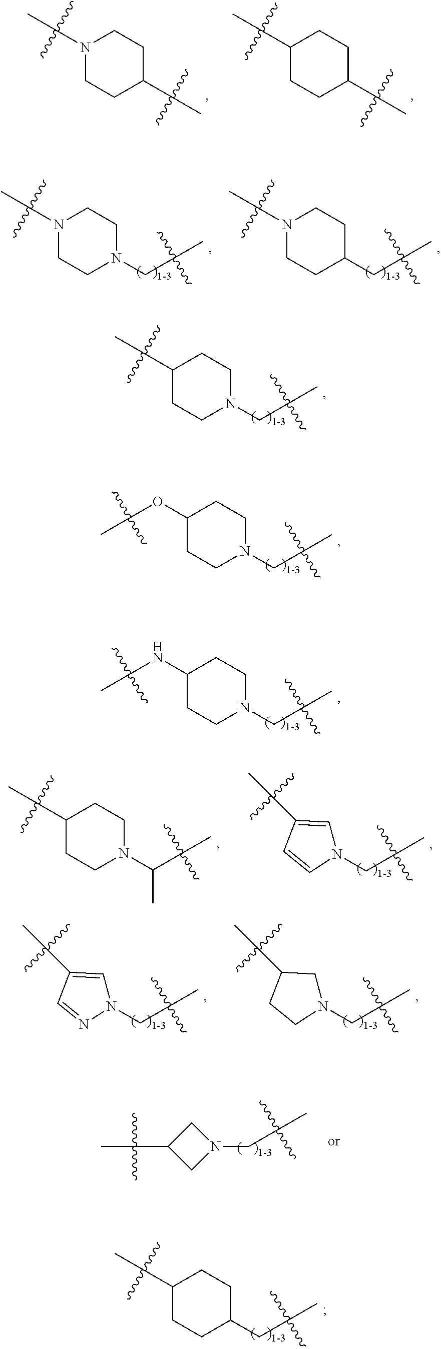

wherein each ring is unsubstituted or substituted with 1 or 2 R_{d}.

In one embodiment of compound of formula (IC), R_{d}, at each occurrence, is independently selected from hydroxy, halogen, alkoxy, alkyl, haloalkyl and amino; or any two R_{d} groups attached with the same C atom together form an oxo group;

In one embodiment of compound of formula (IC),
R₃ represents alkyl;
R₆ is selected from hydrogen and alkyl; and
R₇ represents thiazolyl which is unsubstituted or substituted with alkyl or haloalkyl.

In one embodiment of compound of formula (IC),
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
Rₐ represents halogen;
L is a bond, —O—CH₂—, —O—CH₂CH₂CH₂CH₂—, —O—CH₂—CH₂—O—, —NH—CH₂—CH₂—O—, —N(CH₃)—CH₂CH₂—O—, —C≡C—CH₂—, —C≡C—CH₂CH₂—, —C≡C—CH₂CH₂CH₂—,

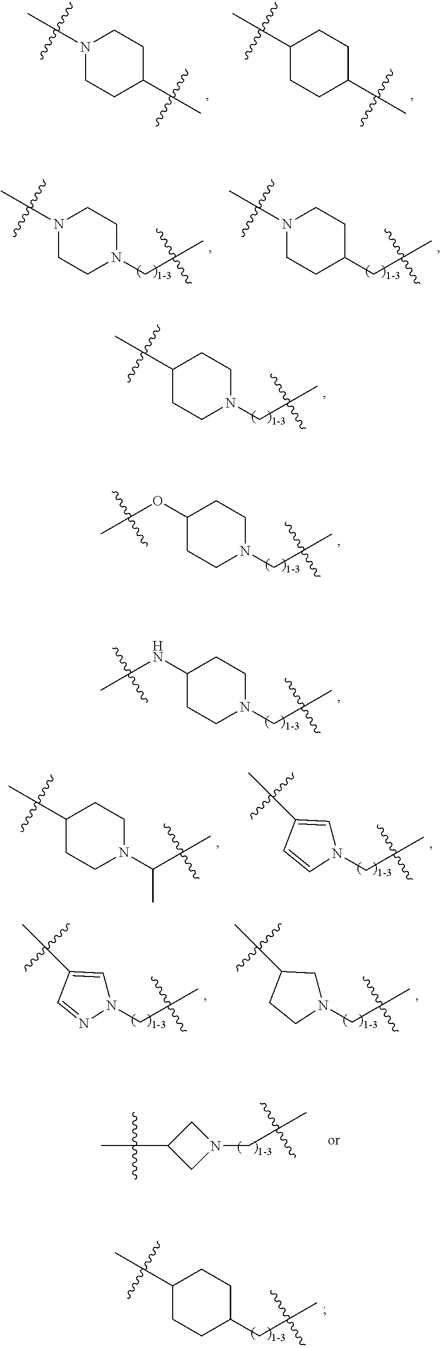

wherein each ring is unsubstituted or substituted with 1 or 2 R_{d};
R_{d}, at each occurrence, is independently selected from alkyl, hydroxy and halogen; or any two R_{d} groups attached with the same C atom together form an oxo group;
R₃ represents alkyl;
R₆ is selected from hydrogen and alkyl; and
R₇ represents thiazolyl which is unsubstituted or substituted with alkyl or haloalkyl.

In one embodiment, the present invention provides compound of formula (ID) or a pharmaceutically acceptable salt or a stereoisomer or a tautomer thereof:

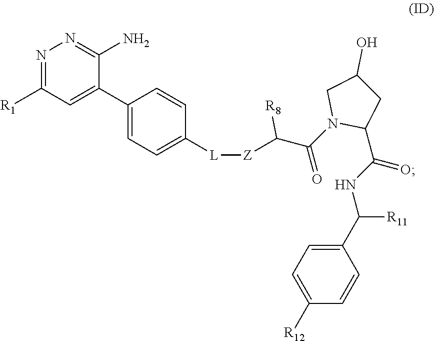

wherein R₁, L, Z, R₈, R₁₁ and R₁₂ are as defined in compound of formula (I).

In one embodiment of compound of formula (IC), R₁ is halogen, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl.

In one embodiment of compound of formula (IA), R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, alkoxy, halogen, alkyl, haloalkyl and amino.

In one embodiment of compound of formula (ID), L is a bond, —O—CH₂—CH₂—O—CH₂—CH₂—O—, —N(CH₃)—CH₂CH₂—O—,

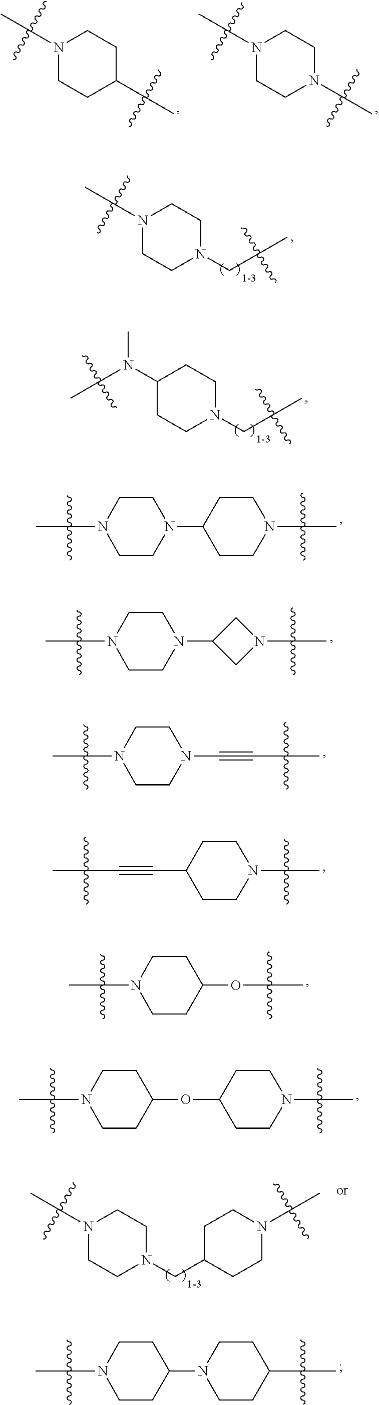

wherein each ring is unsubstituted or substituted with 1 or 2 R_{d};
R_{d}, at each occurrence, is independently selected from alkyl, hydroxy and halogen; or any two R_{d} groups attached with the same C atom together form an oxo group;

In one embodiment of compound of formula (ID), Z is oxazolylenyl, pyrazolylenyl, isoxazolylenyl or piperidinylenyl;
R₈ represents alkyl;
R₁₁ is selected from hydrogen and alkyl;
R₁₂ represents thiazolyl which is unsubstituted or substituted with alkyl or haloalkyl.

In one embodiment of compound of formula (ID),
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
L is a bond, —O—CH₂—CH₂—O—, —NH—CH₂—CH₂—O—, —N(CH₃)—CH₂CH₂—O—,

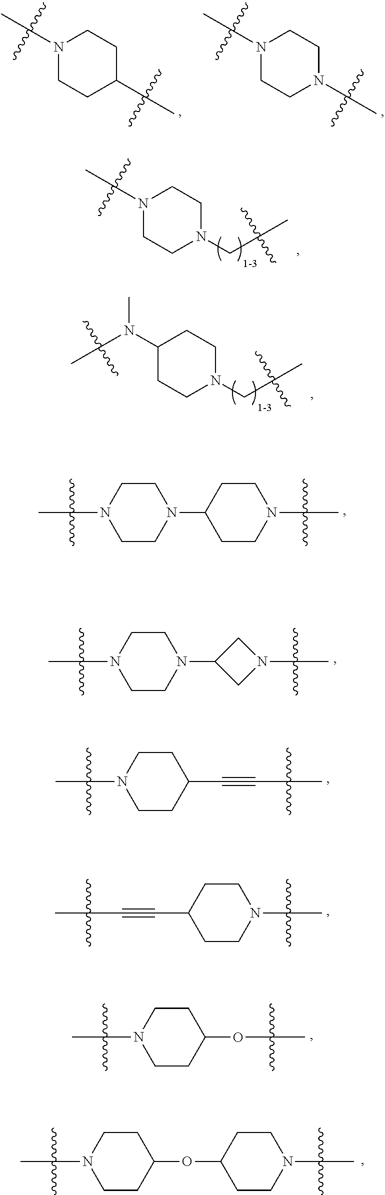

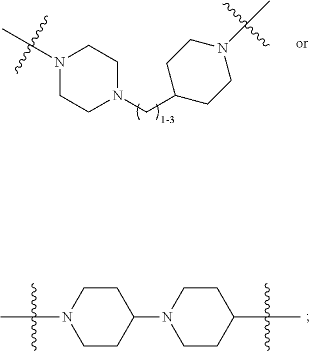

; wherein each ring is unsubstituted or substituted with 1 or 2 R_{d};
R_{d}, at each occurrence, is independently selected from alkyl, hydroxy and halogen; or any two R_{d} groups attached with the same C atom together form an oxo group;
Z is oxazolylenyl, pyrazolylenyl, isoxazolylenyl or piperidinylenyl;
R₈ represents alkyl;
R₁₁ is selected from hydrogen and alkyl; and
R₁₂ represents thiazolyl which is unsubstituted or substituted with alkyl or haloalkyl.

In one embodiment, the present invention provides a compound of formula (IE) or a pharmaceutically acceptable salt or a stereoisomer or a tautomer thereof:

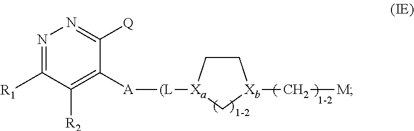

wherein R₁, R₂, A, Q and M are as defined in compound of formula (I); and
Xₐ and X_{b} independently represents CH or N.

In one embodiment of compound of formula (IE), A represents 5- to 6-membered heteroarylenyl which is unsubstituted or substituted with 1 or 2 Rₐ;

In one embodiment of compound of formula (IE), A represents phenylenyl, furanylenyl, thienylenyl, pyrrolylenyl, pyrazolylenyl, imidazolylenyl, oxazolylenyl, isoxazolylenyl, thiazolylenyl, isothiazolylenyl, 1H-tetrazolylenyl, oxadiazolylenyl, triazolylenyl, pyridylenyl, pyrimidinylenyl, pyrazinylenyl, pyridazinylenyl, 1,2,3-triazinylenyl, 1,2,4-triazinylenyl or 1,3,5-triazinylenyl; wherein each group is unsubstituted or substituted with 1 or 2 substituent(s) selected from hydrogen, hydroxy, hydroxyalkyl, halogen, alkoxy, alkyl, haloalkyl and cyano.

In one embodiment of compound of formula (IE), Q is amino, aminoalkyl, hydroxy, hydroxyalkyl, halogen, alkoxy or alkyl.

In one embodiment of compound of formula (IE), R₁ is halogen, alkyl, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen and haloalkyl.

In one embodiment of compound of formula (IE),
R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, alkoxy, halogen, alkyl, haloalkyl and amino;
R₂ is hydrogen;
Q is selected from amino and alkoxy;
A is phenylenyl or pyridylenyl; wherein phenylenyl and pyridylenyl are unsubstituted substituted with 1, 2 or 3 Rₐ;
Xₐ is CH or N; and
X_{b} is N.

In one embodiment, the present invention provides a compound of formula (I) selected from the ones described herein, or a pharmaceutically acceptable salt or a stereoisomer or a tautomer thereof.

### Method of Treatment

In one embodiment, the present invention provides a compound of formula (I) or a pharmaceutically acceptable salt or a stereoisomer or a tautomer thereof for use as a medicament.

In one embodiment, the present invention provides a compound of formula (I) or a pharmaceutical acceptable salt or a stereoisomer or a tautomer thereof, for use in the treatment of a disease or disorder dependent upon SMARCA2 and/or SMARCA4.

In one embodiment, a disease or disorder dependent upon SMARCA2 and/or SMARCA4 is cancer.

In one embodiment, the present invention provides a method of degrading a target protein in a subject comprising administering to a subject in need thereof a therapeutically effective amount of the compound or a pharmaceutically acceptable salt or a stereoisomer or a tautomer thereof.

In one embodiment, the present invention provides a method of degrading a target protein is SMARCA2 and/or SMARCA4.

In one embodiment, the present invention provides a method for treating or delaying progression of a disease or disorder dependent upon SMARCA2 and/or SMARCA4 in a subject comprising administering to the subject, in need thereof, a therapeutically effective amount of a compound of formula (I) or a pharmaceutically acceptable salt or a stereoisomer or a tautomer thereof.

In one embodiment, the present invention provides a method for treating diseases or disorders dependent upon SMARCA2 and/or SMARCA4 in a subject comprising administering to the subject in need thereof a therapeutically effective amount of a compound formula I or a pharmaceutically acceptable salt or a stereoisomer or a tautomer thereof.

In one embodiment, diseases or disorders that are dependent upon SMARCA2 and/or SMARCA4, include cancer.

In one embodiment, the disease or disorder is cancer selected from hematologic cancers, lung cancer (i.e. non-small cell lung cancer), acoustic neuroma, acute leukemia, acute lymphocytic leukemia, acute myelocytic leukemia (adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

In one embodiment, the present invention provides a method for inhibiting tumor growth in a subject afflicted with cancer comprising administering a compound of formula (I) or a pharmaceutically acceptable salt or a stereoisomer or a tautomer thereof to the subject, in need thereof.

In one embodiment, the present invention provides a pharmaceutical composition comprising a compound of formula (I), or a pharmaceutically acceptable salt or a stereoisomer or a tautomer thereof as described herein and at least one pharmaceutically acceptable excipient (such as a pharmaceutically acceptable carrier or diluent). Preferably, the pharmaceutical composition comprises a therapeutically effective amount of at least one compound described herein or a pharmaceutically acceptable salt or a stereoisomer or a tautomer thereof. The compounds described in the present invention may be associated with a pharmaceutically acceptable excipient (such as a carrier or a diluent) or be diluted by a carrier or enclosed within a carrier which can be in the form of a capsule, sachet, paper or other container.

In one embodiment, the present invention provides a pharmaceutical composition comprising a compound of formula (I), for use in degrading a target protein in a subject wherein the target protein is SMARCA2 and/or SMARCA4.

In one embodiment, the subject is afflicted with a disease or disorder dependent upon SMARCA2 and/or SMARCA4.

In one embodiment, the subject is afflicted with cancer mediated by the target protein, wherein the target protein is SMARCA2 and/or SMARCA4.

In one embodiment, the present invention provides a pharmaceutical composition comprising a compound of formula (I) or a pharmaceutically acceptable salt or a stereoisomer or a tautomer thereof as described herein for use as a medicament.

In one embodiment, the present invention provides a pharmaceutical composition comprising a compound of formula (I), for use in treating or delaying progression of a disease or disorder mediated by SMARCA2 and/or SMARCA4.

In one embodiment, diseases or disorders that are dependent upon SMARCA2 and/or SMARCA4, are cancers selected from hematologic cancers, lung cancer (i.e. non-small cell lung cancer), acoustic neuroma, acute leukemia, acute lymphocytic leukemia, acute myelocytic leukemia (adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

In one embodiment, the present invention provides, Use of a compound or a pharmaceutical acceptable salt or a stereoisomer or a tautomer thereof, in the manufacture of a medicament for the treatment of diseases or disorders dependent upon SMARCA2 and/or SMARCA4.

In one embodiment, treatment of diseases or disorders dependent upon SMARCA2 and/or SMARCA4 is a cancer.

In one embodiment, the cancer is selected from hematologic cancers, lung cancer (i.e. non-small cell lung cancer), acoustic neuroma, acute leukemia, acute lymphocytic leukemia, acute myelocytic leukemia (adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor In one embodiment, the cancer dependent upon SMARCA2 and/or SMARCA4 is lung cancer such as NSCLC, i.e. non-small cell lung cancer.

In one embodiment, the cancer dependent upon SMARCA2 and/or SMARCA4 is melanoma.

In one further embodiment, the cancer is a SMARCA2 and/or SMARCA4 dependent cancer.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions can also include adjuvants such as wetting agents, emulsifying and suspending agents, sweetening, flavoring and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatine capsules using such excipients as lactose or milk sugar as well as high molecular weight polyethylene glycols and the like.

The active compounds can also be in micro-encapsulated form with one or more excipients as noted above. The solid dosage forms of tablets, draggers, capsules, pills and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, including but not limited to tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminium hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Administration of the disclosed compounds and pharmaceutical compositions can be accomplished via any mode of administration for therapeutic agents. These modes include systemic or local administration such as oral, nasal, parenteral, intravenous, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form and all using forms well known to those skilled in the pharmaceutical arts.

Illustrative pharmaceutical compositions are tablets and gelatine capsules comprising one or more compounds of the present disclosure and a pharmaceutically acceptable carrier, such as, but not limited to, a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminium silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, one or more disclosed compound is dissolved in or mixed with a pharmaceutically acceptable solvent such as, for example, water, saline, aqueous dextrose, glycerol, ethanol and the like, to thereby form an injectable isotonic solution or suspension. Proteins such as albumin, chylomicron particles, or serum proteins can be used to solubilize the disclosed compounds.

One or more disclosed compounds or compositions can be delivered by parental administration. The parental injectable administration is generally used for subcutaneous, intramuscular or intravenous injections and infusions. Injectables can be prepared in conventional forms, either as liquid solutions or suspensions or solid forms suitable for dissolving in liquid prior to injection.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur and that the description includes instances where the event or circumstance occurs as well as instances in which it does not. For example, “optionally substituted alkyl” refers to an event or circumstance where the alkyl is substituted as well as the event or circumstance where the alkyl is not substituted. The term “optionally substituted alkyl” can also be referred to as ‘unsubstituted or substituted alkyl’ group, wherein the substituent(s) are as stated therein.

As used herein, the term “unsubstituted” means that there is no substituent or that the only substituents are hydrogen.

As used herein, the term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocyclyl, an aralkyl or an aromatic or heteroaromatic moiety. It will be understood by those skilled in the art that substituents can themselves be substituted, if appropriate.

As used herein, the term “alkyl” refers to saturated aliphatic groups, including but not limited to C₁-C₁₀ straight-chain alkyl groups or C₃-C₁₀ branched-chain alkyl groups. Preferably, “alkyl” group refers to C₁-C₆ straight-chain alkyl groups or C₃-C₆ branched-chain alkyl groups. In one embodiment, the “alkyl” group refers to C₁-C₄ alkyl groups. Examples of “alkyl” include, but are not limited to, methyl, ethyl, 1-propyl, 2-propyl, isopropyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. Accordingly, examples of “alkylenyl” include, but are not limited to, —CH₂—, —CH₂CH₂—, —CH(CH₃)CH₂—, —CH₂CH₂CH₂CH₂—, —CH(CH₃)CH₂CH₂CH₂— and —CH₂CH(CH₃)CH₂CH₂—, The “alkyl” group may be optionally substituted. In one embodiment, the term “optionally substituted alkyl” can also be referred to as ‘unsubstituted or substituted alkyl’ group, wherein the substituent(s) are as stated therein.

As used herein, the term “halo” is used herein interchangeably with the term “halogen” to mean F, Cl, Br or I atoms.

As used herein, the term “amino” refers to an —NH₂ group.

As used herein, the term “haloalkyl” refers to alkyl substituted with one or more halogen atoms, wherein the halo and alkyl groups are as defined above. In one embodiment, haloalkyl contains (C₁-C₆)alkyl and preferably (C₁-C₄)alkyl. Examples of “haloalkyl” include but are not limited to fluoromethyl, difluoromethyl, chloromethyl, trifluoromethyl and 2,2,2-trifluoroethyl.

As used herein, the term “aminoalkyl” refers to an alkyl group substituted with an amino group, wherein the amino and alkyl groups are as defined above. In one embodiment, aminoalkyl contains C₁-C₄ alkyl. Examples of “aminoalkyl” include but are not limited to —CH₃—NH₂, —CH₂—CH₂—NH₂, —CH₂—CH₂—CH₂—NH₂, —CH(CH₃)—CH₂—NH₂, —CH₂—CH₂—NH (CH₃) and —CH₂—CH₂—N(CH₃)₂.

As used herein, the term “heteroalkyl” refers to a straight- or branched-chain alkyl group preferably having from 2 to 14 carbons, more preferably 2 to 10 carbons in the chain, one or more of which has been replaced by a heteroatom selected from S, O, P and N. Exemplary heteroalkyls include alkyl ethers, secondary and tertiary alkyl amines, amides, alkyl sulfides, and the like. The group may be a terminal group or a bridging group. As used herein reference to the normal chain when used in the context of a bridging group refers to the direct chain of atoms linking the two terminal positions of the bridging group.

As used herein, the term “hydroxyalkyl” refers to an alkyl group, as defined above, wherein one or more of the alkyl group's hydrogen atoms have been replaced with hydroxyl group. In one embodiment, hydroxyalkyl contains (C₁-C₆)alkyl and preferably (C₁-C₄)alkyl. Examples of hydroxyalkyl moieties include but are not limited to —CH₂OH, —CH₂CH₂OH, —CH₂CH₂CH₂OH, —CH₂CH(OH)CH₂OH, —CH₂CH(OH) CH₃, —CH(CH₃)CH₂OH.

As used herein, the term “cycloalkylenyl” refers to a divalent cycloalkylenyl group as defined herein. The term “cycloalkyl” means C₃-C₁₀ saturated cyclic hydrocarbon ring. A cycloalkyl may be a single ring, which typically contains from 3 to 7 carbon ring atoms. Examples of single ring cycloalkyls include, but are not limited to, cyclopropyl, cyclobutyl, cyclopentyl, cyclohexyl and cycloheptyl. A cycloalkyl may alternatively be polycyclic or contain more than one ring. Examples of polycyclic cycloalkyls include bridged, fused and spirocyclic carbocyclyls. Accordingly, examples of ‘cycloalkylenyl include, but not limited to, cyclopropylenyl, cyclobutylenyl, cyclopentylenyl, cyclohexylenyl and cycloheptylenyl.

As used herein, the term “heterocycloalkylenyl” refers to a divalent heterocycloalkyl group as defined herein. The term “heterocycloalkyl” refers to a non-aromatic, saturated or partially saturated, bridged bicyclic, spirocyclic, monocyclic or polycyclic ring system of 3 to 15 member, unless the ring size is specifically mentioned, having at least one heteroatom or heterogroup selected from O, N and S with the remaining ring atoms being independently selected from the group consisting of carbon, oxygen, nitrogen and sulfur. The term “heterocycloalkyl” also refers to the bridged bicyclic ring system having at least one heteroatom or hetero group selected from O, N and S. Examples of “heterocycloalkyl” include, but not limited to, azetidinyl, oxetanyl, imidazolidinyl, pyrrolidinyl, oxazolidinyl, thiazolidinyl, pyrazolidinyl, tetrahydrofuranyl, piperidinyl, dihydropyridinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, isoindolinyl, oxoisoindolinyl, dioxoisoindolinyl, aza-bicyclooctanyl, diazabicyclooctanyl, azocinyl, chromanyl, isochromanyl, xanthenyl and 2-oxa-6-azaspiro[3.3]heptanyl. Accordingly, examples of ‘heterocycloalkylenyl’ include, but not limited to, azetidinylenyl, oxetanylenyl, pyrrolidinylenyl, piperidinylenyl and piperazinylenyl. All “heterocycloalyl” and “heterocycloalkylenyl” are optionally substituted by one or more aforesaid groups.

As used herein, the term “heteroarylenyl” refers to a divalent heteroaryl group as defined herein. The term “heteroaryl” alone or in combination with other term(s) means a completely unsaturated ring system containing a total of 5 to 14 ring atoms, unless the ring size is specifically mentioned. At least one of the ring atoms is a heteroatom (i.e., O, N or S), with the remaining ring atoms/groups being independently selected from C, N, O or S. A heteroaryl may be a single-ring (monocyclic) or multiple rings (bicyclic, tricyclic or polycyclic) fused together or linked covalently. Preferably, “heteroaryl” is a 5- to 6-membered ring, unless the ring size is specifically mentioned. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl (pyridinyl), 3-fluoropyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, a-carbolinyl, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and the like. Accordingly, examples of heteroarylenyl include, but not limited to, furanylenyl, thienylenyl, pyrrolylenyl, pyrazolylenyl, imidazolylenyl, oxazolylenyl, isoxazolylenyl, thiazolylenyl, isothiazolylenyl, 1H-tetrazolylenyl, oxadiazolylenyl, triazolylenyl, pyridylenyl (pyridinylenyl), pyrimidinylenyl, pyrazinylenyl, pyridazinylenyl, 1,2,3-triazinylenyl, 1,2,4-triazinylenyl and 1,3,5-triazinylenyl. Heteroaryl group may be optionally further substituted.

As used herein, the term “hydroxy” or “hydroxyl” alone or in combination with other term(s) means —OH.

As used herein, the term “oxo” refers to ═O group.

As used herein, the term “alkoxy” refers to the group —O-alkyl, where alkyl groups are as defined above. Exemplary C₁-C₁₀ alkoxy group include but are not limited to methoxy, ethoxy, n-propoxy, n-butoxy or t-butoxy. In one embodiment, the “alkoxy” group refers to C₁-C₆ alkoxy groups. In another embodiment, the “alkoxy” group refers to C₁-C₄ alkoxy groups.

An alkoxy group can be optionally substituted with one or more suitable groups. In some embodiments, alkoxy group can be unsubstituted or substituted with one or more substitutents, wherein the substitutents are described herein.

As used herein, the term “arylenyl” refers to a divalent aryl group as defined herein. The term “aryl”, as employed herein as such or as part of another group, refers to a monocyclic, bicyclic or polycyclic aromatic hydrocarbon ring system of 6 to 14 carbon atoms. Examples of aryl groups include, but are not limited to phenyl, naphthyl, biphenyl, anthryl, biphenylenyl and acenaphthyl. Preferred aryl group is phenyl. The term “arylenyl” refers to a divalent aryl group. Accordingly, examples of arylenyl groups include, but are not limited to phenylenyl, naphthylenyl, biphenylenyl and anthrylenyl. The term “optionally substituted aryl” can also be referred to as ‘unsubstituted or substituted aryl’ group, wherein the substituent(s) are as stated therein.

As used herein, the term “acyl” refers to a group R—CO— or —CO—R wherein R is an optionally substituted alkyl group defined above. Examples of ‘acyl’ groups are, but not limited to, CH₃CO—, CH₃CH₂CO—, CH₃CH₂CH₂CO— or (CH₃)₂CHCO—. The term “—O-acyl” refers to —O—CO—R wherein R is an alkyl as defined above.

As used herein, the term “heteroatom” as used herein designates a sulfur, nitrogen or oxygen atom.

As used herein, the term ‘compound(s)’ comprise(s) the compound(s) disclosed in the present invention.

As used herein, the term “salt/salts” refers to the salts derived from appropriate bases include alkali metal (e.g., sodium and potassium), alkaline earth metal (e.g., magnesium), ammonium and N⁺(C₁₋₄ alkyl)₄ salts.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt thereof; and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the aforementioned range.

The term “tautomer” refers to compounds in which hydrogen atoms are transposed to other parts of the molecules and the chemical bonds between the atoms of the molecules are consequently rearranged. Compounds of the present invention, free form and salts thereof, may exist in multiple tautomeric forms. It is understood that all tautomeric forms, insofar as they may exist, are included within the invention. For example, pyridine or pyridyl can be optionally substituted by oxo to form a respective pyridone or pyridon-yl and may include its tautomeric form such as a respective hydroxy-pyridine or hydroxy-pyridyl, provided said tautomeric form may be obtainable.

As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant or emulsifier that has been approved by the United States Food and Drug Administration as being acceptable for use in humans or domestic animals.

As used herein, the term “administer”, “administering,” or “administration” as used in this disclosure refers to either directly administering one or more disclosed compounds or a pharmaceutically acceptable salt of one or more disclosed compounds or a composition comprising one or more disclosed compounds to a subject or administering a prodrug derivative or analog of the compound or a pharmaceutically acceptable salt of the compound or composition to the subject, which can form an equivalent amount of active compound within the subject's body.

As used herein, the term “carrier” as used in this disclosure, encompasses carriers, excipients and diluents and means a material, composition or vehicle, such as a liquid or solid filler, diluent, excipient, solvent or encapsulating material, involved in carrying or transporting a pharmaceutical agent from one organ or portion of the body to another organ or portion of the body of a subject.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal and most preferably a human.

As used herein, the term, “therapeutically effective amount” refers to an amount of a compound of formula (I) or a pharmaceutically acceptable salt or a stereoisomer or a tautomer thereof; or a composition comprising the compound of formula (I) or a pharmaceutically acceptable salt or a stereoisomer or a tautomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer or a tautomer thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

As used herein, the term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I)) can form pharmaceutically acceptable salts with various organic bases such as lysine, arginine, guanidine, diethanolamine or metformin. Suitable base salts include, but are not limited to, aluminum, calcium, lithium, magnesium, potassium, sodium or zinc salts.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, hematologic cancers and renal cell carcinomas, cancer of the bladder, bowel, breast, cervix, colon, esophagus, head, kidney, liver, lung, neck, ovary, pancreas, prostate and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “stereoisomers” refers to any enantiomers, diastereoisomers or geometrical isomers of the compound of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and i-Isomers and mixtures thereof. Individual stereoisomers of compounds can be prepared synthetically from commercially available starting materials which contain chiral centres or by preparation of mixtures of enantiomeric products followed by separation such as conversion to a mixture of diastereomers followed by separation or recrystallization, chromatographic techniques, direct separation of enantiomers on chiral chromatographic columns or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen I and zusammen (Z) Isomers as well as the appropriate mixtures thereof.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers. The disclosure includes enantiomers of the compounds described herein. Each compound herein disclosed includes all the enantiomers that conform to the general structure of the compound. The compounds may be in a racemic or enantiomerically pure form or any other form in terms of stereochemistry. In some embodiments the compounds are the (S)-enantiomer.

The term “diastereomers” refers to the set of stereoisomers which cannot be made superimposable by rotation around single bonds. For example, cis- and trans-double bonds, endo- and exo-substitution on bicyclic ring systems and compounds containing multiple stereogenic centres with different relative configurations are considered to be diastereomers. The term “diastereomer” refers to any member of this set of compounds. In some examples presented, the synthetic route may produce a single diastereomer or a mixture of diastereomers. The disclosure includes diastereomers of the compounds described herein.

The compounds of the present invention may be used as single drug or as a pharmaceutical composition in which the compound is mixed with various pharmacologically acceptable materials.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

The term, “Prodrug” is meant to indicate a compound that may be converted under physiological conditions or by solvolysis to a biologically active compound described herein (e.g., compound of structure (I)). Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14 and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group or acetamide, formamide and benzamide derivatives of an amine functional group in the active compound and the like.

In some embodiments, prodrugs include compounds of structure (I) having a phosphate, phosphoalkoxy, ester or boronic ester substituent. Without being bound by theory, it is believed that such substituents are converted to a hydroxyl group under physiological conditions.

Accordingly, embodiments include any of the compounds of the present invention, wherein a hydroxyl group has been replaced with a phosphate, phosphoalkoxy, ester or boronic ester group, for example a phosphate or phosphoalkoxy group. For example, in some embodiments a hydroxyl group on the R₄ or R₈ moiety is replaced with a phosphate, phosphoalkoxy, ester or boronic ester group, for example a phosphate or alkoxy phosphate group.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated into compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

### EXPERIMENTAL

The abbreviations used in the entire specification is summarized below with their meaning.

MeOH—Methanol, EtOH—Ethanol, DCM—Dichloromethane, DMF—N,N-Dimethylformamide, EtOAc—Ethyl acetate, THF—Tetrahydrofuran, DME-1,2-Dimethoxyethane, DMSO—Dimethyl sulfoxide DIPEA—N,N-Diisopropylethylamine, NCS—N-chloro succinimide, HATU—(1-[Bis(dimethylamino)methylene]-1H-1,2,3-triazolo[4,5-b]pyridinium 3-oxid hexafluoro phosphate), KOAc—Potassium acetate, Na₂S-4—Sodium sulphate, Na₂CO₃—Sodium carbonate, K₂CO₃—Potassium carbonate, Cs₂CO₃—Cesium carbonate, KO^{t}Bu—Potassium tert-butoxide, Pd(dppf)Cl₂·DCM—[1,1′-Bis(diphenylphosphino)ferrocene]-dichloropalladium (II) dichloromethane complex, NaoMe—Sodium methoxide, BINAP—(2,2′-bis(diphenylphosphino)-1,1′-binaphthyl), H₂SO₄—Sulfuric acid, NaHCO₃—Sodium bicarbonate, TEA—Triethyl amine; DEA—Diethyl amine, LiOH—H₂O Lithium hydroxide monohydrate; EDC.HCl—1-(3-Dimethylaminopropyl)-3-ethylcarbodiimide. hydrochloride, DAST—diethylaminosulfur trifluoride, PPTs—Pyridinium p-toluenesulfonate, NaH—Sodium hydride, NH₄OH—Ammonium hydroxide, NaOH—Sodium hydroxide, HCl—Hydrochloric acid, Pd(pph₃)₂Cl₂. DCM-Bis(triphenylphosphine)-palladium(II) dichloride Dichloromethane complex; Pd(OAc)₂—Palladium (II) acetate, mL—Milliliter, TLC—Thin layer chromatography, RT—Room temperature, h—Hour, N—Normality, M—Molarity, ¹HNMR—Proton nuclear magnetic resonance, DMSO-d₆-Deuterated Dimethyl sulfoxide, CDCl₃—Deuterated chloroform, CD₃OD—Deuterated Methanol, s—Singlet, d—Doublet, t—Triplet, m—Multiplet, H—Proton, MHz—Mega hertz, Hz—Hertz, Ppm—Parts per million, Bs—Broad singlet, HPLC—High-performance liquid chromatography, LCMS—Liquid chromatography Mass spectroscopy, g—Gram, mmol—Milli mol and ° C.—degree centigrade.

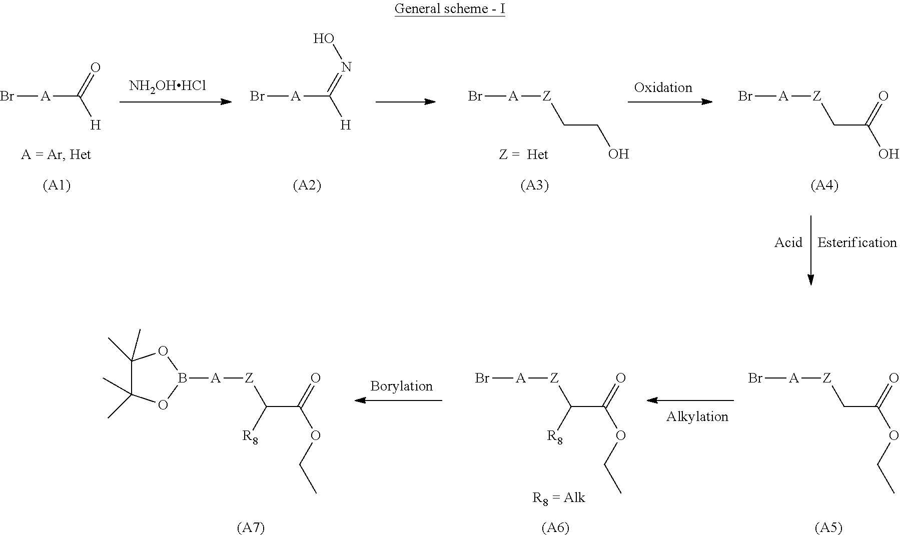

The general scheme for the synthesis of intermediate compound represented by formula (A7) is depicted in above scheme. The compound of formula (A1) can be reacted with hydroxylamine derivatives to yield compound of formula (A2) which upon further cyclization in the presence of base can provide compound of formula (A3). The compound of formula (A3) can undergo oxidation in presence of metal oxides to result in respective acid derivatives of formula (A4) which can be undergo esterification in the presence of an acid and solvent to result in formula (A5) which further undergoes alkylation in the presence of suitable reagent to provide alkyl derivatives of formula (A6). The compound of formula (A6) can undergo suzuki coupling to provide intermediate compound of formula (A7).

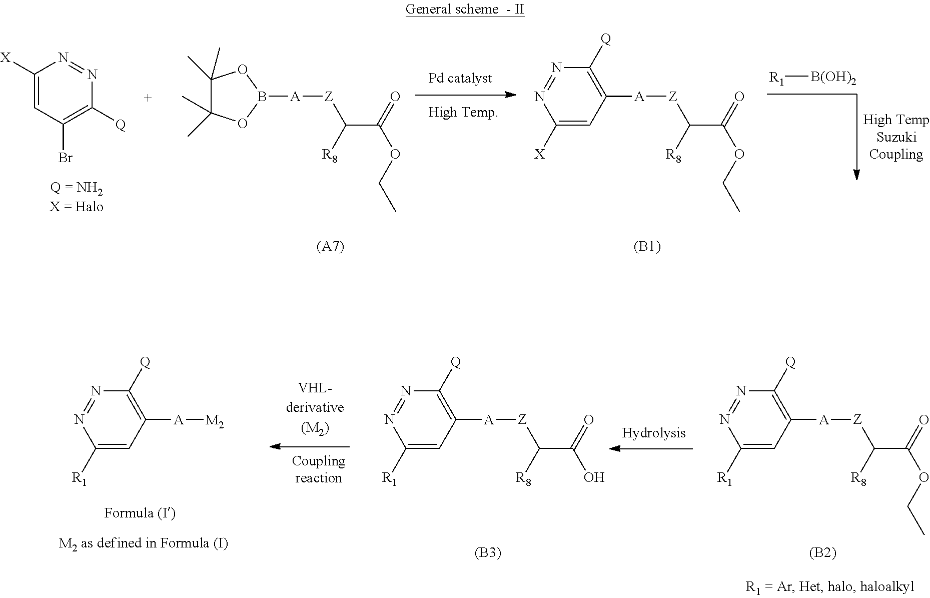

The general scheme for the synthesis of compound represented by formula (I′) is depicted in above scheme. The compound of formula (B1) can be obtained by the reaction of compound of formula (A7) with suitable pyridazine derivatives in a solvent. The formula (B1) compound upon further reaction with Aryl or heteroaryl boronic acid derivatives in a suitable solvent can result in compound of formula (B2) which can undergo hydrolysis in the presence of a base to yield compound of formula (B3). The compound of formula (B3) can be coupled with VHL derivative of M2 in the presence of a solvent to yield compound of formula (I′).

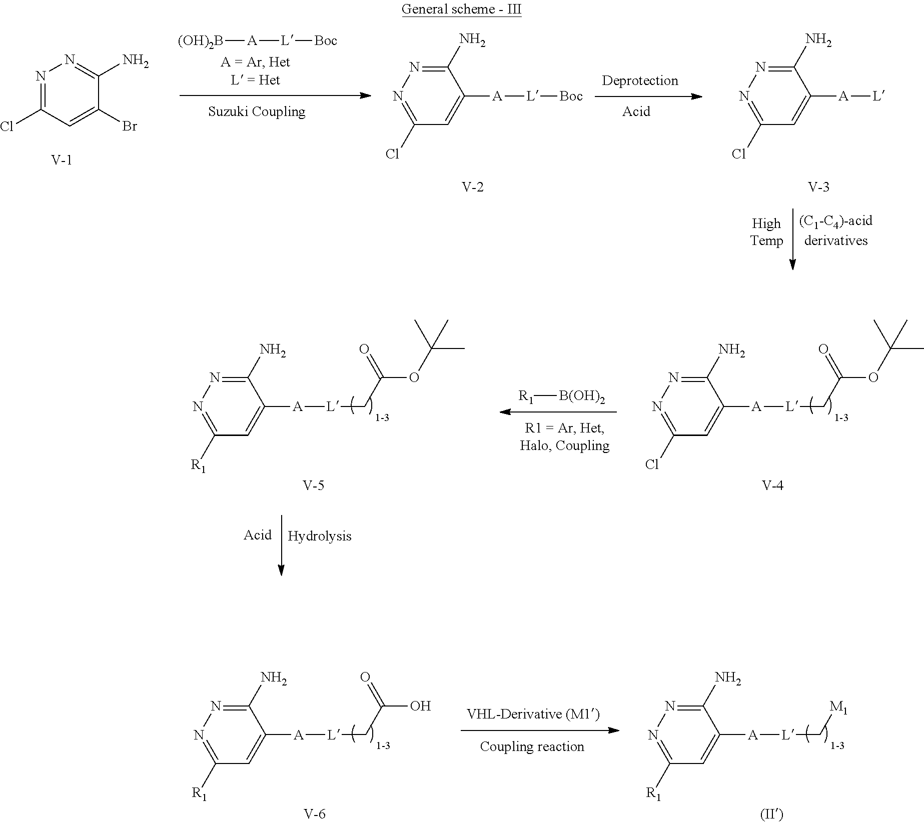

The general scheme for the synthesis of compound represented by formula (II′) is depicted in above scheme. The compound of formula (V-2) can be obtained by treating the appropriate pyridazine derivative (V-1) with aryl- or heteroaryl-borane derivatives ((Boc)-L′-A-B(OH)₂) in a suitable solvent and reagent. The obtained compound of formula (V-2) can be deprotected by hydrolysis in the presence of a solvent to yield compound of formula (V-3) which upon further reaction with corresponding protected acid derivatives at high temperature to result in compound of formula (V-4). The compound of formula (V-4) can be reacted with aryl- or heteroaryl-borane derivatives (R1-B(OH)₂) to provide compound of formula (V-5) which can undergo hydrolysis in the presence of acid to yield compound of formula (V-6). The compound of formula (V-6) upon acid-amine coupling reaction with VHL-derivative of M1′ to provide compound of formula (II′).

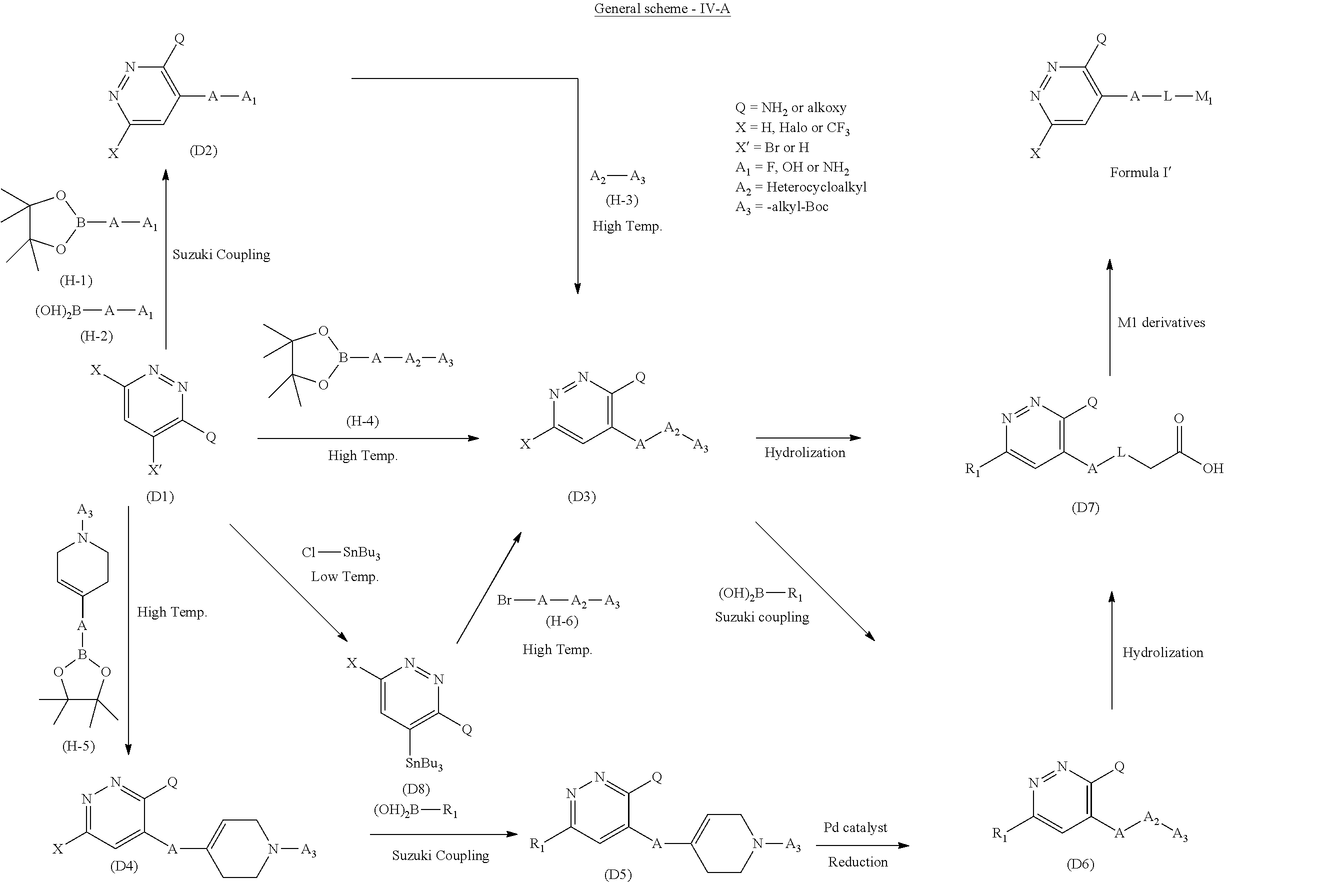

The general scheme for the synthesis of compound represented by formula (I′) is depicted in above scheme IV-A. The compound of formula (D2) can be obtained by treating the appropriate pyridazine derivative (D1) with the compound of formula (H-1) or (H-2) in a suitable solvent. The formula (D2) compounds can further be reacted with the compound of formula (H-3) in suitable solvent and at higher temperature to provide the compound of formula (D3). The formula (D3) compound can also be obtained by reacting compound of formula (D1) with tributylchlorostannane in a suitable solvent to result in the compound formula (D8) and further reaction of so obtained formula (D8) with compound of formula (H-6) in suitable solvent. The compound of formula (D1) can also react with the compound of formula (H-5) with appropriate solvent and at higher temperature can yield compound of formula (D4). The so obtained compound of formula (D3) can react with aryl or heteroaryl-boron derivatives in presence of suitable solvents to provide compound of formula (D6). The compound of formula (D4) can also react with aryl or heteroaryl-boron derivatives in presence of suitable solvents to provide compound of formula (D5) which can also yield formula (D6) compound upon reduction reaction in the presence of a suitable solvent. The formula (D6) compound can further undergo hydrolysis in the presence of acid to yield compound of formula (D7). The compound of formula (D7) upon acid-amine coupling reaction with VHL-derivative of M1′ to provide compound of formula (I′).

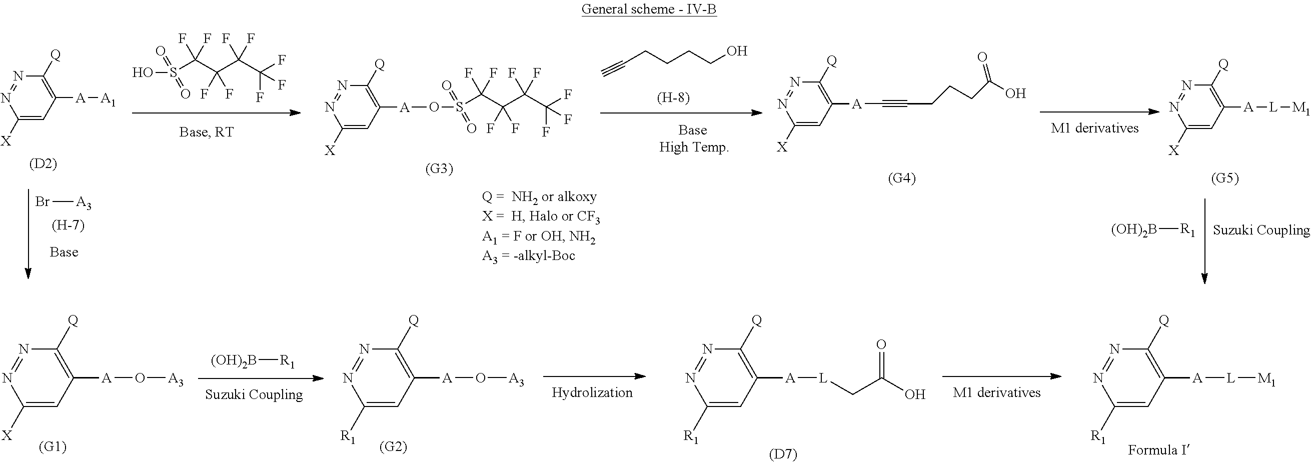

Another general scheme for the synthesis of compound represented by formula (I′) is depicted in above scheme IV-B. The compound of formula (G1) can be obtained by treating the compound of formula (D2) with compound of formula (H-7) in suitable base and solvent. The formula (G1) compound can further react with aryl- or heteroaryl-borane derivatives in a suitable solvent and reagent to provide the compound of formula (G2) which upon hydrolysis in presence of acid can yield the compound of formula (D7). The formula (D7) compound can undergo acid-amine coupling reaction with VHL-derivative of M1′ to provide compound of formula (I′). Alternatively, the compound of formula (G3) can be obtained by treating the compound of formula (D2) with 1,1,2,2,3,3,4,4,4-nonafluorobutane-1-sulfonic acid derivatives in presence of a base, which can further react with the compound of formula (H-8) at higher temperature with suitable base and solvent to provide the compound of formula (G4). The obtained compound of formula (G4) upon acid-amine coupling reaction with VHL-derivative of M1′ to provide compound of formula (G5) which can undergo coupling reaction with aryl- or heteroaryl-borane derivatives to provide compound of formula (I′).

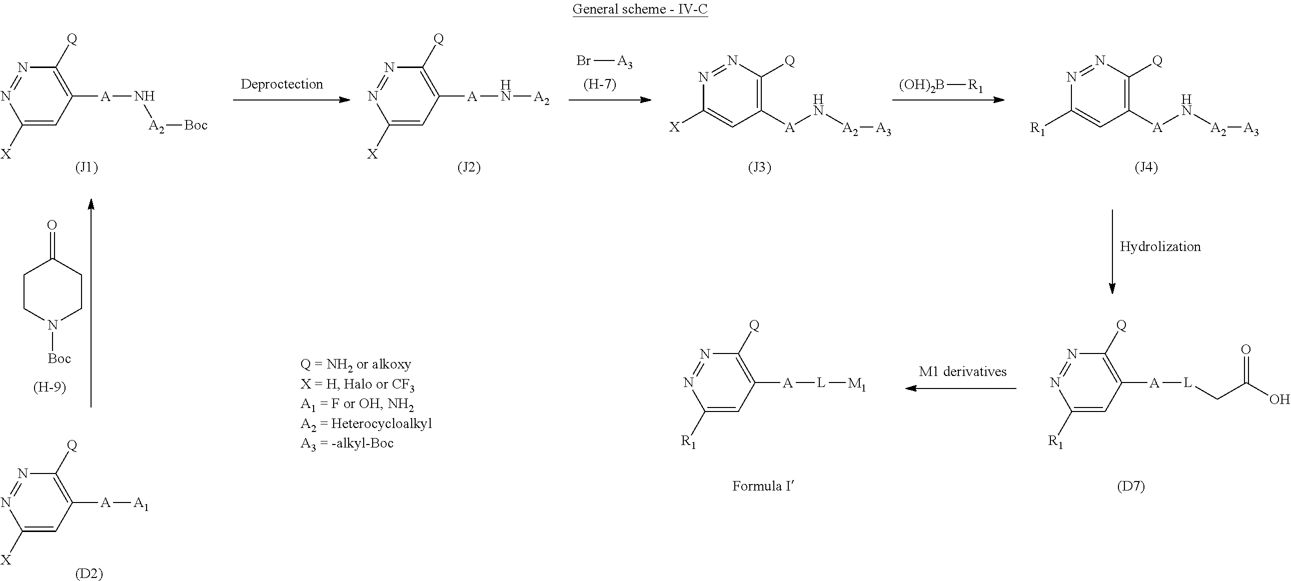

The general scheme for the synthesis of compound represented by formula (I′) is depicted in above scheme IV-C. The compound of formula (J1) can be obtained by treating the compound of formula (D2) with compound of formula (H-9) in suitable solvent, which can further undergo deprotection reaction to provide the compound of formula (J2). The obtained compound of formula (J2) can react with the compound of formula (H-7) in appropriate solvent and reagent to yield the compound of formula (J3). The obtained compound of formula (J3) can react with aryl- or heteroaryl-borane derivatives in a suitable solvent and reagent to provide the compound of formula (J4), which can further undergo hydrolysis reaction to yield compound of formula (D7). The compound of formula (D7) upon acid-amine coupling reaction with VHL-derivative of M1′ to provide compound of formula (I′).

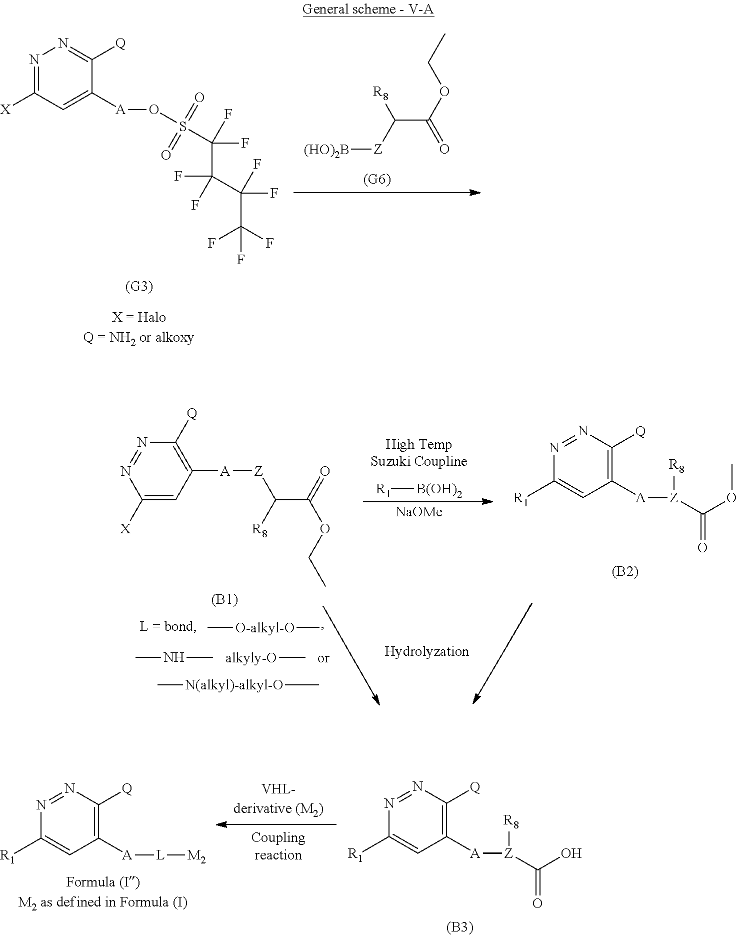

The general scheme for the synthesis of compound represented by formula (I′) is depicted in above scheme V-A. The compound of formula (B1) can be obtained by the reaction of compound of formula (G3) with the compound of formula (G6) derivatives in a suitable solvent. The formula (B1) compounds upon further reaction with Aryl or heteroaryl boronic acid derivatives in a suitable solvent to result in compound of formula (B2). The compound of formula (B2) and (B1) can undergo hydrolysis in the presence of base to yield compound of formula (B3). The compound of formula (B3) can be coupled with VHL derivative of M2′ in the presence of solvent to provide compound of formula (I′).

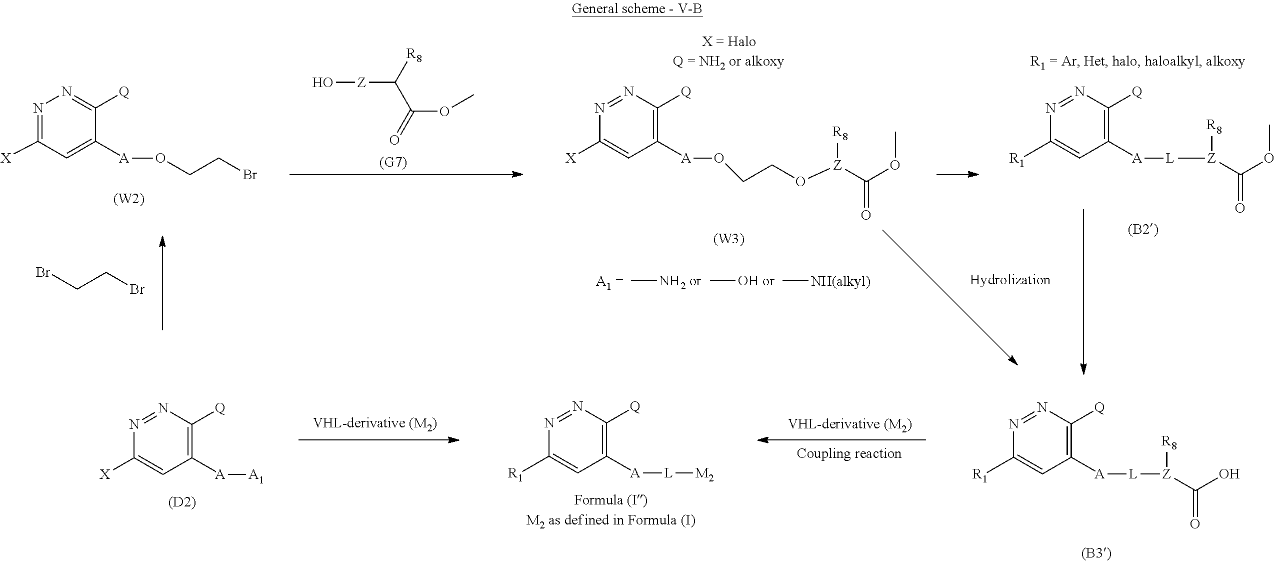

The general scheme for the synthesis of compound represented by formula (I′) is depicted in above scheme V-B. The compound of formula (W2) can be obtained by the reaction of compound of formula (D2) with 1,2-dibromoethane in a solvent which upon further reaction with the compound of formula (G7) derivatives in suitable solvent to result in compound of formula (W3). The compound of formula (W3) can react with Aryl or heteroaryl boronic acid derivatives with suitable solvent and suitable temperature to result in compound of formula (B2′). The compound of formula (B2′) and (W3) can undergo hydrolysis in the presence of base to yield compound of formula (B3′), which further can be coupled with VHL derivative of M2′ in the presence of solvent to provide compound of formula (I′).

### Intermediate-1: Tert-butyl 2-(4-(4-(4,4,5,5-tetramethyl-1,3,2-dioxaborolan-2-yl)phenyl)piperazin-1-yl)acetate

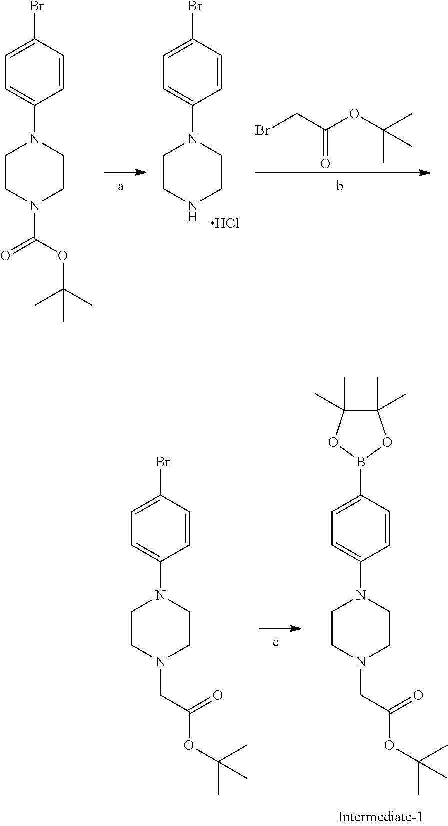

### Step a: Synthesis of 1-(4-bromophenyl)piperazine hydrochloride

To a stirred solution of tert-butyl 4-(4-bromophenyl)piperazine-1-carboxylate (5.0 g, 14.7 mmol) in DCM (50 mL) was added 4N dioxane HCl (50 mL) at 0° C. and the reaction mixture was stirred for 16 h at RT under nitrogen atmosphere. The reaction mixture was concentrated under vacuum to get crude product. The crude product was triturated with n-pentane and then dried under vacuum to afford pure title compound as pale-yellow solid (5 g, crude yield). ¹H NMR (400 MHz, DMSO-d₆): δ 9.29 (bs, 1H), 7.38 (d, J=9.2 Hz, 2H), 6.94 (d, J=9.2 Hz, 2H), 5.06 (m, 4H), 3.35 (m, 4H); LC-MS: m/z 240.03 (M+H).

### Step b: Synthesis of tert-butyl 2-(4-(4-bromophenyl)piperazin-1-yl)acetate

To a stirred solution of 1-(4-bromophenyl)piperazine hydrochloride (5.0 g, 18.1 mmol) in DMF (50 mL) was added DIPEA (4.66 g, 36.2 mmol) at RT and the mixture was stirred for 15 min. Then, tert-butyl 2-bromoacetate (5.3 g, 27.15 mmol) was added to the reaction mixture and stirred for 4 h at RT under nitrogen atmosphere. The reaction mixture was quenched with cold water and stirred for 1 h. The solid was filtered and dried under vacuum to afford the title compound as off-white solid (4.5 g, 70%). ¹H NMR (400 MHz, DMSO-d₆): δ 7.29 (d, J=8.4 Hz, 2H), 6.84 (d, J=8.4 Hz, 2H), 3.11 (s, 2H), 3.07 (m, 4H), 2.58 (m, 4H), 1.37 (s, 9H); LC-MS: m/z 340.8 (M+H).

### Step c: Synthesis of tert-butyl 2-(4-(4-(4,4,5,5-tetramethyl-1,3,2-dioxaborolan-2-yl)phenyl)piperazin-1-yl)acetate

To a stirred solution of tert-butyl 2-(4-(4-bromophenyl piperazin-1-yl)acetate) (4.2 g, 11.8 mmol) in dioxane (80 mL) was added bis(pinacolato)diboron (4.52 g, 17.8 mmol) and KOAc (2.33 g, 23.7 mmol) at RT. The reaction mixture was degassed with nitrogen for 5 min, then Pd(dppf)Cl₂·DCM (0.89 g, 1.18 mmol) was added into the reaction mixture and heated at 100° C. for 4 h. Once the reaction was completed (monitored by TLC), the reaction mixture was diluted with EtOAc. The combined organic layer was washed with water and brine, dried over anhydrous sodium sulphate and concentrated under vacuum to afford the title compound as off-white solid (5 g, 100%). ¹H NMR (400 MHz, DMSO-d₆): δ 7.50 (d, J=8.0 Hz, 2H), 6.89 (d, J=8.8 Hz, 2H), 3.20 (bs, 4H), 3.15 (s, 2H), 2.62 (d, J=4.4 Hz, 4H), 1.41 (s, 9H), 1.26 (s, 12H); LC-MS: m/z 402.7 (M+H).

### Intermediate-2: Ethyl 2-(1-(4-(4,4,5,5-tetramethyl-1,3,2-dioxaborolan-2-yl)phenyl)piperidin-4-yl)acetate

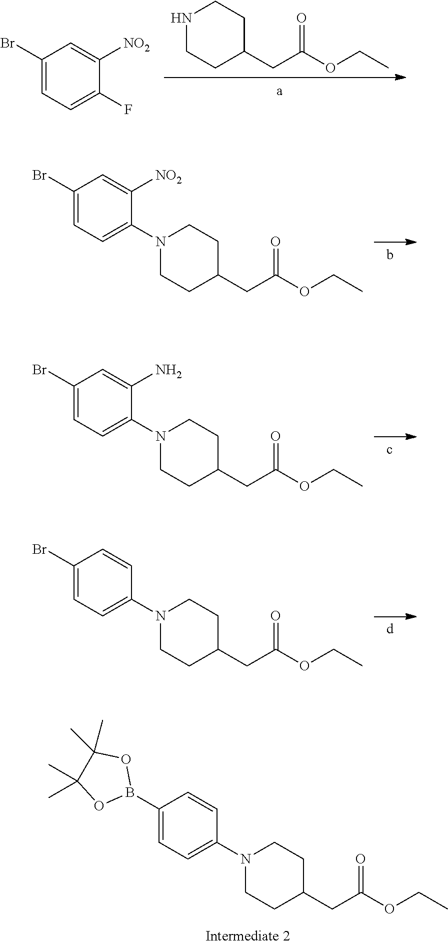

### Step a: Synthesis of ethyl 2-(1-(4-bromo-2-nitrophenyl)piperidin-4-yl)acetate

To a stirred solution of 4-bromo-1-fluoro-2-nitrobenzene (2.0 g, 9.1 mmol) in DMF (20 mL) were added ethyl 2-(piperidin-4-yl)acetate (1.87 g, 10.9 mmol) and DIPEA (3.7 g, 27.4 mmol) at RT and the mixture was heated at 100° C. for 4 h. The reaction mixture was quenched with cold water and extracted with EtOAc. The combined organic layer was washed with water and brine, dried over anhydrous sodium sulphate and concentrated under vacuum to get the crude product. The crude product was purified by combiflash column chromatography using 20-25% ethyl acetate in hexane as eluent to afford the title compounds as yellow solid (1.3 g, 38%). ¹H NMR (400 MHz, DMSO-d₆): δ 7.99 (d, J=2.4 Hz, 1H), 7.70 (dd, J=2.4, 6.4 Hz, 1H), 7.24 (d, J=8.8 Hz, 1H), 4.05 (q, J=5.4 Hz, 2H), 3.12 (d, J=12.4 Hz, 2H), 2.82-2.76 (m, 2H), 2.26 (d, J=7.2 Hz, 2H), 1.84-1.79 (m, 1H), 1.69 (d, J=12.4 Hz, 2H), 1.32-1.22 (m, 2H), 1.17 (t, J=7.4 Hz, 3H); LC-MS: m/z 371.0 (M+H).

### Step b: Synthesis of ethyl 2-(1-(2-amino-4-bromophenyl)piperidin-4-yl)acetate

To a stirred solution of ethyl 2-(1-(4-bromo-2-nitrophenyl)piperidin-4-yl)acetate (1.30 g, 3.5 mmol) in methanol (10 mL) and water (3 mL) mixture were added iron (3.1 g, 28 mmol) and NH₄OH (1.5 g, 28 mmol) and the mixture was stirred at RT for 4 h. After completion of the reaction (monitored by TLC), the reaction mixture was diluted with methanol and filtered.

The filtrate was concentrated and diluted with EtOAc, washed with water and brine, dried over anhydrous sodium sulphate and concentrated under vacuum to afford the title compound as off-white solid (1.2 g, 100%). ¹H NMR (400 MHz, DMSO-d₆): δ 6.80-6.78 (m, 2H), 6.63 (dd, J=2.4, 6.0 Hz, 1H), 4.91 (s, 2H), 4.06 (q, J=7.0 Hz, 2H), 2.97 (d, J=11.6 Hz, 2H), 2.27 (d, J=6.8 Hz, 2H), 1.73-1.69 (m, 5H), 1.40-1.36 (m, 2H), 1.19 (t, J=7.0 Hz, 3H).

### Step c: Synthesis of ethyl 2-(1-(4-bromophenyl)piperidin-4-yl)acetate

To a stirred solution of ethyl 2-(1-(2-amino-4-bromophenyl)piperidin-4-yl)acetate (1.0 g, 2.9 mmol) in THF (10 mL) was added tert-butyl nitrite (0.58 g, 5.1 mmol) and stirred at RT for 3 h. After completion of the reaction (monitored by TLC), the reaction mixture was diluted with EtOAc. The combined organic layer was washed with water and brine, dried over anhydrous sodium sulphate and concentrated under vacuum to get the crude product. The crude product was purified by combiflash column chromatography using 20% ethyl acetate in hexane as eluent to afford the title compound as off-white solid (0.37 g, 34%). ¹H NMR (400 MHz, DMSO-d₆): δ 7.30 (d, J=8.8 Hz, 2H), 6.87 (d, J=9.2 Hz, 2H), 4.06 (q, J=14.4 Hz, 2H), 3.64 (d, J=12.8 Hz, 2H), 2.67-2.62 (m, 2H), 2.25 (d, J=7.2 Hz, 2H), 1.70 (d, J=12.4 Hz, 3H), 1.28-1.25 (m, 2H), 1.17 (t, J=7.2 Hz, 3H). LC-MS: m/z 326.1 (M+H).

### Step d: Synthesis of ethyl 2-(1-(4-(4,4,5,5-tetramethyl-1,3,2-dioxaborolan-2-yl)phenyl)piperidin-4-yl)acetate

To a stirred solution of ethyl 2-(1-(4-bromophenyl)piperidin-4-yl)acetate (0.37 g, 1.13 mmol) in dioxane (10 mL) was added bis(pinacolato)diboron (0.45 g, 1.7 mmol) and KOAc (0.22 g, 2.27 mmol) at RT. The reaction mixture was degassed with nitrogen for 5 min. Pd(dppf)Cl₂·DCM (0.09 g, 0.11 mmol) was added into the reaction mixture, heated at 100° C. for 6 h. Once the reaction was completed (monitored by TLC), the reaction mixture was diluted with EtOAc. The combined organic layer was washed with water and brine, dried over anhydrous sodium sulphate and concentrated under vacuum to get the residue which was purified by combiflash column chromatography using 10-15% ethyl acetate in hexane as eluent to afford the title compound as yellow solid (0.25 g, 58%). ¹H NMR (400 MHz, DMSO-d₆): δ 7.48 (d, J=8.4 Hz, 2H), 6.87 (d, J=8.4 Hz, 2H), 4.06 (q, J=14.4 Hz, 2H), 3.78 (d, J=12.4 Hz, 2H), 2.75-2.66 (m, 1H), 2.24 (d, J=6.8 Hz, 2H), 1.87 (t, J=7.2 Hz, 1H), 1.72-1.68 (m, 2H), 1.29-1.16 (m, 18H). LC-MS: m/z 373.9 (M+H).

### Intermediate-3: Ethyl 3-methyl-2-(3-(4-(4,4,5,5-tetramethyl-1,3,2-dioxaborolan-2-yl)phenyl)isoxazol-5-yl)butanoate

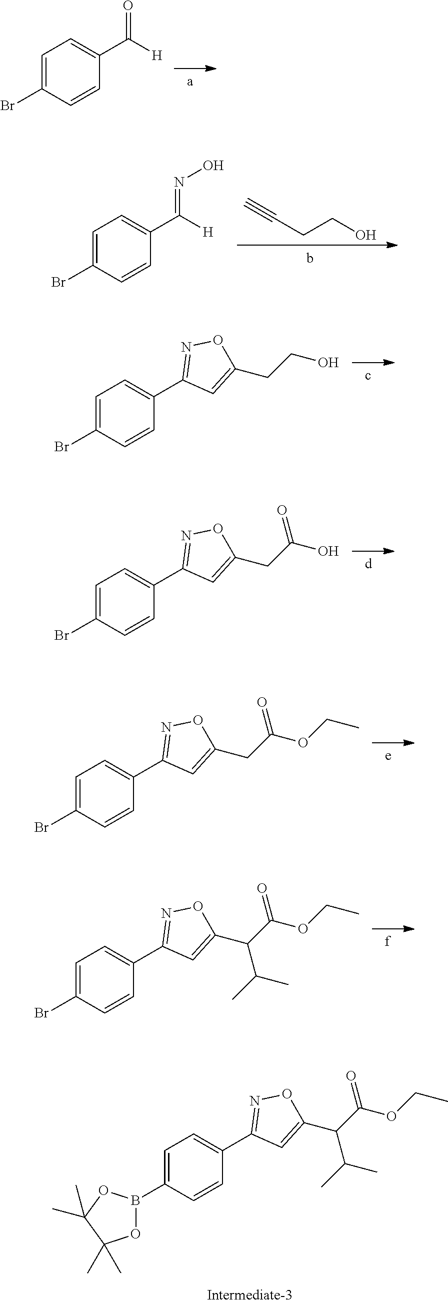

### Step a: Synthesis of 4-bromobenzaldehyde oxime

To a solution of 4-bromobenzaldehyde (30 g, 162.16 mmol) and hydroxylamine hydrochloride (14.64 g, 210.8 mmol) in ethanol (300 mL) was added pyridine (19.21 mL, 243.2 mmol) to the reaction mixture and stirred at RT for 6 h. The reaction mixture was quenched with ice-cold water and stirred for 20 min. The obtained solid was further washed with water and dried under vacuum to get the crude compound. The crude compound was washed with hexane and dried under vacuum to give the title compound as white solid (24 g, 74%). H NMR (400 MHz, DMSO-d₆): δ 11.37 (s, 1H), 8.13 (s, 1H), 7.60 (d, J=10.8 Hz, 2H), 7.54 (d, J=10.4 Hz, 2H); LC-MS: m/z 199.9 (M+H).

### Step b: Synthesis of 2-(3-(4-bromophenyl)isoxazol-5-yl)ethan-1-ol

To a solution of 4-bromobenzaldehyde oxime (15 g, 75 mmol) in DCM (300 mL) were added NCS (12.01 g, 90 mmol) and heated at 45° C. for 30 min. The reaction mixture was cooled to RT. But-3-yn-1-ol (6.3 g, 90 mmol) and TEA (10.52 mL, 75 mmol) were added dropwise into the reaction mixture over the period of 15 min. The reaction mixture was again heated at 45° C. for 7 h. The reaction mixture was quenched with ice-cold water and extracted with EtOAc. The combined organic layer was washed with water and brine, dried over anhydrous sodium sulphate and concentrated under vacuum to get the residue which was purified by combiflash column chromatography using 10% ethyl acetate in hexane as eluent to afford the title compound as white solid (7.5 g, 37%). ¹H NMR (400 MHz, DMSO-d₆): δ 7.80 (d, J=6.4 Hz, 2H), 7.71 (d, J=6.8 Hz, 2H), 6.86 (s, 1H), 4.92 (t, J=10.8 Hz, 1H), 3.75 (q, J=11.6 Hz, 2H), 2.94 (t, J=12.8 Hz, 2H); LC-MS: m/z 267.90 (M+H).

### Step c: Synthesis of 2-(3-(4-bromophenyl)isoxazol-5-yl)acetic acid

To a stirred solution of 2-(3-(4-bromophenyl)isoxazol-5-yl)ethan-1-ol (7.5 g, 27.9 mmol) in acetone (150 mL) at 0° C. was dropwise added Jones reagent (15 mL) and temperature was brought to RT over the period of 2 h. After completion of the reaction, the reaction mixture was diluted with acetone and filtered. The filtrate was concentrated under vacuum to get crude product. The crude product was triturated with hexane and dried under vacuum to afford pure title compound as off-white solid (7 g, 88%). ¹H NMR (400 MHz, DMSO-d₆): δ 7.82 (d, J=8.8 Hz, 2H), 7.71 (d, J=8.4 Hz, 2H), 6.96 (s, 1H), 3.96 (s, 2H); LC-MS: m/z 283.09 (M+H).

### Step d: Synthesis of ethyl 2-(3-(4-bromophenyl)isoxazol-5-yl)acetate

To a stirred solution of 2-(3-(4-bromophenyl)isoxazol-5-yl)acetic acid (7 g, 24.9 mmol) in ethanol (140 mL) at 0° C. was dropwise added H₂SO₄ (7 mL) and heated at 80° C. for 2 h.

After completion of the reaction (monitored by TLC), the reaction mixture was concentrated and diluted with EtOAc. The combined organic layer was washed with water and brine, dried over anhydrous sodium sulphate and concentrated under vacuum to give the residue, which was purified by combiflash column chromatography using 7% ethyl acetate in hexane as eluent to afford the title compound as off-white solid (5.8 g, 76%). ¹H NMR (400 MHz, DMSO-d₆): δ 7.82 (d, J=8.4 Hz, 2H), 7.72 (d, J=8.4 Hz, 2H), 6.99 (s, 1H), 4.17 (q, J=14.4 Hz, 2H), 4.08 (s, 2H), 1.22 (t, J=14.4 Hz, 3H); LC-MS: m/z 309.9 (M+H).

### Step e: Synthesis of ethyl 2-(3-(4-bromophenyl)isoxazol-5-yl)-3-methylbutanoate

To a solution of ethyl 2-(3-(4-bromophenyl)isoxazol-5-yl)acetate (5.8 g, 18.77 mmol), in THF (80 mL) was dropwise added KO^{t}Bu 1M solution (28.1 mL, 28.1 mmol) at 0° C. and stirred for 15 min. 2-iodo propane (2.25 mL, 22.5 mmol) was added into the reaction mixture and stirred at RT for 16 h. After reaction completion, the reaction mixture was quenched with ice-cold water and extracted with EtOAc. The combined organic layer was washed with water and brine, dried over anhydrous sodium sulphate and concentrated under vacuum to give the residue which was purified by combiflash column chromatography using 4-5% ethyl acetate in hexane as eluent to give title compound as colourless liquid (3.8 g, 57%). ¹H NMR (400 MHz, DMSO-d₆): δ 7.84 (d, J=8.8 Hz, 2H), 7.72 (d, J=8.0 Hz, 2H), 7.05 (s, 1H), 4.16 (q, J=7.2 Hz, 2H), 3.85 (d, J=8.0 Hz, 1H), 2.38 (m, 1H), 1.19 (t, J=14.4 Hz, 3H), 0.98 (d, J=6.4 Hz, 3H) 0.89 (d, J=6.8 Hz, 3H); LC-MS: m/z 351.9 (M+H).

### Step f: Synthesis of ethyl 3-methyl-2-(3-(4-(4,4,5,5-tetramethyl-1,3,2-dioxaborolan-2-yl)phenyl)isoxazol-5-yl)butanoate

To a stirred solution of ethyl 2-(3-(4-bromophenyl)isoxazol-5-yl)-3-methylbutanoate (3.8 g, 10.82 mmol) in dioxane (50 mL) was added bis-pinacolato diboron (4.12 g, 16.2 mmol) and KOAc (2.12 g, 21.6 mmol) at RT and degassed with nitrogen for 5 min. Pd(dppf)Cl₂·DCM (0.88 g, 1.08 mmol) was added into the reaction mixture and stirred at 100° C. for 2 h. Once the reaction was completed (monitored by TLC), the reaction mixture was diluted with EtOAc. The combined organic layer was washed with water and brine, dried over anhydrous sodium sulphate and concentrated under vacuum to afford the title compound as colourless liquid (4.1 g, 94%). ¹H NMR (400 MHz, DMSO-d₆): δ 7.90 (d, J=8.0 Hz, 2H), 7.79 (d, J=7.6 Hz, 2H), 7.05 (s, 1H), 4.18 (q, J=11.2 Hz, 2H), 3.85 (d, J=8.0 Hz, 1H), 2.44-2.33 (m, 1H), 1.31 (s, 12H), 1.20 (t, J=7.2 Hz, 3H), 0.98 (d, J=6.8 Hz, 3H), 0.89 (d, J=6.4 Hz, 3H); LC-MS: m/z 400.2 (M+H).

### Intermediate-4: (4-bromo-6-(trifluoromethyl)pyridazin-3-amine

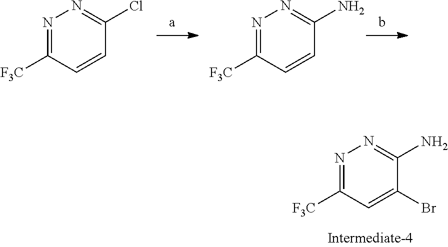 [SMILES: FC(F)(F)c1ccc(Cl)nn1 ; Nc1ccc(C(F)(F)F)nn1 ; Nc1nnc(C(F)(F)F)cc1Br]

### Step a: Synthesis of 6-(trifluoromethyl)pyridazin-3-amine

To a stirred solution of 3-chloro-6-(trifluoromethyl)pyridazine (1.0 g, 5.47 mmol) in methanolic ammonia (10 mL) was heated at 100° C. for 16 h in a steel bomb. The reaction mixture was concentrated under vacuum to get the crude product. The residue was purified by combiflash column chromatography using 50-60% ethyl acetate in hexane as eluent to afford the title compound as off-white solid (0.3 g, 34%). ¹H NMR (400 MHz, DMSO-d₆): δ 7.66 (d, J=9.2 Hz, 1H), 7.13 (bs, 2H), 6.88 (d, J=9.2 Hz, 1H).

### Step-b: Synthesis of (4-bromo-6-(trifluoromethyl)pyridazin-3-amine)

To a stirred solution of 6-(trifluoromethyl)pyridazin-3-amine (0.3 g, 1.8 mmol) in methanol (20 mL) was added NaHCO₃ (0.3 g, 3.6 mmol) at 0° C. and stirred for 5 min. Bromine (0.44 g, 2.7 mmol) wad added dropwise and stirred for 16 h at RT under nitrogen atmosphere. The reaction mixture was quenched with cold water and stirred for 1 h, the solid so formed was filtered and dried under vacuum to afford the title compound as off-white solid (0.25 g, 56%).

¹H NMR (400 MHz, CDCl₃): δ 7.79 (s, 1H), 5.67 (bs, 2H), LC-MS: m/z 242.0 (M+H).

### Intermediate-5: Ethyl 3-methyl-2-(4-(4,4,5,5-tetramethyl-1,3,2-dioxaborolan-2-yl)-1H-pyrazol-1-yl)butanoate

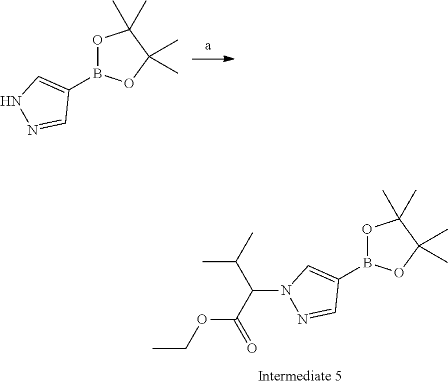 [SMILES: CC1(C)OB(c2cn[nH]c2)OC1(C)C ; CCOC(=O)C(C(C)C)n1cc(B2OC(C)(C)C(C)(C)O2)cn1]

### Step a: Synthesis of ethyl 3-methyl-2-(4-(4,4,5,5-tetramethyl-1,3,2-dioxaborolan-2-yl)-1H-pyrazol-1-yl)butanoate

To a solution of 4-(4,4,5,5-tetramethyl-1,3,2-dioxaborolan-2-yl)-1H-pyrazole (1 g, 5.15 mmol) and ethyl 2-bromo-3-methylbutanoate (1.28 mL, 6.18 mmol) in DMF (10 mL) was added Cs₂CO₃ (5.05 g, 15.45 mmol) and heated at 100° C. and stirred for 4 h. The reaction mixture was quenched with ice-cold water and extracted with EtOAc. The combined organic layer was washed with water and brine, dried over anhydrous sodium sulphate and concentrated under vacuum to give the residue which was purified by combiflash column chromatography using 30-35% ethyl acetate in hexane as eluent to afford the title compound as gray solid (0.45 g, 27%). LC-MS: m/z 323.1 (M+H).

### Intermediate-6: (2S,4R)-1-(L-valyl)-N-(4-(4-(difluoromethyl)thiazol-5-yl)benzyl)-4-hydroxypyrrolidine-2-carboxamide hydrochloride

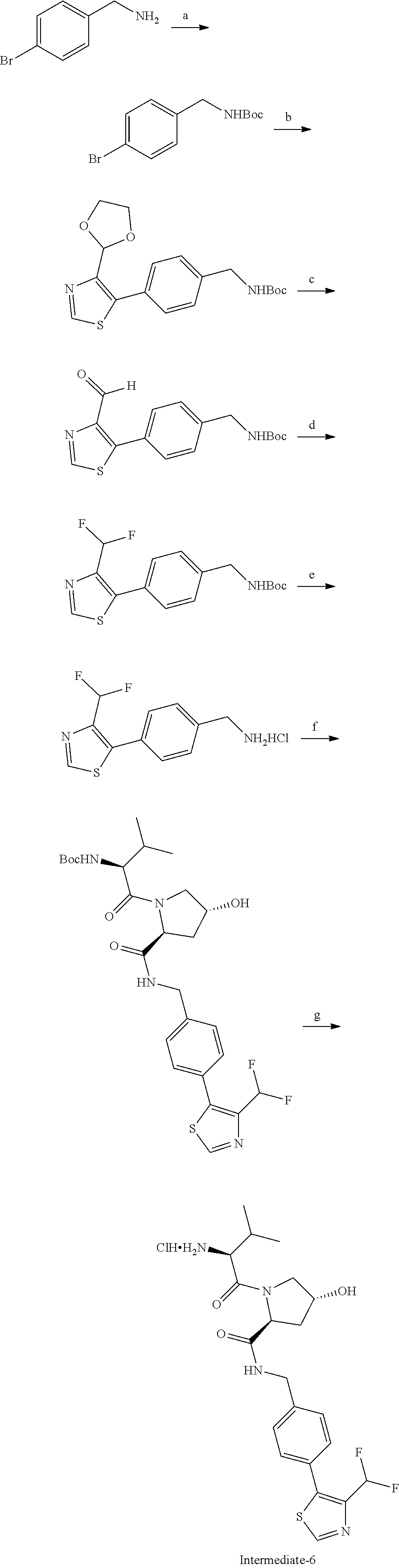

### Step a: Synthesis of tert-butyl (4-bromobenzyl)carbamate

To a solution of (4-bromophenyl)methanamine (5.0 g, 22.52 mmol) in DCM (50 mL) was dropwise added triethyl amine (6.82 g, 67.56 mmol) at 0° C. and stirred for 5 min. Boc anhydride (8.18 mL, 33.78 mmol) was added into the reaction mixture and stirred at RT for 16 h. The reaction mixture was quenched with water and extracted with DCM. The combined organic layer was washed with water and brine, dried over anhydrous sodium sulphate and concentrated under vacuum to give the residue which was stirred in hexane at RT and filtered on Buckner funnel to afford the title compound as off-white solid (6 g, 57%). ¹H NMR (400 MHz, DMSO-d₆): δ 7.50 (d, J=8 Hz, 2H), 7.43 (s, 1H), 7.18 (d, J=8 Hz, 2H), 4.07 (d, J=4 Hz, 2H), 1.38 (s, 9H); LC-MS: m/z 230 (M-56).

### Step b: Synthesis of tert-butyl (4-(4-(1,3-dioxolan-2-yl)thiazol-5-yl)benzyl)carbamate

To a stirred solution of tert-butyl (4-bromobenzyl)carbamate (0.5 g, 1.75 mmol) in DMF (10 mL) was added 4-(1,3-dioxolan-2-yl)thiazole (0.27 g, 1.75 mmol) and KOAc (0.34 g, 3.50 mmol) at RT and degassed with nitrogen for 10 min. Pd(OAc)₂ (0.02 g, 0.08 mmol) was added into the reaction mixture and heated at 100° C. for 16 h. Once the reaction was completed (monitored by TLC), the reaction mixture was diluted with EtOAc and washed with water and brine, dried over anhydrous sodium sulphate and concentrated under vacuum to get crude product. The crude product was purified by combiflash column chromatography using 40% ethyl acetate in hexane as eluent to afford the title compound as light-yellow solid (0.14 g, 17%). ¹H NMR (400 MHz, DMSO-d₆): δ 9.07 (s, 1H), 7.47-7.42 (m, 3H), 7.34 (d, J=8 Hz, 2H), 5.86 (s, 1H), 4.18-4.10 (m, 4H), 3.95-3.93 (m, 2H), 1.40 (s, 9H); LC-MS: m/z 363.1 (M+H).

### Step c: Synthesis of tert-butyl (4-(4-formylthiazol-5-yl)benzyl)carbamate

To a stirred solution of tert-butyl (4-(4-(1,3-dioxolan-2-yl)thiazol-5-yl)benzyl)carbamate (0.55 g, 1.51 mmol) in acetone (5.5 mL) were added PPTs (0.762 g, 3.03 mmol) at RT and heated at 50° C. for 16 h. The reaction mixture was evaporated and diluted with EtOAc. organic layer was washed with water and brine, dried over anhydrous sodium sulphate and concentrated under vacuum to get crude product. The crude product was purified by combiflash column chromatography using 45% ethyl acetate in hexane as eluent to afford the title compounds as yellow sticky material (0.28 g, 63%). ¹H NMR (400 MHz, DMSO-d₆): δ 9.90 (s, 1H), 9.19 (s, 1H), 7.62 (d, J=8 Hz, 2H), 7.49 (s, 1H), 7.37 (d, J=8 Hz, 2H), 4.19 (d, J=4 Hz, 2H), 1.40 (s, 9H). LC-MS: m/z 263 (M-56).

Step d: Synthesis of tert-butyl (4-(4-(difluoromethyl)thiazol-5-yl)benzyl)carbamate

To a stirred solution of tert-butyl (4-(4-formylthiazol-5-yl)benzyl)carbamate (0.57 g, 1.79 mmol) in DCM (15 mL) were added DAST (0.72 g, 4.48 mmol) at 0° C. and the reaction mixture was stirred for 2 h at same temperature. The reaction mixture was diluted with DCM and washed with sat. NaHCO₃ and organic layer was washed with brine, dried over anhydrous sodium sulphate and concentrated under vacuum to get crude product. The crude product was purified by combiflash column chromatography using 40% ethyl acetate in hexane as eluent to afford the title compounds as pale-yellow sticky material (0.34 g, 55%). ¹H NMR (400 MHz, DMSO-d₆): δ 9.20 (s, 1H), 7.47-7.36 (m, 5H), 7.00 (s, 1H), 4.18 (d, J=4 Hz, 2H), 1.40 (s, 9H); LC-MS: m/z 341.1 (M+H).

### Step e: Synthesis of (4-(4-(difluoromethyl)thiazol-5-yl)phenyl)methanamine hydrochloride

To a stirred solution of tert-butyl (4-(4-(difluoromethyl)thiazol-5-yl)benzyl)carbamate (0.34 g, 1.41 mmol) in DCM (2 mL) was added 4M HCl in 1,4-dioxane (2.0 mL) at RT and stirred for 16 h. The reaction mixture was evaporated under reduced pressure, the resultant residue was washed with diethyl ether and dried under vacuum to afford the title compound as off-white solid (0.29 g, crude yield). ¹H NMR (400 MHz, DMSO-d₆): δ 9.25 (d, J=8 Hz, 1H), 8.37 (bs, 2H), 7.68-7.57 (m, 4H), 7.05 (s, 1H), 4.12-4.08 (m, 2H); LC-MS: m/z 241.1 (M+H).

### Step f: Synthesis of tert-butyl((S)-1-((2S,4R)-2-((4-(4-(difluoromethyl)thiazol-5-yl)benzyl)carbamoyl)-4-hydroxypyrrolidin-1-yl)-3-methyl-1-oxobutan-2-yl)carbamate

To a solution of (4-(4-(difluoromethyl)thiazol-5-yl)phenyl)methanamine hydrochloride (0.41 g, 1.26 mmol), (2S,4R)-1-((tert-butoxycarbonyl)-L-valyl)-4-hydroxypyrrolidine-2-carboxylic acid (WO2012/37259) (0.29 g, 1.05 mmol) and HATU (0.6 g, 0.57 mmol) in DMF (10 mL) was dropwise added DIPEA (0.94 mL, 5.25 mmol) at 0° C. and slowly brought to RT. The reaction mixture was stirred for 2 h at RT. The reaction mixture was diluted with ethyl acetate and washed with cold brine, dried over anhydrous sodium sulphate and concentrated under vacuum to give the crude product. The crude was purified by combiflash column chromatography using 2.5% methanol in DCM as eluent to afford the title compound as off-white solid (0.5 g, 83%). ¹H NMR (400 MHz, DMSO-d₆): δ 9.20 (s, 1H), 8.54 (t, J=12 Hz, 1H), 7.45-7.40 (m, 4H), 7.00 (t, J=53.2 Hz, 1H), 5.13 (d, J=3.2 Hz, 1H), 4.42-4.31 (m, 3H), 4.03 (t, J=16 Hz, 1H), 3.67-3.58 (m, 2H), 2.69 (s, 2H), 2.04-1.89 (m, 3H), 1.4 (s, 9H), 0.88 (d, J=8 Hz, 3H), 0.81 (d, J=8 Hz, 3H); LC-MS: m/z 497.1 (M-56).

### Step g: Synthesis of (2S,4R)-1-(L-valyl)-N-(4-(4-(difluoromethyl)thiazol-5-yl)benzyl)-4-hydroxypyrrolidine-2-carboxamide hydrochloride

To a stirred solution of tert-butyl ((S)-1-((2S,4R)-2-((4-(4-(difluoromethyl)thiazol-5-yl)benzyl)carbamoyl)-4-hydroxypyrrolidin-1-yl)-3-methyl-1-oxobutan-2-yl)carbamate (0.5 g, 0.95 mmol) in DCM (2 mL) was added 4 M HCl in 1,4-dioxane (2.0 mL) at RT and stirred for 16 h. The reaction mixture was evaporated under reduced pressure, the resultant residue was washed with diethyl ether and dried under vacuum to afford the title compound as pale-yellow solid (0.45 g, 96%). ¹H NMR (400 MHz, DMSO-d₆): δ 9.21 (s, 1H), 8.96 (t, J=12 Hz, 1H), 8.07 (bs, 2H), 7.43 (bs, 3H), 7.03 (t, J=52.8 Hz, 1H), 4.52 (m, 1H), 4.41-4.32 (m, 2H), 4.04 (bs, 1H), 3.67-3.58 (m, 2H), 2.73 (s, 2H), 2.04-1.89 (m, 2H), 1.90 (m, 1H), 1.01 (d, J=6.8 Hz, 3H), 0.93 (d, J=6.8 Hz, 3H); LC-MS: m/z 453.2 (M+1).

### Intermediate-7: (2S,4R)-1-((S)-2-amino-3,3-dimethylbutanoyl)-N—((S)-1-(4-(4-(difluoromethyl)thiazol-5-yl)phenyl)ethyl)-4-hydroxypyrrolidine-2-carboxamide hydrochloride

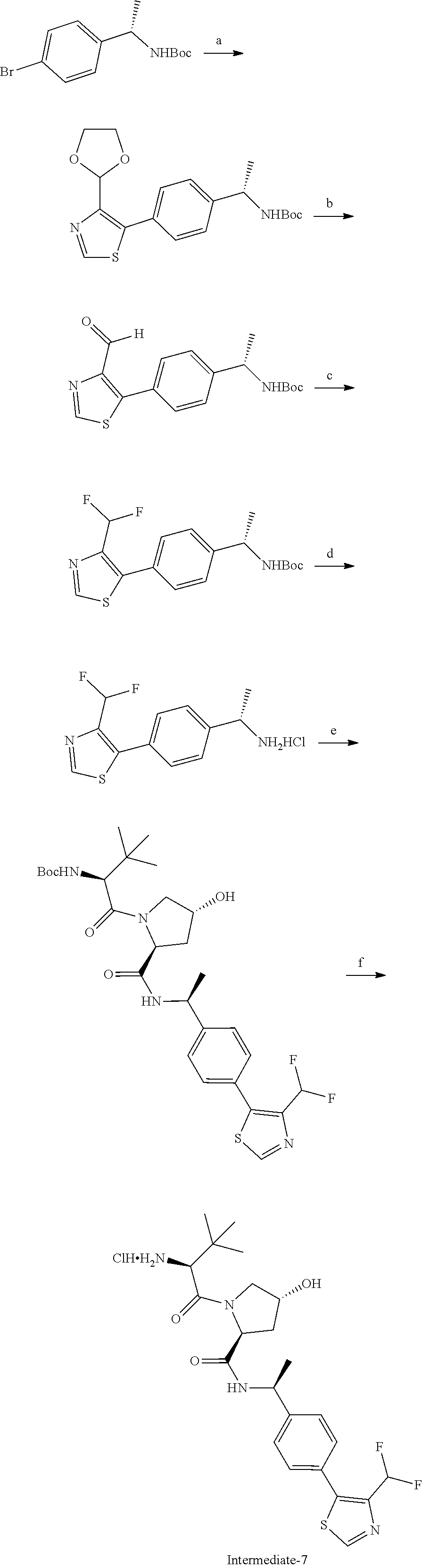

### Step a: Synthesis of tert-butyl (S)-(1-(4-(4-(1,3-dioxolan-2-yl)thiazol-5-yl)phenyl)ethyl)carbamate

To a stirred solution of tert-butyl (S)-(1-(4-bromophenyl)ethyl)carbamate (1.5 g, 5 mmol) in DMF (20 mL) was added 4-(1,3-dioxolan-2-yl)thiazole (0.94 g, 6 mmol) and KOAc (0.98 g, 10 mmol) at RT and degassed with nitrogen for 10 min. Pd(OAc)₂ (0.056 g, 0.25 mmol) was added into the reaction mixture and stirred at 90° C. for 16 h. Once the reaction was completed (monitored by TLC), All batches were mixed together and diluted with EtOAc. The combined organic layer was washed with water and brine, dried over anhydrous sodium sulphate and concentrated under vacuum to get crude product. The crude product was purified by combiflash column chromatography using 40% ethyl acetate in hexane as eluent to afford the title compound as sticky material (0.32 g, 17%). LC-MS: m/z 377.1 (M+H).

### Step b: Synthesis of tert-butyl (S)-(1-(4-(4-formylthiazol-5-yl)phenyl)ethyl)carbamate

To a stirred solution of tert-butyl (S)-(1-(4-(4-(1,3-dioxolan-2-yl)thiazol-5-yl)phenyl)ethyl)carbamate (0.3 g, 0.79 mmol) in acetone (20 mL) were added PPTs (0.4 g, 1.59 mmol) at RT and heated at 50° C. for 16 h. The reaction mixture was quenched with cold water and extracted with EtOAc. The combined organic layer was washed with water and brine, dried over anhydrous sodium sulphate and concentrated under vacuum to get crude product. The crude product was purified by combiflash column chromatography using 20% ethyl acetate in hexane as eluent to afford the title compounds as pale-yellow solid (0.15 g, 56%). LC-MS: m/z 333.1 (M+H).

### Step c: Synthesis of tert-butyl (S)-1-(4-(4-(difluoromethyl)thiazol-5-yl)phenyl)ethylcarbamate

To a stirred solution of tert-butyl (S)-(1-(4-(4-formylthiazol-5-yl)phenyl)ethyl)carbamate (0.15 g, 0.45 mmol) in DCM (20 mL) were added DAST (0.18 g, 1.12 mmol) at 0° C. and reaction mixture stirred for 3 h at the same temperature. The reaction mixture was diluted with DCM and washed with sat. NaHCO₃ and organic layer was washed with brine, dried over anhydrous sodium sulphate and concentrated under vacuum to get crude product. The crude product was purified by combiflash column chromatography using 1.5% methanol in DCM as eluent to afford the title compound as orange solid (0.115 g, 72%). ¹H NMR (400 MHz, DMSO-d₆): δ 9.20 (s, 1H), 7.42-7.50 (m, 5H), 7.01 (t, J=52.8 Hz, 1H), 4.68 (m, 1H), 1.37 (s, 9H), 1.32 (d, J=7.2 Hz, 1H).

### Step d: Synthesis of (S)-1-(4-(4-(difluoromethyl)thiazol-5-yl)phenyl)ethan-1-amine hydrochloride

To a stirred solution of tert-butyl (S)-1-(4-(4-(difluoromethyl)thiazol-5-yl)phenyl)ethylcarbamate (0.15 g, 0.42 mmol) was added 4M HCl in 1,4-dioxane (2.0 mL) at RT and stirred for 2 h. The reaction mixture was evaporated under reduced pressure, the resultant residue was washed with diethyl ether and dried under vacuum to afford the title compound as off-white solid (0.1 g, 81%). ¹H NMR (400 MHz, DMSO-d₆): δ 9.24 (s, 1H), 8.46 (bs, 1H), 7.66-7.48 (m, 4H), 7.05 (t, J=53.2 Hz, 1H), 4.49 (m, 1H), 1.52 (d, J=6.8 Hz, 3H), LC-MS: m/z 255.1 (M+H).

### Step e: Synthesis of tert-butyl ((S)-1-((2S,4R)-2-(((S)-1-(4-(4-(difluoromethyl)thiazol-5-yl)phenyl)ethyl)carbamoyl)-4-hydroxypyrrolidin-1-yl)-3,3-dimethyl-1-oxobutan-2-yl)carbamate

To a solution of (S)-1-(4-(4-(difluoromethyl)thiazol-5-yl)phenyl)ethanamine hydrochloride (0.10 g, 0.29 mmol), (2S,4R)-1-((S)-2-((tert-butoxycarbonyl)amino)-3,3-dimethylbutanoyl)-4-hydroxypyrrolidine-2-carboxylic acid (WO2012/37259) (0.1 g, 0.34 mmol) and HATU (0.16 g, 0.43 mmol) in DCM (5 mL) was dropwise added DIPEA (0.26 mL, 1.45 mmol) at 0° C. and slowly brought to RT. The reaction mixture was stirred for 16 h at RT.

The reaction mixture was diluted with ethyl acetate and washed with cold brine, dried over anhydrous sodium sulphate and concentrated under vacuum to give the crude product. The crude product was purified by combiflash column chromatography using 2.5% methanol in DCM as eluent to afford the title compound as off-white solid (0.1 g, 98%). ¹H NMR (400 MHz, DMSO-d₆): δ 9.19 (s, 1H) 8.42 (d, J=7.2 Hz, 1H), 7.47-7.40 (m, 4H), 7.01 (t, J=53.2 Hz, 1H), 6.34 (d, J=9.2 Hz, 1H), 5.12 (d, J=3.6 Hz, 1H), 4.93 (m, 1H), 4.45 (m, 1H), 4.28 (bs, 1H), 4.13 (d, J=8.8 Hz, 1H), 3.57 (m, 2H), 2.03 (m, 1H), 1.78 (m, 1H), 1.39 (s, 9H), 1.37 (d, J=8 Hz, 3H), 0.92 (s, 9H); LC-MS: m/z 481.2 (M−100).

### Step f: Synthesis of (2S,4R)-1-((S)-2-amino-3,3-dimethylbutanoyl)-N—((S)-1-(4-(4-(difluoromethyl)thiazol-5-yl)phenyl)ethyl)-4-hydroxypyrrolidine-2-carboxamide hydrochloride

To a stirred solution of tert-butyl ((S)-1-((2S,4R)-2-(((S)-1-(4-(4-(difluoromethyl)thiazol-5-yl)phenyl)ethyl)carbamoyl)-4-hydroxypyrrolidin-1-yl)-3,3-dimethyl-1-oxobutan-2-yl)carbamate (0.1 g, 0.17 mmol) in DCM (2 mL) was added 4 M HCl in 1,4-dioxane (2.0 mL) at RT and stirred for 1 h. The reaction mixture was evaporated under reduced pressure, the resultant residue was washed with diethyl ether and dried under vacuum to afford the title compound as off-white solid (0.08 g, 89%). LC-MS: m/z 481.5 (M+1).

### Intermediate 8: Ethyl 1-(4-(4,4,5,5-tetramethyl-1,3,2-dioxaborolan-2-yl)phenyl)piperidine-4-carboxylate

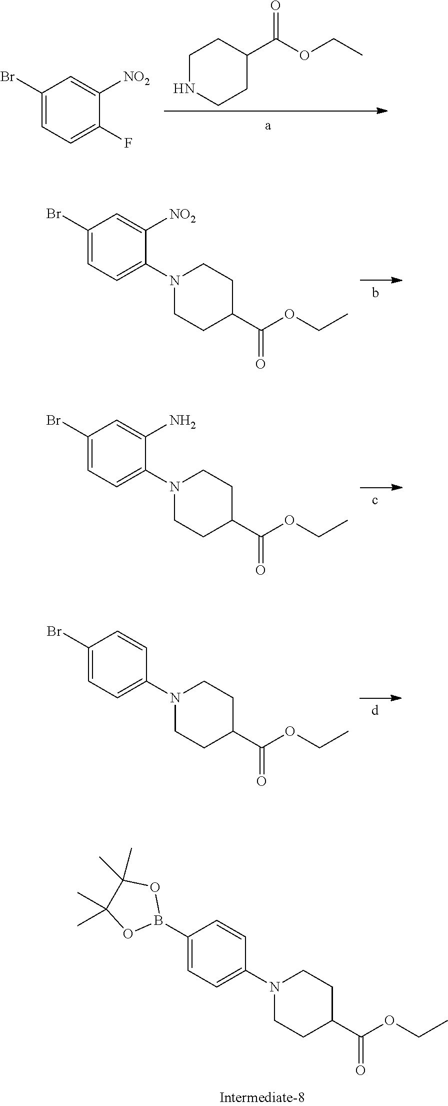

Intermediate-8 was prepared by the procedure similar to the one described in intermediate-2 with appropriate variations in reactants, quantities of reagents, protections and deprotections, solvents and reaction conditions. ¹H NMR (400 MHz, DMSO-d6): δ 7.48 (d, J=8.4 Hz, 2H), 6.88 (d, J=8.4 Hz, 2H), 4.05 (q, J=7.2 Hz, 2H) 3.73 (d, J=13.2 Hz, 2H), 2.83 (td, J=13.2, 2.4 Hz, 2H), 2.54 (m, 1H), 1.86 (m, 2H), 1.60 (m, 2H), 1.24 (s, 12H), 1.17 (t, J=7.2 Hz, 3H).

### Intermediate 9: Tert-butyl 2-(4-(4-(4,4,5,5-tetramethyl-1,3,2-dioxaborolan-2-yl)phenyl)-3,6-dihydropyridin-1(2H)-yl)acetate

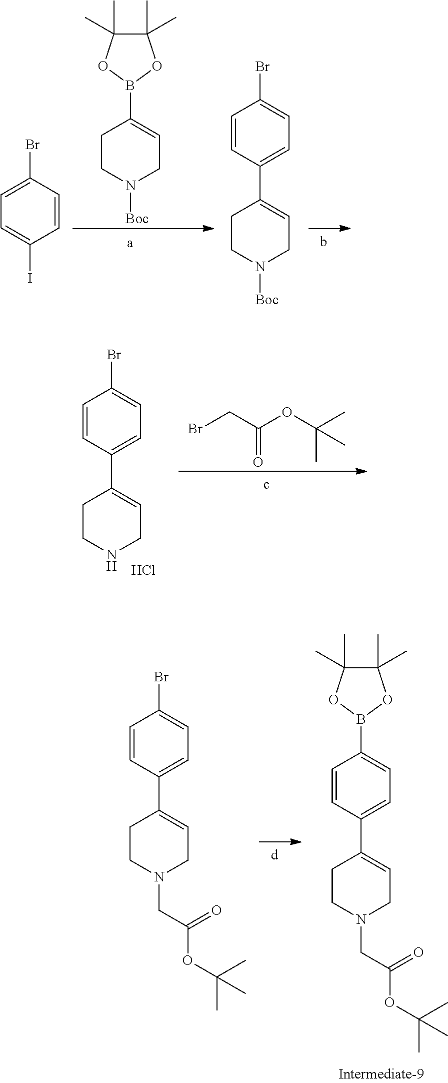

### Step-a: Synthesis of tert-butyl 4-(4-bromophenyl)-3,6-dihydropyridine-1(2H)-carboxylate

To a stirred solution of 1-bromo-4-iodobenzene (1 g, 3.53 mmol), tert-butyl 4-(4,4,5,5-tetramethyl-1,3,2-dioxaborolan-2-yl)-3,6-dihydropyridine-1(2H)-carboxylate (0.87 g, 2.82 mmol) in 1,4-dioxane (10 mL) and water (2.5 mL) was added K₂CO₃ (1.22 g, 8.83 mmol) and degassed with nitrogen for 15 min. Pd(dppf)Cl₂·DCM (0.3 g, 0.35 mmol) was added in to the reaction mixture and heated for 16 h at 100° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 50% ethyl acetate in hexane as eluent to afford the title compound as off-white solid (1.3 g, 83%). ¹H NMR (400 MHz, DMSO-d6): δ 7.53 (d, J=8 Hz, 2H), 7.39 (d, J=8 Hz, 2H), 6.20 (s, 1H), 3.98 (bs, 2H), 3.52 (t, J=5.2 Hz, 2H), 2.43 (bs, 2H), 1.42 (s, 9H); LC-MS: m/z 338.0 (M+H).

### Step-b: synthesis of 4-(4-Bromophenyl)-1,2,3,6-tetrahydropyridine hydrochloride

To a stirred solution of tert-butyl 4-(4-bromophenyl)-3,6-dihydropyridine-1(2H)-carboxylate (1 g, 2.95 mmol) in DCM (15 mL) at 0° C. was added 4M HCl in 1,4-dioxane (5 mL). The reaction mixture was stirred at room temperature for 2 h. After completion of the reaction (monitored by TLC), the reaction mixture was concentrated under reduced pressure and washed with diethyl ether to afford the title compound (0.8 g, 98%).

¹H NMR (400 MHz, DMSO-d6): δ 9.35 (bs, 1H), 7.57 (d, J=8 Hz, 2H), 7.43 (d, J=8 Hz, 2H), 6.24 (s, 1H), 3.72 (bs, 2H), 3.28 (bs, 2H), 2.66 (bs, 2H). LC-MS: m/z 238 (M+1).

### Step-c: Synthesis of tert-butyl 2-(4-(4-bromophenyl)-3,6-dihydropyridin-1(2H)-yl)acetate

To a stirred solution of 4-(4-Bromophenyl)-1,2,3,6-tetrahydropyridine hydrochloride (0.8 g, 2.91 mmol) in DMF (10 mL) was added DIPEA (1.12 g, 8.73 mmol) at RT and stirred for 15 min. Tert-butyl 2-bromoacetate (0.56 g, 2.91 mmol) was added to the reaction mixture and stirred for 4 h at RT under nitrogen atmosphere. The reaction mixture was quenched with cold water and stirred for 1 h. The solid so formed was filtered and dried under vacuum to afford the title compound as yellow solid (1.3 g, 97%).

¹H NMR (400 MHz, DMSO-d₆): δ 7.51 (d, J=8 Hz, 2H), 7.38 (d, J=8 Hz, 2H), 6.19 (s, 1H), 3.22 (s, 2H), 3.20 (m, 2H), 2.74 (m, 2H), 2.44 (bs, 2H), 1.42 (s, 9H): LC-MS: m/z 352.2 (M+H).

### Step-d: Synthesis of tert-butyl 2-(4-(4-(4,4,5,5-tetramethyl-1,3,2-dioxaborolan-2-yl)phenyl)-3,6-dihydropyridin-1(2H)-yl)acetate

To a stirred solution of tert-butyl 2-(4-(4-bromophenyl)-3,6-dihydropyridin-1(2H)-yl) acetate (0.5 g, 1.41 mmol) in dioxane (20 mL) was added bis(pinacolato)diboron (0.43 g, 1.70 mmol) and KOAc (0.28 g, 2.83 mmol) at RT and the mixture was degassed with nitrogen for 5 min. Pd(dppf)Cl₂·DCM (0.11 g, 0.14 mmol) was added into the reaction mixture and heated at 100° C. for 4 h. After completion of the reaction (monitored by TLC), the reaction mixture was diluted with EtOAc. The combined organic layer was washed with water and brine, dried over anhydrous sodium sulphate and concentrated under vacuum to afford the title compound as off-white solid (0.5 g, 88%).

¹H NMR (400 MHz, DMSO-d₆): δ 7.63 (d, J=10.8 Hz, 2H), 7.43 (d, J=10.8 Hz, 2H), 6.22 (s, 1H), 3.22 (bs, 4H), 2.73 (m, 2H), 2.46 (bs, 2H), 1.42 (s, 9H), 1.28 (s, 12H): LC-MS: m/z 400.4 (M+H).

The intermediate compounds listed in below Table-1 were prepared by the procedure similar to the one described in intermediate-9 with appropriate variations in reactants, quantities of reagents, protections and deprotections, solvents and reaction conditions. The characterization data of the compounds are summarized herein the below table.

| Intermediate | Structure | Characterization Data |
|---|---|---|
| Tert-butyl 2-(3-(4-(4,4,5,5- tetramethyl-1,3,2- dioxaborolan-2- yl)phenyl)pyrrolidin-1- yl)acetate | 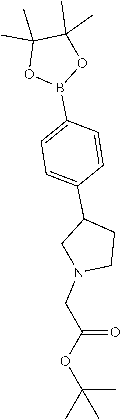 [SMILES: CC(C)(C)OC(=O)CN1CCC(c2ccc(B3OC(C)(C)C(C)(C)O3)cc2)C1] | ¹H NMR (400 MHz, DMSO- d6): δ 7.63 (d, J = 8 Hz, 2H), 7.34 (d, J = 8 Hz, 2H), 3.22 (m, 2H), 2.98 (m, 1H), 2,82 (m, 2H), 2.66 (m, 2H), 2.25 (m, 1H), 1.76 (m, 1H), 1.45 (s, 9H), 1.32 (s, 12H): LC-MS: m/z 388.3 (M + H). |
| Tert-butyl 2-(4-(3-(4,4,5,5- tetramethyl-1,3,2- dioxaborolan-2-yl)phenyl)- 3,6-dihydropyridin-1(2H)- yl)acetate | 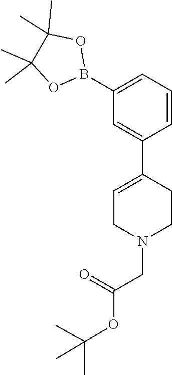 [SMILES: CC(C)(C)OC(=O)CN1CC=C(c2cccc(B3OC(C)(C)C(C)(C)O3)c2)CC1] | LC-MS: m/z 400.3 (M + H). |
| Tert-butyl 2-(4-(4-(4,4,5,5- tetramethyl-1,3,2- dioxaborolan-2- yl)phenoxy)piperidin-1- yl)acetate | 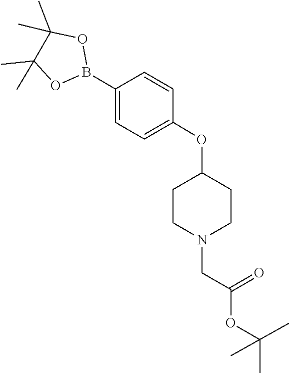 [SMILES: CC(C)(C)OC(=O)CN1CCC(Oc2ccc(B3OC(C)(C)C(C)(C)O3)cc2)CC1] | ¹H NMR (400 MHz, DMSO- d₆): δ 7.59 (d, J = 8 Hz, 2H), 6.93 (d, J = 8 Hz, 2H), 4.42 (m, 1H), 3.11 (s, 2H), 2.75 (m, 2H), 2.44 (m, 2H), 1.90 (m, 2H), 1.62 (m, 2H), 1.41 (s, 9H), 1.26 (s, 12H): LC-MS: m/z 418.3 (M + H). |

### Intermediate 10: Tert-butyl 4-(3-fluoro-4-(4,4,5,5-tetramethyl-1,3,2-dioxaborolan-2-yl)phenyl)piperazine-1-carboxylate

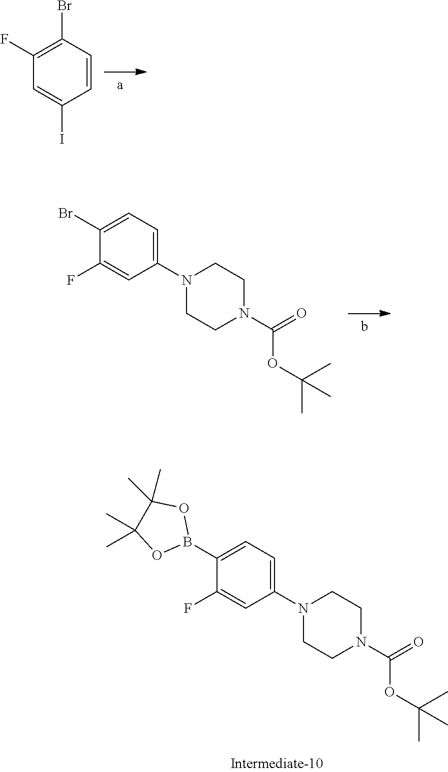 [SMILES: Fc1cc(I)ccc1Br ; CC(C)(C)OC(=O)N1CCN(c2ccc(Br)c(F)c2)CC1 ; CC(C)(C)OC(=O)N1CCN(c2ccc(B3OC(C)(C)C(C)(C)O3)c(F)c2)CC1]

### Step-a: Synthesis of tert-butyl 4-(4-bromo-3-fluorophenyl)piperazine-1-carboxylate

To a stirred solution of 4-bromo-2-fluoro-1-iodobenzene (5 g, 16.72 mmol), N-Boc piperazine (1.24 g, 6.6 mmol) in toluene (50 mL) was added Cs₂CO₃ (8.68 g, 26.6 mmol) and degassed with nitrogen for 10 min followed by Pd₂(dba)₃ (0.6 g, 0.66 mmol) and BINAP (0.41 g, 0.66 mmol) was added and the reaction mixture was heated for 16 h at 100° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 40% ethyl acetate in hexane as eluent to afford the title compound as yellow solid (1.6 g, 26%). ¹H NMR (400 MHz, CDCl₃): δ 7.45 (t, J=8.8 Hz 1H), 6.95 (dd, J=8.8, 2.8 Hz, 1H), 6.75 (dd, J=8.8, 2.8 Hz, 1H), 3.42 (m, 4H), 3.15 (m, 4H), 1.41 (s, 9H); LC-MS: m/z 361.0 (M+H).

### Step-b: Synthesis of tert-butyl 4-(3-fluoro-4-(4,4,5,5-tetramethyl-1,3,2-dioxaborolan-2-yl)phenyl)piperazine-1-carboxylate

To a stirred solution of tert-butyl 4-(4-bromo-3-fluorophenyl)piperazine-1-carboxylate (1.6 g g, 4.46 mmol) in dioxane (10 mL) was added bis(pinacolato)diboron (1.69 g, 6.69 mmol) and KOAc (1.3 g, 13.4 mmol) at RT and degassed with nitrogen for 5 min. Pd(dppf)Cl₂·DCM (0.36 g, 0.44 mmol) was added into the reaction mixture and heated at 90° C. for 16 h. Once the reaction was completed (monitored by TLC), the reaction mixture was diluted with EtOAc. The combined organic layer was washed with water and brine, dried over anhydrous sodium sulphate and concentrated under vacuum to afford the title compound as yellow solid (0.9 g, 50%). ¹H NMR (400 MHz, DMSO-d₆): δ 7.44 (2, J=8.4 Hz, 1H), 6.72 (dd, J=8.4, 2 Hz, 1H), 6.64 (dd, J=9.2, 2 Hz, 1H), 3.42 (m, 4H), 3.24 (m, 4H), 1.41 (s, 9H), 1.25 (s, 12H); LC-MS: m/z 406.9 (M+H).

### Intermediate 11: Ethyl 2-(4-(4-(4,4,5,5-tetramethyl-1,3,2-dioxaborolan-2-yl)phenyl)-3,6-dihydropyridin-1(2H)-yl)propanoate

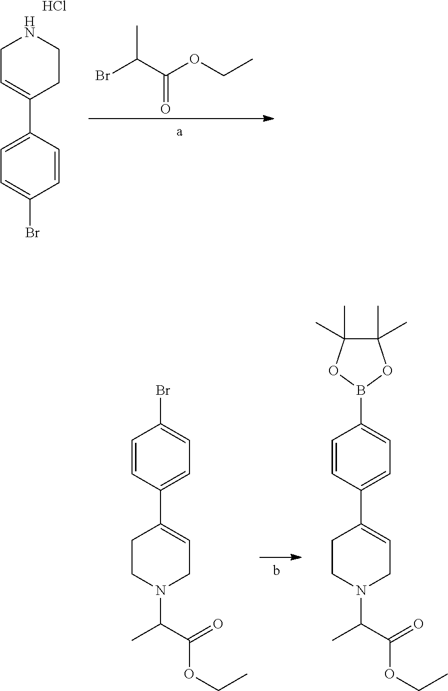

### Step-a: Synthesis of ethyl 2-(4-(4-bromophenyl)-3,6-dihydropyridin-1(2H)-yl)propanoate

To a stirred solution of 4-(4-Bromophenyl)-1,2,3,6-tetrahydropyridine hydrochloride (1 g, 3.64 mmol) in DMF (10 mL) was added DIPEA (2.35 g, 18.2 mmol) at RT and stirred for 15 min. Ethyl 2-bromopropanoate (0.79 g, 4.37 mmol) was added to the reaction mixture and stirred for 4 h at RT under nitrogen atmosphere. The reaction mixture was quenched with cold water and stirred for 1 h, the solid so formed was filtered and dried under vacuum to afford the title compound as off-white solid (0.8 g, 56%).

¹H NMR (400 MHz, DMSO-d₆): δ 7.46 (d, J=8 Hz, 2H), 7.33 (d, J=8 Hz, 2H), 6.15 (s, 1H), 4.07 (m, 2H), 3.39 (q, J=6.8 Hz, H), 3.22 (bs, 2H), 2.78 (m, 2H), 2.38 (bs, 2H), 1.18 (m, 6H): LC-MS: m/z 338 (M+H).

### Step-b: Synthesis of ethyl 2-(4-(4-(4,4,5,5-tetramethyl-1,3,2-dioxaborolan-2-yl)phenyl)-3,6-dihydropyridin-1(2H)-yl)propanoate

To a stirred solution of ethyl 2-(4-(4-Bromophenyl)-3,6-dihydropyridin-1(2H)-yl)propanoate (0.8 g, 2.36 mmol) in dioxane (15 mL) was added bis(pinacolato)diboron (0.9 g, 3.54 mmol) and KOAc (0.69 g, 7.09 mmol) at RT and degassed with nitrogen for 5 min. Pd(dppf)Cl₂·DCM (0.19 g, 0.23 mmol) was added into the reaction mixture and heated at 80° C. for 16 h. Once the reaction was completed (monitored by TLC), the reaction mixture was diluted with EtOAc. The combined organic layer was washed with water and brine, dried over anhydrous sodium sulphate and concentrated under vacuum to afford the title compound as off-white solid (0.9 g, 98%).

¹H NMR (400 MHz, DMSO-d₆): δ 7.62 (d, J=10.8 Hz, 2H), 7.42 (d, J=10.8 Hz, 2H), 6.22 (s, 1H), 4.11 (m, 2H), 3.43 (q, J=6.8 Hz, 1H), 3.28 (bs, 2H), 2.84 (m, 2H), 2.45 (bs, 2H), 1.28 (s, 12H), 1.21 (m 6H): LC-MS: m/z 386.3 (M+H).

### Intermediate 12: Ethyl 2-(4′-(4,4,5,5-tetramethyl-1,3,2-dioxaborolan-2-yl)-2,3,4,5-tetrahydro-[1,1′-biphenyl]-4-yl)acetate

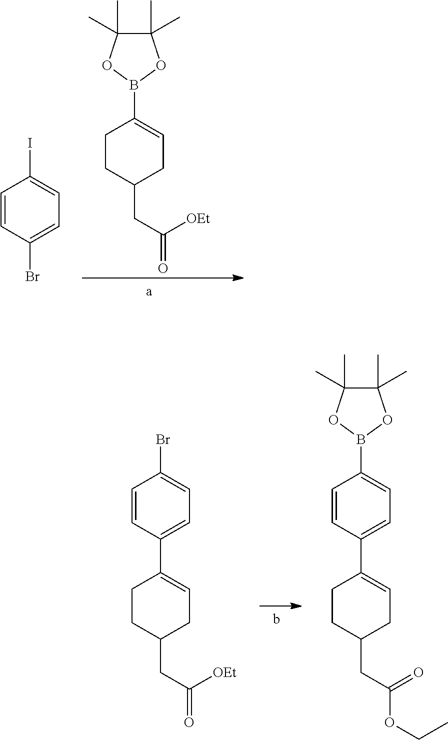 [SMILES: Brc1ccc(I)cc1 ; CCOC(=O)CC1CC=C(B2OC(C)(C)C(C)(C)O2)CC1 ; CCOC(=O)CC1CC=C(c2ccc(Br)cc2)CC1 ; CCOC(=O)CC1CC=C(c2ccc(B3OC(C)(C)C(C)(C)O3)cc2)CC1]

### Step-a: Synthesis of ethyl 2-(4′-bromo-2,3,4,5-tetrahydro-[1,1′-biphenyl]-4-yl)acetate

To a stirred solution of 1-bromo-4-iodobenzene (0.8 g, 2.82 mmol) and ethyl 2-(4-(4,4,5,5-tetramethyl-1,3,2-dioxaborolan-2-yl)cyclohex-3-en-1-yl)acetate (0.99 g, 3.39 mmol) in 1,4-dioxane (8 mL) and water (1 mL) was added K₂CO₃ (0.97 g, 7.07 mmol) and degassed with nitrogen for 15 min followed by Pd(dppf)Cl₂·DCM (0.23 g, 0.28 mmol) was added and the reaction mixture was heated for 6 h at 100° C. in a sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 50% ethyl acetate in hexane as eluent to afford the title compound as colourless liquid (0.48 g, 52%). ¹H NMR (400 MHz, DMSO-d₆): δ 7.49 (d, J=8.4 Hz, 2H), 7.36 (d, J=8.4 Hz, 2H), 6.16 (d, J=2 Hz, 1H), 4.08 (q, J=6.8 Hz, 2H), 2.39 (bs, 2H), 2.31 (d, J=7.2 Hz, 2H), 2.28 (m, 1H), 1.83-2.05 (m, 3H), 1.38 (m, 1H), 1.19 (t, J=6.8 Hz, 3H).

### Step-b: Synthesis of ethyl 2-(4′-(4,4,5,5-tetramethyl-1,3,2-dioxaborolan-2-yl)-2,3,4,5-tetrahydro-[1,1′-biphenyl]-4-yl)acetate

To a stirred solution of ethyl 2-(4′-bromo-2,3,4,5-tetrahydro-[1,1′-biphenyl]-4-yl)acetate (0.5 g, 1.54 mmol) in dioxane (5 mL) was added bis(pinacolato)diboron (0.58 g, 2.32 mmol) and KOAc (0.38 g, 3.86 mmol) at RT and degassed with nitrogen for 5 min. Pd(dppf)Cl₂·DCM (0.12 g, 0.15 mmol) was added into the reaction mixture and heated at 100° C. for 3 h. Once the reaction was completed (monitored by TLC), the reaction mixture was diluted with EtOAc. The combined organic layer was washed with water and brine, dried over anhydrous sodium sulphate and concentrated under vacuum to afford the title compound as colourless sticky material (0.48 g, 83%).

¹H NMR (400 MHz, DMSO-d₆): δ 7.61 (d, J=8 Hz, 2H), 7.41 (d, J=8 Hz, 2H), 6.19 (bs, 1H), 4.07 (q, J=6.8 Hz, 2H), 2.41 (bs, 2H), 2.31 (m, 3H), 1.85-2.04 (m, 3H), 1.40 (m, 1H), 1.28 (s, 12H), 1.22 (t, J=6.8 Hz, 3H).

### Intermediate 13: Ethyl 4′-(4,4,5,5-tetramethyl-1,3,2-dioxaborolan-2-yl)-2,3,4,5-tetrahydro-[1,1′-biphenyl]-4-carboxylate

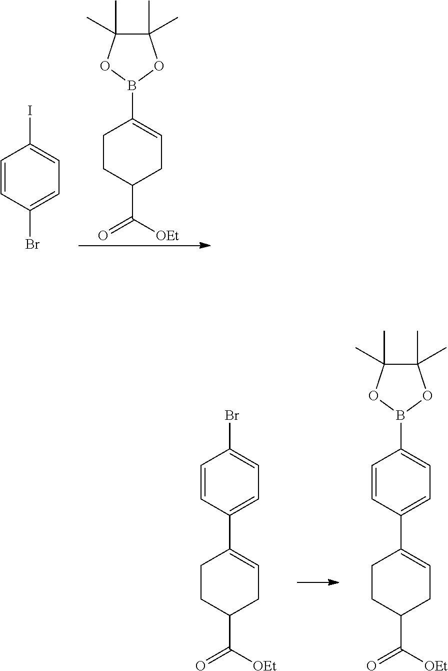

Intermediate-13 was prepared by the procedure similar to the one described in intermediate-12 with appropriate variations in reactants, quantities of reagents, protections and deprotections, solvents and reaction conditions. ¹H NMR (400 MHz, DMSO-d₆): δ 7.61 (d, J=8 Hz, 2H), 7.41 (d, J=8 Hz, 2H), 6.22 (bs, 1H), 4.09 (q, J=6.8 Hz, 2H), 2.57 (m, 1H), 2.30-2.50 (m, 4H), 2.08 (m, 1H), 1.71 (m, 1H), 1.28 (s, 12H), 1.20 (t, J=6.8 Hz, 3H); LC-MS: m/z 357.3 (M+H).

### Intermediate 14: Methyl 5-((4-(4,4,5,5-tetramethyl-1,3,2-dioxaborolan-2-yl)phenyl)amino)pentanoate

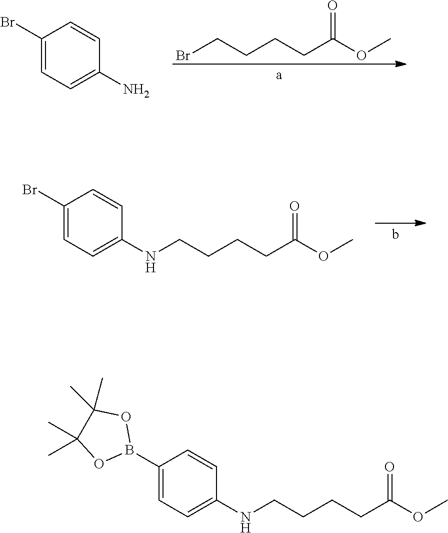

### Step-a: Synthesis of methyl 5-((4-bromophenyl)amino)pentanoate

To a stirred solution of 4-bromo aniline (0.5 g, 2.90 mmol) in DMF (5 mL) was added K₂CO₃ (0.14 g, 2.90 mmol) at RT and stirred for 15 min. Methyl-2-bromopropanoate (1.13 g, 5.81 mmol) was added into the reaction mixture and stirred for 24 h at 60° C. under nitrogen atmosphere. Once the reaction was completed (monitored by TLC), the reaction mixture was diluted with ice cold water and extracted with EtOAc. The combined organic layer was washed with water and brine, dried over anhydrous sodium sulphate and concentrated under vacuum to give the residue which was purified by combiflash column chromatography using 10% ethyl acetate in hexane as eluent to afford the title compound as off-white solid (0.28 g, 33%). ¹H NMR (400 MHz, DMSO-d₆): δ 7.22 (dd, J=8.8, 1.6 Hz, 2H), 6.53 (dd, J=8.8, 1.6 Hz, 2H), 3.61 (s, 3H), 3.00 (m, 2H), 2.37 (t, J=7.6 Hz, 2H), 1.52-1.68 (m, 4H): LC-MS: m/z 288.0 (M+H).

### Step-b: Synthesis of methyl 5-((4-(4,4,5,5-tetramethyl-1,3,2-dioxaborolan-2-yl)phenyl)amino)pentanoate

To a stirred solution of methyl 5-((4-bromophenyl)amino)pentanoate (0.28 g, 0.97 mmol) in dioxane (5 mL) was added bis(pinacolato)diboron (0.32 g, 1.26 mmol) and KOAc (0.29 g, 2.93 mmol) at RT and degassed with nitrogen for 5 min. Pd(dppf)Cl₂·DCM (0.08 g, 0.09 mmol) was added into the reaction mixture and heated at 80° C. for 16 h. Once the reaction was completed (monitored by TLC), the reaction mixture was filtered through celite and washed with EtOAc. The combined organic layer was concentrated under vacuum to give the residue which was purified by combiflash column chromatography using 10% ethyl acetate in hexane as eluent to afford the title compound as yellow solid (0.15 g, 46%).

¹H NMR (400 MHz, DMSO-d₆): δ 7.41 (d, J=8 Hz, 2H), 6.54 (d, J=8 Hz, 2H), 6.04 (m, 1H), 3.61 (s, 3H), 3.05 (m, 2H), 2.37 (t, J=7.6 Hz, 2H), 1.56-1.68 (m, 4H): 1.28 (s, 12H): LC-MS: m/z 334.05 (M+H).

### Intermediate 15: Tert-butyl 2-(3-(4-(4,4,5,5-tetramethyl-1,3,2-dioxaborolan-2-yl)phenyl)azetidin-1-yl)acetate

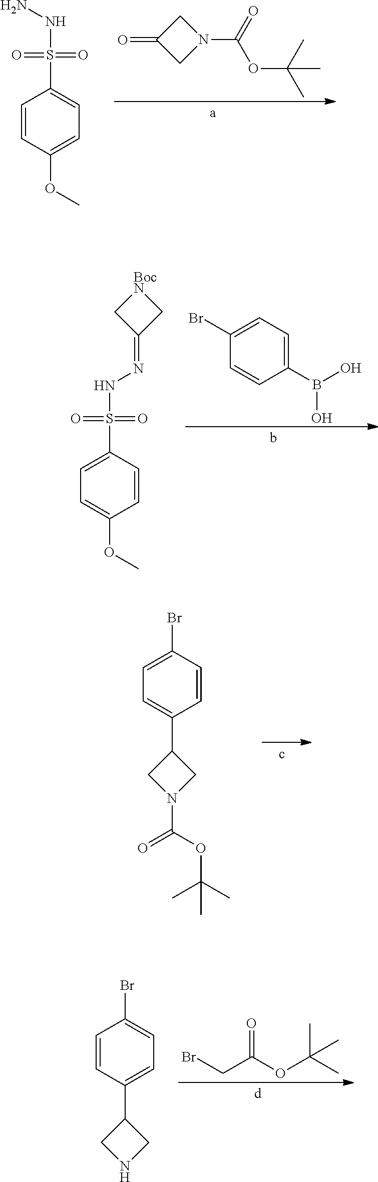

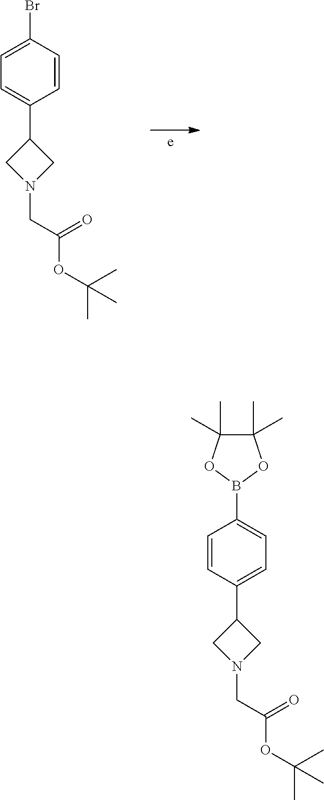 [SMILES: CC(C)(C)OC(=O)CN1CC(c2ccc(Br)cc2)C1 ; CC(C)(C)OC(=O)CN1CC(c2ccc(B3OC(C)(C)C(C)(C)O3)cc2)C1]

### Step-a: Synthesis of tert-butyl 3-(2-((4-methoxyphenyl)sulfonyl)hydrazineylidene)azetidine-1-carboxylate

To a stirred solution of 4-methoxybenzenesulfonohydrazide (2 g, 11.68 mmol) and tert-butyl 3-oxoazetidine-1-carboxylate (2.36 g, 11.68 mmol) in toluene (40 mL) and the reaction mixture was heated for 16 h at 50° C. in sealed tube. Once the reaction was completed (monitored by TLC), reaction mass was concentrated under vacuum to get the residue which was purified by combiflash column chromatography using 40% ethyl acetate in hexane as eluent to afford the title compound as white solid (1.75 g, 42%). ¹H NMR (400 MHz, DMSO-d₆): δ 10.81 (bs, 1H), 7.78 (dd, J=6.8, 2 Hz, 2H), 7.16 (dd, J=6.8, 2 Hz, 2H), 4.50 (bs, 4H), 3.88 (s, 3H), 1.41 (s, 9H); LC-MS: m/z 353.9 (M−H).

### Step-b: Synthesis of tert-butyl 3-(4-bromophenyl)azetidine-1-carboxylate

To a stirred solution of tert-butyl 3-(2-((4-methoxyphenyl)sulfonyl)hydrazineylidene)azetidine-1-carboxylate (1.2 g, 3.37 mmol), (4-bromophenyl)boronic acid (1.01 g, 5.06 mmol) in dioxane (35 mL) was added Cs₂CO₃ (1.65 g, 5.06 mmol) and degassed with nitrogen for 30 min. The reaction mixture was heated for 16 h at 110° C. in sealed tube. Once the reaction was completed (monitored by TLC), the reaction mixture was diluted with EtOAc and filtered. The filtrate was concentrated under vacuum to give the residue which was purified by combiflash column chromatography using 20% ethyl acetate in hexane as eluent to afford the title compound as colourless liquid (0.36 g, 34%). ¹H NMR (400 MHz, CDCl₃): δ 7.47 (d, J=8.4 Hz, 2H), 7.19 (d, J=8.8 Hz, 2H), 4.32 (m, 2H), 3.92 (m, 2H), 3.67 (m, 1H), 1.46 (s, 9H); LC-MS: m/z 212 (M−100).

### Step-c: Synthesis of 3-(4-bromophenyl)azetidine

To a stirred solution of tert-butyl 3-(4-bromophenyl)azetidine-1-carboxylate (0.36 g, 1.15 mmol) in DCM (3 mL) was added trifluoro acetic acid (0.3 mL) at 0° C. and then slowly brought to RT and stirred at RT for 1 h. The reaction mixture was evaporated under reduced pressure, the resultant residue was washed with diethyl ether and dried under vacuum to afford the title compound off-white solid (0.28 g, 97.7%). ¹H NMR (400 MHz, CDCl₃): δ 9.85 (bs, 1H), 7.53 (d, J=7.6 Hz, 2H), 7.25 (d, J=7.6 Hz, 2H), 4.35 (m, 2H), 4.20 (m, 3H); LC-MS: m/z 212 (M+H).

### Step-d: Synthesis of tert-butyl 2-(3-(4-bromophenyl)azetidin-1-yl)acetate

To a stirred solution of 3-(4-bromophenyl)azetidine (0.28 g, 1.12 mmol) in DMF (5 mL) was added DIPEA (0.72 g, 5.63 mmol) at RT and stirred for 15 min. Tert-butyl 2-bromoacetate (0.24 g, 1.24 mmol) was added to the reaction mixture and stirred for 2 h at RT under nitrogen atmosphere. The reaction mixture was quenched with cold water and stirred for 1 h, the solid so formed was filtered and dried under vacuum to afford the title compound as pale-yellow sticky solid (0.28 g, 76%). ¹H NMR (400 MHz, CDCl₃): δ 7.40 (d, J=7.6 Hz, 2H), 7.14 (d, J=7.6 Hz, 2H), 3.85 (m, 2H), 3.70 (m, 1H), 3.22 (m, 4H), 1.44 (s, 9H); LC-MS: m/z 326.1 (M+H).

### Step-e: Synthesis of tert-butyl 2-(3-(4-(4,4,5,5-tetramethyl-1,3,2-dioxaborolan-2-yl)phenyl)azetidin-1-yl)acetate

To a stirred solution of tert-butyl 2-(3-(4-bromophenyl)azetidin-1-yl)acetate (0.28 g, 0.85 mmol) in dioxane (5 mL) was added bis(pinacolato)diboron (0.32 g, 1.28 mmol) and KOAc (0.21 g, 2.14 mmol) at RT and degassed with nitrogen for 5 min. Pd(dppf)Cl₂·DCM (0.07 g, 0.08 mmol) was added into the reaction mixture and heated at 110° C. for 16 h. Once the reaction was completed (monitored by TLC), the reaction mixture was diluted with EtOAc. The combined organic layer was washed with water and brine, dried over anhydrous sodium sulphate and concentrated under vacuum to afford the title compound as white sticky solid (0.3 g, 93%). ¹H NMR (400 MHz, CDCl₃): δ 7.76 (d, J=8 Hz, 2H), 7.28 (d, J=8 Hz, 2H), 3.93 (m, 2H), 3.83 (m, 1H), 3.28 (m, 2H), 3.24 (s, 2H), 1.33 (s, 9H), 1.25 (s, 12H); LC-MS: m/z 374.4 (M+H).

### Intermediate 16: Tert-butyl 6-(4-(4,4,5,5-tetramethyl-1,3,2-dioxaborolan-2-yl)phenyl)-2-azaspiro[3.3]heptane-2-carboxylate

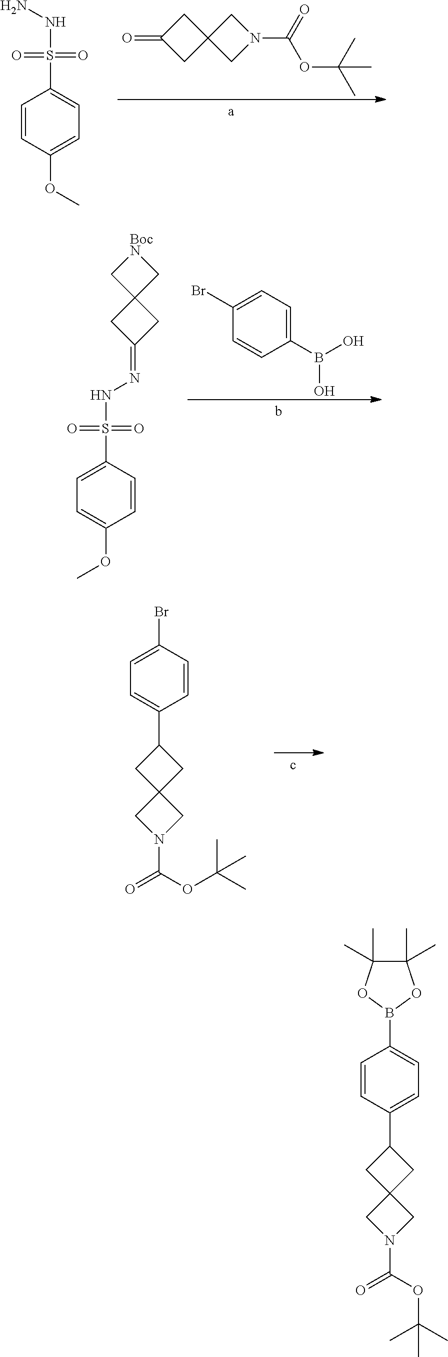 [SMILES: COc1ccc(S(=O)(=O)NN)cc1 ; CC(C)(C)OC(=O)N1CC2(CC(=O)C2)C1 ; COc1ccc(S(=O)(=O)NN=C2CC3(C2)CN(C(=O)OC(C)(C)C)C3)cc1 ; OB(O)c1ccc(Br)cc1 ; CC(C)(C)OC(=O)N1CC2(CC(c3ccc(Br)cc3)C2)C1 ; CC(C)(C)OC(=O)N1CC2(CC(c3ccc(B4OC(C)(C)C(C)(C)O4)cc3)C2)C1]

### Step-a: Synthesis of tert-butyl 6-(2-((4-methoxyphenyl)sulfonyl)hydrazineylidene)-2-azaspiro[3.3]heptane-2-carboxylate

To a stirred solution of 4-methoxybenzenesulfonohydrazide (1.5 g, 7.41 mmol) and tert-butyl 6-oxo-2-azaspiro[3.3]heptane-2-carboxylate (1.56 g, 7.41 mmol) in toluene (30 mL), the reaction mixture was heated for 16 h at 50° C. in sealed tube. Once the reaction was completed (monitored by TLC), reaction mass was concentrated under vacuum to give the residue which was purified by combiflash column chromatography using 35% ethyl acetate in hexane as eluent to afford the title compound as white solid (2.8 g, 95%). ¹H NMR (400 MHz, DMSO-d₆): δ 10.21 (bs, 1H), 7.68 (d, J=8.4 Hz, 2H), 7.05 (d, J=8.4 Hz, 2H), 3.82 (bs, 4H), 3.78 (s, 3H), 2.95 (m, 4H), 1.31 (s, 9H); LC-MS: m/z 394.1 (M−H).

### Step-b: Synthesis of tert-butyl 6-(4-bromophenyl)-2-azaspiro[3.3]heptane-2-carboxylate

To a stirred solution of tert-butyl 6-(2-((4-methoxyphenyl)sulfonyl)hydrazineylidene)-2-azaspiro[3.3]heptane-2-carboxylate (2.8 g, 7.08 mmol) and (4-bromophenyl)boronic acid (2.13 g, 10.6 mmol) in dioxane (60 mL) was added Cs₂CO₃ (4.6 g, 14.16 mmol) and degassed with nitrogen for 30 min. The reaction mixture was heated for 16 h at 110° C. in sealed tube. Once the reaction was completed (monitored by TLC), the reaction mixture was diluted with EtOAc and filtered. The filtrate was concentrated under vacuum to give the residue which was purified by combiflash column chromatography using 20% ethyl acetate in hexane as eluent to afford the title compound as colourless liquid (1.3 g, 52%). ¹H NMR (400 MHz, DMSO-d₆): δ 7.38 (d, J=8.4 Hz, 2H), 7.01 (d, J=8.4 Hz, 2H), 4.02 (s, 2H), 3.81 (s, 2H), 3.18 (m, 1H), 2.54 (m, 2H), 2.21 (m, 2H), 1.42 (s, 9H).

### Step-c: Synthesis of tert-butyl 6-(4-(4,4,5,5-tetramethyl-1,3,2-dioxaborolan-2-yl)phenyl)-2-azaspiro[3.3]heptane-2-carboxylate

To a stirred solution of tert-butyl 6-(4-bromophenyl)-2-azaspiro[3.3]heptane-2-carboxylate (1.3 g, 3.69 mmol) in dioxane (35 mL) was added bis(pinacolato)diboron (1.4 g, 5.53 mmol) and KOAc (0.90 g, 9.22 mmol) at RT and degassed with nitrogen for 5 min. Pd(dppf)Cl₂·DCM (0.30 g, 0.36 mmol) was added into the reaction mixture and heated at 110° C. for 16 h. Once the reaction was completed (monitored by TLC), the reaction mixture was diluted with EtOAc. The combined organic layer was washed with water and brine, dried over anhydrous sodium sulphate and concentrated under vacuum to afford the title compound as creamy sticky solid (0.3 g, 93%). ¹H NMR (400 MHz, DMSO-d₆): δ 7.74 (d, J=8 Hz, 2H), 7.16 (d, J=8 Hz, 2H), 4.05 (s, 2H), 3.83 (s, 2H), 3.38 (m, 1H), 2.56 (m, 2H), 2.58 (m, 2H), 1.33 (s, 9H), 1.25 (s, 12H). LC-MS: m/z 300.3 (M+H-100).

### Intermediate 17: Methyl 3,3-dimethyl-2-(4-oxopiperidin-1-yl)butanoate

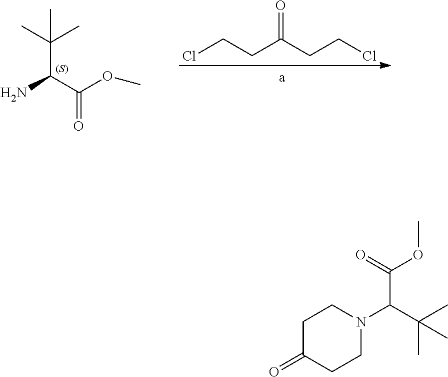

### Step-a: Synthesis of Methyl 3,3-dimethyl-2-(4-oxopiperidin-1-yl)butanoate

To a stirred solution of methyl (S)-2-amino-3,3-dimethylbutanoate (3.7 g, 25.48 mmol) in acetonitrile (40 mL) was added DIPEA (9.88 g, 76.44 mmol) dropwise followed by 1,5-dichloropentan-3-one (3.16 g, 20.38 mmol) was added into the reaction mixture. Then the reaction mixture was heated at 70° C. temperature for 16 hrs. After completion of the reaction the reaction mixture was diluted with water and extracted with ethyl acetate then the combined organic layer was dried over anhydrous sodium sulphate and concentrated to get crude product. The crude product which was purified first by combi flash column chromatography using 20-30% Ethyl acetate in hexane to afford the title compound as pale-yellow liquid (1.95 g, 39%).

¹H NMR (400 MHz, DMSO-d₆): 3.63 (s, 3H), 3.08 (s, 1H), 3.03-2.97 (m, 2H), 2.70-2.64 (m, 2H), 2.36-2.30 (m, 4H), 1.00 (s, 9H); LCMS: m/z 228.2 (M−H).

### Intermediate-18: Methyl 3-methyl-2-(3-(4-oxopiperidin-1-yl)isoxazol-5-yl)butanoate

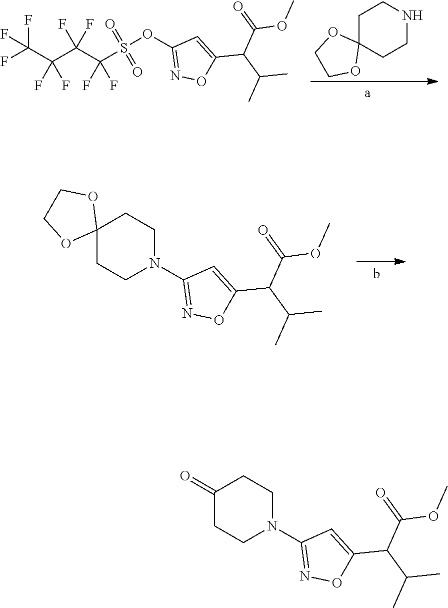

### Step-a: Synthesis of methyl 2-(3-(1,4-dioxa-8-azaspiro[4.5]decan-8-yl)isoxazol-5-yl)-3-methylbutanoate

To a stirred solution of methyl 3-methyl-2-(3-(((perfluorobutyl)sulfonyl)oxy)isoxazol-5-yl)butanoate (0.4 g, 0.83 mmol) and 1,4-dioxa-8-azaspiro[4.5]decane (0.18 g, 1.24 mmol) in DMF (6 mL) was added DIPEA (0.32 g, 2.5 mmol) and reaction mass was heated at 80° C. temperature for 16 hrs. After completion of the reaction the reaction mixture was poured into ice cold water and extracted with ethyl acetate and evaporated. The obtained crude product was purified first by combi flash column chromatography using 25% ethyl acetate in hexane to afford the title compound colourless solid (0.16 g, 59.3%). ¹H NMR (400 MHz, DMSO-d₆): □ 5.93 (s, 1H), 4.00 (s, 4H), 3.74 (s, 3H), 3.49 (d, J=7.2 Hz, 1H), 3.41 (m, 4H), 2.37-2.32 (m, 1H), 1.80 (m, 4H), 1.02 (d, J=6.8 Hz, 3H), 0.95 (d, J=6.8 Hz, 3H); LCMS: m/z 325.2 (M−H).

### Step-b: Synthesis of methyl 3-methyl-2-(3-(4-oxopiperidin-1-yl)isoxazol-5-yl)butanoate

To a stirred solution of methyl 2-(3-(1,4-dioxa-8-azaspiro[4.5]decan-8-yl)isoxazol-5-yl)-3-methylbutanoate (0.1 g, 0.81 mmol) in THF (4 mL) was added 4 N dioxane hydrochloride (0.4 mL) and slowly heated at 90° C. for 6 h. The reaction mixture was evaporated under reduced pressure, the resultant residue was washed with diethyl ether and dried under vacuum to afford the title compound as yellowish solid (0.05 g, 57.9%). ¹H NMR (400 MHz, DMSO-d₆): 6.03 (s, 1H), 3.76 (s, 3H), 3.66 (m, 4H), 3.54 (d, J=8.8 Hz, 1H), 3.55 (m, 4H), 2.40-2.34 (m, 1H), 1.03 (d, J=6.8 Hz, 3H), 0.96 (d, J=6.8 Hz, 3H); LCMS: m/z 281.1 (M+H).

### Example-1: (2S,4R)-1-((S)-2-(2-(4-(3-amino-6-(5-fluoro-2-hydroxyphenyl)pyridazin-4-yl)phenoxy)acetamido)-3,3-dimethylbutanoyl)-4-hydroxy-N—((S)-1-(4-(4-methylthiazol-5-yl)phenyl)ethyl)pyrrolidine-2-carboxamide (Compound 1)

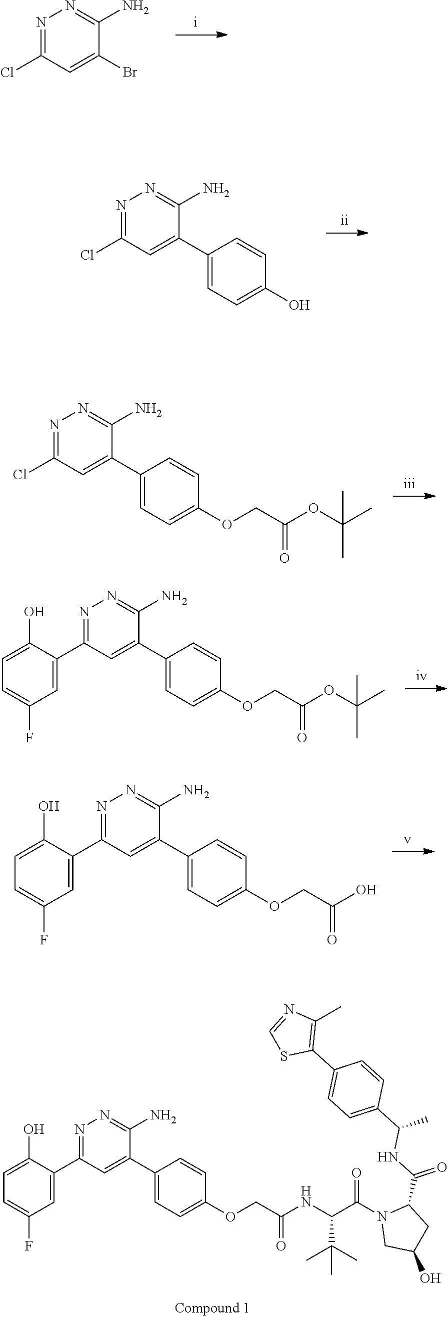 [SMILES: Nc1nnc(Cl)cc1Br ; Nc1nnc(Cl)cc1-c1ccc(O)cc1 ; CC(C)(C)OC(=O)COc1ccc(-c2cc(Cl)nnc2N)cc1 ; CC(C)(C)OC(=O)COc1ccc(-c2cc(-c3cc(F)ccc3O)nnc2N)cc1 ; Nc1nnc(-c2cc(F)ccc2O)cc1-c1ccc(OCC(=O)O)cc1 ; Cc1ncsc1-c1ccc([C@H](C)NC(=O)[C@@H]2C[C@@H](O)CN2C(=O)[C@@H](NC(=O)COc2ccc(-c3cc(-c4cc(F)ccc4O)nnc3N)cc2)C(C)(C)C)cc1]

### Step i: Synthesis of 4-(3-amino-6-chloropyridazin-4-yl)phenol

To a stirred solution of 4-bromo-6-chloropyridazin-3-amine (3 g, 14.39 mmol) and (4-hydroxyphenyl)boronic acid (2.97 g, 21.58 mmol) in 1,4-dioxane (45 mL) and water (15 mL), was added Na₂CO₃ (3.81 g, 35.9 mmol). The reaction mixture was degassed with nitrogen for 15 min, followed by addition of Pd(dppf)Cl₂·DCM (1.17 g, 1.43 mmol) and heated for 18 h at 120° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 35% ethyl acetate in hexane as eluent to afford the title compound as off-white solid (1.3 g, 40%). ¹H NMR (400 MHz, DMSO-d₆): δ 9.85 (s, 1H), 7.38 (d, J=8.4 Hz, 2H), 7.27 (s, 1H), 6.89 (d, J=8.4 Hz, 2H), 6.26 (bs, 2H). LC-MS: m/z 222 (M+H).

### Step ii: Synthesis of tert-butyl 2-(4-(3-amino-6-chloropyridazin-4-Yl)phenoxy)acetate

To a stirred solution of 4-(3-amino-6-chloropyridazin-4-yl)phenol (0.4 g, 1.8 mmol) and tert-butyl 2-bromoacetate (0.42 g, 2.17 mmol) in DMF (10 mL) was added K₂CO₃ (0.37 mL, 2.7 mmol) at RT and heated at 50° C. for 1 h. The reaction mixture was quenched with cold water and extracted with EtOAc. The combined organic layer was washed with water and brine, dried over anhydrous sodium sulphate and concentrated under vacuum to afford the title compound as brown solid (0.2 g, 33%). ¹H NMR (400 MHz, DMSO-d₆): δ 7.23 (d, J=9.6 Hz, 2H), 7.04 (d, J=8.8 Hz, 2H), 6.79 (s, 1H), 6.29 (s, 2H), 4.70 (s, 2H), 1.42 (s, 9H). LC-MS: m/z 336.1 (M+H).

### Step iii: Synthesis of tert-butyl 2-(4-(3-amino-6-(5-fluoro-2-hydroxyphenyl)pyridazin-4-yl)phenoxy)acetate

To a stirred solution of tert-butyl 2-(4-(3-amino-6-chloropyridazin-4-yl)phenoxy)acetate (0.08 g, 0.23 mmol) and (4-fluoro-2-hydroxyphenyl)boronic acid (0.07 g, 0.47 mmol) in 1,4-dioxane (1.6 mL) and water (0.4 mL) was added K₂CO₃ (0.1 g, 0.69 mmol). The reaction mixture was degassed with nitrogen for 15 min. Pd(dppf)Cl₂·DCM (0.02 g, 0.023 mmol) was added to the reaction mixture and heated for 1 h at 120° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 35% ethyl acetate in hexane as eluent to afford the title compound as off-white solid (0.025 g, 25%). LC-MS: m/z 412.2 (M+H).

### Step iv: Synthesis of 2-(4-(3-amino-6-(5-fluoro-2-hydroxyphenyl)pyridazin-4-yl)phenoxy)acetic acid

To a stirred solution of tert-butyl 2-(4-(3-amino-6-(5-fluoro-2-hydroxyphenyl)pyridazin-4-yl)phenoxy)acetate (0.025 g, 0.21 mmol) in DCM (2 mL) was added 4 N dioxane hydrochloride (0.25 mL) at 0° C. Then the reaction mixture was slowly brought to RT and stirred for 3 h. The reaction mixture was evaporated under reduced pressure, the resultant residue was washed with diethyl ether and dried under vacuum to afford the title compound as brown solid (0.02 g, 92%). LC-MS: m/z 356 (M+H).

### Step v: Synthesis of (2S,4R)-1-((S)-2-(2-(4-(3-amino-6-(5-fluoro-2-hydroxyphenyl)pyridazin-4-yl)phenoxy)acetamido)-3,3-dimethylbutanoyl)-4-hydroxy-N—((S)-1-(4-(4-methylthiazol-5-yl)phenyl)ethyl)pyrrolidine-2-carboxamide

To a stirred solution of 2-(4-(3-amino-6-(5-fluoro-2-hydroxyphenyl)pyridazin-4-yl)phenoxy)acetic acid (0.02 g, 0.056 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (WO2019207538) (0.03 g, 0.06 mmol) and HATU (0.04 g, 0.076 mmol) in DMF (2 mL) was dropwise added DIPEA (0.04 mL, 0.2 mmol) at 0° C. and slowly brought to RT. The reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 3% methanol in DCM as eluent to afford the title compound as off-white solid (0.020 g, 45%). ¹H NMR (400 MHz, DMSO-d₆): δ 13.5 (s, 1H), 8.98 (s, 1H), 8.43 (d, J=7.2 Hz, 1H), 8.03 (s, 1H), 7.95 (d, J=9.2 Hz, 1H), 7.88-7.85 (m, 1H), 7.61 (d, J=8.8 Hz, 2H), 7.45-7.36 (m, 4H), 7.13-7.09 (m, 3H), 6.94-6.90 (m, 1H), 6.51 (s, 2H), 5.14 (d, J=2.3 Hz, 1H), 4.93 (t, J=14.4 Hz, 1H), 4.72 (s, 2H), 4.60 (d, J=9.2 Hz, 1H), 4.47 (t, J=17.6 Hz, 1H), 4.29 (s, 1H), 3.61 (t, J=7.6 Hz, 2H), 2.50-2.30 (m, 3H), 2.04 (s, 1H), 1.78 (s, 1H), 1.39 (d, J=6.8 Hz, 3H), 0.96 (s, 9H). LC-MS: m/z 782.25 (M+H).

### Example-2: (2S,4R)-1-((S)-2-(2-(4-(3-amino-6-(5-fluoro-2-hydroxyphenyl)pyridazin-4-yl)phenoxy)acetamido)-3,3-dimethylbutanoyl)-N—((S)-1-(4-(4-(difluoromethyl)thiazol-5-yl)phenyl)ethyl)-4-hydroxypyrrolidine-2-carboxamide (Compound 2)

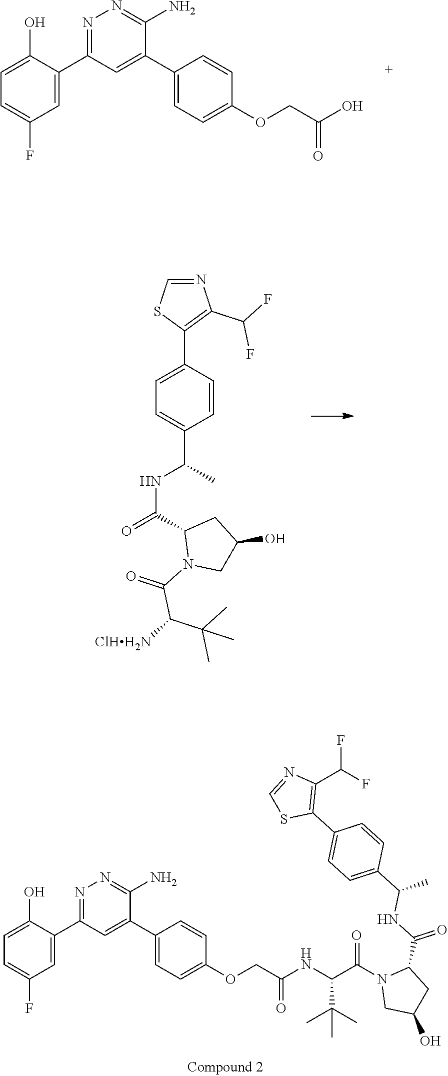

Compound 2 was synthesized using the same procedure as mentioned in step v of example-1 by using (2S,4R)-1-((S)-2-amino-3,3-dimethylbutanoyl)-N—((S)-1-(4-(4-(difluoromethyl)thiazol-5-yl)phenyl)ethyl)-4-hydroxypyrrolidine-2-carboxamide hydrochloride to obtain title compound as pale brown solid (0.02 g, 32%). ¹H NMR (400 MHz, DMSO-d₆): δ 13.47 (s, 1H), 9.20 (s, 1H), 8.48 (d, J=8.0 Hz, 1H), 8.04 (s, 1H), 7.96 (d, J=9.6 Hz, 1H), 7.87 (dd, J=7.6 Hz & 3.2 Hz, 1H), 7.61 (d, J=8.8 Hz, 2H), 7.47-7.41 (m, 4H), 7.13-7.10 (m, 3H), 7.01 (t, J=52.8 Hz, 1H), 6.94-6.91 (m, 1H), 6.52 (s, 2H), 5.16 (d, J=3.2 Hz, 1H), 4.94 (s, 1H), 4.72 (s, 2H), 4.59 (d, J=9.6 Hz, 1H), 4.46 (s, 1H), 4.29 (s, 1H), 3.61 (d, J=6.0 Hz, 2H), 2.10-2.02 (m, 1H), 1.83-1.75 (m, 1H), 1.39 (d, J=6.8 Hz, 3H), 0.92 (s, 9H). LC-MS: m/z 818.2 (M+H).

### Example-3: (2S4R)-1-((2-(4-(3-amino-6-(2-hydroxyphenyl)pyridazin-4-yl)phenoxy)acetyl)-L-valyl)-N-(4-(4-(difluoromethyl)thiazol-5-yl)benzyl)-4-hydroxypyrrolidine-2-carboxamide (Compound 3)

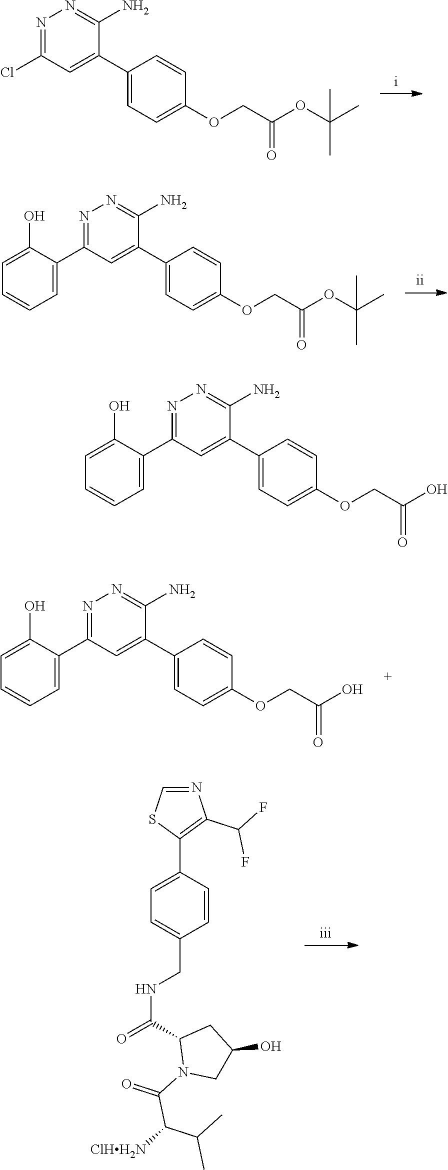

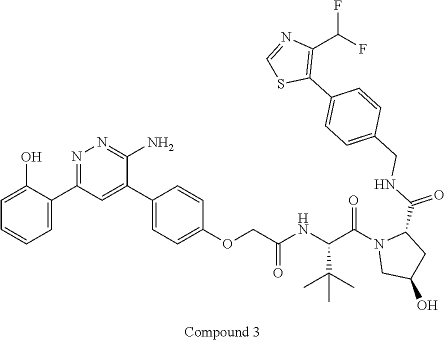 [SMILES: CC(C)(C)[C@H](NC(=O)COc1ccc(-c2cc(-c3ccccc3O)nnc2N)cc1)C(=O)N1C[C@H](O)C[C@H]1C(=O)NCc1ccc(-c2scnc2C(F)F)cc1]

### Step i: Synthesis of tert-butyl 2-(4-(3-amino-6-(2-hydroxyphenyl)pyridazin-4-yl)phenoxy)acetate

To a stirred solution of tert-butyl 2-(4-(3-amino-6-chloropyridazin-4-yl)phenoxy)acetate (0.27 g, 0.8 mmol) and (2-hydroxyphenyl)boronic acid (0.22 g, 1.6 mmol) in 1,4-dioxane (3.6 mL) and water (1.2 mL) was added K₂CO₃ (0.33 g, 2.4 mmol). The reaction mixture was degassed with nitrogen for 15 min. Pd(dppf)Cl₂·DCM (0.065 g, 0.08 mmol) was added to the reaction mixture and heated for 1 h at 120° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 35% ethyl acetate in hexane as eluent to afford the title compound as off-white solid (0.12 g, 38%). ¹H NMR ¹H NMR (400 MHz, DMSO-d₆): δ 13.62 (s, 1H), 7.98 (s, 1H), 7.95 (d, J=8.0 Hz, 1H), 7.59 (d, J=8.8 Hz, 2H), 7.24 (d, J=7.2 Hz, 1H), 7.07 (d, J=8.4 Hz, 2H), 6.93-6.83 (m, 2H), 6.42 (s, 1H), 4.74 (s, 2H), 1.46 (s, 9H). LC-MS: m/z 394.3 (M+H).

### Step ii: Synthesis of 2-(4-(3-amino-6-(2-hydroxyphenyl)pyridazin-4-yl)phenoxy)acetic acid

To a stirred solution of tert-butyl 2-(4-(3-amino-6-(2-hydroxyphenyl)pyridazin-4-yl)phenoxy)acetate (0.12 g, 0.35 mmol) in DCM (2 mL) was added 4N dioxane hydrochloride (2 mL) at 0° C. and slowly brought to RT and stirred for 3 h. The reaction mixture was evaporated under reduced pressure, the resultant residue was washed with diethyl ether and dried under vacuum to afford the title compound as yellow solid (0.08 g, 77%). ¹H NMR (400 MHz, DMSO-d₆): δ 13.15 (s, 1H), 8.09 (s, 1H), 7.80 (s, 2H), 7.68 (d, J=7.6 Hz, 1H), 7.59 (d, J=8.4 Hz, 2H), 7.34 (d, J=7.6 Hz, 1H), 7.12 (t, J=8.0 Hz, 2H), 7.02 (d, J=8.0 Hz, 1H), 6.96 (t, J=7.2 Hz, 1H), 4.78 (s, 1H). LC-MS: m/z 338.1 (M+H).

### Step iii: Synthesis of (2S,4R)-1-((2-(4-(3-amino-6-(2-hydroxyphenyl)pyridazin-4-yl)phenoxy)acetyl)-L-valyl)-N-(4-(4-(difluoromethyl)thiazol-5-yl)benzyl)-4-hydroxypyrrolidine-2-carboxamide

To a solution of 2-(4-(3-amino-6-(2-hydroxyphenyl)pyridazin-4-yl)phenoxy)acetic acid (0.04 g, 0.10 mmol), (2S,4R)-1-(L-valyl)-N-(4-(4-(difluoromethyl)thiazol-5-yl)benzyl)-4-hydroxypyrrolidine-2-carboxamide hydrochloride (0.06 g, 0.12 mmol) and HATU (0.06 g, 0.16 mmol) in DMF (2 mL) was dropwise added DIPEA (0.1 mL, 0.53 mmol) at 0° C. and slowly brought to RT. The reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 2-3% methanol in DCM as eluent to afford the title compound as light brown solid (0.02 g, 22%). ¹H NMR (400 MHz, DMSO-d₆): δ 13.62 (s, 1H), 9.19 (s, 1H), 8.57 (s, 1H), 8.18 (d, J=8.8 Hz, 1H), 7.95 (t, J=8.0 Hz, 2H), 7.58 (d, J=8.4 Hz, 2H), 7.43 (s, 4H), 7.25 (s, 1H), 7.11 (d, J=8.8 Hz, 2H), 6.93-6.86 (m, 2H), 6.42 (s, 2H), 5.15 (d, J=2.8 Hz, 1H), 4.68 (s, 2H), 4.49-4.37 (m, 3H), 4.37-4.33 (m, 3H), 3.67-3.65 (m, 2H), 2.11-2.02 (m, 2H), 1.94-1.85 (m, 1H), 0.92 (d, J=6.4 Hz, 3H), 0.85 (d, J=6.8 Hz, 3H). LC-MS: m/z 772.2 (M+H).

### Example-4: (2S,4R)-1-(2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 4)

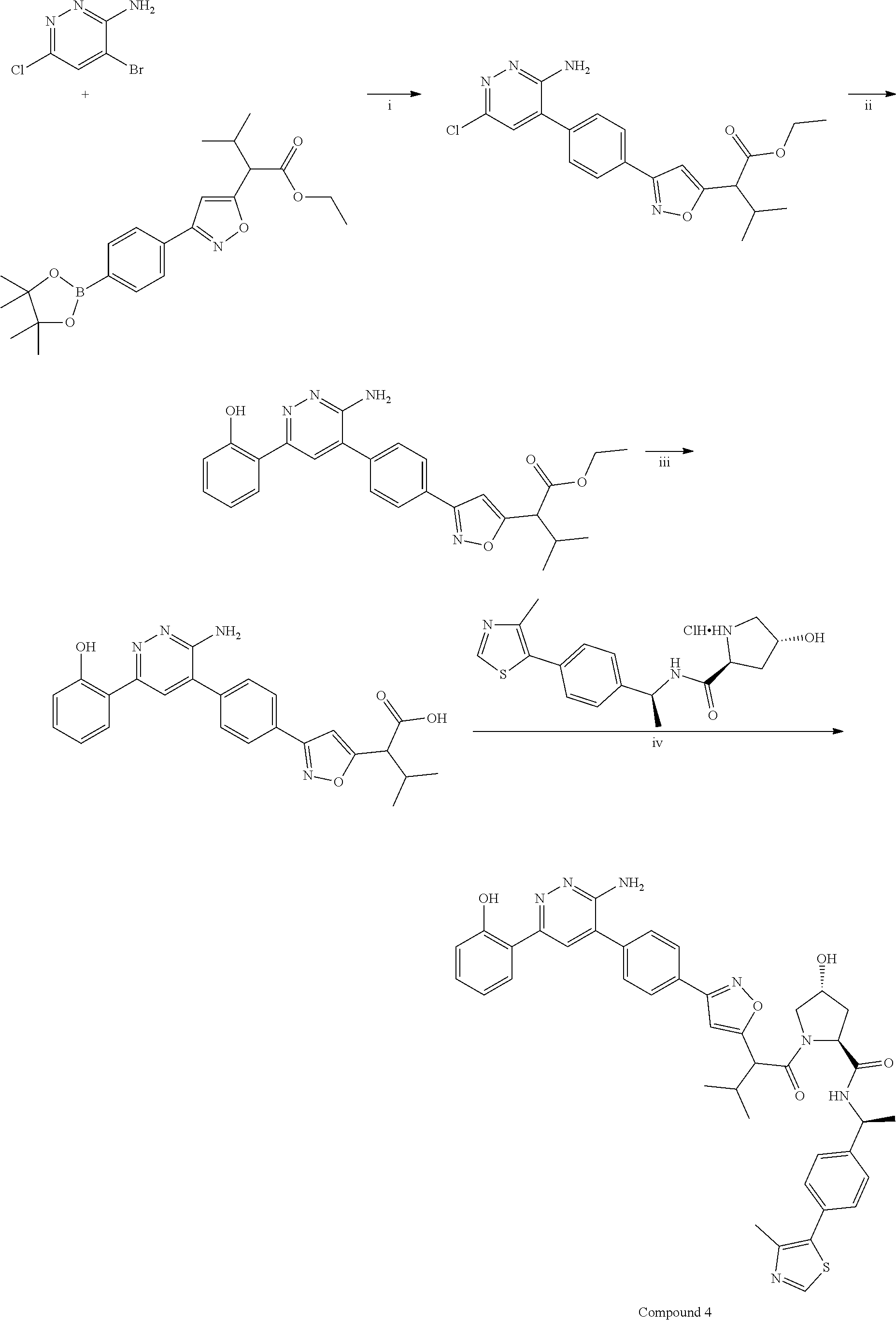

### Step i: Synthesis of ethyl 2-(3-(4-(3-amino-6-chloropyridazin-4-yl)phenyl)isoxazol-5-yl)-3-methylbutanoate

To a stirred solution of ethyl 3-methyl-2-(3-(4-(4,4,5,5-tetramethyl-1,3,2-dioxaborolan-2-yl)phenyl)isoxazol-5-yl)butanoate (0.2 g, 0.50 mmol) and 4-bromo-6-chloropyridazin-3-amine (0.15 g, 0.75 mmol) in 1,4-dioxane (8 mL) and water (0.8 mL) was added K₂CO₃ (0.17 g, 1.25 mmol). The reaction mixture was degassed with nitrogen for 15 min, followed by Pd(dppf)Cl₂·DCM (0.04 g, 0.05 mmol) was added and heated for 1 h at 100° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 35% ethyl acetate in hexane as eluent to afford the title compound as off-white solid (0.12 g, 60%). ¹H NMR (400 MHz, DMSO-d₆): δ 8.02 (d, J=8 Hz, 2H), 7.68 (d, J=8 Hz, 2H), 7.43 (s, 1H), 7.11 (s, 1H), 6.45 (bs, 2H), 4.17 (q, J=8 Hz, 2H), 3.87 (d, J=8 Hz, 1H), 2.40 (m, 1H), 1.22 (t, J=12 Hz, 3H), 0.99 (d, J=4 Hz, 3H), 0.90 (d, J=4 Hz, 3H); LC-MS: m/z 401.1 (M+H).

### Step ii: Synthesis of ethyl 2-(3-(4-(3-amino-6-(2-hydroxyphenyl)pyridazin-4-yl)phenyl)isoxazol-5-yl)-3-methylbutanoate

To a stirred solution of ethyl 2-(3-(4-(3-amino-6-chloropyridazin-4-yl)phenyl)isoxazol-5-yl)-3-methylbutanoate (0.11 g, 0.27 mmol) and (2-hydroxyphenyl)boronic acid (0.075 g, 0.54 mmol) in 1,4-dioxane (9 mL) and water (2 mL) was added K₂CO₃ (0.11 g, 0.82 mmol). The reaction mixture was degassed with nitrogen for 15 min. Pd(dppf)Cl₂·DCM (0.02 g, 0.027 mmol) was added into the reaction mixture and heated for 1 h at 120° C. in microwave. Once the reaction was completed (monitored by TLC), the reaction mixture was diluted with EtOAc. The combined organic layer was washed with water and brine, dried over anhydrous sodium sulphate and concentrated under vacuum to get the residue which was purified by combiflash column chromatography using 50% ethyl acetate in hexane as eluent to afford the title compound as off-white solid (0.085 g, 67%). ¹H NMR (400 MHz, DMSO-d₆): δ 13.55 (s, 1H), 8.06 (d, J=12 Hz, 3H), 7.97 (d, J=4 Hz, 1H), 7.77 (d, J=8 Hz, 2H), 7.26 (m, 1H), 7.14 (s, 1H), 6.94-6.90 (m, 2H), 6.57 (bs, 2H), 4.19-4.16 (m, 2H), 3.88 (d, J=12 Hz, 1H), 1.21 (t, J=16 Hz, 3H), 1.00 (d, J=8 Hz, 3H), 0.91 (d, J=4 Hz, 3H); LC-MS: m/z 459.1 (M+H).

### Step iii: Synthesis of 2-(3-(4-(3-amino-6-(2-hydroxyphenyl)pyridazin-4-yl)phenyl)isoxazol-5-yl)-3-methylbutanoic acid

To a stirred solution of ethyl 2-(3-(4-(3-amino-6-(2-hydroxyphenyl)pyridazin-4-yl)phenyl)isoxazol-5-yl)-3-methylbutanoate (0.08 g, 0.18 mmol) in THF (0.5 mL) and H₂O (0.5 mL) mixture was added LiOH·H₂O (0.023 g, 0.55 mmol) at 0° C. The reaction mixture was stirred for 1 h at RT. The reaction mixture was evaporated under reduced pressure and the resultant residue was diluted with methanol and acidified to pH-6 using Amberlite® IT120 and filtered. The filtrate was concentrated under vacuum to afford the title compound as brown solid (0.05 g, 66%). ¹H NMR (400 MHz, DMSO-d₆): δ 8.09-7.96 (m, 4H), 7.74 (d, J=8 Hz, 2H), 7.25 (d, J=20 Hz, 1H), 6.94-6.89 (m, 2H), 6.79 (bs, 1H), 6.56 (bs, 2H), 3.20 (d, J=12 Hz, 1H), 2.32 (m, 1H), 0.97 (d, J=8 Hz, 3H), 0.79 (d, J=4 Hz, 3H); LC-MS: m/z 431.15 (M+H).

### Step-iv: Synthesis of (2S,4R)-1-(2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(4-(3-amino-6-(2-hydroxyphenyl)pyridazin-4-yl)phenyl)isoxazol-5-yl)-3-methylbutanoic acid (0.05 g, 0.11 mmol), (2S,4R)-4-hydroxy-N—((S)-1-(4-(4-methylthiazol-5-yl)phenyl)ethyl)pyrrolidine-2-carboxamide hydrochloride (0.05 g, 0.14 mmol) and HATU (0.06 g, 0.17 mmol) in DMF (5 mL) was dropwise added DIPEA (0.1 mL, 0.58 mmol) at 0° C. and slowly brought to RT. The reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 3% methanol in DCM as eluent to afford the title compound as off-white solid (0.025 g, 29%). ¹H NMR (400 MHz, DMSO-d₆): δ 13.55 (t, J=12 Hz, 1H), 8.96 (d, J=4 Hz, 1H), 8.44 (d, J=8 Hz, 1H), 8.08-7.98 (m, 4H), 7.78-7.72 (m, 2H), 7.46 (d, J=4 Hz, 1H), 7.38 (d, J=8 Hz, 2H), 7.31 (m, 2H), 7.04 (d, J=12 Hz, 1H), 6.94-6.88 (m, 2H), 6.57 (bs, 2H), 5.14 (q, J=8 Hz, 1H), 4.96-4.91 (m, 1H), 4.46-4.38 (m, 1H), 4.31 (bs, 1H), 3.98 (d, J=8 Hz, 1H), 3.75 (m, 2H), 2.47 (s, 3H), 2.07 (t, J=12 Hz, 1H), 1.79 (t, J=8 Hz, 1H), 1.50 (d, J=4 Hz, 1H), 1.39 (d, J=8 Hz, 3H), 1.04 (d, J=20 Hz, 3H), 0.88 (d, J=8 Hz, 3H); LC-MS: m/z 744.2 (M+H).

### Example-5: (2S,4R)-1-((S)-2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 5) and (2S,4R)-1-((R)-2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 6)

Compounds 5 and 6 were obtained by chiral HPLC of compound 4 using following method. Chiral HPLC method DILUTION: IPA:DCM(90:10); MOBILE PHASE A: 0.1% TFA in MTBE; MOBILE PHASE B: 100% Ethanol; ISOCRATIC: A:B(80:20); FLOW: 15 mL/min,COLUMN: Chiralpak IA (250×20×5 μm) (2S,4R)-1-((S)-2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 5): ¹H NMR (400 MHz, DMSO-d₆): δ 8.98 (s, 1H), 8.42 (d, J=8 Hz, 1H), 8.04 (d, J=8 Hz, 4H), 7.96 (d, J=8 Hz, 1H), 7.44 (d, J=8 Hz, 2H), 7.38 (d, J=8 Hz, 2H), 7.25 (bs, 1H), 7.04 (s, 1H), 6.91-6.88 (m, 2H), 6.53 (bs, 2H), 4.93 (t, J=12 Hz, 1H), 4.41 (t, J=16 Hz, 1H), 4.31 (bs, 1H), 3.89 (d, J=12 Hz, 1H), 3.76 (d, J=4 Hz, 1H), 3.70-3.50 (m, 2H), 2.44 (s, 3H), 2.05 (d, J=12 Hz, 1H), 1.81 (s, 1H), 1.39 (d, J=8 Hz, 3H), 1.03 (d, J=8 Hz, 3H), 0.86 (d, J=8 Hz, 3H); LC-MS: m/z 744.2 (M+H). (2S,4R)-1-((R)-2-(3-(4-(3-amino-6-(2-hydroxyphenyl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 6): ¹H NMR (400 MHz, DMSO-d₆): δ 13.54 (s, 1H), 8.93 (s, 1H), 8.31 (d, J=8 Hz, 1H), 8.06-7.96 (m, 4H), 7.76 (d, J=16 Hz, 2H), 7.49 (q, J=16 Hz, 1H), 7.36 (d, J=8 Hz, 2H), 7.31-7.24 (m, 2H), 7.01 (d, J=8 Hz, 1H), 6.92 (d, J=16 Hz, 2H), 6.53 (bs, 2H), 5.12 (d, J=4 Hz, 1H), 4.87 (t, J=12 Hz, 1H), 4.46 (t, J=16 Hz, 1H), 4.29 (bs, 1H), 3.97 (d, J=12 Hz, 1H), 3.65-3.45 (m, 2H), 2.32 (s, 3H), 2.07 (s, 1H), 1.80 (d, J=8 Hz, 1H), 1.50 (d, J=8 Hz, 1H), 1.35 (d, J=8 Hz, 3H), 1.03 (d, J=8 Hz, 3H), 0.88 (d, J=8 Hz, 3H); LC-MS: m/z 745.0 (M+H).

The compounds listed in below Table-2 were prepared by the procedure similar to the one described in Example-4 with appropriate variations in reactants, quantities of reagents, protections and deprotections, solvents and reaction conditions. The characterization data of the compounds are summarized herein the below table.

| Com- | | |
|---|---|---|
| pound | Structure | Characterization Data |
| 7 | 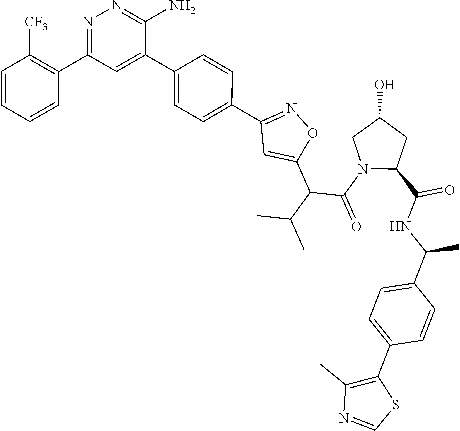 [SMILES: Cc1ncsc1-c1ccc([C@H](C)NC(=O)[C@@H]2C[C@@H](O)CN2C(=O)C(c2cc(-c3ccc(-c4cc(-c5ccccc5C(F)(F)F)nnc4N)cc3)no2)C(C)C)cc1] | ¹H NMR (400 MHz, DMSO-d₆): δ 8.99- 8.91 (m, 1H), 8.42-8.29 (m, 1H), 8.01 (d, J = 8 Hz, 2H), 7.87 (d, J = 8 Hz, 1H), 7.78 (d, J = 16 Hz, 1H), 7.72-7.64 (m, 4H), 7.48-7.29 (m, 5H), 7.00 (d, J = 12 Hz, 1H), 6.41 (bs, 2H), 5.10 (d, J = 12 Hz, 1H), 4.93-4.85 (m, 1H), 4.48-4.38 (m, 1H), 4.29 (bs, 1H), 3.97-3.99 (m, 1H), 3.74-3.43 (m, 2H), 2.43 (s, 3H), 2.06- 2.03 (m, 1H), 1.80-1.78 (m, 1H), 1.49 (d, 1H) 1.40-1.33 (m, 3H), 1.02 (d, J = 7.6 Hz, 3H), 0.84 (m, J = 7.6 Hz, 3H); LC- MS: m/z 796.3 (M + H). |
| | (2S,4R)-1-(2-(3-(4-(3-amino-6-(2- | |
| | (trifluoromethyl)phenyl)pyridazin- | |
| | 4-yl)phenyl)isoxazol-5-yl)-3- | |
| | methylbutanoyl)-4-hydroxy-N- | |
| | ((S)-1-(4-(4-methylthiazol-5- | |
| | yl)phenyl)ethyl)pyrrolidine-2- | |
| | carboxamide | |
| 8 | 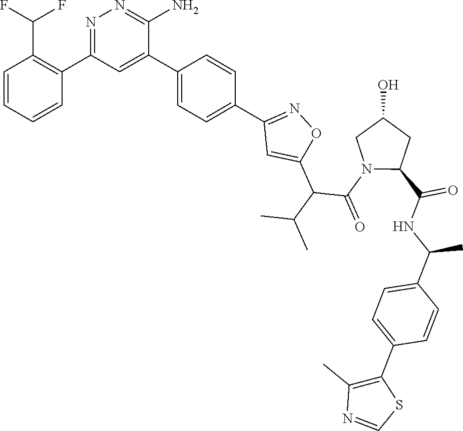 [SMILES: Cc1ncsc1-c1ccc([C@H](C)NC(=O)[C@@H]2C[C@@H](O)CN2C(=O)C(c2cc(-c3ccc(-c4cc(-c5ccccc5C(F)F)nnc4N)cc3)no2)C(C)C)cc1] | ¹H NMR (400 MHz, DMSO-d₆): δ 8.98 (d, J = 4 Hz, 1H), 8.41 (m, 1H), 8.03-7.96 (m, 2H), 7.80-7.58 (m, 6H), 7.53-7.29 (m, 6H), 7.00 (d, J = 4 Hz, 1H), 6.45 (bs, 2H), 5.11 (bs, 1H), 4.95-4.85 (m, 1H), 4.48-4.38 (m, 1H), 4.29 (bs, 1H), 3.99- 3.87 (m, 1H), 3.64-3.44 (m, 2H), 2.34 (s, 3H), 2.04 (d, J = 4 Hz, 1H), 1.79 (d, J = 4 Hz, 1H), 1.49 (d, J = 4 Hz, 1H), 1.38 (m, 3H), 1.01 (d, J = 8 Hz, 3H), 0.88 (6, J = 78 Hz, 3H); LC-MS: m/z 778.2 (M + H).LC/MS: 778.2 (M + H). |
| | (2S,4R)-1-(2-(3-(4-(3-amino-6-(2- | |
| | (difluoromethyl)phenyl)pyridazin- | |
| | 4-yl)phenyl)isoxazol-5-yl)-3- | |
| | methylbutanoyl)-4-hydroxy-N- | |
| | ((S)-1-(4-(4-methylthiazol-5- | |
| | yl)phenyl)ethyl)pyrrolidine-2- | |
| | carboxamide | |
| 9 | 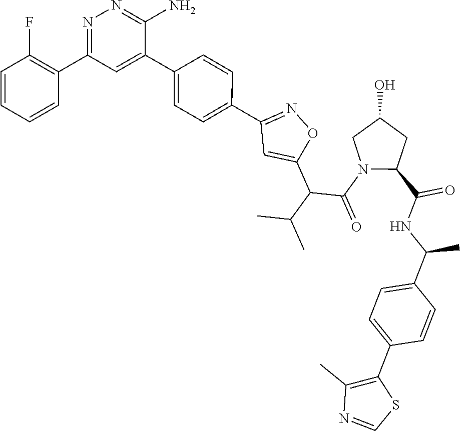 [SMILES: Cc1ncsc1-c1ccc([C@H](C)NC(=O)[C@@H]2C[C@@H](O)CN2C(=O)C(c2cc(-c3ccc(-c4cc(-c5ccccc5F)nnc4N)cc3)no2)C(C)C)cc1] | ¹H NMR (400 MHz, DMSO-d₆): δ 8.98- 8.92 (m, 1H), 8.43-8.33 (m, 1H), 8.03- 7.90 (m, 3H), 7.73-7.67 (m, 2H), 7.58 (bs, 1H), 7.48-7.29 (m, 7H), 7.00 (d, J = 8 Hz, 1H), 6.50 (bs, 2H), 5.12-5.10 (m, 1H), 4.95-4.87 (m, 1H), 4.46-4.40 (m, 1H), 4.30 (bs, 1H), 3.99-3.88 (m, 1H), 3.62- 3.52 (m, 2H), 2.43 (s, 3H), 2.08-1.98 (m, 1H), 1.79 (d, J = 4 Hz, 1H), 1.49 (d, J = 4 Hz, 1H), 1.40-1.33 (m, 3H), 1.02 (d, J = 8 Hz, 3H), 0.86 (d, J = 8 Hz, 3H); LC- MS: m/z 746.3 (M + H). |
| | (2S,4R)-1-(2-(3-(4-(3-amino-6-(2- | |
| | fluorophenyl)pyridazin-4- | |
| | yl)phenyl)isoxazol-5-yl)-3- | |
| | methylbutanoyl)-4-hydroxy-N- | |
| | ((S)-1-(4-(4-methylthiazol-5- | |
| | yl)phenyl)ethyl)pyrrolidine-2- | |
| | carboxamide | |

### Example-6: (2S,4R)-1-(2-(3-(4-(3-amino-6-methoxypyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 10)

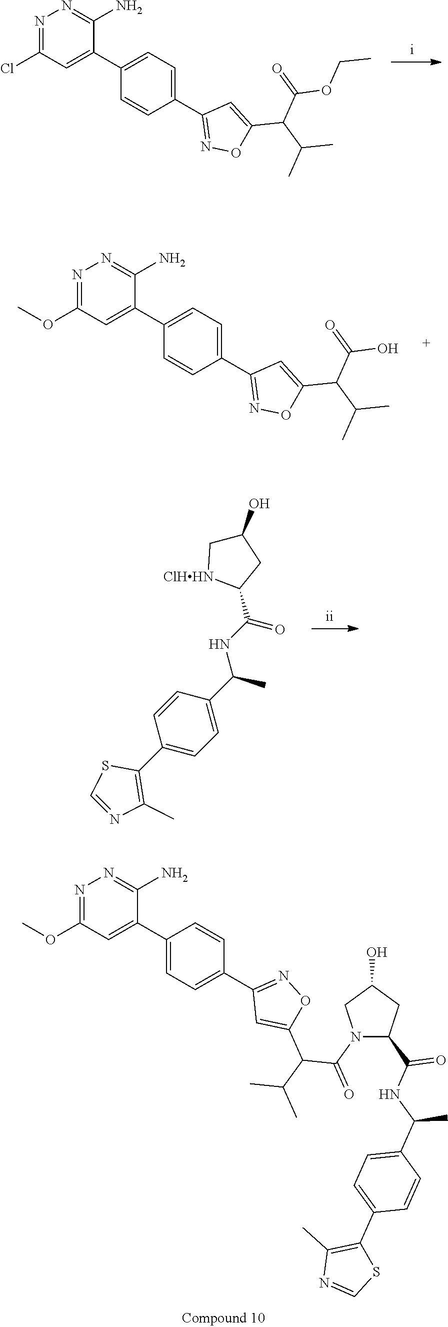

### Step-i: Synthesis of 2-(3-(4-(3-amino-6-methoxypyridazin-4-yl)phenyl)isoxazol-5-yl)-3-methylbutanoic acid

To a stirred solution of ethyl 2-(3-(4-(3-amino-6-chloropyridazin-4-yl)phenyl)isoxazol-5-yl)-3-methylbutanoate (0.05 g, 0.12 mmol) in methanol (3 mL) was added NaOMe (0.02 g, 0.36 mmol) and the reaction mixture was heated for 1 h at 120° C. in microwave. The reaction mixture was evaporated under reduced pressure and the resultant residue was diluted with methanol and acidified to pH-6 using Amberlite® IT120 (acidic resin) and filtered. The filtrate was concentrated under vacuum to afford the title compound as yellow solid (0.04 g, 87%). ¹H NMR (400 MHz, DMSO-d₆): δ 7.95 (d, J=8.0 Hz, 2H), 7.64 (d, J=8.0 Hz, 2H), 6.90 (s, 1H), 6.69 (s, 1H), 5.63 (bs, 2H), 3.91 (s, 3H), 2.25 (m, 1H), 0.96 (d, J=6.8 Hz, 3H), 0.87 (d, J=6.8 Hz, 3H). LC-MS: m/z 369.2 (M+H).

### Step-ii: Synthesis of (2S,4R)-1-(2-(3-(4-(3-amino-6-methoxypyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(4-(3-amino-6-methoxypyridazin-4-yl)phenyl)isoxazol-5-yl)-3-methylbutanoic acid (0.04 g, 0.11 mmol), (2R,4S)-4-hydroxy-N—((S)-1-(4-(4-methylthiazol-5-yl)phenyl)ethyl)pyrrolidine-2-carboxamide hydrochloride (0.05 g, 0.14 mmol) and HATU (0.06 g, 0.17 mmol) in DMF (3 mL) was dropwise added DIPEA (0.1 mL, 0.58 mmol) at 0° C. and slowly brought to RT. The reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 3% methanol in DCM as eluent to afford the title compound as off-white solid (0.01 g, 13%). ¹H NMR (400 MHz, DMSO-d₆): δ 8.99-8.93 (m, 1H), 8.42-8.29 (m, 1H), 7.99 (d, J=20 Hz, 2H), 7.67-7.62 (m, 2H), 7.48-7.36 (m, 3H), 7.30 (d, J=8 Hz, 1H), 6.99 (d, J=8 Hz, 1H), 6.90 (d, J=8 Hz, 1H), 5.65 (bs, 2H), 5.11 (bs, 1H), 4.95-4.85 (m, 1H), 4.48-4.38 (m, 1H), 4.29 (bs, 1H), 3.97 (d, 1H), 3.91 (s, 3H), 3.64-3.44 (m, 2H), 2.43 (s, 3H), 2.04 (m, 1H), 1.79 (m, 1H), 1.64 (bs, 1H) 1.36 (m, 3H), 1.02 (d, J=6.4 Hz, 3H), 0.88 (d, J=6.4 Hz, 3H); LC-MS: m/z 682.3 (M+H).

### Example-7: (2S,4R)-1-(2-(4-(4-(3-amino-6-(2-hydroxyphenyl)pyridazin-4-yl)phenyl)-1H-pyrazol-1-yl)-3-methylbutanoyl)-4-hydroxy-N—((S)-1-(4-(4-methylthiazol-5-yl)phenyl)ethyl)pyrrolidine-2-carboxamide (Compound 11)_{P}

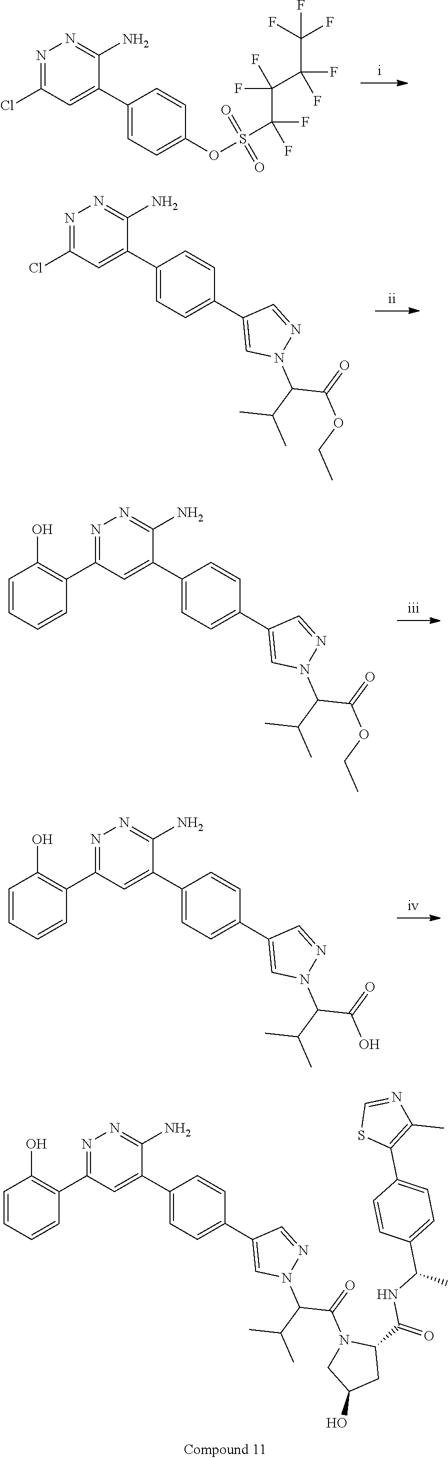

### Step i: Synthesis of ethyl 2-(4-(4-(3-amino-6-chloropyridazin-4-yl)phenyl)-1H-pyrazol-1-yl)-3-methylbutanoate

To a stirred solution of 4-(3-amino-6-chloropyridazin-4-yl)phenyl 1,1,2,2,3,3,4,4,4-nonafluorobutane-1-sulfonate (0.2 g, 0.39 mmol), (1-(1-ethoxy-3-methyl-1-oxobutan-2-yl)-1H-pyrazol-4-yl)boronic acid (0.15 g, 0.44 mmol), Na₂CO₃ (0.12 g, 1.19 mmol) and Lithium chloride (0.02 g, 0.47 mmol), in toluene (6 mL), ethanol (3 mL) and water (mL). The reaction mixture was degassed with nitrogen for 15 min, followed by Pd(PPh₃)₄ (0.045 g, 0.04 mmol) was added and heated at 130° C. for 1 h in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 60% ethyl acetate in hexane as eluent to afford the title compound as yellow solid (0.1 g, 63%). ¹H NMR (400 MHz, CDCl₃): δ 8.01 (s, 1H), 7.83 (s, 1H), 7.65 (d, J=8.4 Hz, 2H), 7.48 (d, J=8.4 Hz, 2H) 7.17 (s, 1H), 4.90 (bs, 2H), 4.70 (d, J=9.2 Hz, 1H), 4.25 (q, J=7.2 Hz, 2H), 2.54 (m, 1H), 1.30 (t, J=7.2 Hz, 3H), 1.06 (d, J=6.4 Hz, 3H), 0.88 (d, J=6.4 Hz, 3H); LC-MS: m/z 400.2 (M+H).

### Step ii: Synthesis of ethyl 2-(4-(4-(3-amino-6-(2-hydroxyphenyl)pyridazin-4-yl)phenyl)-1H-pyrazol-1-yl)-3-methylbutanoate

To a stirred solution of ethyl 2-(4-(4-(3-amino-6-chloropyridazin-4-yl)phenyl)-1H-pyrazol-1-yl)-3-methylbutanoate (0.1 g, 0.25 mmol), 2-hydroxyphenylboronic acid (0.04 g, 0.3 mmol) and Na₂CO₃ (0.08 g, 0.75 mmol) in 1,4-dioxane (5 mL) and water (0.5 mL). The reaction mixture degassed with nitrogen for 15 min, followed by Pd(dppf)Cl₂·DCM (0.02 g, 0.025 mmol) was added and heated at 130° C. for 4 h in sealed tube. Once the reaction was completed (monitored by TLC), the reaction mixture was diluted with EtOAc. The combined organic layer was washed with water and brine, dried over anhydrous sodium sulphate and concentrated under vacuum to give the residue. The residue was purified by combiflash column chromatography using 45% ethyl acetate in hexane as eluent to afford the title compound as yellow solid (0.055 g, 48%). ¹H NMR (400 MHz, CDCl₃): δ 8.03 (s, 1H), 7.85 (s, 1H), 7.77 (s, 1H), 7.69 (d, J=8.4 Hz, 2H), 7.63-7.65 (m, 1H), 7.56 (d, J=8.4 Hz, 2H) 7.28-7.33 (m, 1H), 7.08 (d, J=8 Hz, 1H), 6.94-6.89 (m, 1H), 4.96 (bs, 2H), 4.71 (d, J=9.6 Hz, 1H), 4.26 (q, J=7.2 Hz, 2H), 2.54-2.57 (m, 1H), 1.31 (t, J=7.2 Hz, 3H), 1.07 (d, J=6.8 Hz, 3H), 0.90 (d, J=6.8 Hz, 3H); LC-MS: m/z 458.2 (M+H).

### Step iii: Synthesis of 2-(4-(4-(3-amino-6-(2-hydroxyphenyl)pyridazin-4-yl)phenyl)-1H-pyrazol-1-yl)-3-methylbutanoic acid

To a stirred solution of ethyl 2-(4-(4-(3-amino-6-(2-hydroxyphenyl)pyridazin-4-yl)phenyl)-1H-pyrazol-1-yl)-3-methylbutanoate (0.05 g, 0.11 mmol) in methanol:THF:H₂O (3 mL:3 mL:0.5 mL) mixture was added LiOH·H₂O (0.01 g, 0.22 mmol) at 0° C. The reaction mixture was stirred for 3 h at RT. The reaction mixture was evaporated under reduced pressure and the resultant residue was diluted with methanol and acidified to pH 6 using amberlite IT120 (acidic resin) and filtered and the filtrate was concentrated under vacuum to afford the title compound as off-white solid (0.04 g, 85%). LC-MS: m/z 4 30.2 (M+H).

### Step iv: Synthesis of (2S,4R)-1-(2-(4-(4-(3-amino-6-(2-hydroxyphenyl)pyridazin-4-yl)phenyl)-1H-pyrazol-1-yl)-3-methylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(2-hydroxyphenyl)pyridazin-4-yl)phenyl)-1H-pyrazol-1-yl)-3-methylbutanoic acid (0.04 g, 0.093 mmol) and (2S,4R)-4-hydroxy-N—((S)-1-(4-(4-methylthiazol-5-yl)phenyl)ethyl)pyrrolidine-2-carboxamide hydrochloride (0.04 g, 0.10 mmol) in DMF (2 mL) at 0° C. was added HATU (0.05 g, 0.14 mmol) followed by the dropwise addition of DIPEA (0.05 mL, 0.27 mmol) and the reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 5% methanol in DCM as eluent to afford the title compound as off-white solid (0.018 g, 26%). ¹H NMR (400 MHz, DMSO-d₆): δ 13.64 (s, 1H), 8.99 (s, 1H), 8.39 (d, J=12 Hz, 1H), 8.05-7.96 (m, 3H), 7.79 (d, J=8 Hz, 2H), 7.63 (d, J=8 Hz, 2H), 7.44 (d, J=12 Hz, 1H) 7.30-7.36 (m, 4H), 7.27 (m, 1H), 6.94-6.88 (m, 2H), 6.48 (bs, 2H), 5.15 (d, J=12 Hz, 1H) 5.01-4.85 (m, 1H), 4.46-4.22 (m, 2H), 3.99-3.75 (m, 2H), 3.61 (d, J=8 Hz, 1H), 2.46 (s, 3H), 2.10-1.97 (m, 1H), 1.75-1.85 (m, 1H), 1.55-1.64 (m, 1H), 1.39 (d, J=8 Hz, 3H), 1.04 (d, J=8 Hz, 3H), 0.73 (d, J=12 Hz, 3H); LC-MS: m/z 743.3 (M+H).

### Example-8: (2S,4R)-1-((S)-2-(2-(4-(4-(3-amino-6-(2-hydroxyphenyl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 12)

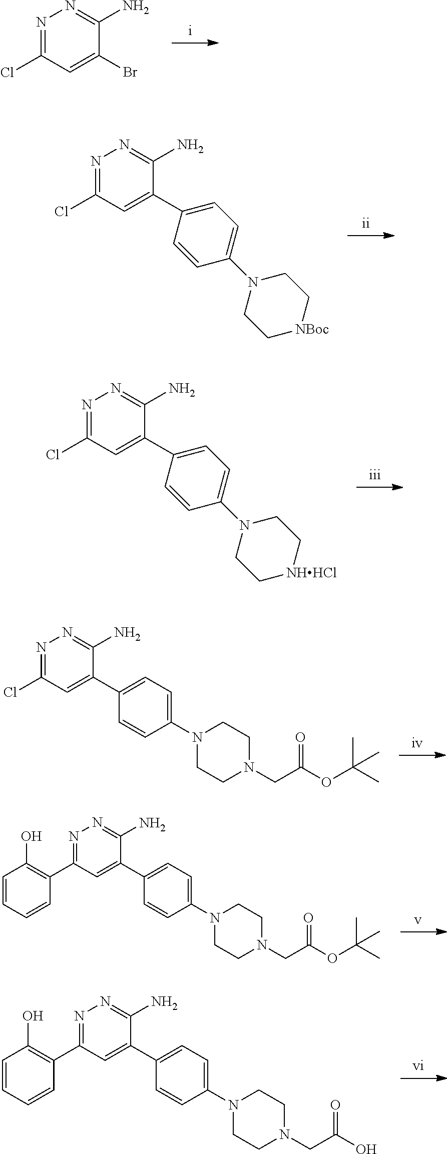

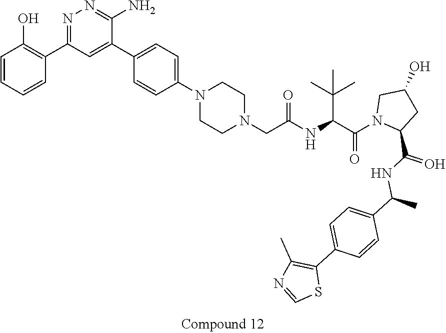 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)cc3)CC2)C(C)(C)C)cc1]

### Step i: Synthesis of tert-butyl 4-(4-(3-amino-6-chloropyridazin-4-yl)phenyl)piperazine-1-carboxylate

To a stirred solution of 4-bromo-6-chloropyridazin-3-amine (0.21 g, 1.0 mmol) and 4-(4-(4,4,5,5-tetramethyl-1,3,2-dioxaborolan-2-yl)phenyl)piperazine-1-carboxylate (0.58 g, 1.5 mmol) in 1,4-dioxane (4 mL) and water (2 mL) was added Na₂CO₃ (0.32 g, 3.0 mmol). The reaction mixture was degassed with nitrogen for 15 min. Pd(dppf)Cl₂·DCM (0.08 g, 0.1 mmol) was added to the reaction mixture and heated for 16 h at 100° C. in a sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 70% ethyl acetate in hexane as eluent to afford the title compound as off-white solid (0.18 g, 46%). ¹H NMR (400 MHz, DMSO-d₆): δ 7.45 (d, J=8.8 Hz, 2H), 7.29 (s, 1H), 7.05 (d, J=8.8 Hz, 2H), 6.28 (s, 2H), 3.47 (t, J=5.2 Hz, 4H), 3.22 (t, J=5.2 Hz, 4H), 1.42 (s, 9H). LC-MS: m/z 390.1 (M+H).

### Step ii: Synthesis of 6-chloro-4-(4-(piperazin-1-yl)phenyl)pyridazin-3-amine hydrochloride

To a stirred solution of tert-butyl 4-(4-(3-amino-6-chloropyridazin-4-yl)phenyl)piperazine-1-carboxylate (0.24 g, 0.61 mmol) in DCM (5 mL) was added 4 N dioxane hydrochloride (1 mL) at 0° C. and slowly brought to RT and stirred for 4 h. The reaction mixture was evaporated under reduced pressure, the resultant residue was washed with diethyl ether and dried under vacuum to afford the title compound as sticky material (0.23 g, crude yield). ¹H NMR (400 MHz, DMSO-d₆): δ 9.32 (s, 2H), 7.67 (s, 1H), 7.50 (d, J=8.8 Hz, 2H), 7.15 (d, J=8.0 Hz, 2H), 3.57-3.52 (m, 4H), 3.21 (s, 4H). LC-MS: m/z 290.1 (M+H).

### Step iii: Synthesis of tert-butyl 2-(4-(4-(3-amino-6-chloropyridazin-4-yl)phenyl)piperazin-1-yl)acetate

To a stirred solution of 6-chloro-4-(4-(piperazin-1-yl)phenyl)pyridazin-3-amine hydrochloride (0.23 g, 0.7 mmol) in DMF (2 mL) was added tert-butyl 2-bromoacetate (0.15 mL, 1.05 mmol) and DIPEA (0.36 mL, 2.11 mmol) at RT and stirred for 16 h at RT. The reaction mixture was quenched with cold water and extracted with EtOAc. The combined organic layer was washed with water and brine, dried over anhydrous sodium sulphate and concentrated under vacuum to afford the title compound as pale-yellow solid (0.28 g, 98%). ¹H NMR (400 MHz, DMSO-d₆): δ 7.43 (d, J=9.2 Hz, 2H), 7.28 (s, 1H), 7.05 (d, J=8.8 Hz, 2H), 6.27 (s, 2H), 3.24 (t, J=5.2 Hz, 4H), 3.17 (s, 2H), 2.65 (t, J=5.2 Hz, 4H), 1.43 (s, 9H). LC-MS: m/z 404.05 (M+H).

### Step iv: Synthesis of tert-butyl 2-(4-(4-(3-amino-6-(2-hydroxyphenyl)pyridazin-4-yl)phenyl)piperazin-1-yl)acetate

A mixture of 1,4-dioxane (4 mL) and water (2 mL) were taken in microwave vial and degassed with nitrogen for 5 min. Tert-butyl 2-(4-(4-(3-amino-6-chloropyridazin-4-yl)phenyl)piperazin-1-yl)acetate (0.27 g, 0.71 mmol) and (2-hydroxyphenyl)boronic acid (0.2 g, 1.42 mmol) were added into the reaction mixture, followed by addition of K₂CO₃ (0.2 g, 3.51 mmol) and Pd(dppf)Cl₂·DCM (0.06 g, 0.071 mmol). The reaction mixture was heated at 120° C. for 1 h in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 60% ethyl acetate in hexane as eluent to afford the title compound off-white solid (0.1 g, 32%). ¹H NMR (400 MHz, DMSO-d₆): δ 13.74 (s, 1H), 7.94 (d, J=3.6 Hz, 2H), 7.53 (d, J=8.4 Hz, 2H), 7.25 (s, 1H), 7.09 (d, J=8.8 Hz, 2H), 6.91 (t, J=7.6 Hz, 2H), 6.38 (s, 2H), 3.26 (s, 4H), 3.19 (s, 2H), 2.67 (s, 4H), 1.43 (s, 9H). LC-MS: m/z 462.3 (M+H).

### Step v: Synthesis of 2-(4-(4-(3-amino-6-(2-hydroxyphenyl)pyridazin-4-yl)phenyl)piperazin-1-yl)acetic acid

To a stirred solution of tert-butyl 2-(4-(4-(3-amino-6-(2-hydroxyphenyl)pyridazin-4-yl)phenyl)piperazin-1-yl)acetate (0.1 g, 0.21 mmol) in dioxane (2 mL) was added 4 N dioxane hydrochloride (0.5 mL) at 0° C. and slowly brought to RT and stirred for 16 h. The reaction mixture was evaporated under reduced pressure, the resultant residue was washed with diethyl ether and dried under vacuum to afford the title compound as yellow solid (0.08 g, 84%). ¹H NMR (400 MHz, DMSO-d₆): δ 10.8 (s, 1H), 8.08 (s, 2H), 7.66-7.56 (m, 3H), 7.36 (t, J=7.6 Hz, 1H), 7.19 (d, J=8.8 Hz, 2H), 7.07-6.95 (m, 2H), 4.23 (s, 2H), 3.57 (s, 8H).

### Step vi: Synthesis of (2S,4R)-1-((S)-2-(2-(4-(4-(3-amino-6-(2-hydroxyphenyl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(2-hydroxyphenyl)pyridazin-4-yl)phenyl)piperazin-1-yl)acetic acid (0.08 g, 0.18 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.13 g, 0.27 mmol) and HATU (0.1 g, 0.27 mmol) in DMF (2 mL) was dropwise added DIPEA (0.16 mL, 0.9 mmol) at 0° C. and slowly brought to RT and the reaction mixture was stirred for 1 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 5% methanol in DCM as eluent to afford the title compound as a brown solid (0.04 g, 26%). ¹H NMR (400 MHz, DMSO-d₆): δ 13.74 (s, 1H), 8.98 (s, 1H), 8.42 (d, J=7.6 Hz, 1H), 7.94 (d, J=8.4 Hz, 2H), 7.79 (d, J=9.2 Hz, 1H), 7.54 (d, J=8.4 Hz, 2H), 7.42-7.35 (m, 4H), 7.25 (t, J=8.0 Hz, 1H), 7.13 (d, J=8.0 Hz, 2H), 6.89 (q, J=8.0 Hz, 2H), 6.37 (s, 2H), 5.12 (d, J=2.4 Hz, 1H), 4.89 (t, J=6.8 Hz, 1H), 4.54-4.42 (m, 2H), 4.29 (s, 1H), 3.59 (s, 2H), 3.18-3.02 (m, 2H), 2.67 (bs, 4H), 2.45 (bs, 7H), 2.03-2.10 (m, 1H), 1.72-1.80 (m, 1H), 1.37 (d, J=8 Hz, 3H), 0.96 (s, 9H). LC-MS: m/z 832.4 (M+H).

### Example-9: (2S,4R)-1-((S)-2-(2-(4-(4-(3-amino-6-chloropyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 13)

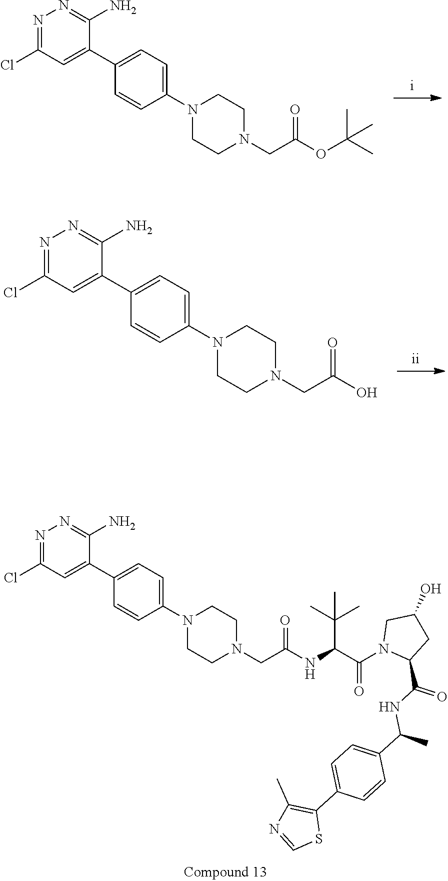 [SMILES: CC(C)(C)OC(=O)CN1CCN(c2ccc(-c3cc(Cl)nnc3N)cc2)CC1 ; Nc1nnc(Cl)cc1-c1ccc(N2CCN(CC(=O)O)CC2)cc1 ; Cc1ncsc1-c1ccc([C@H](C)NC(=O)[C@@H]2C[C@@H](O)CN2C(=O)[C@@H](NC(=O)CN2CCN(c3ccc(-c4cc(Cl)nnc4N)cc3)CC2)C(C)(C)C)cc1]

### Step i: Synthesis of 2-(4-(4-(3-amino-6-chloropyridazin-4-yl)phenyl)piperazin-1-yl)acetic acid

To a stirred solution of tert-butyl 2-(4-(4-(3-amino-6-chloropyridazin-4-yl)phenyl)piperazin-1-yl)acetate (0.3 g, 0.74 mmol) in DCM (10 mL) was added 4 N dioxane hydrochloride (2 mL) at 0° C. and then slowly brought to RT and stirred for 16 h. The reaction mixture was evaporated under reduced pressure, the resultant residue was washed with diethyl ether and dried under vacuum to afford the title compound as sticky material (0.3 g, crude yield). ¹H NMR (400 MHz, DMSO-d₆): δ 11.0 (s, 1H), 7.72 (s, 1H), 7.51 (d, J=8.8 Hz, 2H), 7.16 (d, J=8.8 Hz, 2H), 4.38 (s, 4H), 4.24 (s, 4H), 3.56 (s, 2H).

### Step ii: Synthesis of (2S,4R)-1-((S)-2-(2-(4-(4-(3-amino-6-chloropyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-chloropyridazin-4-yl)phenyl)piperazin-1-yl)acetic acid (0.04 g, 0.10 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.06 g, 0.12 mmol) and HATU (0.05 g, 0.12 mmol) in DMF (5 mL) was dropwise added DIPEA (0.1 mL, 0.52 mmol) at 0° C. and slowly brought to RT and the reaction mixture was stirred for 1 h. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 6-7% methanol in DCM as eluent to afford the title compound as pale-yellow solid (0.02 g, 22%). ¹H NMR (400 MHz, DMSO-d₆): δ 8.98 (s, 1H), 8.44 (d, J=7.6 Hz, 1H), 7.79 (d, J=9.6 Hz, 1H), 7.45-7.42 (m, 4H), 7.36 (d, J=8.0 Hz, 2H), 7.29 (s, 1H), 7.08 (d, J=8.8 Hz, 2H), 6.28 (s, 2H), 5.13 (d, J=3.6 Hz, 1H), 4.88-4.78 (m, 1H), 4.52 (d, J=10.0 Hz, 1H), 4.44 (s, 1H), 4.29 (s, 1H), 3.59 (d, J=3.6 Hz, 2H), 3.36-3.29 (m, 4H), 2.67-2.64 (m, 6H), 2.45 (s, 3H), 2.03-2.10 (m, 1H), 1.72-1.80 (m, 1H), 1.23 (d, J=9.2 Hz, 3H), 0.95 (s, 9H). LC-MS: m/z 774.3 (M+H).

### Example-10: (2S,4R)-1-((S)-2-(2-(4-(4-(3-amino-6-(trifluoromethyl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 14)

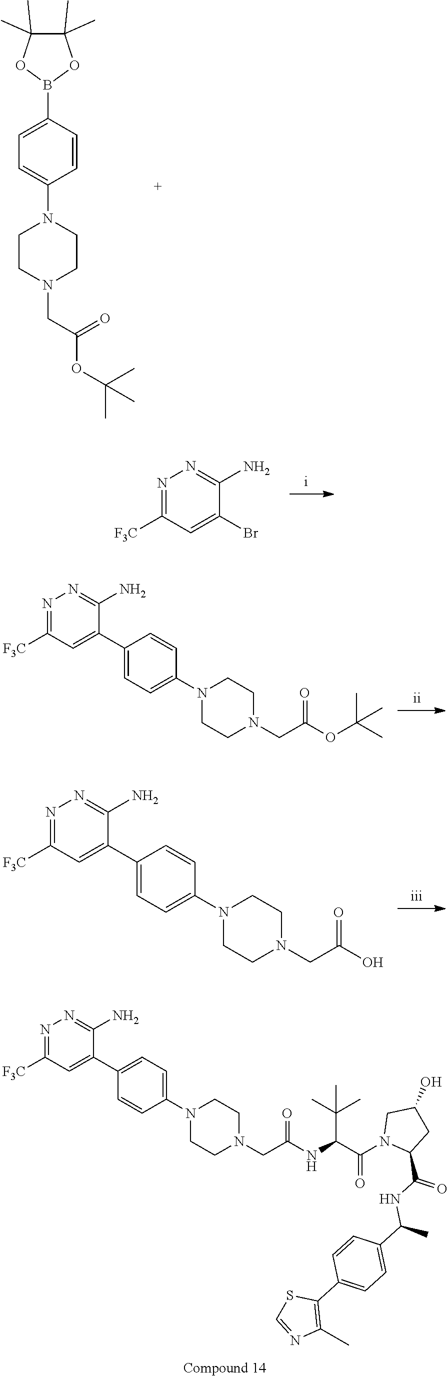 [SMILES: CC(C)(C)OC(=O)CN1CCN(c2ccc(B3OC(C)(C)C(C)(C)O3)cc2)CC1 ; Nc1nnc(C(F)(F)F)cc1Br ; CC(C)(C)OC(=O)CN1CCN(c2ccc(-c3cc(C(F)(F)F)nnc3N)cc2)CC1 ; Nc1nnc(C(F)(F)F)cc1-c1ccc(N2CCN(CC(=O)O)CC2)cc1 ; Cc1ncsc1-c1ccc([C@H](C)NC(=O)[C@@H]2C[C@@H](O)CN2C(=O)[C@@H](NC(=O)CN2CCN(c3ccc(-c4cc(C(F)(F)F)nnc4N)cc3)CC2)C(C)(C)C)cc1]

### Step i: Synthesis of tert-butyl 2-(4-(4-(3-amino-6-(trifluoromethyl)pyridazin-4-yl)phenyl)piperazin-1-yl)acetate

To a stirred solution of 4-bromo-6-(trifluoromethyl)pyridazin-3-amine (0.15 g, 0.61 mmol), tert-butyl 2-(4-(4-(4,4,5,5-tetramethyl-1,3,2-dioxaborolan-2-yl)phenyl)piperazin-1-yl)acetate (0.027 g, 0.68 mmol) and K₂CO₃ (0.25 g, 1.85 mmol) in 1,4-dioxane (4 mL) and water (1 mL). The reaction mixture was degassed with nitrogen for 15 min. Pd(dppf)Cl₂·DCM (0.05 g, 0.0061 mmol) was added to the reaction mixture and heated at 110° C. for 1 h in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 45% ethyl acetate in hexane as eluent to afford the title compound off-white solid (0.15 g, 96%). ¹H NMR (400 MHz, DMSO-d₆): δ 7.49 (s, 1H), 7.46-7.44 (m, 2H), 7.05 (d, J=8.8 Hz, 2H), 6.83 (s, 2H), 3.91 (s, 2H), 3.25-3.23 (m, 4H), 2.66-2.64 (m, 4H), 1.42 (s, 9H); LC-MS: m/z 438.2 (M+H).

### Step ii: Synthesis of 2-(4-(4-(3-amino-6-(trifluoromethyl)pyridazin-4-yl)phenyl)piperazin-1-yl)acetic acid hydrochloride

To a stirred solution of tert-butyl 2-(4-(4-(3-amino-6-(trifluoromethyl)pyridazin-4-yl)phenyl)piperazin-1-yl)acetate (0.15 g, 0.34 mmol) in DCM (10 mL) was added 4N dioxane hydrochloride (0.75 mL) at 0° C. and then slowly brought to RT and stirred for 16 h. The reaction mixture was evaporated under reduced pressure, the resultant residue was washed with diethyl ether and dried under vacuum to afford the title compound as sticky material (0.11 g, crude yield). LC-MS: m/z 382.1 (M+H).

### Step iii: Synthesis of ((2S,4R)-1-((S)-2-(2-(4-(4-(3-amino-6-(2-methoxypyridin-3-yl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(trifluoromethyl)pyridazin-4-yl)phenyl)piperazin-1-yl)acetic acid hydrochloride (0.05 g, 0.11 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.07 g, 0.14 eq.) in DMF (2 mL) at 0° C. was added HATU (0.055 g, 0.14 mmol) followed by the dropwise addition of DIPEA (0.1 mL, 0.59 mmol) and the reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product which was purified by purified by preparative HPLC to afford the title compound off-white solid (0.015 g, 11%). ¹H NMR (400 MHz, DMSO-d₆): δ 8.95 (s, 1H), 8.39 (d, J=7.2 Hz, 1H), 7.76 (d, J=10.0 Hz, 1H), 7.48-7.33 (m, 7H), 7.07 (d, J=8.8 Hz, 2H), 6.82 (s, 2H), 5.10 (s, 1H), 4.86 (t, J=7.2 Hz, 1H), 4.5 (d, J=10.0 Hz, 1H), 4.42 (t, J=8.4 Hz, 1H), 4.26 (s, 1H), 3.58-3.57 (m, 2H), 3.14-3.1 (m, 1H), 3.02-2.98 (m, 1H), 2.63 (bs, 6H), 2.47-2.43 (m, 2H), 2.42 (s, 3H), 2.03-2.02 (m, 1H), 1.75-1.74 (m, 1H), 1.34 (d, J=8.0 Hz, 3H), 0.93 (s, 9H); LC-MS: m/z 808.3 (M+H).

### Example-11: (2S,4R)-1-((S)-2-(2-(4-(4-(3-amino-6-(2-methoxypyridin-3-yl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 15)

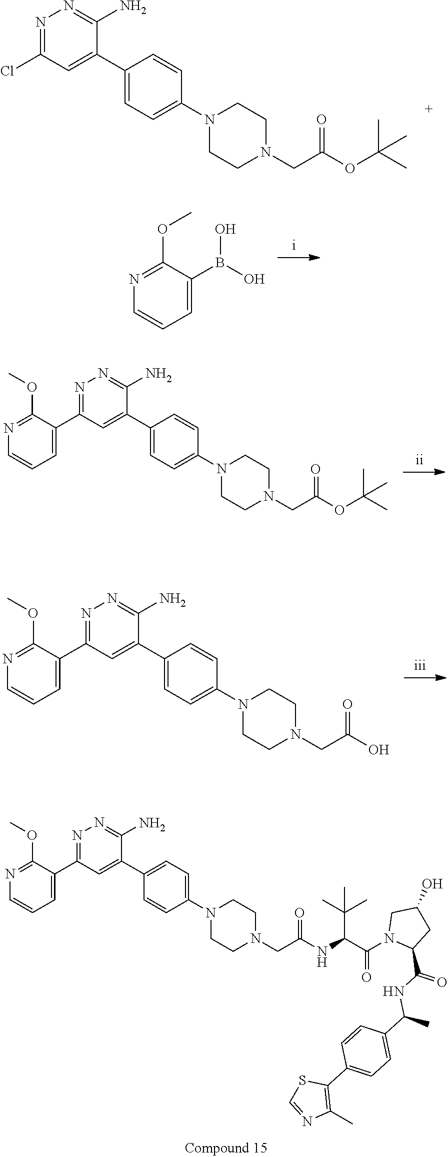 [SMILES: CC(C)(C)OC(=O)CN1CCN(c2ccc(-c3cc(Cl)nnc3N)cc2)CC1 ; COc1ncccc1B(O)O ; COc1ncccc1-c1cc(-c2ccc(N3CCN(CC(=O)OC(C)(C)C)CC3)cc2)c(N)nn1 ; COc1ncccc1-c1cc(-c2ccc(N3CCN(CC(=O)O)CC3)cc2)c(N)nn1 ; COc1ncccc1-c1cc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)cc2)c(N)nn1]

### Step i: Synthesis of tert-butyl 2-(4-(4-(3-amino-6-(2-methoxypyridin-3-yl)pyridazin-4-yl)phenyl)piperazin-1-yl)acetate

To a stirred solution of tert-butyl 2-(4-(4-(3-amino-6-chloropyridazin-4-yl)phenyl)piperazin-1-yl)acetate (0.22 g, 0.54 mmol) and (2-methoxypyridin-3-yl)boronic acid (0.17 g, 1.08 mmol) in DME (10 mL) and water (1 mL) was added K₂CO₃ (0.22 g, 1.63 mmol). The reaction mixture degassed with nitrogen for 15 min. Pd(dppf)Cl₂·DCM (0.045 g, 0.054 mmol) was added into the reaction mixture and heated for 6 h at 120° C. in a sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 2-3% methanol in DCM as eluent to afford the title compound as yellow solid (0.22 g, 84%). ¹H NMR (400 MHz, DMSO-d₆): δ 8.24-8.22 (m, 1H), 8.15-8.13 (m, 1H), 7.77 (s, 1H), 7.39-7.37 (m, 2H), 6.97-6.95 (m, 3H), 4.91 (bs, 2H), 3.91 (s, 3H), 3.29-3.26 (m, 4H), 3.13 (s, 2H), 2.71-2.68 (m, 4H), 1.42 (s, 9H); LC-MS: m/z 477.2 (M+H).

### Step ii: Synthesis of 2-(4-(4-(3-amino-6-(2-methoxypyridin-3-yl)pyridazin-4-yl)phenyl)piperazin-1-yl)acetic acid

To a stirred solution of tert-butyl 2-(4-(4-(3-amino-6-(2-methoxypyridin-3-yl)pyridazin-4-yl)phenyl)piperazin-1-yl)acetate (0.11 g, 0.23 mmol) in DCM (10 mL) was added 4 N dioxane hydrochloride (0.33 mL) at 0° C. and slowly brought to RT and stirred for 2 h. The reaction mixture was evaporated under reduced pressure, the resultant residue was washed with diethyl ether and dried under vacuum to afford the title compound as sticky material (0.1 g, crude yield). LC-MS: m/z 421.2 (M+H).

### Step iii: Synthesis of ((2S,4R)-1-((S)-2-(2-(4-(4-(3-amino-6-(2-methoxypyridin-3-yl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(2-methoxypyridin-3-yl)pyridazin-4-yl)phenyl)piperazin-1-yl)acetic acid (0.1 g, 0.22 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.13 g, 0.27 mmol) in DMF (2 mL) at 0° C. was added HATU (0.1 g, 0.27 mmol) followed by the dropwise addition of DIPEA (0.2 mL, 1.12 mmol) and the reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to get the crude product. The crude product was purified by preparative HPLC to afford the title compound yellow solid (0.02 g, 10%). ¹H NMR (400 MHz, DMSO-d₆): δ 8.98 (s, 1H), 8.45 (d, J=8.0 Hz, 1H), 8.24-8.22 (m, 1H), 8.17-8.14 (m, 1H), 7.79 (d, J=9.2 Hz, 1H), 7.61 (s, 1H), 7.47-7.37 (m, 6H), 7.16-7.09 (m, 3H), 6.19 (s, 2H), 5.15 (d, J=3.2 Hz, 1H), 4.88 (t, J=7.2 Hz, 1H), 4.52 (d, J=9.6 Hz, 1H), 4.44 (t, J=8.4 Hz, 1H), 4.28 (s, 1H), 3.90 (s, 3H), 3.60-3.59 (m, 2H), 3.31-3.30 (m, 4H), 3.17-3.13 (m, 1H), 3.04-3.00 (m, 1H), 2.67-2.66 (m, 4H), 2.45 (s, 3H), 2.05-2.03 (m, 1H), 1.80-1.75 (m, 1H), 1.36 (d, J=7.2 Hz, 3H), 0.95 (s, 9H); LC-MS: m/z 847.40 (M+H).

### Example-12: (2S,4R)-1-((S)-2-(2-(4-(4-(3-amino-6-(2-hydroxypyridin-3-yl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 16)

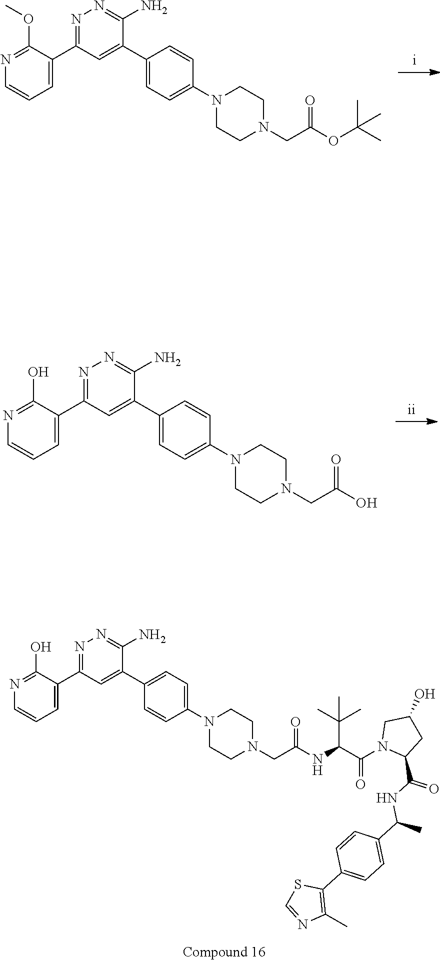 [SMILES: COc1ncccc1-c1cc(-c2ccc(N3CCN(CC(=O)OC(C)(C)C)CC3)cc2)c(N)nn1 ; Nc1nnc(-c2cccnc2O)cc1-c1ccc(N2CCN(CC(=O)O)CC2)cc1 ; Cc1ncsc1-c1ccc([C@H](C)NC(=O)[C@@H]2C[C@@H](O)CN2C(=O)[C@@H](NC(=O)CN2CCN(c3ccc(-c4cc(-c5cccnc5O)nnc4N)cc3)CC2)C(C)(C)C)cc1]

### Step i: Synthesis of 2-(4-(4-(3-amino-6-(2-hydroxypyridin-3-yl)pyridazin-4-yl)phenyl)piperazin-1-yl)acetic acid

To a stirred solution of tert-butyl 2-(4-(4-(3-amino-6-(2-methoxypyridin-3-yl)pyridazin-4-yl)phenyl)piperazin-1-yl)acetate (0.11 g, 0.23 mmol) in DCM (10 mL) was added 4 N dioxane hydrochloride (1 mL) at 0° C. and slowly brought to RT and stirred for 16 h. The reaction mixture was evaporated under reduced pressure, the resultant residue was washed with diethyl ether and dried under vacuum to afford the title compound as sticky material (0.1 g, crude yield). LC-MS: m/z 407.1 (M+H).

### Step ii: Synthesis of (2S,4R)-1-(2-(3-(4-(3-amino-6-(2-oxo-1,2-dihydropyridin-3-yl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(2-hydroxypyridin-3-yl)pyridazin-4-yl)phenyl)piperazin-1-yl)acetic acid (0.1 g, 0.22 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.13 g, 0.27 mmol) in DMF (2 mL) at 0° C. was added HATU (0.1 g, 0.27 mmol), followed by the dropwise addition of DIPEA (0.2 mL, 1.12 mmol). The reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product. The crude product was purified by preparative HPLC to afford the title compound yellow solid (0.02 g, 10%). ¹H NMR (400 MHz, DMSO-d₆): δ 11.91 (s, 1H), 8.97 (s, 1H), 8.42 (d, J=7.2 Hz, 1H), 8.25 (d, J=5.6 Hz, 1H), 8.13 (s, 1H), 7.78 (d, J=10.0 Hz, 1H), 7.46-7.35 (m, 7H), 7.10 (d, J=8.8 Hz, 2H), 6.38 (t, J=6.0 Hz, 1H), 6.06 (s, 2H), 5.12 (d, J=2.4 Hz, 1H), 4.89 (t, J=6.8 Hz, 1H), 4.53-4.30 (m, 2H), 4.29 (bs, 1H), 3.59-3.56 (m, 2H), 2.66-2.61 (m, 6H), 3.16-3.03 (m, 2H), 2.5-2.45 (m, 5H), 2.32 (s, 2H), 2.08-2.03 (m, 1H), 1.79-1.74 (m, 1H), 1.36 (d, J=6.8 Hz, 3H), 0.96 (s, 9H); LC-MS: m/z 833.35 (M+H)

### Example-13: ((2S,4R)-1-(2-(3-(4-(3-amino-6-(2-oxo-1,2-dihydropyridin-3-yl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 17)

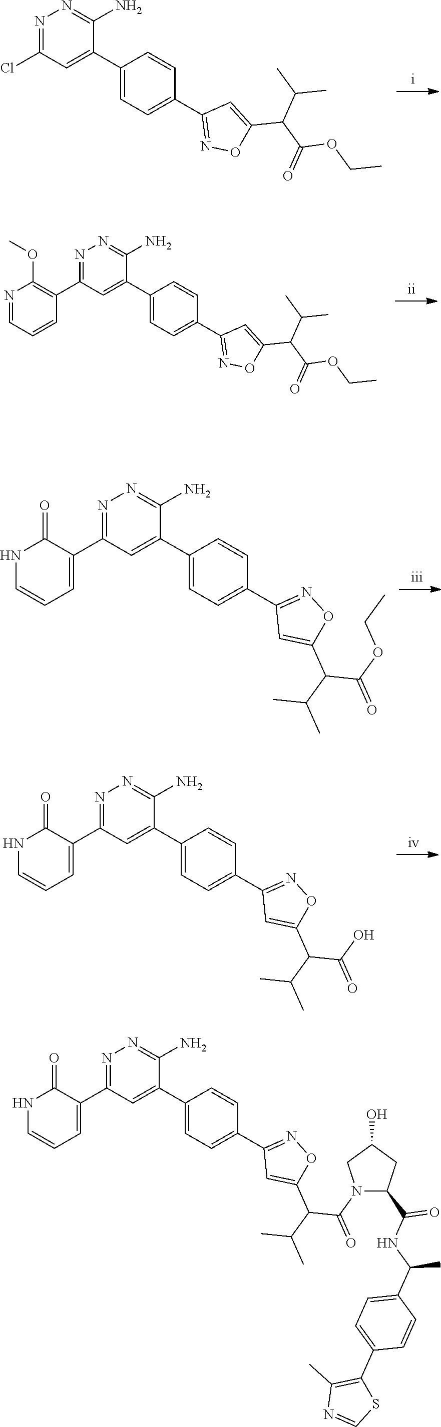 [SMILES: CCOC(=O)C(c1cc(-c2ccc(-c3cc(Cl)nnc3N)cc2)no1)C(C)C ; CCOC(=O)C(c1cc(-c2ccc(-c3cc(-c4cccnc4OC)nnc3N)cc2)no1)C(C)C ; CCOC(=O)C(c1cc(-c2ccc(-c3cc(-c4ccc[nH]c4=O)nnc3N)cc2)no1)C(C)C ; CC(C)C(C(=O)O)c1cc(-c2ccc(-c3cc(-c4ccc[nH]c4=O)nnc3N)cc2)no1 ; Cc1ncsc1-c1ccc([C@H](C)NC(=O)[C@@H]2C[C@@H](O)CN2C(=O)C(c2cc(-c3ccc(-c4cc(-c5ccc[nH]c5=O)nnc4N)cc3)no2)C(C)C)cc1]

### Step i: Synthesis of ethyl 2-(3-(4-(3-amino-6-(2-methoxypyridin-3-yl)pyridazin-4-yl)phenyl)isoxazol-5-yl)-3-methylbutanoate

To a stirred solution of ethyl 2-(3-(4-(3-amino-6-chloropyridazin-4-yl)phenyl)isoxazol-5-yl)-3-methylbutanoate (0.12 g, 0.29 mmol) and (2-methoxypyridin-3-yl)boronic acid (0.09 g, 0.59 mmol) in DME (4 mL) and water (2 mL) was added K₂CO₃ (0.08 g, 0.59 mmol). The rection mixture was degassed with nitrogen for 15 min. Pd(dppf)Cl₂·DCM (0.024 g, 0.029 mmol) was added to the reaction mixture and heated for 1 h at 120° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 3% methanol in DCM as eluent to afford the title compound as off-white solid (0.05 g, 35%). LC-MS: m/z 474.3 (M+H).

### Step ii: Synthesis of ethyl 2-(3-(4-(3-amino-6-(2-oxo-1,2-dihydropyridin-3-yl)pyridazin-4-yl)phenyl)isoxazol-5-yl)-3-methylbutanoate

To a stirred solution of compound ethyl 2-(3-(4-(3-amino-6-(2-methoxypyridin-3-yl)pyridazin-4-yl)phenyl)isoxazol-5-yl)-3-methylbutanoate (0.05 g, 0.1 mmol) in DMF (2 mL) mixture was added PTSA (0.09 g, 0.5 mmol) and LiCl (0.02 g, 0.5 mmol) and the reaction mixture was heated at 120° C. for 1 h. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 6% methanol in DCM as eluent to afford the title compound as yellow solid (0.04 g, 83%). %). ¹H NMR (400 MHz, DMSO-d₆): δ 11.97 (s, 1H), 8.27-8.29 (m, 1H), 8.22 (s, 1H), 8.04 (d, J=8.0 Hz, 1H), 7.67 (d, J=8.0 Hz, 2H), 7.51 (bs, 1H), 7.11 (s, 1H), 6.4 (d, J=6.4 Hz, 1H), 6.30 (bs, 2H), 4.16 (q, J=8.0 Hz, 2H), 3.88 (d, J=8.4 Hz, 1H), 2.41 (m, 1H), 1.22 (t, J=3.6 Hz, 3H), 1.00 (d, J=6.4 Hz, 3H), 0.91 (d, J=6.4 Hz, 3H); LC-MS: m/z 460.2 (M+H).

### Step iii: Synthesis of 2-(3-(4-(3-amino-6-(2-oxo-1,2-dihydropyridin-3-yl)pyridazin-4-yl)phenyl)isoxazol-5-yl)-3-methylbutanoic acid

To a stirred solution of compound ethyl 2-(3-(4-(3-amino-6-(2-oxo-1,2-dihydropyridin-3-yl)pyridazin-4-yl)phenyl)isoxazol-5-yl)-3-methylbutanoate (0.04 g, 0.087 mmol) in methanol:THF:H₂O (1 mL: 2 mL: 1 mL) mixture was added LiOH·H₂O (0.01 g, 0.26 mmol) at 0° C. The reaction mixture was stirred for 16 h at RT. The reaction mixture was evaporated under reduced pressure and the resultant residue was diluted with methanol and acidified to pH 6 using amberlite IT120 (acidic rasin) and filtered. The filtrate was concentrated under vacuum to afford the title compound as a sticky solid (0.04 g, crude yield). LC-MS: m/z 432.1 (M+H).

### Step iv: Synthesis of ((2S,4R)-1-(2-(3-(4-(3-amino-6-(2-oxo-1,2-dihydropyridin-3-yl)pyridazin-4-yl)phenyl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(4-(3-amino-6-(2-oxo-1,2-dihydropyridin-3-yl)pyridazin-4-yl)phenyl)isoxazol-5-yl)-3-methylbutanoic acid (0.038 g, 0.086 mmol) and (2S,4R)-4-hydroxy-N—((S)-1-(4-(4-methylthiazol-5-yl)phenyl)ethyl)pyrrolidine-2-carboxamide hydrochloride (0.038 g, 0.1 mmol) in DMF (5 mL) at 0° C. was added HATU (0.04 g, 0.1 mmol) followed by the dropwise addition of DIPEA (0.08 mL, 0.4 mmol) and the reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 12% methanol in DCM as eluent to afford the title compound off-white solid (0.005 g, 7%). ¹H NMR (400 MHz, DMSO-d₆): δ 11.96 (bs, 1H), 8.98 (s, 1H), 8.28 (d, J=7.2 Hz, 2H), 8.23 (s, 1H), 8.02 (d, J=8.0 Hz, 1H), 7.98-7.95 (m, 1H), 7.68-7.62 (m, 2H), 7.48-7.29 (m, 5H), 7.00 (d, J=7.2 Hz, 1H), 6.41-6.38 (m, 1H), 6.30 (bs, 2H), 5.13-5.1 (m, 1H), 4.95-4.93 (m, 1H), 4.46-4.41 (m, 1H), 4.3 (bs, 1H), 3.99-3.88 (m, 1H), 3.62-3.51 (m, 2H), 2.44 (s, 3H), 2.06-2.04 (m, 1H), 1.85-1.80 (m, 1H), 1.49 (d, J=6.8 Hz, 1H), 1.02 (d, J=6.4 Hz, 3H), 0.88-0.80 (m, 6H); LC-MS: m/z 745.2 (M+H).

### Example-14: (2S,4R)-1-((S)-2-(3-(4-(4-(3-amino-6-(2-hydroxyphenyl)pyridazin-4-yl)phenyl)piperazin-1-yl)propanamido)-3,3-dimethylbutanoyl)-4-hydroxy-N—((S)-1-(4-(4-methylthiazol-5-yl)phenyl)ethyl)pyrrolidine-2-carboxamide (Compound 18)

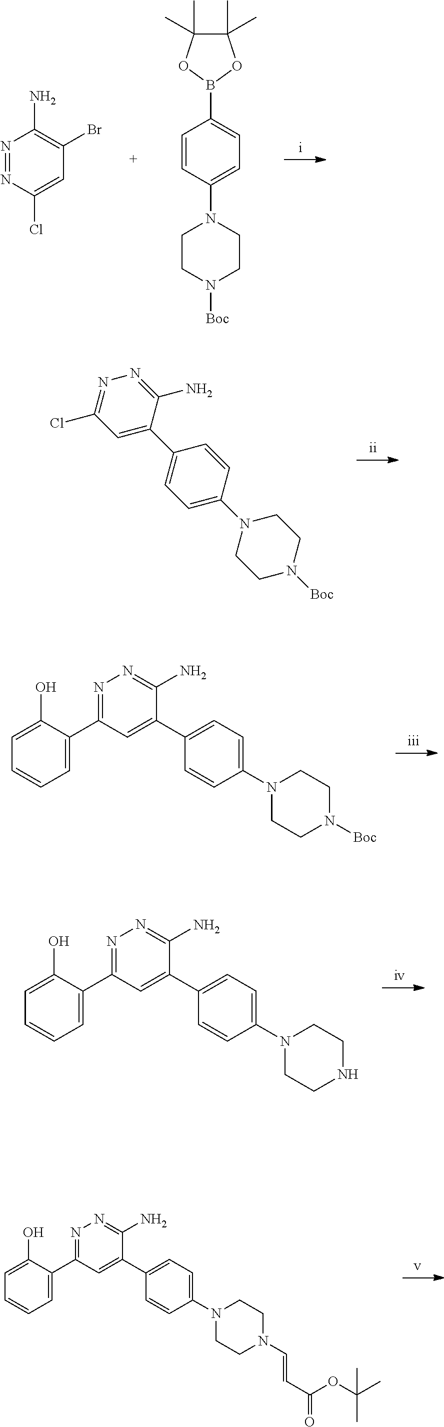

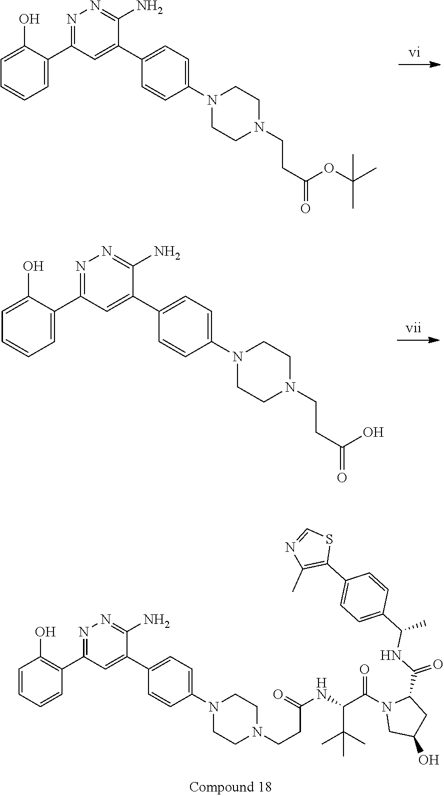 [SMILES: CC(C)(C)OC(=O)CCN1CCN(c2ccc(-c3cc(-c4ccccc4O)nnc3N)cc2)CC1 ; Nc1nnc(-c2ccccc2O)cc1-c1ccc(N2CCN(CCC(=O)O)CC2)cc1 ; Cc1ncsc1-c1ccc([C@H](C)NC(=O)[C@@H]2C[C@@H](O)CN2C(=O)[C@@H](NC(=O)CCN2CCN(c3ccc(-c4cc(-c5ccccc5O)nnc4N)cc3)CC2)C(C)(C)C)cc1]

### Step i: Synthesis of tert-butyl 4-(4-(3-amino-6-chloropyridazin-4-yl)phenyl)piperazine-1-carboxylate

To a stirred solution of tert-butyl 4-(4-(4,4,5,5-tetramethyl-1,3,2-dioxaborolan-2-yl)phenyl)piperazine-1-carboxylate (2.0 g, 5.15 mmol) and 4-bromo-6-chloropyridazin-3-amine (1.6 g, 7.7 mmol) in dioxane (20 mL) and water (10 mL) was added K₂CO₃ (1.6 g, 15.4 mmol). The reaction mixture degassed with nitrogen for 15 min. Pd(dppf)Cl₂·DCM (0.42 g, 0.51 mmol) was added to the reaction mixture and heated for 16 h at 120° C. in a sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 80% ethyl acetate in hexane as eluent to afford the title compound as off-white solid (1 g, 50%). ¹H NMR (400 MHz, DMSO-d₆): δ 7.45 (d, J=8.8 Hz, 2H), 7.29 (s, 1H), 7.05 (d, J=8.8 Hz, 2H), 6.28 (s, 2H), 3.47 (t, J=5.2 Hz, 4H), 3.22 (t, J=5.2 Hz, 4H), 1.42 (s, 9H). LC-MS: m/z 390.2 (M+H).

### Step ii: Synthesis of tert-butyl 4-(4-(3-amino-6-(2-hydroxyphenyl)pyridazin-4-yl)phenyl)piperazine-1-carboxylate

To a stirred solution of ethyl tert-butyl 4-(4-(3-amino-6-chloropyridazin-4-yl)phenyl)piperazine-1-carboxylate (1.0 g, 2.56 mmol) and (2-hydroxyphenyl)boronic acid (0.7 g, 5.1 mmol) in dioxane (20 mL) and water (10 mL) was added K₂CO₃ (0.7 g, 0.59 mmol) and degassed with nitrogen for 15 min. Pd(dppf)Cl₂·DCM (0.024 g, 0.029 mmol) was added to the reaction mixture and heated for 6 h at 120° C. in a sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 60% ethyl acetate in hexane as eluent to afford the title compound as yellow solid (0.2 g, 17%). ¹H NMR (400 MHz, DMSO-d₆): δ 13.72 (s, 1H), 7.93 (d, J=4.4 Hz, 2H), 7.54 (d, J=8.8 Hz, 2H), 7.24 (m, 1H), 7.10 (d, J=8.8 Hz, 2H), 7.86-7.92 (m, 2H), 6.36 (s, 2H), 3.48 (t, J=5.2 Hz, 4H), 3.23 (t, J=5.2 Hz, 4H), 1.43 (s, 9H). LC-MS: m/z 448.2 (M+H).

### Step iii: Synthesis of 2-(6-amino-5-(4-(piperazin-1-yl)phenyl)pyridazin-3-yl)phenol

To a stirred solution of tert-butyl 4-(4-(3-amino-6-chloropyridazin-4-yl)phenyl)piperazine-1-carboxylate (0.2 g, 0.41 mmol) in DCM (5 mL) was added 4 N dioxane hydrochloride (1 mL) at 0° C. and slowly brought to RT and stirred for 2 h. The reaction mixture was evaporated under reduced pressure, the resultant residue was washed with diethyl ether and dried under vacuum to afford the title compound as sticky material (0.2 g, crude yield). LC-MS: m/z 348.2 (M+H).

### Step iv: Synthesis of tert-butyl (E)-3-(4-(4-(3-amino-6-(2-hydroxyphenyl)pyridazin-4-yl)phenyl)piperazin-1-yl)acrylate

To a stirred solution of 2-(6-amino-5-(4-(piperazin-1-yl)phenyl)pyridazin-3-yl)phenol (0.2 g, 0.52 mmol) in DMF (5 mL) cooled to 0° C. and stirred for 5 min. DIPEA (0.45 g, 2.6 mmol) and tert-butyl propiolate (0.14 g, 1.04 mmol) was added to the reaction mixture and stirred for 16 h at RT under nitrogen atmosphere. The reaction mixture was quenched with cold water and stirred for 1 h; the solid so formed was filtered and dried under vacuum to afford the title compound as off-white solid (0.15 g, 60%). ¹H NMR (400 MHz, DMSO-d₆): δ 13.79 (s, 1H), 7.94 (d, J=5.2 Hz, 2H), 7.55 (d, J=8.8 Hz, 2H), 7.33 (d, J=13.2 Hz, 1H), 7.25 (t, J=7.6 Hz, 1H), 7.12 (d, J=8.4 Hz, 2H), 6.90 (q, J=8.4 Hz, 2H), 6.38 (s, 2H), 3.39 (s, 4H), 3.29 (s, 4H), 1.39 (s, 9H). LC-MS: m/z 474.3 (M+H).

### Step v: Synthesis of tert-butyl 3-(4-(4-(3-amino-6-(2-hydroxyphenyl)pyridazin-4-yl)phenyl)piperazin-1-yl)propanoate

To a stirred solution of tert-butyl (E)-3-(4-(4-(3-amino-6-(2-hydroxyphenyl)pyridazin-4-yl)phenyl)piperazin-1-yl)acrylate (0.15 g, 0.31 mmol) in methanol (10 mL) was added Pd—C (0.1 g,) at RT and stirred for 4 h under hydrogen atmosphere. Once the reaction was completed (monitored by TLC), the reaction mixture was diluted with methanol and filtered through Celite®. The filtrate was concentrated under vacuum to afford the title compound as off-white solid (0.03 g, 20%). LC-MS: m/z 476.25 (M+H).

### Step vi: Synthesis of 3-(4-(4-(3-amino-6-(2-hydroxyphenyl)pyridazin-4-yl)phenyl)piperazin-1-yl)propanoic acid

To a stirred solution of tert-butyl 3-(4-(4-(3-amino-6-(2-hydroxyphenyl)pyridazin-4-yl)phenyl)piperazin-1-yl)propanoate (0.03 g, 0.063 mmol) in DCM (5 mL) was added 4 N dioxane hydrochloride (1 mL) at 0° C. and slowly brought to RT and stirred for 16 h. The reaction mixture was evaporated under reduced pressure, the resultant residue was washed with diethyl ether and dried under vacuum to afford the title compound as sticky material (0.025 g, crude yield).

### Step vii: Synthesis of (2S,4R)-1-((S)-2-(3-(4-(4-(3-amino-6-(2-hydroxyphenyl)pyridazin-4-yl)phenyl)piperazin-1-yl)propanamido)-3,3-dimethylbutanoyl)-4-hydroxy-N—((S)-1-(4-(4-methylthiazol-5-yl)phenyl)ethyl)pyrrolidine-2-carboxamide

To a solution of 3-(4-(4-(3-amino-6-(2-hydroxyphenyl)pyridazin-4-yl)phenyl)piperazin-1-yl)propanoic acid (0.025 g, 0.059 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.034 g, 0.071 mmol) and HATU (0.027 g, 0.071 mmol) in DMF (5 mL) was dropwise added DIPEA (0.05 mL, 0.29 mmol) at 0° C. and slowly brought to RT and the reaction mixture was stirred for 1 h at RT. The reaction mixture was poured into ice cold water and extracted with ethyl acetate. The combined organic layer was washed with water and brine, dried over anhydrous sodium sulphate and concentrated under vacuum to give the crude product which was purified by combiflash column chromatography using 6-7% methanol in DCM as eluent to afford the title compound as a pale yellow solid (0.01 g, 20%). ¹H NMR (400 MHz, DMSO-d₆): δ 13.77 (s, 1H), 8.97 (s, 1H), 8.45-8.31 (m, 2H), 7.93 (d, J=4.4 Hz, 2H), 7.53 (d, J=8.4 Hz, 2H), 7.43-7.35 (m, 4H), 7.29-7.20 (m, 1H), 7.11 (d, J=8.4 Hz, 2H), 6.91 (t, J=9.6 Hz, 2H), 6.37 (s, 2H), 5.10 (d, J=2.8 Hz, 1H), 4.59-4.51 (m, 1H), 4.48-4.41 (m, 1H), 4.32-4.28 (m, 1H), 3.59 (s, 3H), 3.21-3.40 (m, 6H), 2.60-2.42 (m, 9H), 2.06-1.98 (m, 1H), 1.83-1.75 (m, 1H), 1.36 (d, J=7.2 Hz, 3H), 0.95 (s, 9H). LC-MS: m/z 846.45 (M+H).

### Example-15: (2S,4R)-1-((S)-2-(6-(4-(3-amino-6-(2-hydroxyphenyl)pyridazin-4-yl)phenyl)hex-5-ynamido)-3,3-dimethylbutanoyl)-4-hydroxy-N—((S)-1-(4-(4-methylthiazol-5-yl)phenyl)ethyl)pyrrolidine-2-carboxamide (Compound 19)

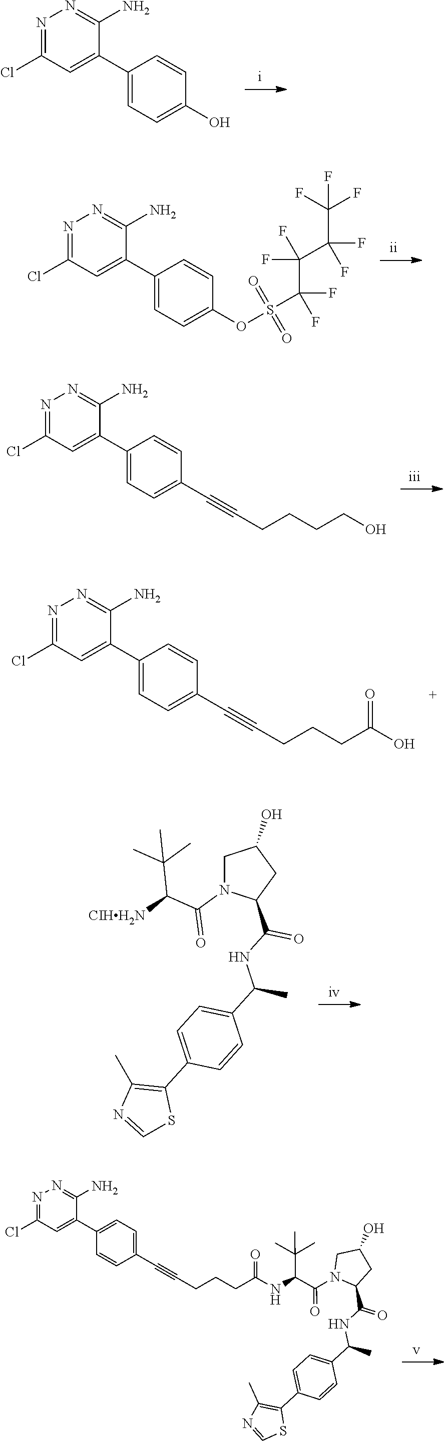

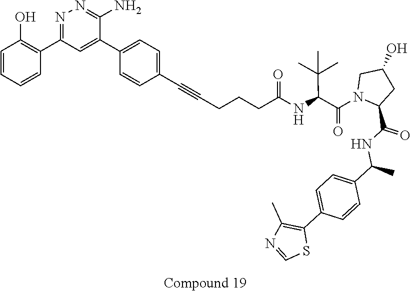 [SMILES: Cc1ncsc1-c1ccc([C@H](C)NC(=O)[C@@H]2C[C@@H](O)CN2C(=O)[C@@H](NC(=O)CCCC#Cc2ccc(-c3cc(-c4ccccc4O)nnc3N)cc2)C(C)(C)C)cc1]

### Step i: Synthesis of 4-(3-amino-6-chloropyridazin-4-yl)phenyl 1,1,2,2,3,3,4,4,4-nonafluorobutane-1-sulfonate

To a stirred solution of 4-(3-amino-6-chloropyridazin-4-yl)phenol (0.6 g, 2.71 mmol), nonaflurobutanesulfonyl fluoride (1.2 ml, 2 vol) in THF (10 mL) was added K₂CO₃ (0.75 g, 5.42 mmol) and the reaction mixture was stirred at RT for 16 h. Once the reaction was completed (monitored by TLC), the reaction mixture was diluted with EtOAc. The combined organic layer was washed with water and brine, dried over anhydrous sodium sulphate and concentrated under vacuum to give the residue. The residue was purified by combiflash column chromatography using 40% ethyl acetate in hexane as eluent to afford the title compound off-white solid (1.2 g, 88%). ¹H NMR (400 MHz, DMSO-d₆): δ 7.74-7.72 (m, 2H), 7.55 (d, J=8.8 Hz, 2H), 7.45 (s, 1H), 6.51 (s, 2H); LC-MS: m/z 503.9 (M+H).

### Step ii: Synthesis of 6-(4-(3-amino-6-chloropyridazin-4-yl)phenyl)hex-5-yn-1-ol

To a stirred solution of 4-(3-amino-6-chloropyridazin-4-yl)phenyl 1,1,2,2,3,3,4,4,4-nonafluorobutane-1-sulfonate (1.2 g, 2.38 mmol), hex-5-yn-1-ol (2.33 g, 23.8 mmol) and TEA (0.8 g, 5.95 mmol) in DMF (15 mL). The reaction mixture degassed with nitrogen for 5 min. Pd(PPh₃)₂Cl₂ (0.08 g, 0.119 mmol) and CuI (0.045 g, 0.238 mmol) was added and heated for 2 h at 120° C. in microwave. After completion of the reaction mixture was diluted with water and extracted with EtOAc. The combined organic layer was washed with water and brine, dried over anhydrous sodium sulphate and concentrated under vacuum to give the residue. The residue was purified by combiflash column chromatography using 50% ethyl acetate in hexane as eluent to afford the title compound yellow solid (0.45 g, 63%). ¹H NMR (400 MHz, DMSO-d6): δ 7.79-7.72 (m, 2H), 7.52-7.46 (m, 4H), 7.36 (s, 1H), 6.41 (s, 2H), 4.45-4.39 (m, 1H), 3.45-3.36 (m, 2H), 3.15 (d, J=5.2 Hz, 2H), 2.51-2.45 (m, 2H). LC-MS: m/z 301.9 (M+H).

### Step iii: Synthesis of 6-(4-(3-amino-6-chloropyridazin-4-yl)phenyl)hex-5-ynoic acid

To a stirred solution of 6-(4-(3-amino-6-chloropyridazin-4-yl)phenyl)hex-5-yn-1-ol (0.45 g, 1.49 mmol) in acetone (4 mL) at 0° C. was dropwise added Jones reagent (0.5 mL) and temperature was brought to RT over the period of 2 h. After reaction completion, the reaction mixture was diluted with acetone and filtered. The filtrate was concentrated under vacuum. To the residue was added 1 mL water and extracted with 5% methanol in DCM, organic layer was concentrated to get crude product. The crude product was triturated with diethyl ether and dried under vacuum to afford pure title compound as off-white solid (0.35 g, 94%). LC/MS: m/z 315.9 (M+1).

### Step iv: Synthesis of (2S,4R)-1-((S)-2-(6-(4-(3-amino-6-chloropyridazin-4-yl)phenyl)hex-5-ynamido)-3,3-dimethylbutanoyl)-4-hydroxy-N—((S)-1-(4-(4-methylthiazol-5-yl)phenyl)ethyl)pyrrolidine-2-carboxamide

To a solution of 6-(4-(3-amino-6-chloropyridazin-4-yl)phenyl)hex-5-ynoic acid (0.38 g, 1.2 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.58 g, 1.2 mmol) in DMF (4 mL) at 0° C. was added HATU (0.54 g, 1.44 mmol) followed by the dropwise addition of DIPEA (0.4 mL, 2.4 mmol) and the reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 5% methanol in DCM as eluent to afford the title compound pale yellow solid (0.24 g, 27%). ¹H NMR (400 MHz, DMSO-d₆): δ 8.97 (s, 1H), 8.32 (s, 1H), 7.82 (s, 1H), 7.51 (s, 3H), 7.43-7.41 (m, 3H), 7.37-7.34 (m, 3H0, 6.40 (s, 2H), 5.15-5.09 (m, 1H), 4.54 (d, J=9.2 Hz, 1H), 4.43 (t, J=8.0 Hz, 1H), 4.28 (s, 1H), 3.60 (s, 2H), 2.66-2.44 (m, 6H), 2.01 (s, 1H), 1.79 (t, J=7.6 Hz, 3H), 1.37 (d, J=6.8 Hz, 3H), 1.23 (s, 1H), 0.95 (s, 9H). LC/MS: m/z 742.3 (M+1).

### Step v: Synthesis of (2S,4R)-1-((S)-2-(6-(4-(3-amino-6-(2-hydroxyphenyl)pyridazin-4-yl)phenyl)hex-5-ynamido)-3,3-dimethylbutanoyl)-4-hydroxy-N—((S)-1-(4-(4-methylthiazol-5-yl)phenyl)ethyl)pyrrolidine-2-carboxamide

A mixture of 1,4-dioxane (4 mL) and water (2 mL) were taken in microwave vial and degassed with nitrogen for 5 min. (2S,4R)-1-((S)-2-(6-(4-(3-amino-6-chloropyridazin-4-yl)phenyl)hex-5-ynamido)-3,3-dimethylbutanoyl)-4-hydroxy-N—((S)-1-(4-(4-methylthiazol-5-yl)phenyl)ethyl)pyrrolidine-2-carboxamide (0.24 g, 0.32 mmol) and (2-hydroxyphenyl)boronic acid (0.067 g, 0.48 mmol) were added to the reaction mixture, followed by addition of K₂CO₃ (0.09 g, 3.51 mmol) and Pd(dppf)Cl₂·DCM (0.065 g, 0.08 mmol). The reaction mixture was heated for 1 h at 120° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue. The residue was purified by combiflash column chromatography using 5-7% methanol in DCM as eluent to afford the title compound pale yellow solid (0.08 g, 31%). ¹H NMR (400 MHz, DMSO-d₆): δ 13.5 (s, 1H), 8.34 (d, J=8.0 Hz, 1H), 8.01 (s, 1H), 7.93 (d, J=8.4 Hz, 2H), 7.61 (d, J=8.4 Hz, 3H), 7.56 (d, J=8.4 Hz, 2H), 7.43 (d, J=8.4 Hz, 2H), 7.37 (d, J=8.4 Hz, 2H), 7.25 (t, J=7.6 Hz, 1H), 6.93-6.86 (m, 2H), 6.48 (s, 2H), 5.09 (d, J=3.6 Hz, 1H), 4.92 (t, J=7.6 Hz, 1H), 4.54 (d, J=9.2 Hz, 1H), 4.43 (t, J=8.0 Hz, 1H), 4.28 (s, 1H), 3.81 (s, 2H), 2.66-2.45 (m, 5H), 2.01 (m, 1H), 1.79 (m, 3H), 1.37 (d, J=6.8 Hz, 3H), 1.23 (bs, 2H), 0.95 (s, 9H). LC/MS: m/z 800.4 (M+1).

The compounds listed in below Table-3 were prepared by the procedure similar to the one described in Example-15 with appropriate variations in reactants, quantities of reagents, protections and deprotections, solvents and reaction conditions. The characterization data of the compounds are summarized herein the below table.

| Com- | | |
|---|---|---|
| pound | Structure | Characterization Data |
| 20 | 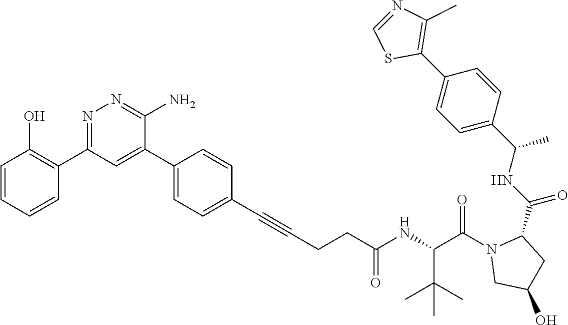 [SMILES: Cc1ncsc1-c1ccc([C@H](C)NC(=O)[C@@H]2C[C@@H](O)CN2C(=O)[C@@H](NC(=O)CCC#Cc2ccc(-c3cc(-c4ccccc4O)nnc3N)cc2)C(C)(C)C)cc1] | ¹H NMR (400 MHz, DMSO-d₆): δ 13.5 (s, 1H), 8.98 (s, 1H), 8.36 (d, J = 7.2 Hz, 1H), 8.00 (d, J = 7.6 Hz, 2H), 7.94 (d, J = 7.6 Hz, 1H), 7.61 (d, J = 8.4 Hz, 2H), 7.50 (d, J = 8.0 Hz, 2H), 7.43 (d, J = 8.4 Hz, 2H), 7.38 (d, J = 8.4 Hz, 2H), 7.25 (t, J = 7.2 Hz, 1H), 694-6.86 (m, 2H), 6.49 (s, 2H), 5.12 (s, 1H), 4.92 (t, J = 7.2 Hz, 1H), 4.58 (d, J = 9.6 Hz, 1H), 4.43 (t, J = 8.4 Hz, 1H), 4.29 (s, 1H), 3.66-3.61 (m, 2H), 2.69-2.59 (m, 4H), 2.45 (s, 3H), 2.01 (m, 1H), 1.83-1.78 (m, 1H), 1.38 (d, J = 6.8 Hz, 3H), 0.97 (s, 9H). LC/MS: m/z 786.3(M + 1)⁺. |
| | (2S,4R)-1-((S)-2-(5-(4-(3-amino-6-(2- | |
| | hydroxyphenyl)pyridazin-4- | |
| | yl)phenyl)pent-4-ynamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 21 | 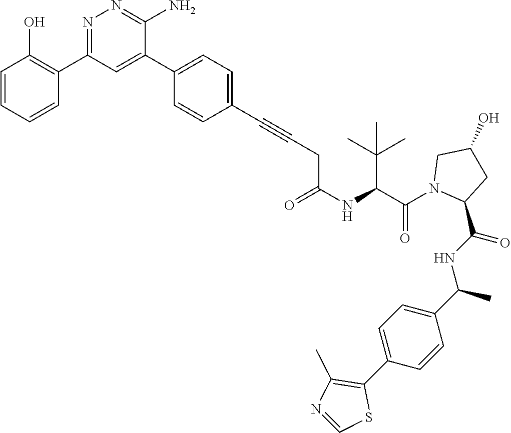 [SMILES: Cc1ncsc1-c1ccc([C@H](C)NC(=O)[C@@H]2C[C@@H](O)CN2C(=O)[C@@H](NC(=O)CC#Cc2ccc(-c3cc(-c4ccccc4O)nnc3N)cc2)C(C)(C)C)cc1] | ¹H NMR (400 MHz, DMSO-d₆): δ 13.5 (s, 1H), 8.97 (s, 1H), 8.41 (d, J = 7.2 Hz, 1H), 8.01 (t, J = 6.8 Hz, 2H), 7.94 (d, J = 7.6 Hz, 1H), 7.63 (d, J = 8.4 Hz, 2H), 7.57 (d, J = 8.4 Hz, 2H), 7.48-7.35 (m, 4H), 7.25 (t, J = 7.2 Hz, 1H), 6.96-6.86 (m, 2H), 6.51 (s, 2H), 5.10 (s, 1H), 4.91 (t, J = 7.0 Hz, 1H), 4.59-4.43 (m, 2H), 4.45 (t, J = 8.4 Hz, 1H), 3.61-3.52 (m, 4H), 2.43 (s, 3H), 2.00 (t, J = 9.6 Hz, 1H), 1.83-1.78 (m, 1H), 1.49 (d, J = 7.2 Hz, 3H), 0.97 (s, 9H).LC/MS: m/z 772.2 (M + 1). |
| | (2S,4R)-1-((S)-2-(4-(4-(3-amino-6-(2- | |
| | hydroxyphenyl)pyridazin-4- | |
| | yl)phenyl)but-3-ynamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 22 | 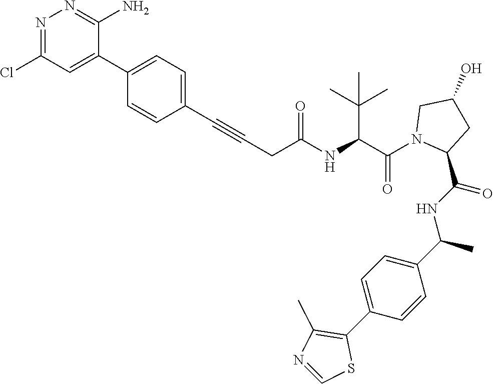 | ¹H NMR (400 MHz, DMSO-d₆): δ 8.97 (s, 1H), 8.32 (s, 1H), 7.82 (s,1H), 7.51 (s, 3H), 7.43-7.41 (m, 3H), 7.37-7.34 (m, 3H), 6.40 (s, 2H), 5.15-5.09 (m, 1H), 4.95-4.86 (m, 1H), 4.54 (d, J = 9.2 Hz, 1H), 4.43 (t, J = 8.0 Hz, 1H), 4.28 (bs, 1H), 3.75-3.42 (m, 4H), 2.44 (m, 3H), 2.01 (m, 1H), 1.79 (m, 1H), 1.37 (d, J = 6.8 Hz, 3H), 0.95 (s, 9H). LC/MS: m/z 742.3 (M + 1). |
| | (2S,4R)-1-((S)-2-(4-(4-(3-amino-6- | |
| | chloropyridazin-4-yl)phenyl)but-3- | |
| | ynamido)-3,3-dimethylbutanoyl)-4- | |
| | hydroxy-N-((S)-1-(4-(4-methylthiazol- | |
| | 5-yl)phenyl)ethyl)pyrrolidine-2- | |
| | carboxamide | |
| 23 | 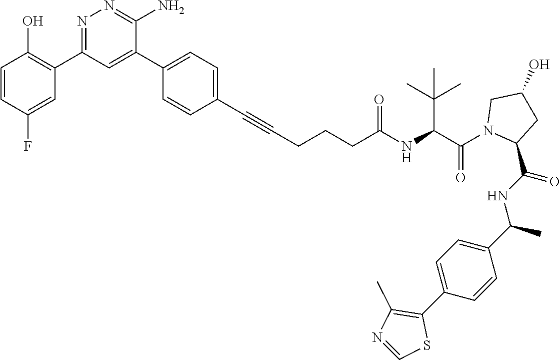 [SMILES: Cc1ncsc1-c1ccc([C@H](C)NC(=O)[C@@H]2C[C@@H](O)CN2C(=O)[C@@H](NC(=O)CCCC#Cc2ccc(-c3cc(-c4cc(F)ccc4O)nnc3N)cc2)C(C)(C)C)cc1] | ¹H NMR (400 MHz, DMSO-d₆): δ 13.36 (s, 1H), 8.98 (s, 1H), 8.40 (d, J = 7.6 Hz, 1H), 8.07 (s, 1H), 7.95 (d, J = 9.6 Hz, 1H), 7.88-7.85 (m, 1H), 7.63-7.56 (m, 4H), 7.45-7.37 (m, 4H), 7.10-7.08 (m, 1H), 6.93 (q, J = 5.2 Hz, 1H), 6.62 (s, 2H), 5.13 (d, J = 3.2 Hz, 1H), 4.92 (t, J = 7.2 Hz, 1H), 4.54 (d, J = 9.2 Hz, 1H), 4.43 (t, J = 8.0 Hz, 1H), 4.28 (d, J = 5.6 Hz, 1H), 3.61 (s, 2H), 2.64-2.32 (m, 7H), 2.01-1.98 (m, 1H), 1.78 (d, J = 8.4 Hz, 3H), 1.37 (d, J = 7.2 Hz, 3H), 0.96 (s, 9H). LC/MS: m/z 818.35 (M + H). |
| | (2S,4R)-1-((S)-2-(6-(4-(3-amino-6-(5- | |
| | fluoro-2-hydroxyphenyl)pyridazin-4- | |
| | yl)phenyl)hex-5-ynamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |

### Example-16: (2S,4R)-1-(2-(3-(2-(4-(3-amino-6-(2-hydroxyphenyl)pyridazin-4-yl)phenoxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (compound 24)

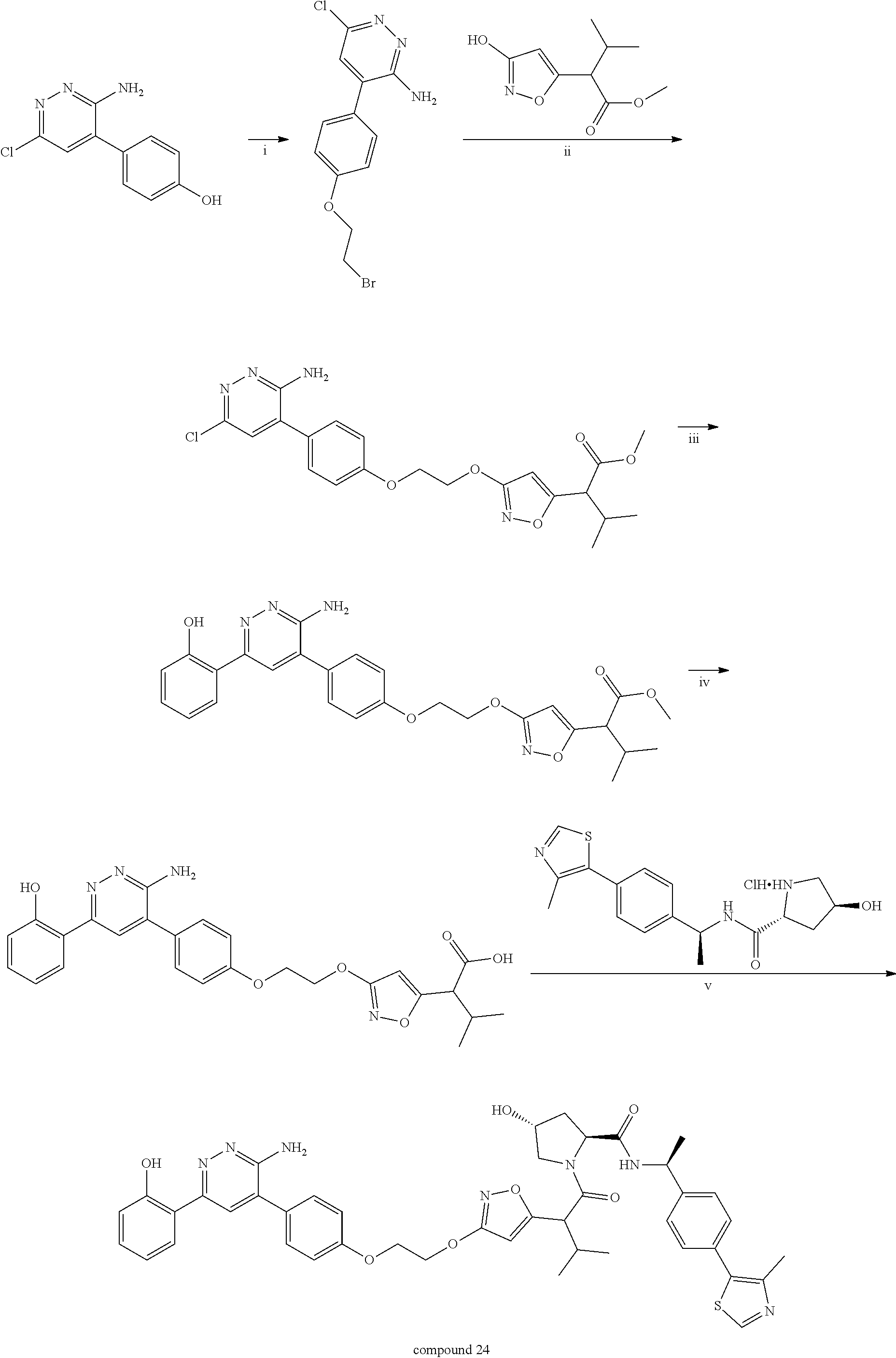

### Step i: Synthesis of 4-(4-(2-bromoethoxy)phenyl)-6-chloropyridazin-3-amine

To a stirred solution of 4-(3-amino-6-chloropyridazin-4-yl)phenol (1 g, 4.52 mmol) in ethanol (20 mL) was added K₂CO₃ (3.8 g, 27.14 mmol) and 1,2-dibromoethane (2 mL, 22.6 mmol) at RT and the reaction mixture was heated at 100° C. for 12 h. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 60-70% ethyl acetate in hexane as eluent to afford the title compound as pale-yellow solid (0.5 g, 33%). ¹H NMR (400 MHz, DMSO-d₆): δ 7.51 (d, J=8.4 Hz, 2H), 7.31 (s, 1H), 7.11 (d, J=8.8 Hz, 2H), 6.31 (s, 2H), 4.40 (t, J=10.8 Hz, 2H), 3.84 (t, J=10.4 Hz, 2H). LC-MS: m/z 330.0 (M+H).

### Step ii: Synthesis of methyl 2-(3-(2-(4-(3-amino-6-chloropyridazin-4-yl)phenoxy)ethoxy)isoxazol-5-yl)-3-methylbutanoate

To a stirred solution of 4-(4-(2-bromoethoxy)phenyl)-6-chloropyridazin-3-amine (0.35 g, 1.06 mmol) and methyl 2-(3-hydroxyisoxazol-5-yl)-3-methylbutanoate (0.21 g, 2.06 mmol) in DMF (8 mL) was added K₂CO₃ (0.44 g, 3.18 mmol) at RT and the reaction mixture was stirred for 16 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 75% ethyl acetate in hexane as eluent to afford the title compound as off-white solid (0.35 g, 73%). ¹H NMR (400 MHz, DMSO-d₆): δ 7.51 (d, J=8.4 Hz, 2H), 7.31 (s, 1H), 7.12 (d, J=8.8 Hz, 2H), 6.30 (d, J=14.0 Hz, 3H), 4.51 (t, J=3.6 Hz, 2H), 4.38 (d, J=2.0 Hz, 2H), 3.67 (s, 3H), 2.33-2.27 (m, 1H), 0.94 (d, J=6.4 Hz, 3H), 0.86 (d, J=6.4 Hz, 3H). LC-MS: m/z 447.2 (M+H).

### Step iii: Synthesis of methyl 2-(3-(2-(4-(3-amino-6-(2-hydroxyphenyl)pyridazin-4-yl)phenoxy)ethoxy)isoxazol-5-yl)-3-methylbutanoate

A mixture of 1,4-dioxane (13 mL), water (2 mL) were taken in microwave vial and degassed with nitrogen for 5 min. Methyl 2-(3-(2-(4-(3-amino-6-chloropyridazin-4-yl)phenoxy)ethoxy)isoxazol-5-yl)-3-methylbutanoate (0.3 g, 0.67 mmol) and 2-hydroxyphenylboronic acid (0.19 g, 1.34 mmol) were added to the reaction mixture followed by addition of K₂CO₃ (0.28 g, 2.01 mmol) and Pd(dppf)Cl₂·DCM (0.055 g, 0.067 mmol). The reaction mixture was heated for 1 min. at 120° C. in microwave. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 60% ethyl acetate in hexane as eluent to afford the title compounds as off-white solid (0.14 g, 41%). LC-MS: m/z 505.2 (M+H).

### Step iv: Synthesis of 2-(3-(2-(4-(3-amino-6-(2-hydroxyphenyl)pyridazin-4-yl)phenoxy)ethoxy)isoxazol-5-yl)-3-methylbutanoic acid

To a stirred solution of methyl 2-(3-(2-(4-(3-amino-6-(2-hydroxyphenyl)pyridazin-4-yl)phenoxy)ethoxy)isoxazol-5-yl)-3-methylbutanoate (0.14 g, 0.27 mmol) in THF:H₂O (6 mL:1 mL) mixture was added LiOH·H₂O (0.035 g, 0.83 mmol) at 0° C. The reaction mixture was stirred for 12 h at RT. The reaction mixture was evaporated under reduced pressure and the resultant residue was diluted with methanol and acidified to pH 6 using amberlite IT120 (acidic resin) and filtered and filtrate was concentrated under vacuum to afford the title compound as a brown solid (0.12 g, 88%). LC-MS: m/z 491.2 (M+H).

### Step v: Synthesis of (2S4R)-1-(2-(3-(2-(4-(3-amino-6-(2-hydroxyphenyl)pyridazin-4-yl)phenoxy)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(2-(4-(3-amino-6-(2-hydroxyphenyl)pyridazin-4-yl)phenoxy)ethoxy)isoxazol-5-yl)-3-methylbutanoic acid (0.12 g, 0.24 mmol), (2R,4S)-4-hydroxy-N—((S)-1-(4-(4-methylthiazol-5-yl)phenyl)ethyl)pyrrolidine-2-carboxamide hydrochloride (0.09 g, 0.244 mmol) in DMF (5 mL) at 0° C. was added HATU (0.14 g, 0.36 mmol) followed by the dropwise addition of DIPEA (0.21 mL, 3.84 mmol) and the reaction mixture was stirred for 3 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 5-7% methanol in DCM as eluent to afford the title compound pale yellow solid (0.05 g, 25%). ¹H NMR (400 MHz, DMSO-d₆): δ 13.80 (s, 1H), 8.96 (d, J=11.6 Hz, 1H), 8.41-8.32 (m, 1H), 7.97-7.93 (m, 2H), 7.58 (t, J=7.2 Hz, 2H), 7.47-7.23 (m, 5H), 7.14 (t, J=10.0 Hz, 2H), 6.96-6.83 (m, 2H), 6.40 (s, 2H), 6.16 (d, J=8.0 Hz, 1H), 5.09 (s, 1H), 4.91-4.88 (m, 1H), 4.52-4.49 (m, 2H), 4.45-4.36 (m, 3H), 4.27 (s, 1H), 3.77 (d, J=8.8 Hz, 1H), 3.68-3.46 (m, 1H), 2.45 (s, 3H), 2.29-2.26 (m, 1H), 2.07-2.03 (m, 1H), 1.80-1.47 (m, 1H), 1.46 (d, J=6.8 Hz, 1H), 1.38-1.34 (m, 3H), 0.98-0.96 (m, 3H), 0.85-0.76 (m, 3H). LC-MS: m/z 804.3 (M+H).

### Example-17: (2S,4R)-1-((S)-2-(2-(4-(5-(3-amino-6-(2-hydroxyphenyl)pyridazin-4-yl)pyridin-2-yl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound 25)

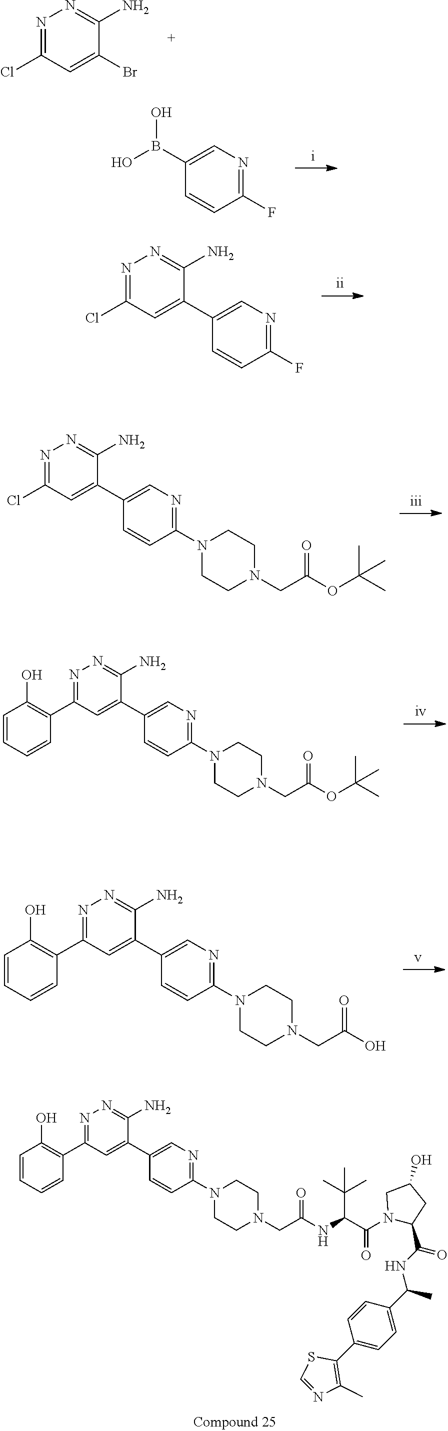

### Step i: Synthesis of 6-chloro-4-(6-fluoropyridin-3-yl)pyridazin-3-amine

To a stirred solution of 4-bromo-6-chloropyridazin-3-amine (1.5 g, 7.24 mmol), tert-butyl 6-fluoropyridin-3-yl-3-boronic acid (1.6 g, 7.99 mmol) and Na₂CO₃ (3.09 g, 29.16 mmol) in 1,4-dioxane (30 mL) and water (14 mL). The reaction mixture degassed with nitrogen for 15 min. Pd(dppf)Cl₂·DCM (0.6 g, 0.72 mmol) was added and the reaction mixture was heated at 120° C. for 1 h in microwave. Once the reaction was completed (monitored by TLC), the reaction mixture was diluted with EtOAc. The combined organic layer was washed with water and brine, dried over anhydrous sodium sulphate and concentrated under vacuum to give the residue. The residue was purified by combiflash column chromatography using 45% ethyl acetate in hexane as eluent to afford the title compound brown solid (0.75 g, 46%). ¹H NMR (400 MHz, DMSO-d₆): δ 8.37 (d, J=1.6 Hz, 1H), 8.14 (dt, J=8.4, 2.4 Hz, 1H), 7.47 (s, 1H), 7.33 (dd, J=8.4, 2.4 Hz, 1H), 6.53 (bs, 2H); LC-MS: m/z 225.1 (M+H).

### Step ii: Synthesis of tert-butyl 2-(4-(5-(3-amino-6-chloropyridazin-4-yl)pyridin-2-yl)piperazin-1-yl)acetate

To a stirred solution of 6-chloro-4-(6-fluoropyridin-3-yl)pyridazin-3-amine (0.55 g, 2.44 mmol) in DMF (10 mL) was added DIPEA (1.74 mL, 9.76 mmol) at RT and stirred for 15 min then added tert-butyl 2-(piperazin-1-yl)acetate (0.97 g, 4.88 mmol) and the reaction mixture was heated at 120° C. for 16 h. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 3-4% methanol in DCM as eluent to afford the title compound brown solid (0.3 g, 30%). ¹H NMR (400 MHz, DMSO-d₆): δ 8.29 (d, J=2.4 Hz, 1H), 7.72 (dd, J=8.8, 2 Hz, 1H), 7.33 (s, 1H), 6.93 (d, J=8.8 Hz, 1H), 6.36 (bs, 2H) 3.58 (bs, 4H), 3.17 (s, 2H), 2.60 (bs, 4H), 1.42 (s, 9H); LC-MS: m/z 405.2 (M+H).

### Step iii: Synthesis of tert-butyl 2-(4-(5-(3-amino-6-(2-hydroxyphenyl)pyridazin-4-yl)pyridin-2-yl)piperazin-1-yl)acetate

To a stirred solution of tert-butyl 2-(4-(5-(3-amino-6-chloropyridazin-4-yl)pyridin-2-yl)piperazin-1-yl)acetate (0.15 g, 0.37 mmol), 2-hydroxyphenylboronic acid (0.08 g, 0.55 mmol) and Na₂CO₃ (0.15 g, 1.48 mmol) in 1,4-dioxane (5 mL) and water (1 mL). The reaction mixture degassed with nitrogen for 15 min. Pd(dppf)Cl₂·DCM (0.03 g, 0.037 mmol) was added to the reaction mixture and heated at 120° C. for 2 h in sealed tube. Once the reaction was completed (monitored by TLC), the reaction mixture was diluted with EtOAc. The combined organic layer was washed with water and brine, dried over anhydrous sodium sulphate and concentrated under vacuum to get the residue which was purified by combiflash column chromatography using 5% methanol in DCM as eluent to afford the title compound as brown solid (0.09 g, 52%). ¹H NMR (400 MHz, DMSO-d₆): δ 13.64 (s, 1H), 8.39 (s, 1H), 7.99 (s, 1H), 7.94-7.98 (m, 1H), 7.81 (d, J=8.4 Hz, 1H), 7.24 (d, J=7.6 Hz, 1H), 6.97 (d, J=9.2 Hz, 1H), 6.87-6.98 (m, 1H), 6.48 (bs, 2H) 3.60 (bs, 4H), 3.18 (s, 2H), 2.62 (bs, 4H), 1.42 (s, 9H); LC-MS: m/z 463.2 (M+H).

### Step iv: Synthesis of 2-(4-(5-(3-amino-6-(2-hydroxyphenyl)pyridazin-4-yl)pyridin-2-yl)piperazin-1-yl)acetic acid

To a stirred solution of tert-butyl 2-(4-(5-(3-amino-6-(2-hydroxyphenyl)pyridazin-4-yl)pyridin-2-yl)piperazin-1-yl)acetate (0.09 g, 0.15 mmol) in DCM (3 mL) was added 4 N dioxane hydrochloride (1 mL) at 0° C. and then slowly brought to RT and stirred for 3 h. The reaction mixture was evaporated under reduced pressure, the resultant residue was washed with diethyl ether and dried under vacuum to afford the title compound as brown solid (0.08 g, crude yield). LC-MS: m/z 407.0 (M+H).

### Step v: Synthesis of (2S,4R)-1-((S)-2-(2-(4-(5-(3-amino-6-(2-hydroxyphenyl)pyridazin-4-yl)pyridin-2-yl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(5-(3-amino-6-(2-hydroxyphenyl)pyridazin-4-yl)pyridin-2-yl)piperazin-1-yl)acetic acid (0.04 g, 0.096 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.05 g, 0.096 mmol) in DMF (3 mL) at 0° C. was added HATU (0.054 g, 0.144 mmol) followed by the dropwise addition of DIPEA (0.07 mL, 0.38 mmol) and the reaction mixture was stirred for 16 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 5-6% methanol in DCM as eluent to afford the title compound brown solid (0.02 g, 24%). ¹H NMR (400 MHz, DMSO-d₆): δ 11.91 (s, 1H), 8.97 (s, 1H), 8.42 (d, J=7.2 Hz, 1H), 8.25 (d, J=5.6 Hz, 1H), 8.13 (s, 1H), 7.78 (d, J=10.0 Hz, 1H), 7.46-7.35 (m, 7H), 7.10 (d, J=8.8 Hz, 2H), 6.38 (t, J=6.0 Hz, 1H), 6.06 (s, 2H), 5.12 (d, J=2.4 Hz, 1H), 4.89 (t, J=6.8 Hz, 1H), 4.53-4.30 (m, 2H), 4.29 (bs, 1H), 3.59-3.56 (m, 2H), 2.66-2.61 (m, 6H), 3.16-3.03 (m, 2H), 2.5-2.45 (m, 5H), 2.32 (s, 2H), 2.08-2.03 (m, 1H), 1.79-1.74 (m, 1H), 1.36 (d, J=6.8 Hz, 3H), 0.96 (s, 9H); LC-MS: m/z 833.35 (M+H).

### Example-18: (2S,4R)-4-hydroxy-1-((S)-2-(2-(4-(4-(6-(2-hydroxyphenyl)-3-methoxypyridazin-4-yl)phenyl)piperazin-1-yl)acetamido)-3,3-dimethylbutanoyl)-N—((S)-1-(4-(4-methylthiazol-5-yl)phenyl)ethyl)pyrrolidine-2-carboxamide (Compound 26)

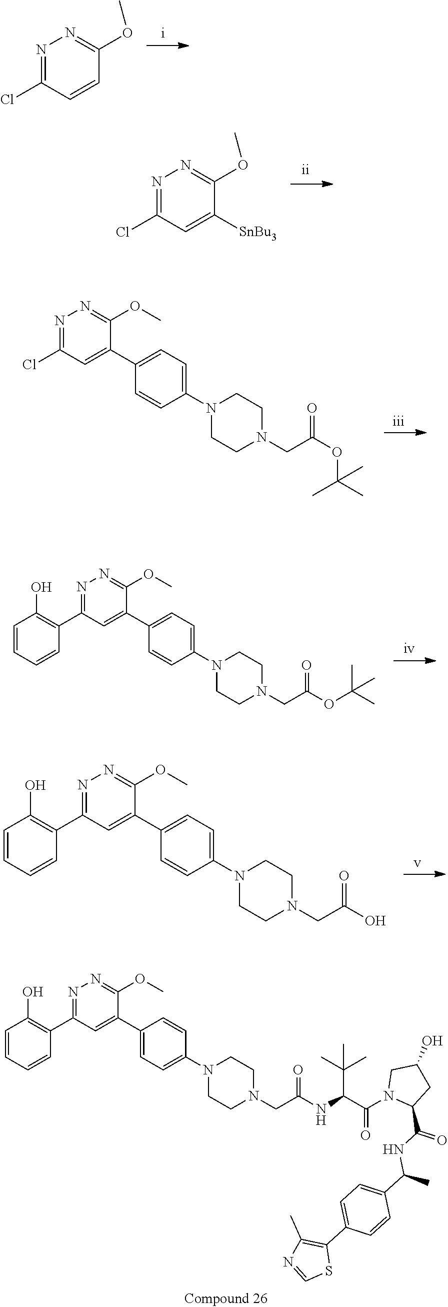

### Step i: Synthesis of 4-(tributylstannyl)-6-chloro-3-methoxypyridazine

To a solution of LDA (2M) (8.3 mL, 16.66 mmol) in THF (30 mL) at −78° C. under nitrogen atmosphere, was added a solution of 3-chloro-6-methoxypyridazine (2 g, 13.88 mmol) and tributyltin chloride (4.51 g, 13.88 mmol) in tetrahydrofuran (20 ml) dropwise at −78° C. and the mixture was stirred for 1 hour. Then, water was added thereto and the mixture was extracted with ethyl acetate. The organic layer was washed with water, dried and concentrated to get crude residue. The residue was purified by combiflash column chromatography using 10% ethyl acetate in hexane to give the title compound as a pale yellow oil (0.4 g, 6%). ¹H-NMR (400 MHz, CDCl₃); δ (ppm) 0.89 (s, 9H), 1.10-1.15 (m, 6H), 1.27-1.36 (m, 6H), 1.48-1.53 (m, 6H), 4.06 (s, 3H), 7.38 (s, 1H).

### Step ii: Synthesis of tert-butyl 2-(4-(4-(6-chloro-3-methoxypyridazin-4-yl)phenyl)piperazin-1-yl)acetate

To a stirred solution of 4-(tributylstannyl)-6-chloro-3-methoxypyridazine (0.2 g, 0.46 mmol), tert-butyl 2-(4-(4-bromophenyl)piperazin-1-yl)acetate (0.21 g, 0.59 mmol) in xylene (15 mL) was added CuI (0.01 g, 0.046). The reaction mixture was degassed with nitrogen for 15 min. Pd(PPh₃)₄ (0.053 g, 0.046 mmol) was added to the reaction mixture and heated at 140° C. for 4 h in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 40-50% ethyl acetate in hexane as eluent to afford the title compound as yellow solid (0.03 g, 15%). LC-MS: m/z 419.2 (M+H).

### Step iii: Synthesis of tert-butyl 2-(4-(4-(6-(2-hydroxyphenyl)-3-methoxypyridazin-4-yl)phenyl)piperazin-1-yl)acetate

To a stirred solution of tert-butyl 2-(4-(4-(6-chloro-3-methoxypyridazin-4-yl)phenyl)piperazin-1-yl)acetate (0.06 g, 0.143 mmol), 2-hydroxyphenylboronic acid (0.025 g, 0.186 mmol) and Na₂CO₃ (0.045 g, 0.43 mmol) in 1,4-dioxane (10 mL) and water (0.5 mL) and degassed with nitrogen for 15 min. Pd(dppf)Cl₂·DCM (0.011 g, 0.014 mmol) was added to the reaction mixture and the reaction mixture was heated at 140° C. for 5 h in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 50% ethyl acetate in hexane as eluent to afford the title compound as off-white solid (0.017 g, 25%). ¹H NMR (400 MHz, DMSO-d₆): δ 7.95 (s, 1H), 7.66 (d, J=8.8 Hz, 2H), 7.36-7.31 (m, 1H), 7.21 (d, J=7.2 Hz, 1H), 7.11 (d, J=7.6 Hz, 2H), 6.99-6.92 (m, 4H), 4.29 (s, 3H), 3.39 (t, J=10.4 Hz, 4H), 3.19 (s, 2H), 2.77 (t, J=10.0 Hz, 4H), 1.49 (s, 9H). LC-MS: m/z 477.2 (M+H).

### Step iv: Synthesis of 2-(4-(4-(6-(2-hydroxyphenyl)-3-methoxypyridazin-4-yl)phenyl)piperazin-1-yl)acetic acid

To a stirred solution of tert-butyl 2-(4-(4-(6-(2-hydroxyphenyl)-3-methoxypyridazin-4-yl)phenyl)piperazin-1-yl)acetate (0.015 g, 0.031 mmol) in DCM (2 mL) was added 4 N dioxane hydrochloride (0.5 mL) at 0° C. and slowly brought to RT and stirred for 16 h. The reaction mixture was evaporated under reduced pressure, the resultant residue was washed with diethyl ether and dried under vacuum to afford the title compound as sticky material (0.015 g, crude yield). LC-MS: m/z 421.1 (M+H).

### Step v: Synthesis of (2S,4R)-1-((S)-2-(2-(4-(5-(3-amino-6-(2-hydroxyphenyl)pyridazin-4-yl)pyridin-2-yl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(6-(2-hydroxyphenyl)-3-methoxypyridazin-4-yl)phenyl)piperazin-1-yl)acetic acid (0.015 g, 0.035 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.02 g, 0.039 mmol) in DMF (1 mL) at 0° C. was added HATU (0.02 g, 0.052 mmol) followed by the dropwise addition of DIPEA (0.02 mL, 0.1 mmol) and the reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product which was purified by preparative HPLC to afford the title compound yellow solid (0.008 g, 26%). ¹H NMR (400 MHz, DMSO-d₆): δ 8.99 (s, 1H), 8.88 (s, 1H), 8.43 (d, J=8.0 Hz, 1H), 8.34 (s, 1H), 8.09 (d, J=8.0 Hz, 1H), 7.80 (d, J=9.2 Hz, 2H), 7.44 (d, J=8.0 Hz, 2H), 7.39-7.33 (m, 4H), 7.13 (d, J=9.2 Hz, 2H), 6.99-6.95 (m, 2H), 4.92 (s, 1H), 4.60 (d, J=8.8 Hz, 1H), 4.40 (s, 1H), 4.31 (s, 1H), 4.12 (d, J=3.2 Hz, 5H), 4.05 (s, 3H), 3.67-3.61 (m, 4H), 2.47-2.46 (m, 7H), 2.09-2.04 (m, 1H), 1.90-1.80 (m, 1H), 1.39 (d, J=7.2 Hz, 3H), 0.98 (s, 9H). LC-MS: m/z 847.45 (M+H).

### Example-19: (2S,4R)-1-((S)-2-(2-(4-(4-(3-amino-6-(2-fluorophenyl)pyridazin-4-yl)phenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 27)

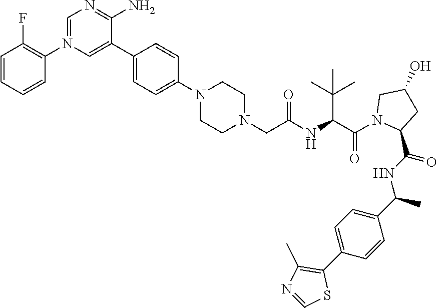

Compound-27 was prepared by the procedure similar to the one described in example-8 with appropriate variations in reactants, quantities of reagents, protections and deprotections, solvents and reaction conditions. ¹H NMR (400 MHz, DMSO-d₆): δ 8.98 (s, 1H), 8.44 (d, J=7.6 Hz, 1H), 7.91 (t, J=6.4 Hz, 1H), 7.79 (d, J=9.6 Hz, 1H), 7.78-7.42 (m, 6H), 7.37-7.30 (m, 4H), 7.15 (d, J=8.8 Hz, 2H), 6.26 (bs, 2H), 5.14 (d, J=3.2 Hz, 1H), 4.89 (d, J=8.0 Hz, 1H), 4.52 (d, J=9.6 Hz, 1H), 4.43 (d, J=7.6 Hz, 1H), 4.35 (d, J=1.2 Hz, 1H), 3.59 (d, J=3.6 Hz, 2H), 3.36 (m, 4H), 3.13 (d, J=16.0 Hz, 1H), 3.02 (d, J=16.0 Hz, 1H), 2.67-2.26 (m, 4H), 2.52 (s, 3H), 1.90 (m, 1H), 1.75 (m, 1H), 1.36 (d, J=7.2 Hz, 3H), 0.95 (s, 9H). LCMS: m/z 834.4 (M+H)

### Example-20: (2S,4R)-1-((S)-2-(2-(4-(4-(3-amino-6-(2-hydroxyphenyl)pyridazin-4-yl)-3-fluorophenyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 28)

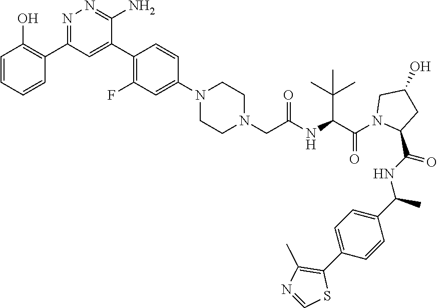 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)c(F)c3)CC2)C(C)(C)C)cc1]

Compound-28 was prepared by the procedure similar to the one described in example-8 with appropriate variations in reactants, quantities of reagents, protections and deprotections, solvents and reaction conditions. ¹H NMR (400 MHz, DMSO-d₆): δ 13.6 (s, 1H), 8.90 (s, 1H), 8.42 (d, J=7.2 Hz, 1H), 7.99 (s, 1H), 7.89 (d, J=8 Hz, 1H), 7.78 (d, J=8.8 Hz, 1H), 7.44-7.33 (m, 5H), 7.42 (t, J=7.2 Hz, 1H), 6.96-6.85 (m, 4H), 6.40 (bs, 2H), 5.13 (d, J=3.6 Hz, 1H), 4.89 (t, J=8.0 Hz, 1H), 4.53 (d, J=10 Hz, 1H), 4.44 (t, J=8.4 Hz, 1H), 4.29 (bs, 1H), 3.59 (bs, 2H), 3.35 (s, 4H), 3.16 (d, J=15.6 Hz, 1H), 3.03 (d, J=15.6 Hz, 1H), 2.66 (m, 4H), 2.45 (s, 3H), 2.04 (m, 1H), 1.76 (m, 1H), 1.46 (d, J=6.8 Hz, 3H), 0.96 (s, 9H). LCMS: m/z 850.7 (M+H)

### Example-21: (2S,4R)-1-((S)-2-(2-(4-(4-(3-amino-6-(2-hydroxyphenyl)pyridazin-4-yl)phenyl)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 29)

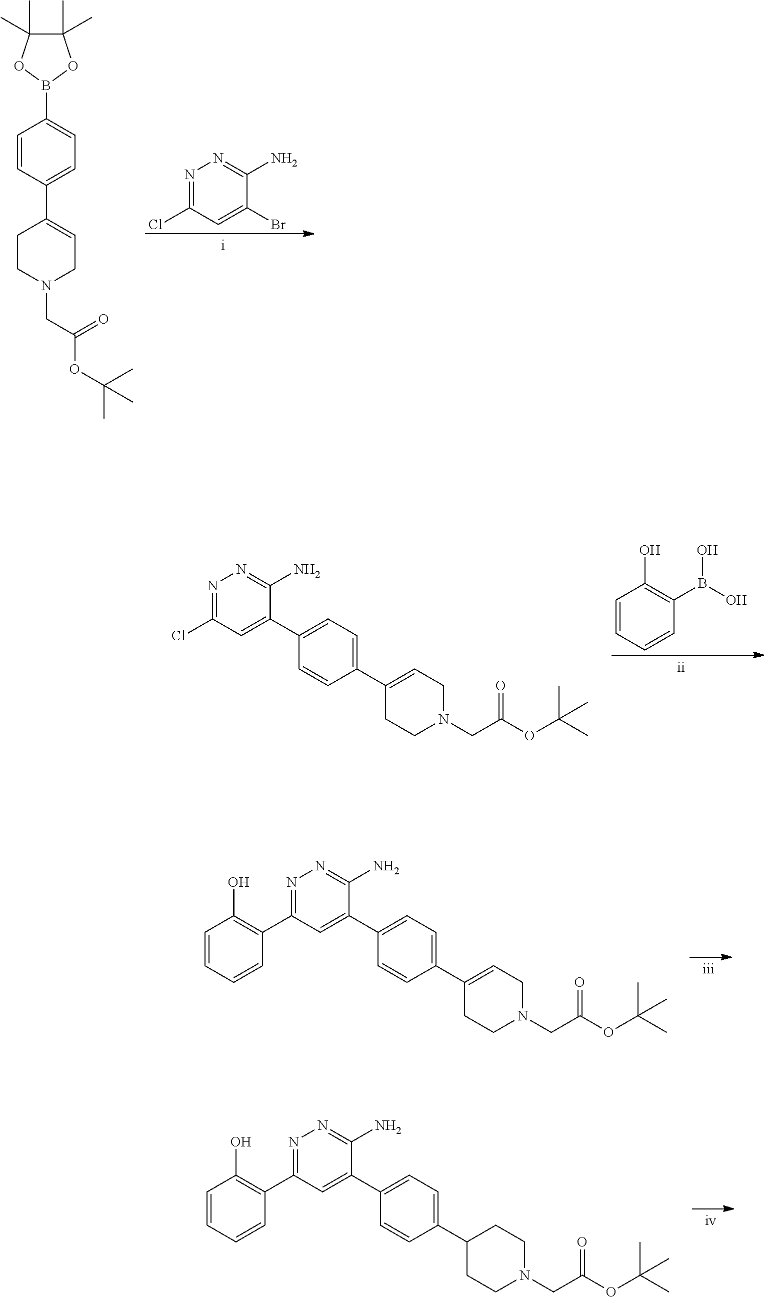 [SMILES: CC(C)(C)OC(=O)CN1CC=C(c2ccc(B3OC(C)(C)C(C)(C)O3)cc2)CC1 ; Nc1nnc(Cl)cc1Br ; CC(C)(C)OC(=O)CN1CC=C(c2ccc(-c3cc(Cl)nnc3N)cc2)CC1 ; OB(O)c1ccccc1O ; CC(C)(C)OC(=O)CN1CC=C(c2ccc(-c3cc(-c4ccccc4O)nnc3N)cc2)CC1 ; CC(C)(C)OC(=O)CN1CCC(c2ccc(-c3cc(-c4ccccc4O)nnc3N)cc2)CC1]

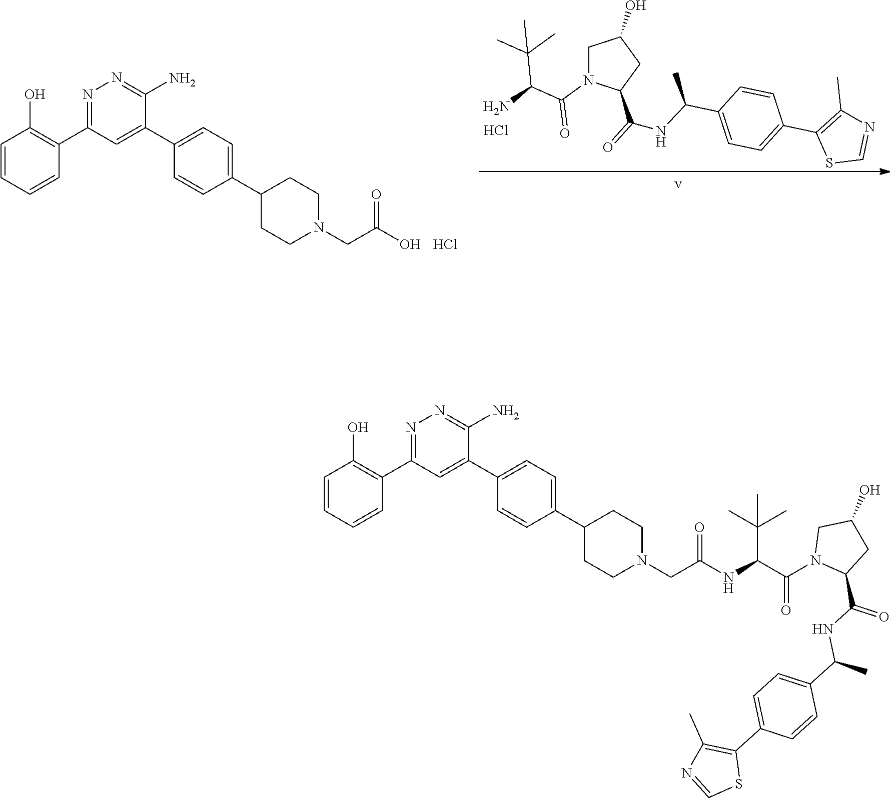 [SMILES: Nc1nnc(-c2ccccc2O)cc1-c1ccc(C2CCN(CC(=O)O)CC2)cc1 ; Cl ; Cc1ncsc1-c1ccc([C@H](C)NC(=O)[C@@H]2C[C@@H](O)CN2C(=O)[C@@H](N)C(C)(C)C)cc1 ; Cl ; Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)CC2)C(C)(C)C)cc1]

### Step-i: Synthesis of tert-butyl 2-(4-(4-(3-amino-6-chloropyridazin-4-yl)phenyl)-3,6-dihydropyridin-1(2H)-yl)acetate

To a stirred solution of 4-bromo-6-chloropyridazin-3-amine (0.7 g, 1.75 mmol), tert-butyl 2-(4-(4-(4,4,5,5-tetramethyl-1,3,2-dioxaborolan-2-yl)phenyl)-3,6-dihydropyridin-1(2H)-yl)acetate (0.43 g, 2.1 mmol) in 1,4-dioxane (15 mL) and water (2.5 mL) was added K₂CO₃ (0.72 g, 5.25 mmol) and degassed with nitrogen for 10 min. Pd(dppf)Cl₂·DCM (0.14 g, 0.17 mmol) was added to the reaction mixture and heated for 16 h at 100° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 40-80% ethyl acetate in hexane as eluent to afford the title compound off-white solid (0.3 g, 42%). ¹H NMR (400 MHz, DMSO-d₆): δ 7.57 (d, J=8.8 Hz, 2H), 7.52 (d, J=8.8 Hz, 2H), 7.36 (s, 1H), 6.38 (bs, 2H), 6.26 (s, 1H), 3.23 (s, 4H), 2.77 (m, 2H), 2.50 (m, 2H), 1.42 (s, 9H); LC-MS: m/z 401.2 (M+H).

### Step-ii: Synthesis of tert-butyl 2-(4-(4-(3-amino-6-(2-hydroxyphenyl)pyridazin-4-yl)phenyl)-3,6-dihydropyridin-1(2H)-yl)acetate

To a stirred solution of tert-butyl 2-(4-(4-(3-amino-6-chloropyridazin-4-yl)phenyl)-3,6-dihydropyridin-1(2H)-yl)acetate (0.3 g, 0.74 mmol) and (2-hydroxyphenyl)boronic acid (0.12 g, 0.89 mmol) in 1,4-dioxane (8 mL) and water (2 mL) was added K₂CO₃ (0.31 g, 2.24 mmol) and degassed with nitrogen for 10 min. Pd(dppf)Cl₂·DCM (0.06 g, 0.07 mmol) was added to the reaction mixture and heated for 16 h at 110° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 60% ethyl acetate in hexane as eluent to afford the title compound off-white solid (0.18 g, 52%). ¹H NMR (400 MHz, DMSO-d₆): δ 13.62 (s, 1H), 8.01 (s, 1H), 7.95 (d, J=7.2 Hz, 1H), 7.61 (s, 4H), 7.25 (m, 1H), 6.91 (m, 2H), 6.48 (bs, 2H), 6.27 (s, 1H), 3.25 (s, 4H), 2.29 (m, 2H), 2.55 (m, 2H), 1.43 (s, 9H). LC-MS: m/z 459.3 (M+H).

### Step-iii: tert-butyl 2-(4-(4-(3-amino-6-(2-hydroxyphenyl)pyridazin-4-yl)phenyl)piperidin-1-yl)acetate

To a stirred solution of tert-butyl 2-(4-(4-(3-amino-6-(2-hydroxyphenyl)pyridazin-4-yl)phenyl)-3,6-dihydropyridin-1(2H)-yl)acetate (0.18 g, 0.39 mmol) in ethanol (5 mL) was added Pd—C(7 mL) at 0° C. and then slowly brought to RT and stirred for 6 h. The reaction mixture was evaporated under reduced pressure, the resultant residue was washed with diethyl ether and dried under vacuum to afford the title compound pale yellow solid (0.15 g, 83%). ¹H NMR (400 MHz, DMSO-d₆): δ 13.65 (s, 1H), 8.00 (s, 1H), 7.95 (d, J=7.6 Hz, 1H), 7.57 (d, J=8 Hz, 2H), 7.43 (d, J=8 Hz, 2H), 7.24 (m, 1H), 6.89 (m, 2H), 6.46 (bs, 2H), 3.14 (s, 2H), 2.95 (m, 2H), 2.57 (m, 1H), 2.32 (m, 2H), 1.76 (m, 4H), 1.43 (s, 9H); LC-MS: m/z 461.1 (M+H).

### Step-iv: 2-(4-(4-(3-amino-6-(2-hydroxyphenyl)pyridazin-4-yl)phenyl)piperidin-1-yl)acetic acid hydrochloride

To a stirred solution of tert-butyl 2-(4-(4-(3-amino-6-(2-hydroxyphenyl)pyridazin-4-yl)phenyl)piperidin-1-yl)acetate (0.12 g, 0.26 mmol) in DCM (5 mL) was added 4N dioxane hydrochloride (1 mL) at 0° C. and slowly brought to RT and stirred for 1 h. The reaction mixture was evaporated under reduced pressure, the resultant residue was washed with diethyl ether and dried under vacuum to afford the title compound orange solid (0.1 g, 94%). ¹H NMR (400 MHz, DMSO-d₆): δ 8.11 (s, 1H), 8.02 (bs, 1H), 7.66 (m, 3H), 7.46 (m, 2H), 7.33 (m, 1H), 7.04 (d, J=6.8 Hz, 1H), 6.94 (m, 1H), 4.16 (bs, 2H), 3.62 (m, 2H), 3.21 (m, 2H), 2.91 (m, 1H), 1.92-2.18 (m, 4H); LC-MS: m/z 405.2 (M+H).

### Step-v: (2S,4R)-1-((S)-2-(2-(4-(4-(3-amino-6-(2-hydroxyphenyl)pyridazin-4-yl)phenyl)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-(4-(3-amino-6-(2-hydroxyphenyl)pyridazin-4-yl)phenyl)piperidin-1-yl)acetic acid hydrochloride (0.07 g, 0.17 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.08 g, 0.17 mmol) in DMF (5 mL) at 0° C. was added HATU (0.1 g, 0.25 mmol) followed by the dropwise addition of DIPEA (0.067 mL, 0.51 mmol) and the reaction mixture was stirred for 4 h at RT. The reaction mixture was poured into ice cold water and extracted with ethyl acetate. The combined organic layer was washed with water and brine, dried over anhydrous sodium sulphate and concentrated under vacuum to get the crude product which was purified first by combiflash column chromatography using 8% methanol in DCM system followed by prep HPLC, pure fractions were dried under lyophilization to afford the title compound off-white solid (0.04 g, 27.8%). ¹H NMR (400 MHz, DMSO-d₆): δ 13.69 (bs, 1H), 8.97 (s, 1H), 8.45 (d, J=7.6 Hz, 1H), 8.00 (s, 1H), 7.94 (d, J=8.0 Hz, 1H), 7.80 (d, J=9.6 Hz, 1H), 7.59 (d, J=8.4 Hz, 2H), 7.47-7.42 (m, 4H), 7.36 (d, J=8.4 Hz, 2H), 7.24 (t, J=8 Hz, 1H), 6.93-6.86 (m, 2H), 6.44 (bs, 2H), 5.13 (d, J=3.6 Hz, 1H), 4.89 (d, J=8.8 Hz, 1H), 4.53 (d, J=9.6 Hz, 1H), 4.44 (t, J=8 Hz, 1H), 4.28 (bs, 2H), 3.59 (bs, 2H), 3.12 (d, J=16.0 Hz, 1H), 2.96 (m, 3H), 2.64 (m, 1H), 2.44 (s, 3H), 2.36-2.23 (m, 1H), 2.06 (m, 1H), 1.87 (m, 2H), 1.75 (m, 3H), 1.37 (d, J=6.8 Hz, 3H), 0.96 (s, 9H); LCMS: m/z 831.7 (M+H).

The compounds listed in below Table-4 were prepared by the procedure similar to the one described in Example-21 with appropriate variations in reactants, quantities of reagents, protections and deprotections, solvents and reaction conditions. The characterization data of the compounds are summarized herein the below table.

| Com- | | |
|---|---|---|
| pound | Structure | Characterization Data |
| 30 | 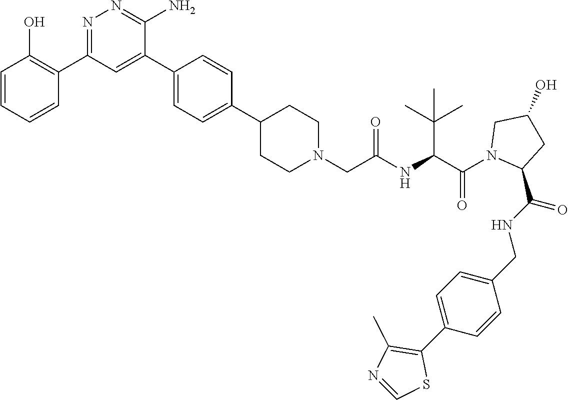 [SMILES: Cc1ncsc1-c1ccc(CNC(=O)[C@@H]2C[C@@H](O)CN2C(=O)[C@@H](NC(=O)CN2CCC(c3ccc(-c4cc(-c5ccccc5O)nnc4N)cc3)CC2)C(C)(C)C)cc1] | ¹H NMR (400 MHz, DMSO-d₆): δ 13.63 (s, 1H), 8.90 (s, 1H), 8.59 (s, 1H), 7.97 (s, 2H), 7.93 (d, J = 8.0 Hz, 1H), 7.52 (d, J = 7.2 Hz, 2H), 7.43-7.39 (m, 6H), 7.24 (s, 1H), 6.92-6.87 (m, 2H), 6.44 (bs, 2H), 5.14 (d, J = 3.2 Hz, 1H), 4.51 (m, 1H) 4.43 (m, 1H), 4.35 (bs, 2H), 4.21 (bs, 1H), 3.62 (bs, 2H), 3.21 (s, 2H), 2.97 (d, J = 14.4 Hz, 3H), 2.44 (s, 3H), 2.31 (m, 1H), 2.12 (m, 2H), 1.87 (d, J = 13.2 Hz, 3H), 1.79 (m, 2H), 0.96 (s, 9H); LCMS: m/z 818.5 (M + H) |
| | (2S,4R)-1-((S)-2-(2-(4-(4-(3-amino-6-(2- | |
| | hydroxyphenyl)pyridazin-4- | |
| | yl)phenyl)piperidin-1-yl)acetamido)-3,3- | |
| | dimethylbutanoyl)-4-hydroxy-N-(4-(4- | |
| | methylthiazol-5-yl)benzyl)pyrrolidine-2- | |
| | carboxamide | |
| 31 | 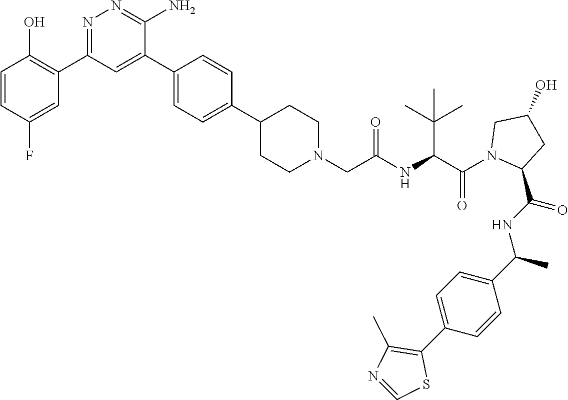 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5cc(F)ccc5O)nnc4N)cc3)CC2)C(C)(C)C)cc1] | ¹H NMR (400 MHz, DMSO-d₆): δ 13.44 (s, 1H), 8.97 (s, 1H), 8.45 (d, J = 8.0 Hz, 1H), 8.06 (s, 1H), 7.87 (d, J = 4.0 Hz, 1H), 7.80 (d, J = 8.0 Hz, 1H), 7.59 (d, J = 8.0 Hz, 2H), 7.47-7.42 (m, 4H), 7.35 (s, 2H), 7.11-7.06 (m, 1H), 6.90 (d, J = 4.0 Hz, 1H), 6.56 (bs, 2H), 5.13 (d, J = 4.0 Hz, 1H), 4.90 (t, J = 4.0 Hz, 1H), 4.52 (d, J = 12.0 Hz, 1H), 4.45 (t, J = 8.0 Hz, 1H), 4.28 (bs, 1H), 3.59 (t, J = 4.0 Hz, 2H), 2.95-2.66 (m, 3H), 2.64-2.61 (m, 2H), 2.49 (s, 3H), 2.33-2.23 (m, 2H), 2.07 (d, J = 8.0 Hz, 1H), 1.89-1.81 (m, 2H), 1.79-1.72 (m, 3H), 1.37 (d, J = 8.0 Hz, 3H), 0.95 (s, 9H); LCMS: m/z 849.1 (M + H) |
| | (2S,4R)-1-((S)-2-(2-(4-(4-(3-amino-6-(5- | |
| | fluoro-2-hydroxyphenyl)pyridazin-4- | |
| | yl)phenyl)piperidin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 32 Racemic | 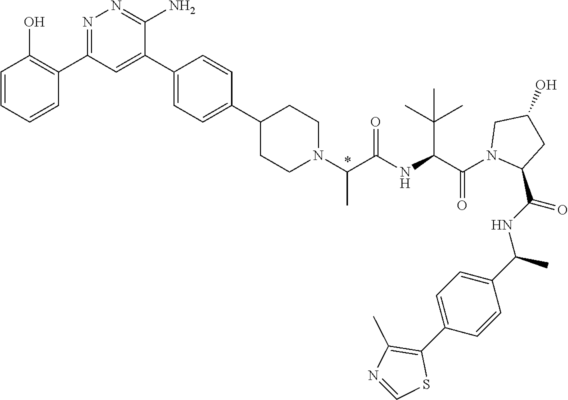 [SMILES: Cc1ncsc1-c1ccc([C@H](C)NC(=O)[C@@H]2C[C@@H](O)CN2C(=O)[C@@H](NC(=O)C(C)N2CCC(c3ccc(-c4cc(-c5ccccc5O)nnc4N)cc3)CC2)C(C)(C)C)cc1] | ¹H NMR (400 MHz, DMSO-d₆): δ 13.64 (s, 1H), 8.97 (s, 1H), 8.42 (m, 1H), 8.00 (s, 1H), 7.93 (d, J = 8.4 Hz, 2H), 7.59 (d, J = 7.6 Hz, 2H), 7.45-7.32 (m, 5H), 7.24 (m, 2H), 6.93-6.85 (m, 2H), 6.47 (bs, 2H), 5.13 (bs, 1H), 4.88 (m, 2H), 4.44 (m, 2H), 4.28 (bs, 1H), 3.60 (bs, 2H), 3.29-3.24 (m, 1H), 2.93 (m, 1H), 2.80 (m, 1H), 2.66-2.60 (m, 1H), 2.32 (s, 3H), 2.29 (bs, 1H), 2.30-2.15 (m, 1H), 2.05 (m, 1H), 1.88 (m, 2H), 1.80-1.62 (m, 3H), 1.37 (m, 2H), 1.12 (m, 3H), 0.94 (s, 9H); LCMS: m/z 845.6 (M + H) |
| | (2S,4R)-1-((2S)-2-(2-(4-(4-(3-amino-6- | |
| | (2-hydroxyphenyl)pyridazin-4- | |
| | yl)phenyl)piperidin-1-yl)propanamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 33 Isomer-1 | 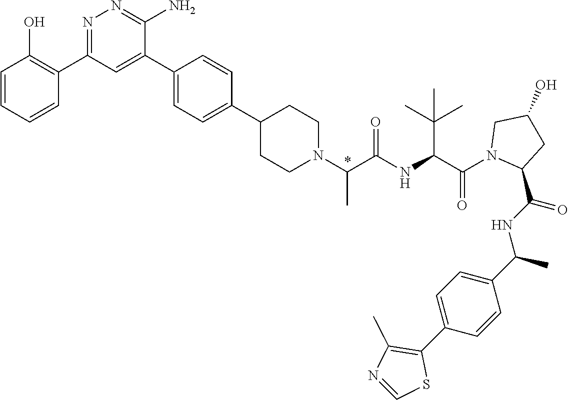 [SMILES: Cc1ncsc1-c1ccc([C@H](C)NC(=O)[C@@H]2C[C@@H](O)CN2C(=O)[C@@H](NC(=O)C(C)N2CCC(c3ccc(-c4cc(-c5ccccc5O)nnc4N)cc3)CC2)C(C)(C)C)cc1] | ¹H NMR (400 MHz, DMSO-d₆): δ 13.45 (bs, 1H), 8.97 (s, 1H), 8.42 (d, J = 7.2 Hz, 1H), 8.01 (s, 1H), 7.94-7.89 (m, 2H), 7.59 (d, J = 7.6 Hz, 2H), 7.45-7.41 (m, 4H), 7.36 (d, J = 8.8 Hz, 2H), 7.25-7.21 (m, 1H), 6.92-6.86 (m, 3H), 6.45 (bs, 2H), 4.90-4.86 (m, 1H), 4.47-4.44 (m, 2H), 4.28 (bs, 1H), 3.60 (s, 2H), 3.24 (d, J = 6.8 Hz, 1H), 2.94- 2.92 (m, 1H), 2.84-2.83 (m, 1H), 2.64-2.60 (m, 1H), 2.45 (s, 3H), 2.26-2.22 (m, 2H), 2.07-2.05 (m, 1H), 1.87-1.85 (m, 2H), 1.77-1.70 (m, 3H), 1.37 (d, J = 7.2 Hz, 3H), 1.12 (d, J = 6.8 Hz, 3H), 0.96 (s, 9H); LCMS: m/z 845.6 (M + H) |
| | (2S,4R)-1-((2S)-2-(2-(4-(4-(3-amino-6- | |
| | (2-hydroxyphenyl)pyridazin-4- | |
| | yl)phenyl)piperidin-1-yl)propanamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 34 Isomer-2 | 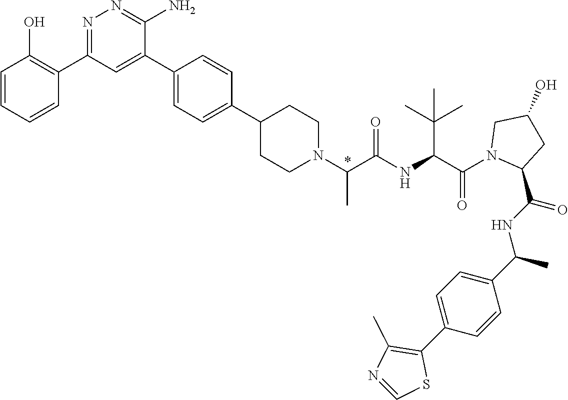 [SMILES: Cc1ncsc1-c1ccc([C@H](C)NC(=O)[C@@H]2C[C@@H](O)CN2C(=O)[C@@H](NC(=O)C(C)N2CCC(c3ccc(-c4cc(-c5ccccc5O)nnc4N)cc3)CC2)C(C)(C)C)cc1] | ¹H NMR (400 MHz, DMSO-d₆): δ 13.45 (bs, 1H), 8.97 (s, 1H), 8.42 (d, J = 7.2 Hz, 1H), 8.01 (s, 1H), 7.94-7.89 (m, 2H), 7.59 (d, J = 7.6 Hz, 2H), 7.45-7.41 (m, 4H), 7.36 (d, J = 8.8 Hz, 2H), 7.25-7.21 (m, 1H), 6.92-6.86 (m, 3H), 6.45 (bs, 2H), 4.90-4.86 (m, 1H), 4.47-4.44 (m, 2H), 4.28 (bs, 1H), 3.60 (s, 2H), 3.24 (d, J = 6.8 Hz, 1H), 2.94- 2.92 (m, 1H), 2.84-2.83 (m, 1H), 2.64-2.60 (m, 1H), 2.45 (s, 3H), 2.26-2.22 (m, 2H), 2.07-2.05 (m, 1H), 1.87-1.85 (m, 2H), 1.77-1.70 (m, 3H), 1.37 (d, J = 7.2 Hz, 3H), 1.12 (d, J = 6.8 Hz, 3H), 0.96 (s, 9H); LCMS: m/z 845.6 (M + H) |
| | (2S,4R)-1-((2S)-2-(2-(4-(4-(3-amino-6- | |
| | (2-hydroxyphenyl)pyridazin-4- | |
| | yl)phenyl)piperidin-1-yl)propanamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 35 Racemic | 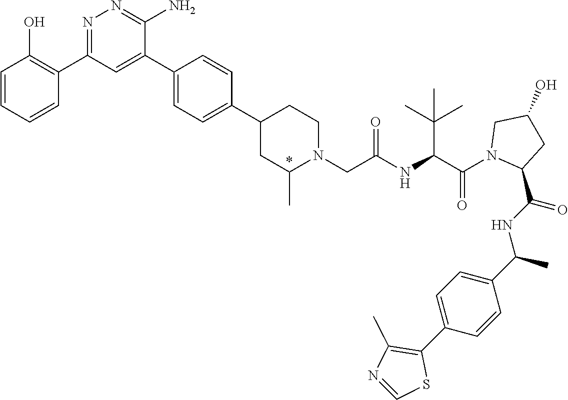 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)CC2C)C(C)(C)C)cc1] | ¹H NMR (400 MHz, DMSO-d₆): δ 13.63 (brs, 1H), 8.97 (s, 1H), 8.47- 8.42 (m, 1H), 8.00-7.93 (m, 3H), 7.59 (d, J = 8.0 Hz, 2H), 7.45-7.35 (m, 6H), 7.26-7.23 (m, 1H), 6.93- 6.87 (m, 2H), 6.45 (bs, 2H), 5.15 (bs, 1H), 4.92-4.84 (m, 1H), 4.52- 4.43 (m, 2H), 4.30 (bs, 1H), 3.59 (bs, 2H), 3.02-2.67 (m, 4H), 2.44 (s, 3H), 2.10-2.05 (m, 1H), 1.90- 1.63 (m, 4H), 1.38-1.37 (m, 4H), 1.26-1.23 (m, 1H), 1.08-1.05 (m, 3H), 0.96 (s, 9H), 0.87-0.83 (m, 1H); LCMS: m/z 845.6 (M + H) |
| | (2S,4R)-1-((2S)-2-(2-(4-(4-(3-amino-6- | |
| | (2-hydroxyphenyl)pyridazin-4- | |
| | yl)phenyl)-2-methylpiperidin-1- | |
| | yl)acetamido)-3,3-dimethylbutanoyl)-4- | |
| | hydroxy-N-((S)-1-(4-(4-methylthiazol-5- | |
| | yl)phenyl)ethyl)pyrrolidine-2- | |
| | carboxamide | |
| 36 Isomer-1 | 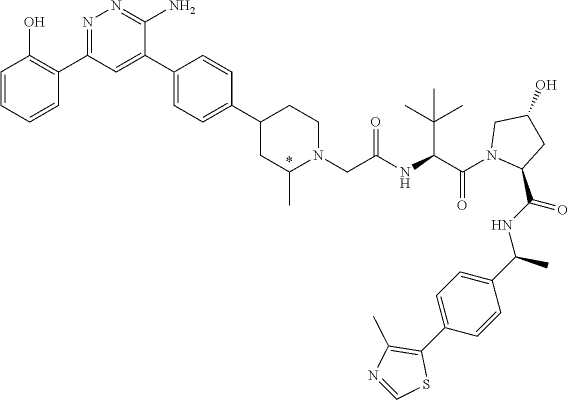 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)CC2C)C(C)(C)C)cc1] | ¹H NMR (400 MHz, DMSO-d₆): δ 13.63 (bs, 1H), 8.98 (s, 1H), 8.47 (d, J = 9.6 Hz, 1H), 8.00 (s, 1H), 7.96-7.91 (m, 1H), 7.60 (d, J = 11.2 Hz, 2H), 7.46-7.42 (m, 5H), 7.37 (d, J = 11.2 Hz, 2H), 7.27-7.22 (m, 1H), 6.93-6.85 (m, 2H), 6.46 (bs, 2H), 5.14 (bs, 1H), 4.91-4.86 (m, 1H), 4.52-4.42 (m, 2H), 4.29 (bs, 1H), 3.59 (bs, 2H), 3.41-3.38 (m, 2H), 2.91-2.73 (m, 3H), 2.45 (s, 3H), 2.08-2.03 (m, 1H), 1.88- 1.71 (m, 4H), 1.47-1.42 (m, 1H), 1.39 (d, J = 9.2 Hz, 3H), 1.11-1.03 (m, 4H), 0.97 (s, 9H); LCMS: m/z 845.5 (M + H) |
| | (2S,4R)-1-((2S)-2-(2-(4-(4-(3-amino-6- | |
| | (2-hydroxyphenyl)pyridazin-4- | |
| | yl)phenyl)-2-methylpiperidin-1- | |
| | yl)acetamido)-3,3-dimethylbutanoyl)-4- | |
| | hydroxy-N-((S)-1-(4-(4-methylthiazol-5- | |
| | yl)phenyl)ethyl)pyrrolidine-2- | |
| | carboxamide | |
| 37 Isomer-2 | 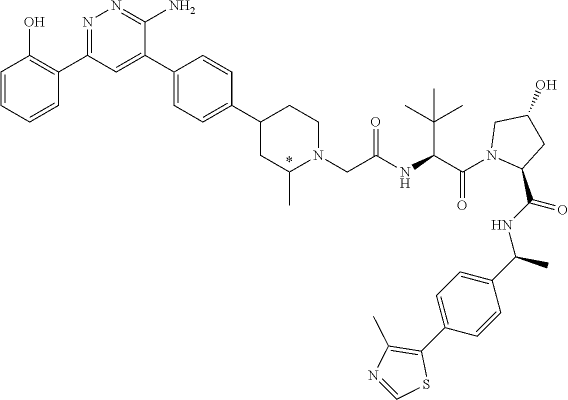 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)CC2C)C(C)(C)C)cc1] | ¹H NMR (400 MHz, DMSO-d₆): δ 13.63 (brs, 1H), 8.98 (s, 1H), 8.47 (d, J = 9.6 Hz, 1H), 8.00 (s, 1H), 7.98-7.91 (m, 2H), 7.60 (d, J = 11.2 Hz, 2H), 7.46-7.42 (m, 4H), 7.37 (d, J = 11.2 Hz, 2H), 7.27-7.22 (m, 1H), 6.93-6.85 (m, 2H), 6.46 (brs, 2H), 5.14 (brs, 1H), 4.91-4.86 (m, 1H), 4.52-4.42 (m, 2H), 4.29 (brs, 1H), 3.59 (brs, 2H), 3.41-3.38 (m, 2H), 2.91-2.73 (m, 3H), 2.45 (s, 3H), 2.08-2.03 (m, 1H), 1.88- 1.71 (m, 4H), 1.47-1.42 (m, 1H), 1.39 (d, J = 9.2 Hz, 3H), 1.11-1.03 (m, 4H), 0.97 (s, 9H); LCMS: m/z 845.5 (M + H) |
| | (2S,4R)-1-((2S)-2-(2-(4-(4-(3-amino-6- | |
| | (2-hydroxyphenyl)pyridazin-4- | |
| | yl)phenyl)-2-methylpiperidin-1- | |
| | yl)acetamido)-3,3-dimethylbutanoyl)-4- | |
| | hydroxy-N-((S)-1-(4-(4-methylthiazol-5- | |
| | yl)phenyl)ethyl)pyrrolidine-2- | |
| | carboxamide | |
| 38 | 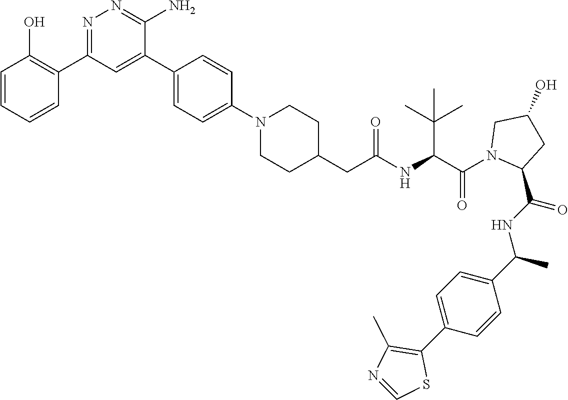 [SMILES: Cc1ncsc1-c1ccc([C@H](C)NC(=O)[C@@H]2C[C@@H](O)CN2C(=O)[C@@H](NC(=O)CC2CCN(c3ccc(-c4cc(-c5ccccc5O)nnc4N)cc3)CC2)C(C)(C)C)cc1] | ¹H NMR (400 MHz, CD₃OD): δ 8.87 (s, 1H), 7.89 (bs, 1H), 7.80 (m, 1H), 7.51-7.40 (m, 6H), 7.25 (m, 1H), 7.12 (d, J = 8.8 Hz, 2H), 6.95-6.92 (m, 2H), 5.00 (d, J = 7.2 Hz, 1H), 4.65 (bs, 1H), 4.57 (m, 1H), 4.44 (bs, 1H), 3.86 (m, 2H), 3.76 (m, 1H), 2.81 (m, 2H), 2.48 (s, 3H), 2.28-2.15 (m, 3H), 1.97 (m, 2H), 1.83 (m, 1H), 1.59 (m, 1H), 1.51 (d, J = 6.8 Hz, 2H), 1.42 (m, 1H), 1.20 (d, J = 6.4 Hz, 3H), 1.06 (s, 9H); LCMS: m/z 832.4 (M + H) |
| | (2S,4R)-1-((S)-2-(2-(1-(4-(3-amino-6-(2- | |
| | hydroxyphenyl)pyridazin-4- | |
| | yl)phenyl)piperidin-4-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 39 | 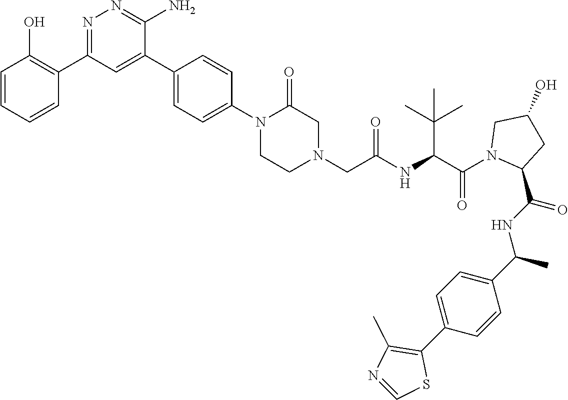 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)cc3)C(=O)C2)C(C)(C)C)cc1] | ¹H NMR (400 MHz, DMSO-d₆): δ 13.54 (bs, 1H), 8.97 (s, 1H), 8.43 (d, J = 7.6 Hz, 1H), 8.03 (s, 1H), 7.95 (d, J = 8.0 Hz, 1H), 7.82-7.78 (m, 1H), 7.68 (d, J = 8.4 Hz, 2H), 7.57 (d, J = 8.4 Hz, 2H), 7.43 (d, J = 8.0 Hz, 2H), 7.36-7.32 (m, 2H) 7.26-7.22 (m, 1H), 6.92-6.85 (m, 2H), 6.54 (bs, 2H), 5.12 (bs, 1H), 4.91-4.87 (m, 1H), 4.55 (d, J = 11.2 Hz, 1H), 4.45-4.41 (m, 2H)4.28 (bs, 1H), 3.75-3.74 (m, 2H), 3.59-3.56 (m, 2H), 3.39 (bs, 2H), 3.19 (bs, 1H), 2.96 (bs, 2H), 2.44 (s, 3H), 2.06-2.01 (m, 1H), 1.79-1.73 (m, 1H), 1.37 (d, J = 7.2 Hz, 3H), 0.96 (s, 9H); LCMS: m/z 848.3 (M + H) |
| | (2S,4R)-1-((S)-2-(2-(4-(4-(3-amino-6-(2- | |
| | hydroxyphenyl)pyridazin-4-yl)phenyl)-3- | |
| | oxopiperazin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 40 Racemic | 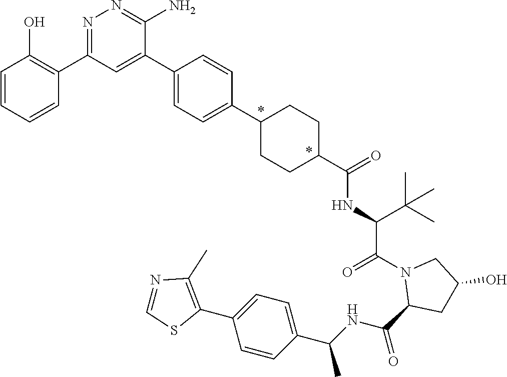 [SMILES: Cc1ncsc1-c1ccc([C@H](C)NC(=O)[C@@H]2C[C@@H](O)CN2C(=O)[C@@H](NC(=O)C2CCC(c3ccc(-c4cc(-c5ccccc5O)nnc4N)cc3)CC2)C(C)(C)C)cc1] | ¹H NMR (400 MHz, DMSO-d₆): δ 13.66 (bs, 1H), 8.98 (s, 1H), 8.40 (d, J = 8.0 Hz, 1H), 8.01 (d, J = 2.0 Hz, 1H), 7.95 (d, J = 8.4 Hz, 1H), 7.64 (d, J = 9.6 Hz, 1H), 7.57 (d, J = 8.0 Hz, 2H), 7.44-7.36 (m, 4H), 7.25 (t, J = 8.0 Hz, 1H), 6.93-6.85 (m, 2H), 6.46 (bs, 2H), 5.13 (d, J = 3.6 Hz, 1H), 4.93 (t, J = 6.8 Hz, 2H), 4.44 (t, J = 8.0 Hz, 1H), 4.29 (s, 1H), 3.62 (s, 2H), 2.45 (s, 3H), 2.02-2.00 (m, 4H), 1.89 (bs, 2H), 1.81-1.79 (m, 2H), 1.68 (m, 2H), 1.52 (m, 4H), 1.37 (d, J = 8.0 Hz, 3H), 0.85 (s, 9H); LCMS: m/z 817.4 (M + H) |
| | (2S,4R)-1-((S)-2-(4-(4-(3-amino-6-(2- | |
| | hydroxyphenyl)pyridazin-4- | |
| | yl)phenyl)cyclohexane-1-carboxamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 41 Isomer-1 | 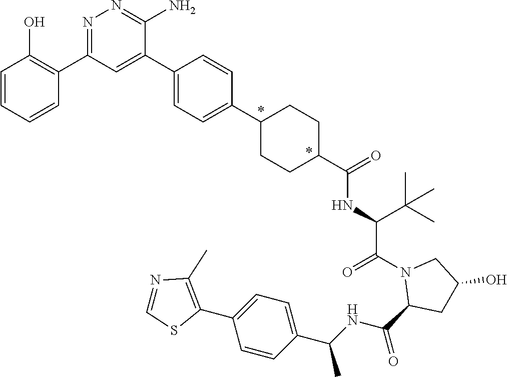 [SMILES: Cc1ncsc1-c1ccc([C@H](C)NC(=O)[C@@H]2C[C@@H](O)CN2C(=O)[C@@H](NC(=O)C2CCC(c3ccc(-c4cc(-c5ccccc5O)nnc4N)cc3)CC2)C(C)(C)C)cc1] | ¹H NMR (400 MHz, DMSO-d₆): δ 13.65 (bs, 1H), 8.97 (s, 1H), 8.39 (d, J = 7.6 Hz, 1H), 8.00 (s, 1H), 7.95 (d, J = 9.6 Hz, 1H), 7.63 (d, J = 9.2 Hz, 1H), 7.57 (d, J = 8.4 Hz, 2H), 7.48-7.35 (m, 5H), 7.27-7.21 (m, 2H), 6.91-6.81 (m, 2H), 6.44 (bs, 2H), 5.01 (s, 1H), 4.95-4.89 (m, 2H), 4.57-4.40 (m, 2H), 4.28 (bs, 1H), 3.62 (s, 2H), 2.66 (bs. 2H), 2.45 (s, 3H), 2.12-1.76 (m, 5H), 1.69 (bs, 2H), 1.56-1.50 (m, 2H), 1.35 (d, J = 7.2 Hz, 3H), 0.96 (s, 9H); LCMS: m/z 816.6 (M + H) |
| | (2S,4R)-1-((S)-2-(4-(4-(3-amino-6-(2- | |
| | hydroxyphenyl)pyridazin-4- | |
| | yl)phenyl)cyclohexane-1-carboxamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 42 Isomer-2 | 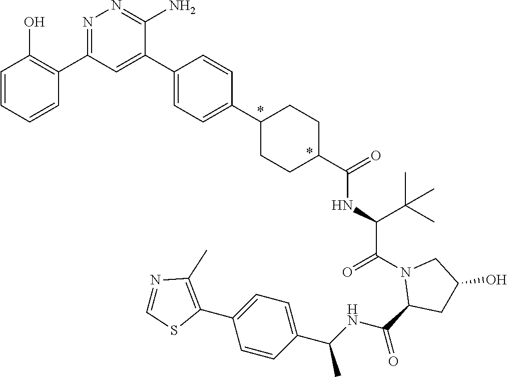 [SMILES: Cc1ncsc1-c1ccc([C@H](C)NC(=O)[C@@H]2C[C@@H](O)CN2C(=O)[C@@H](NC(=O)C2CCC(c3ccc(-c4cc(-c5ccccc5O)nnc4N)cc3)CC2)C(C)(C)C)cc1] | ¹H NMR (400 MHz, DMSO-d₆): δ 13.65 (bs, 1H), 8.97 (s, 1H), 8.39 (d, J = 7.6 Hz, 1H), 8.00 (s, 1H), 7.95 (d, J = 9.6 Hz, 1H), 7.63 (d, J = 9.2 Hz, 1H), 7.57 (d, J = 8.4 Hz, 2H), 7.48-7.35 (m, 5H), 7.27-7.21 (m, 2H), 6.91-6.81 (m, 2H), 6.44 (bs, 2H), 5.01 (s, 1H), 4.95-4.89 (m, 2H), 4.57-4.40 (m, 2H), 4.28 (bs, 1H), 3.62 (s, 2H), 2.66 (bs. 2H), 2.45 (s, 3H), 2.12-1.76 (m, 5H), 1.69 (bs, 2H), 1.56-1.50 (m, 2H), 1.35 (d, J = 7.2 Hz, 3H), 0.96 (s, 9H); LCMS: m/z 816.6 (M + H) |
| | (2S,4R)-1-((S)-2-(4-(4-(3-amino-6-(2- | |
| | hydroxyphenyl)pyridazin-4- | |
| | yl)phenyl)cyclohexane-1-carboxamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |
| 43 Racemic | 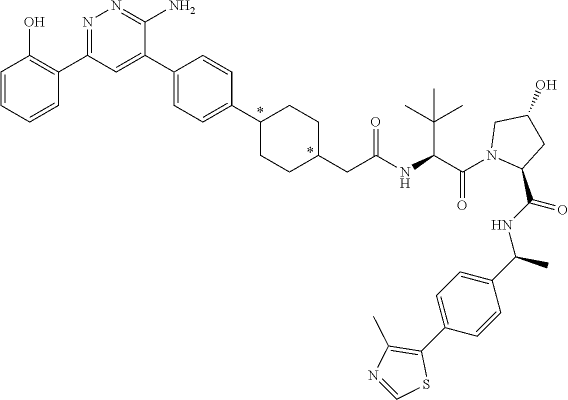 [SMILES: Cc1ncsc1-c1ccc([C@H](C)NC(=O)[C@@H]2C[C@@H](O)CN2C(=O)[C@@H](NC(=O)CC2CCC(c3ccc(-c4cc(-c5ccccc5O)nnc4N)cc3)CC2)C(C)(C)C)cc1] | ¹H NMR (400 MHz, DMSO-d₆): δ 13.66 (d, J = 5.6 Hz, 1H), 8.98 (s, 1H), 8.39 (d, J = 8.0 Hz, 1H), 8.05- 8.00 (m, 1H), 7.98-7.95 (m, 1H), 7.84-7.82 (m, 1H), 7.57 (t, J = 8.4 Hz, 2H), 7.50 (d, J = 8.0 Hz, 1H), 7.45-7.37 (m, 5H), 7.25 (t, J = 7.6 Hz, 1H), 6.93-6.86 (m, 2H), 6.46 (d, J = 7.2 Hz, 2H), 5.11 (t, J = 7.2 Hz, 1H), 4.92-4.90 (m, 1H), 4.58- 4.53 (m, 1H), 4.43 (t, J = 8.0 Hz, 1H), 4.29 (bs, 1H), 3.62 (bs, 2H), 3.42-3.40 (m, 3H), 2.32-2.19 (m, 2H), 2.45 (s, 3H), 2.01-1.99 (m, 2H), 1.85-1.79 (m, 3H), 1.57-1.48 (m, 2H), 1.37 (d, J = 6.8 Hz, 3H), 1.27-1.24 (m, 2H), 0.95 (s, 9H).; LCMS: m/z 831.4 (M + H) |
| | (2S,4R)-1-((S)-2-(2-(4-(4-(3-amino-6-(2- | |
| | hydroxyphenyl)pyridazin-4- | |
| | yl)phenyl)cyclohexyl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 44 Isomer-1 | 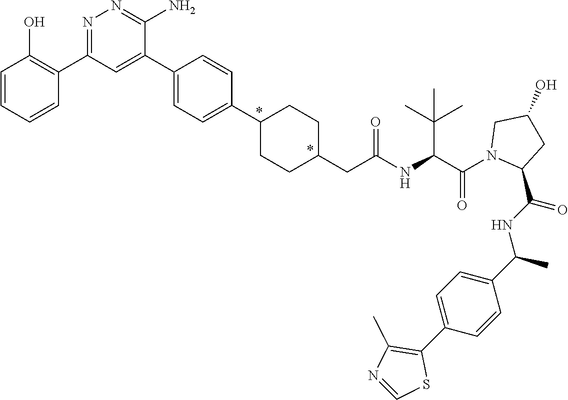 [SMILES: Cc1ncsc1-c1ccc([C@H](C)NC(=O)[C@@H]2C[C@@H](O)CN2C(=O)[C@@H](NC(=O)CC2CCC(c3ccc(-c4cc(-c5ccccc5O)nnc4N)cc3)CC2)C(C)(C)C)cc1] | ¹H NMR (400 MHz, DMSO-d₆): δ 13.63 (bs, 1H), 8.98 (s, 1H), 8.41 (d, J = 10.4 Hz, 1H), 8.02 (s, 1H), 7.97 (d, J = 10.8 Hz, 1H), 7.59 (d, J = 10.4 Hz, 2H), 7.51 (d. J = 11.2 Hz, 2H), 7.45-7.36 (m, 5H), 7.27-7.22 (m, 1H), 6.93-6.85 (m, 2H), 6.46 (bs, 2H), 5.13 (bs, 1H), 4.92-4.90 (m, 1H), 4.59 (d, J = 12 Hz, 1H), 4.43-4.40 (m, 1H), 4.29 (bs, 1H), 3.62 (bs, 2H), 2.72-2.53 (m, 3H), 2.45 (s, 3H), 2.20-2.16 (m, 3H), 2.01 (bs, 1H), 1.84-1.75 (m, 1H), 1.59 (bs, 6H), 1.39 (d, J = 9.2 Hz, 3H), 0.94 (s, 9H); LCMS: m/z 831.2 (M + H) |
| | (2S,4R)-1-((S)-2-(2-(4-(4-(3-amino-6-(2- | |
| | hydroxyphenyl)pyridazin-4- | |
| | yl)phenyl)cyclohexyl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 45 Isomer-2 | 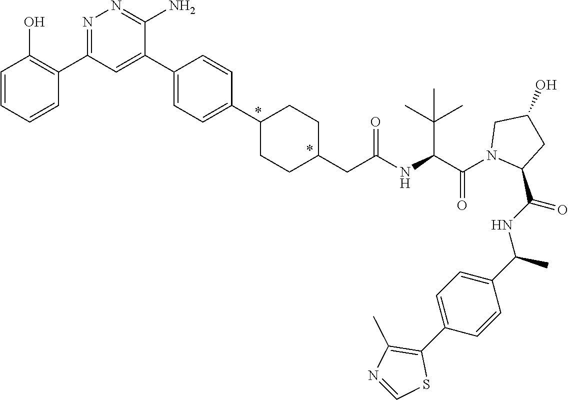 [SMILES: Cc1ncsc1-c1ccc([C@H](C)NC(=O)[C@@H]2C[C@@H](O)CN2C(=O)[C@@H](NC(=O)CC2CCC(c3ccc(-c4cc(-c5ccccc5O)nnc4N)cc3)CC2)C(C)(C)C)cc1] | ¹H NMR (400 MHz, DMSO-d₆): δ 13.63 (bs, 1H), 8.98 (s, 1H), 8.41 (d, J = 10.4 Hz, 1H), 8.00 (s, 1H), 7.97 (d, J = 10.8 Hz, 1H), 7.85 (d, J = 12.4 Hz, 1H), 7.57 (d, J = 10.4 Hz, 2H), 7.42-7.36 (m, 6H), 7.27-7.22 (m, 1H), 6.93-6.85 (m, 2H), 6.45 (bs, 2H), 5.11 (bs, 1H), 4.94-4.90 (m, 1H), 4.55 (d, J = 11.6 Hz, 1H), 4.45-4.40 (m, 1H), 4.29 (bs, 1H), 3.62 (bs, 2H), 2.59 (bs, 1H), 2.45 (s, 3H), 2.26-2.19 (m, 2H), 2.13- 2.05 (m, 2H), 1.82-1.79 (bs, 6H), 1.55-1.43 (m, 3H), 1.39 (d, J = 8.8 Hz, 3H), 0.94 (s, 9H); LCMS: m/z 831.2 (M + H) |
| | (2S,4R)-1-((S)-2-(2-(4-(4-(3-amino-6-(2- | |
| | hydroxyphenyl)pyridazin-4- | |
| | yl)phenyl)cyclohexyl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 46 | 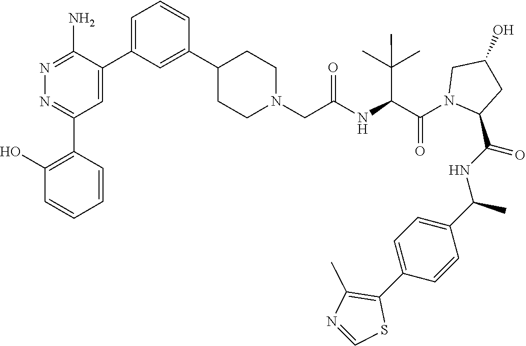 [SMILES: Cc1ncsc1-c1ccc([C@H](C)NC(=O)[C@@H]2C[C@@H](O)CN2C(=O)[C@@H](NC(=O)CN2CCC(c3cccc(-c4cc(-c5ccccc5O)nnc4N)c3)CC2)C(C)(C)C)cc1] | ¹H NMR (400 MHz, DMSO-d₆): δ 13.64 (bs, 1H), 8.97 (s, 1H), 8.43 (d, J = 7.6 Hz, 1H), 8.01 (s, 1H), 7.96 (d, J = 7.6 Hz, 1H), 7.76-7.74 (m, 1H), 7.79-7.41 (m, 6H), 7.35 (d, J = 8.0 Hz, 2H) 7.26-7.22 (m, 1H), 6.92-6.85 (m, 2H), 6.44 (bs, 2H), 5.12 (d, J = 3.6 Hz, 1H), 4.88- 4.85 (m, 1H), 4.53 (d, J = 10 Hz, 1H), 4.44-4.42 (m, 1H), 4.27 (bs, 1H), 3.57 (bs, 1H), 3.12-3.08 (m, 1H), 2.96 (bs, 3H), 2.65-2.59 (m, 2H), 2.44 (s, 3H), 2.31-2.23 (m, 2H), 2.06-2.015 (m, 2H), 1.89- 1.74 (m, 4H), 1.35 (d, J = 6.8 Hz, 3H), 0.94 (s, 9H); LCMS: m/z 832.3 (M + H) |
| | (2S,4R)-1-((S)-2-(2-(4-(3-(3-amino-6-(2- | |
| | hydroxyphenyl)pyridazin-4- | |
| | yl)phenyl)piperidin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 47 | 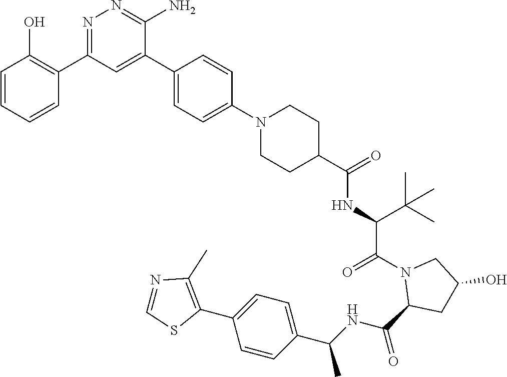 [SMILES: Cc1ncsc1-c1ccc([C@H](C)NC(=O)[C@@H]2C[C@@H](O)CN2C(=O)[C@@H](NC(=O)C2CCN(c3ccc(-c4cc(-c5ccccc5O)nnc4N)cc3)CC2)C(C)(C)C)cc1] | ¹H NMR (400 MHz, DMSO-d⁶): δ 13.77 (bs, 1H), 8.97 (s, 1H), 8.39 (d, J = 8.0 Hz, 1H), 7.94-7.93 (m, 2H), 7.85 (d, J = 10.0 Hz, 1H), 7.57 (d, J = 8.40 Hz, 2H), 7.43 (d, J = 8.4 Hz, 2H), 7.38 (d, J = 8.4 Hz, 2H), 7.25-7.21 (m, 1H), 7.08 (d, J = 8.8 Hz, 2H), 6.91-6.85 (m, 2H), 6.38 (bs, 2H), 5.10 (d, J = 3.6 Hz, 1H), 4.92-4.89 (m, 1H), 4.50 (d, J = 9.2 Hz, 1H), 4.43-4.39 (m, 1H), 4.26 (bs, 1H), 3.91-3.87 (m, 2H), 3.59-3.57 (m, 2H), 3.36-3.35 (m, 1H), 2.83-2.78 (m, 2H), 2.65 (bs, 1H), 2.44 (s, 3H), 1.99-1.97 (m, 2H), 1.80-1.77 (m, 1H), 1.66-1.60 (m, 2H), 1.37 (d, J = 3.6 Hz, 3H), 0.94 (s, 9H); LCMS: m/z 817.05 (M + H) |
| | 1-(4-(3-amino-6-(2- | |
| | hydroxyphenyl)pyridazin-4-yl)phenyl)- | |
| | N-((S)-1-((2S,4R)-4-hydroxy-2-(((S)-1- | |
| | (4-(4-methylthiazol-5- | |
| | yl)phenyl)ethyl)carbamoyl)pyrrolidin-1- | |
| | yl)-3,3-dimethyl-1-oxobutan-2- | |
| | yl)piperidine-4-carboxamide | |
| 48 | 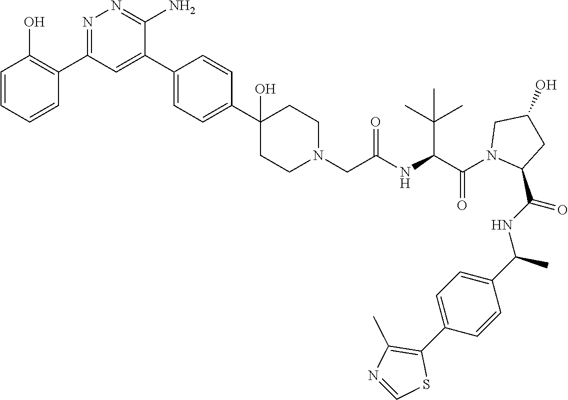 [SMILES: Cc1ncsc1-c1ccc([C@H](C)NC(=O)[C@@H]2C[C@@H](O)CN2C(=O)[C@@H](NC(=O)CN2CCC(O)(c3ccc(-c4cc(-c5ccccc5O)nnc4N)cc3)CC2)C(C)(C)C)cc1] | ¹H NMR (400 MHz, DMSO-d₆): δ 13.64 (bs, 1H), 8.98 (s, 1H), 8.46 (d, J = 10 Hz, 1H), 8.01 (s, 1H), 7.97 (d, J = 10.4 Hz, 1H), 7.87 (d, J = 12.8 Hz, 1H), 7.69-7.61 (m, 5H), 7.45 (d, J = 10.8 Hz, 2H), 7.37 (d, J = 11.2 Hz, 2H), 6.94-6.85 (m, 2H), 6.48 (bs, 2H), 5.15 (d, J = 4.4 Hz, 1H), 5.07 (bs, 1H), 4.91-4.86 (m, 1H), 4.54-4.41 (m, 2H), 4.28 (bs, 1H), 3.59 (bs, 1H), 3.17-2.95 (m, 2H), 2.69-2.62 (m, 4H), 2.45 (s, 3H), 2.10-2.00 (m, 4H), 1.75 (bs, 3H), 1.39 (d, J = 9.2 Hz, 3H), 0.94 (s, 9H); LCMS: m/z 848.3 (M + H) |
| | (2S,4R)-1-((S)-2-(2-(4-(4-(3-amino-6-(2- | |
| | hydroxyphenyl)pyridazin-4-yl)phenyl)-4- | |
| | hydroxypiperidin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 49 | 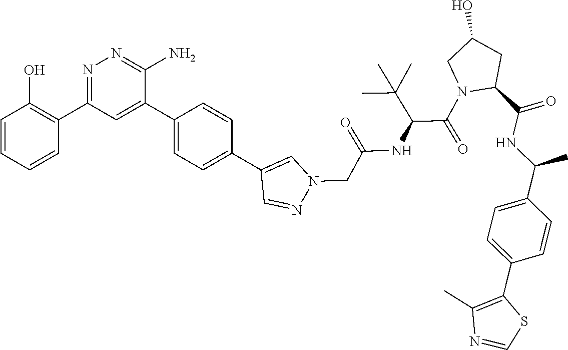 [SMILES: Cc1ncsc1-c1ccc([C@H](C)NC(=O)[C@@H]2C[C@@H](O)CN2C(=O)[C@@H](NC(=O)Cn2cc(-c3ccc(-c4cc(-c5ccccc5O)nnc4N)cc3)cn2)C(C)(C)C)cc1] | ¹H NMR (400 MHz, DMSO-d₆): δ 13.64 (bs, 1H), 8.97 (s, 1H), 8.42 (d, J = 7.2 Hz, 1H), 8.29 (s, 1H), 8.12 (m, 1H), 8.02 (s, 1H), 7.98- 7.96 (m, 1H), 7.76 (d, J = 8.4 Hz, 2H), 7.64 (d, J = 8.4 Hz, 2H), 7.41- 7.37 (m, 4H), 7.29-7.22 (m, 1H), 6.92-6.87 (m, 2H), 6.50 (bs, 2H), 5.31 (bs, 1H), 5.11 (bs, 1H), 4.97- 4.88 (m, 3H), 4.53-4.40 (m, 2H), 4.25 (bs, 2H), 3.58-3.54 (m, 1H), 2.08-1.98 (m, 1H), 1.75 (bs, 1H), 1.37 (d, J = 6.8 Hz, 3H), 1.12 (d, J = 6.8 Hz, 3H), 0.94 (s, 9H); LCMS: m/z 814.6 (M + H) |
| | (2S,4R)-1-((S)-2-(2-(4-(4-(3-amino-6-(2- | |
| | hydroxyphenyl)pyridazin-4-yl)phenyl)- | |
| | 1H-pyrazol-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 50 | 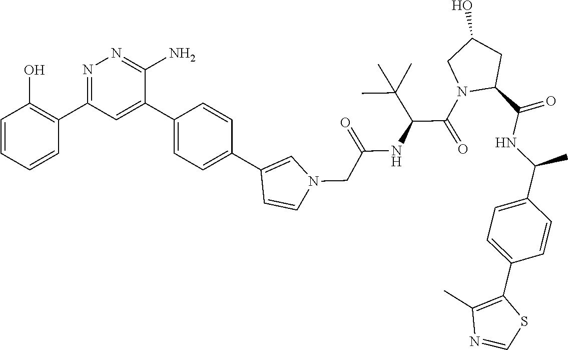 [SMILES: Cc1ncsc1-c1ccc([C@H](C)NC(=O)[C@@H]2C[C@@H](O)CN2C(=O)[C@@H](NC(=O)Cn2ccc(-c3ccc(-c4cc(-c5ccccc5O)nnc4N)cc3)c2)C(C)(C)C)cc1] | ¹H NMR (400 MHz, CD₃OD): δ 8.86 (s, 1H), 7.95 (s, 1H), 7.80 (d, J = 8.00 Hz, 1H), 7.75-7.72 (m, 2H), 7.75-7.54 (m, 2H), 7.43-7.24 (m, 6H), 6.96-6.90 (m, 2H), 6.82 (d, J = 2 Hz, 1H), 6.60 (bs, 1H), 4.98 (m, 1H), 4.74 (bs, 2H), 4.60 (s, 1H), 4.56 (m, 1H), 4.42 (bs, 1H), 3.85 (m, 1H), 3.73 (m, 1H), 2.46 (s, 3H), 2.23-2.15 (m, 1H), 1.90-1.95 (m, 1H), 1.49 (d, J = 7.2 Hz, 3H), 0.99 (s, 9H); LCMS: m/z 814.3 (M + H) |
| | (2S,4R)-1-((S)-2-(2-(3-(4-(3-amino-6-(2- | |
| | hydroxyphenyl)pyridazin-4-yl)phenyl)- | |
| | 1H-pyrrol-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 51 | 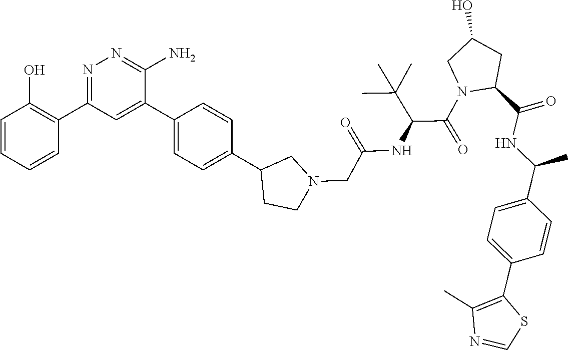 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)C2)C(C)(C)C)cc1] | ¹H NMR (400 MHz, DMSO-d₆): δ 13.64 (d, J = 12.4 Hz, 1H), 8.97 (s, 1H), 8.47-8.36 (m, 1H), 8.01-7.93 (m, 2H), 7.75 (brs, 1H), 7.58-7.24 (m, 8H), 6.93-6.87 (m, 3H), 6.43 (d, J = 6.8 Hz, 2H), 5.13 (brs, 1H), 4.88 (brs, 1H), 4.53-4.40 (m, 2H), 4.28 (brs, 1H), 3.58 (brs, 1H), 3.48-3.46 (m, 1H), 3.23 (m, 1H), 3.12-3.04 (m, 1H), 2.96-2.89 (m, 1H), 2.81-2.72 (m, 2H), 2.44 (s, 3H), 2.33-2.30 (m, 1H), 2.07-2.01 (m, 1H), 1.91-1.86 (m, 1H), 1.77- 1.72 (m, 1H), 1.35-1.23 (m, 2H), 1.12 (d, J = 6.8 Hz, 3H), 0.96 (s, 9H); LCMS: m/z 818.3 (M + H) |
| | (2S,4R)-1-((2S)-2-(2-(3-(4-(3-amino-6- | |
| | (2-hydroxyphenyl)pyridazin-4- | |
| | yl)phenyl)pyrrolidin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 52 | 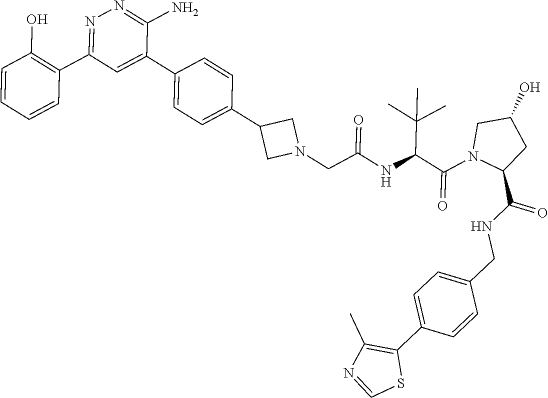 | ¹H NMR (400 MHz, DMSO-d₆): δ 13.61 (bs, 1H), 8.97 (s, 1H), 8.44 (d, J = 8.0 Hz, 1H), 7.99 (s, 1H), 7.94 (d, J = 8.0 Hz, 1H), 7.70-7.54 (m, 5H), 7.43 (d, J = 8.0 Hz, 2H), 7.36 (d, J = 7.6 Hz, 2H), 6.92-6.87 (m, 3H), 6.45 (bs, 2H), 5.13 (d, J = 3.2 Hz, 1H), 4.86-4.84 (m, 1H), 4.52 (d, J = 9.6 Hz, 1H), 4.44-4.43 (m, 1H), 3.81 (bs, 2H), 3.58-3.56 (m, 3H), 3.31 (bs, 1H), 3.20 (bs, 2H), 2.44 (s, 3H), 2.08-2.04 (m, 2H), 1.77-1.73 (m, 2H), 1.37 (d, J = 7.2 Hz, 3H), 0.94 (s, 9H); LCMS: m/z 803.6 (M + H) |
| | (2S,4R)-1-((S)-2-(2-(3-(4-(3-amino-6-(2- | |
| | hydroxyphenyl)pyridazin-4- | |
| | yl)phenyl)azetidin-1-yl)acetamido)-3,3- | |
| | dimethylbutanoyl)-4-hydroxy-N-((S)-1- | |
| | (4-(4-methylthiazol-5- | |
| | yl)phenyl)ethyl)pyrrolidine-2- | |
| | carboxamide | |
| 53 | 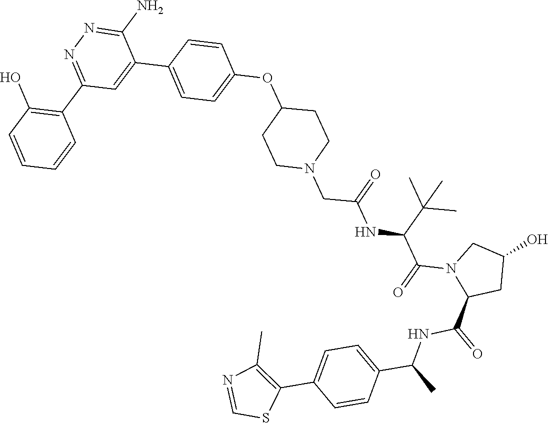 [SMILES: Cc1ncsc1-c1ccc([C@H](C)NC(=O)[C@@H]2C[C@@H](O)CN2C(=O)[C@@H](NC(=O)CN2CCC(Oc3ccc(-c4cc(-c5ccccc5O)nnc4N)cc3)CC2)C(C)(C)C)cc1] | ¹H NMR (400 MHz, DMSO-d₆): δ 13.44 (s, 1H), 8.97 (s, 1H), 8.45 (d, J = 8.0 Hz, 1H), 8.06 (s, 1H), 7.87 (d, J = 4.0 Hz, 1H), 7.80 (d, J = 8.0 Hz, 1H), 7.59 (d, J = 8.0 Hz, 2H), 7.47-7.42 (m, 5H), 7.35 (s, 2H), 7.11-7.06 (m, 1H), 6.90 (d, J = 4.0 Hz, 1H), 6.56 (bs, 2H), 5.13 (d, J = 4.0 Hz, 1H), 4.90 (t, J = 4.0 Hz, 1H), 4.52 (d, J = 12.0 Hz, 1H), 4.45 (t, J = 8.0 Hz, 1H), 4.28 (bs, 1H), 3.59 (t, J = 4.0 Hz, 2H), 2.95-2.66 (m, 3H), 2.64-2.61 (m, 2H), 2.49 (s, 3H), 2.33-2.23 (m, 2H), 2.07 (d, J = 8.0 Hz, 1H), 1.89-1.81 (m, 2H), 1.79-1.72 (m, 3H), 1.37 (d, J = 8.0 Hz, 3H), 0.95 (s, 9H); LCMS: m/z 849.1 (M + H) |
| | (2S,4R)-1-((S)-2-(2-(4-(4-(3-amino-6-(2- | |
| | hydroxyphenyl)pyridazin-4- | |
| | yl)phenoxy)piperidin-1-yl)acetamido)- | |
| | 3,3-dimethylbutanoyl)-4-hydroxy-N-((S)- | |
| | 1-(4-(4-methylthiazol-5- | |
| | yl)phenyl)ethyl)pyrrolidine-2- | |
| | carboxamide | |

### Example-22: Chiral separation of (2S,4R)-1-((2S)-2-(2-(4-(4-(3-amino-6-(2-hydroxyphenyl)pyridazin-4-yl)phenyl)piperidin-1-yl)propanamido)-3,3-dimethylbutanoyl)-4-hydroxy-N—((S)-1-(4-(4-methylthiazol-5-yl)phenyl)ethyl)pyrrolidine-2-carboxamide (Compound 32)

Compounds 33 (Isomer-1 of compound 32) and 34 (Isomer-2 of compound 32) were obtained by chiral HPLC of Compound 32 by using the following method. Chiral HPLC method Mobile Phase: A=Acetonitrile B=0.1% DEA in ethanol. Flow:15 ml/min, Column: Chiral pak-IG (250 mm×21.2 mm×5.0μ)

### Example-23: Chiral separation of (2S,4R)-1-((2S)-2-(2-(4-(4-(3-amino-6-(2-hydroxyphenyl)pyridazin-4-yl)phenyl)-2-methylpiperidin-1-yl)acetamido)-3,3-dimethylbutanoyl)-4-hvdroxy-N—((S)-1-(4-(4-methylthiazol-5-yl)phenyl)ethyl)pyrrolidine-2-carboxamide (Compound 35)

Compounds 36 (Isomer-1 of compound 35) and 37 (Isomer-2 of compound 35) were obtained by chiral HPLC of Compound 35 using the following method. Chiral HPLC method Mobile Phase: A: n-HEXANE, B=0.1% DEA in ethanol. Flow:17 ml/min, Column: Regis, Reflect, i-Cellulose-C(250×20.2 mm), 5 μm.

Similarly, compound 41 (Isomer-1 of compound 40), 42 (Isomer-2 of compound 40), 44 (Isomer-1 of compound 43) and 45 (Isomer-2 of compound 43) isomers were separated according to the procedure described in Example 23.

### Example-24: (2S,4R)-1-((S)-2-(2-(4-((4-(3-amino-6-(2-hydroxyphenyl)pyridazin-4-yl)phenyl)amino)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (Compound 54)

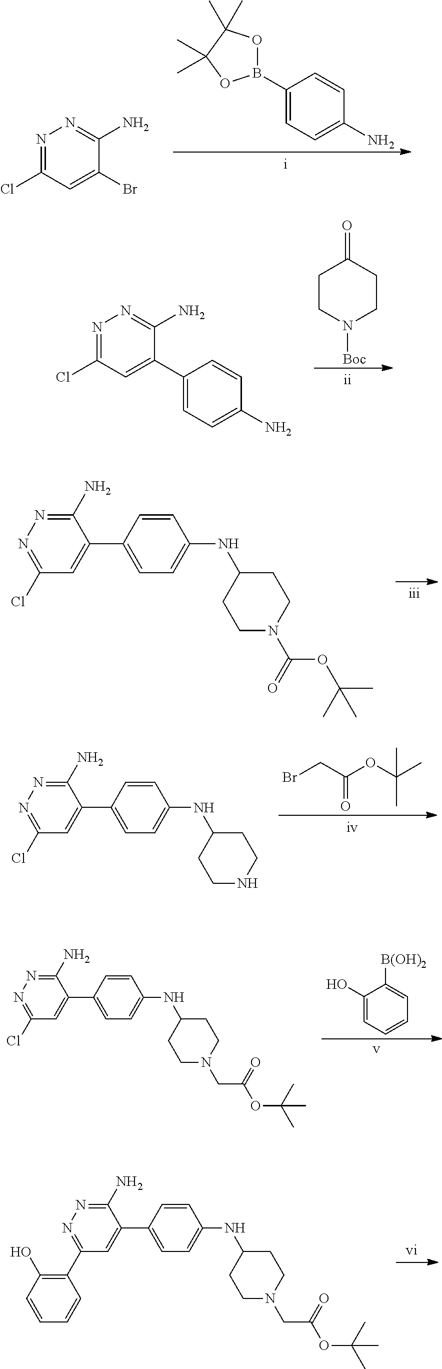

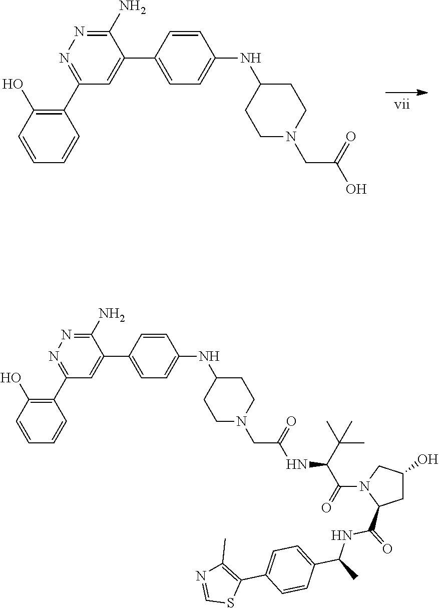 [SMILES: Nc1nnc(-c2ccccc2O)cc1-c1ccc(NC2CCN(CC(=O)O)CC2)cc1 ; Cc1ncsc1-c1ccc([C@H](C)NC(=O)[C@@H]2C[C@@H](O)CN2C(=O)[C@@H](NC(=O)CN2CCC(Nc3ccc(-c4cc(-c5ccccc5O)nnc4N)cc3)CC2)C(C)(C)C)cc1]

### Step-i: Synthesis of 4-(4-aminophenyl)-6-chloropyridazin-3-amine

To a stirred solution of 4-bromo-6-chloropyridazin-3-amine (2 g, 9.61 mmol), 4-(4,4,5,5-tetramethyl-1,3,2-dioxaborolan-2-yl)aniline (2.3 g, 10.5 mmol) in 1,4-dioxane (40 mL) and water (4 mL) was added K₂CO₃ (3.97 g, 28.8 mmol) and degassed with nitrogen for 15 min. Pd(dppf)Cl₂·DCM (0.78 g, 0.96 mmol) was added in the reaction mixture and heated for 16 h at 100° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 35% ethyl acetate in hexane as eluent to afford the title compound as pale brown solid (1.3 g, 64%). ¹H NMR (400 MHz, DMSO-d6): δ 7.29 (d, J=8.8 Hz, 2H), 7.24 (s, 1H), 6.69 (d, J=8.8 Hz, 2H), 6.23 (bs, 2H), 5.59 (bs, 2H); LC-MS: m/z 221.0 (M+H).

### Step-ii: Synthesis of tert-butyl 4-((4-(3-amino-6-chloropyridazin-4-yl)phenyl)amino)piperidine-1-carboxylate

To a stirred solution of 4-(4-aminophenyl)-6-chloropyridazin-3-amine (0.4 g, 1.81 mmol) and tert-butyl 4-oxopiperidine-1-carboxylate (0.72 g, 3.62 mmol) in methanol (30 mL) was added acetic acid (0.2 mL) and stirred at RT for 2 hrs. Sodium cyanoborohydride (0.22 g, 3.6 mmol) was added into the reaction mixture and stirred at RT for 18 hrs. (confirmed by TLC). The reaction mixture concentrated under reduced pressure was diluted with EtOAc and the combined organic layer was washed with water and brine, dried over anhydrous sodium sulphate and concentrated under vacuum to give the residue which was purified by combiflash column chromatography using 40% ethyl acetate in hexane as eluent to afford the title compound as pale-yellow solid (0.45 g, 61%). ¹H NMR (400 MHz, DMSO-d₆): δ 7.36 (d, J=8.8 Hz, 2H), 7.26 (s, 2H), 6.74 (d, J=8.8 Hz, 2H), 6.25 (s, 2H), 6.15 (d, J=8.8 Hz, 1H) 3.90 (m, 2H), 3.54 (m, 1H), 2.96 (bs, 2H), 1.90 (m, 2H), 1.44 (s, 9H), 1.26 (m, 2H); LC-MS: m/z 404.0 (M+H).

### Step-iii: Synthesis of 6-chloro-4-(4-(piperidin-4-ylamino)phenyl)pyridazin-3-amine

To a stirred solution of tert-butyl 4-((4-(3-amino-6-chloropyridazin-4-yl)phenyl)amino)piperidine-1-carboxylate (0.25 g, 0.61 mmol) in DCM (2 mL) was added dioxane HCl (5 mL) at 0° C. and then slowly brought to RT and stirred at RT for 3 h. The reaction mixture was evaporated under reduced pressure, the resultant residue was washed with diethyl ether and dried under vacuum to afford the title compounds reddish solid (0.25 g, crude). ¹H NMR (400 MHz, DMSO-d6): δ 9.23 (bs, 2H), 8.41 (bs, 1H), 7.79 (s, 1H), 7.42 (d, J=8.8 Hz, 2H), 6.87 (d, J=8.8 Hz, 2H), 3.71 (m, 1H), 3.35 (m, 2H), 3.06 (m, 2H), 2.08 (m, 2H), 1.72 (m, 2H); LC-MS: m/z 304.2 (M+H).

### Step-iv: Synthesis of tert-butyl 2-(4-((4-(3-amino-6-chloropyridazin-4-yl)phenyl)amino)piperidin-1-yl)acetate

To a stirred solution of 6-chloro-4-(4-(piperidin-4-ylamino)phenyl)pyridazin-3-amine (0.25 g, 0.82 mmol) in DMF (5 mL) was added DIPEA (0.53 g, 4.11 mmol) at RT and stirred for 15 min. Tert-butyl 2-bromoacetate (0.16 g, 0.82 mmol) was added in the reaction mixture and stirred for 4 h at RT under nitrogen atmosphere. The reaction mixture was quenched with cold water and stirred for 1 h, the solid so formed was filtered and dried under vacuum to afford the title compound as brown solid (0.21 g, 61%). ¹H NMR (400 MHz, CDCl₃): δ 7.30 (d, J=8.8 Hz, 2H), 7.21 (s, 1H), 6.67 (d, J=8.8 Hz, 2H), 6.20 (bs, 2H), 5.95 (d, J=8.8 Hz, 1H), 3.24 (m, 1H), 3.09 (s, 2H), 2.81 (m, 2H), 2.30 (m, 2H), 1.89 (m, 2H), 1.41 (s, 9H), 1.36 (n, 2H); LC-MS: m/z 418.2 (M+H).

### Step-v: Synthesis of tert-butyl 2-(4-((4-(3-amino-6-(2-hydroxyphenyl)pyridazin-4-yl)phenyl)amino)piperidin-1-yl)acetate

To a stirred solution of tert-butyl 2-(4-((4-(3-amino-6-chloropyridazin-4-yl)phenyl)amino)piperidin-1-yl)acetate (0.16 g, 0.38 mmol), 2-hydroxyphenyl boronic acid (0.06 g, 0.46 mmol) in 1,4-dioxane (4 mL) and water (1 mL) was added K₂CO₃ (0.16 g, 1.14 mmol) and degassed with nitrogen for 10 min. Pd(dppf)Cl₂·DCM (0.03 g, 0.03 mmol) was added to the reaction mixture and heated for 16 h at 120° C. in sealed tube. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 50% ethyl acetate in hexane as eluent to afford the title compounds as pale yellow solid (0.06 g, 33%). LC-MS: m/z 476.4 (M+H).

### Step-vi: Synthesis of 2-(4-((4-(3-amino-6-(2-hydroxyphenyl)pyridazin-4-yl)phenyl)amino)piperidin-1-yl)acetic acid

To a stirred solution of tert-butyl 2-(4-((4-(3-amino-6-(2-hydroxyphenyl)pyridazin-4-yl)phenyl)amino)piperidin-1-yl)acetate (0.06 g, 0.12 mmol) in DCM (1 mL) was added 4 N dioxane hydrochloride (3 mL) at 0° C. and then slowly brought to RT and stirred at RT for 3 h. The reaction mixture was evaporated under reduced pressure, the resultant residue was washed with diethyl ether and dried under vacuum to afford the title compounds as brownish solid (0.06 g, crude. m/z 420.8 (M+H).

### Step-vii: Synthesis of (2S,4R)-1-((S)-2-(2-(4-((4-(3-amino-6-(2-hydroxyphenyl)pyridazin-4-yl)phenyl)amino)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide

To a solution of 2-(4-((4-(3-amino-6-(2-hydroxyphenyl)pyridazin-4-yl)phenyl)amino)piperidin-1-yl)acetic acid (0.04 g, 0.095 mmol) and ((2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (0.046 g, 0.10 mmol) in DMF (1 mL) at 0° C. was added HATU (0.05 g, 0.14 mmol) followed by the dropwise addition of DIPEA (0.08 g, 0.66 mmol) and the reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 8-10% methanol in DCM as eluent to afford the title compound light brown solid (0.03 g, 37%). ¹H NMR (400 MHz, DMSO-d₆): δ 13.64 (bs, 1H), 8.98 (s, 1H), 8.45-8.44 (m, 1H), 7.94-7.80 (m, 3H), 7.42-7.35 (m, 6H), 7.25-7.23 (m, 1H), 6.92-6.88 (m, 2H), 6.74 (d, J=8.8 Hz, 2H), 6.33 (bs, 2H), 5.14 (bs, 1H), 4.92-4.89 (m, 1H), 4.52-4.42 (m, 2H), 4.29 (bs, 1H), 3.58 (bs, 2H), 3.13-3.04 (m, 1H), 2.96-2.94 (m, 1H), 2.88-2.79 (bs, 2H), 2.45 (s, 3H), 2.38-2.26 (m, 3H), 2.10-1.93 (m, 3H), 1.79-1.76 (m, 1H), 1.51 (bs, 3H), 1.38 (d, J=6.4 Hz, 3H), 0.95 (s, 9H); LCMS: m/z 846.6 (M+1).

### Example-25: (2S,4R)-1-((S)-2-(2-(4-((4-(3-amino-6-(2-hydroxyphenyl)pyridazin-4-yl)phenyl)(methyl)amino)piperidin-1-yl)acetamido)-3,3-dimethylbutanoyl)-4-hydroxy-N—((S)-1-(4-(4-methylthiazol-5-yl)phenyl)ethyl)pyrrolidine-2-carboxamide (compound 55)

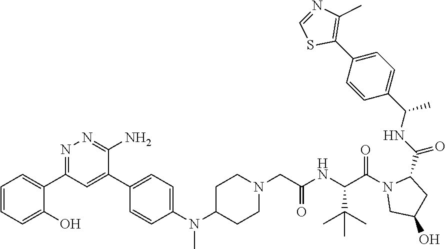 [SMILES: Cc1ncsc1-c1ccc([C@H](C)NC(=O)[C@@H]2C[C@@H](O)CN2C(=O)[C@@H](NC(=O)CN2CCC(N(C)c3ccc(-c4cc(-c5ccccc5O)nnc4N)cc3)CC2)C(C)(C)C)cc1]

Example-55 was prepared by the procedure similar to the one described in Example-24 with appropriate variations in reactants, quantities of reagents, protections and deprotections, solvents and reaction conditions. δ 13.33 (s, 1H), 9.59 (m, 1H), 8.97 (m, 1H), 8.67 (bs, 1H), 8.24 (d, J=8 Hz, 1H), 8.06-8.10 (m, 2H), 7.30-7.44 (m, 6H), 7.15-7.19 (m, 2H), 6.99-7.03 (m, 2H), 6.10 (d, J=4 Hz, 1H), 5.12 (bs, 1H), 4.90 (m, 1H), 4.32-4.36 (m, 3H), 4.20-4.24 (m, 3H), 3.64 (m, 2H), 3.44-3.50 (m, 2H), 2.45 (m, H), 2.22 (m, 3H), 2.01 (m, 1H), 1.7 (s, 1H), 1.67 (s, 1H), 1.45 (m, 1H), 1.35-1.38 (m, 3H), 0.95-096 (m, 3H), 0.78-0.84 (m, 3H) LC-MS: m/z 803.7 (M+H).

### Example-26: (2S,4R)-1-(2-(3-(2-((4-(3-amino-6-(2-hydroxyphenyl)pyridazin-4-yl)phenyl)amino)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 56)

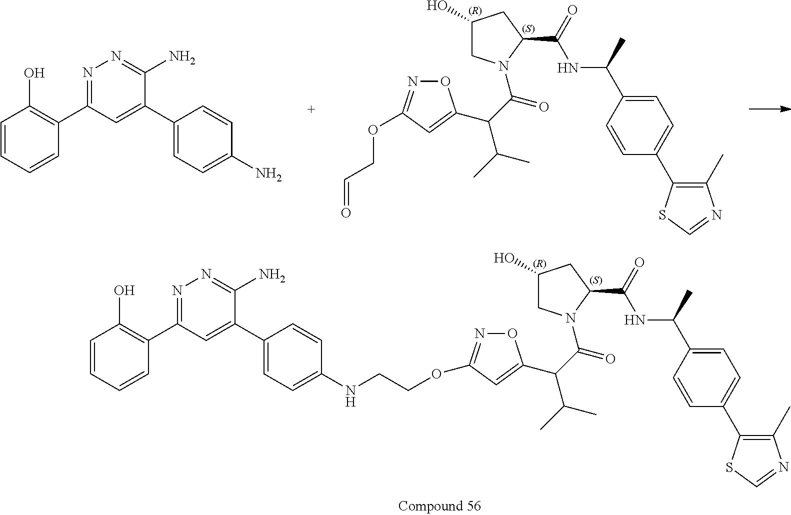

### Step-i: Synthesis of (2S,4R)-1-(2-(3-(2-((4-(3-amino-6-(2-hydroxyphenyl)pyridazin-4-yl)phenyl)amino)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

To a stirred solution of 2-(6-amino-5-(4-aminophenyl)pyridazin-3-yl)phenol (10 mg, 0.036 mmol), (2S,4R)-4-hydroxy-1-(3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (prepared following Ref. US2019/300521, 2019, A1) (20 mg, 0.036 mmol) in methanol (1 mL) was added acetic acid (0.1 mL) at 0° C. and stirred at RT for 2 hrs. Sodium cyanoborohydride (5 mg, 0.070 mmol) was added into the reaction mixture at 0° C. and stirred at RT for 2 hrs. Once the reaction was completed (monitored by TLC), the reaction mixture was quenched with sodium bicarbonate and diluted with water. The reaction mass was extracted with EtOAc and combined organic layer was washed with water and brine, dried over anhydrous sodium sulphate and concentrated under vacuum to give the residue which was purified by preparative TLC using 5% methanol in DCM as eluent to afford the title compound as pale yellow solid (10 g, 34%).

¹H NMR (400 MHz, DMSO-d₆): δ 13.85 (br, 1H), 8.99-8.96 (m, 1H), 8.41-8.36 (m, 1H), 7.96-7.82 (m, 1H), 7.90 (s, 1H), 7.42-7.39 (m, 3H), 7.38-7.30 (m, 2H), 7.27-7.22 (m, 2H), 6.92-6.87 (m, 2H), 6.82-6.77 (m, 2H), 6.34 (br, 3H), 6.02 (br, 1H), 5.21 (br, 1H) 4.93-4.85 (m, 1H), 4.44-4.37 (m, 1H), 4.35-4.25 (m, 2H), 3.79-3.75 (m, 2H), 3.55-3.45 (m, 3H), 2.44 (s, 3H), 2.05-1.96 (m, 2H), 1.80-1.72 (m, 1H), 1.48-1.42 (m, 1H), 1.49-1.42 (m, 3H), 0.957 (br, 3H), 0.88-0.78 (m, 3H); LC-MS: m/z 804.3 (M+H).

### Example-27: (2S,4R)-1-(2-(3-(2-((4-(3-amino-6-(2-hydroxyphenyl)pyridazin-4-yl)phenyl)(methyl)amino)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 57)

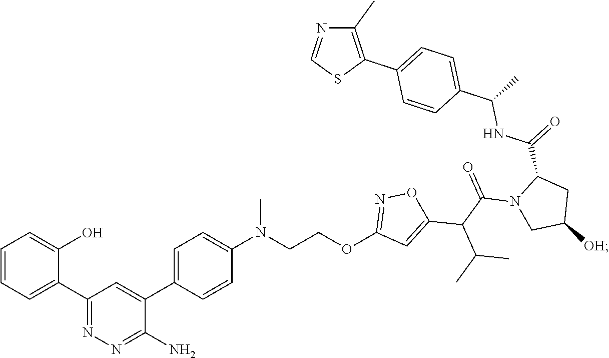 [SMILES: Cc1ncsc1-c1ccc([C@H](C)NC(=O)[C@@H]2C[C@@H](O)CN2C(=O)C(c2cc(OCCN(C)c3ccc(-c4cc(-c5ccccc5O)nnc4N)cc3)no2)C(C)C)cc1]

Compound-57 was prepared by the procedure similar to the one described in Example-26 with appropriate variations in reactants, quantities of reagents, protections and deprotections, solvents and reaction conditions. ¹H NMR (400 MHz, CDCl3): δ 8.85 (brs, 1H), 7.85 (brs, 1H), 7.60-7.56 (m, 1H), 7.53-7.48 (m, 1H), 7.38-7.36 (m, 3H), 7.36-7.35 (m, 1H), 7.35-7.34 (m, 1H), 7.33-7.34 (m, 1H), 7.09-7.05 (m, 2H), 6.98-6.93 (m, 1H), 6.83-6.71 (m, 2H), 5.72 (brs, 1H) 5.45-5.40 (m, 1H), 4.99-4.92 (m, 1H), 4.71-4.68 (m, 1H), 4.62-4.58 (m, 1H), 4.48-4.42 (m, 2H), 3.91-3.85 (m, 1H), 3.72-3.68 (m, 1H), 3.55-3.49 (m, 2H), 3.05 (bs, 3H), 2.50 (m, 3H), 2.42-1.98 (m, 3H), 1.61 (m, 1H), 1.37 (d, J=6.8 Hz, 3H), 1.02-0.95 (m, 3H), 0.91-0.85 (m, 3H); LC-MS: m/z 817.5 (M+H).

### Example-28: (2S,4R)-1-((S)-2-(5-(4-(3-amino-6-(2-hydroxyphenyl)pyridazin-4-yl)phenoxy)pentanamido)-3,3-dimethylbutanoyl)-4-hydroxy-N—((S)-1-(4-(4-methylthiazol-5-yl)phenyl)ethyl)pyrrolidine-2-carboxamide (Compound 58)

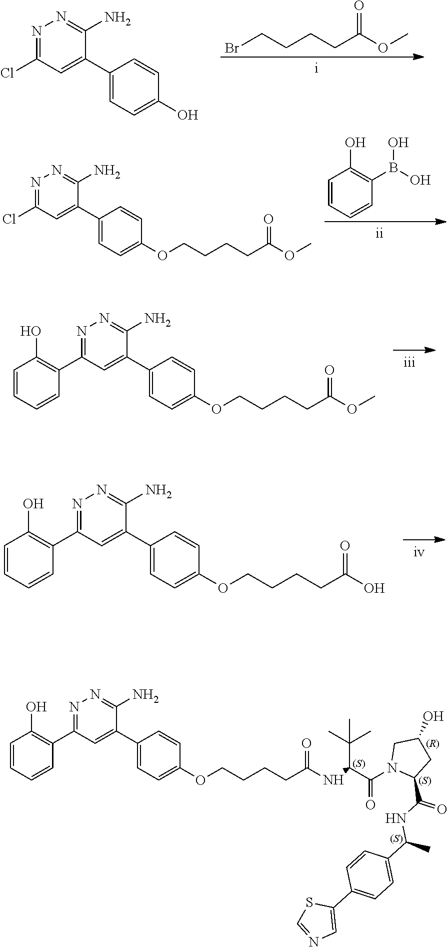

### Step-i: Synthesis of methyl 5-(4-(3-amino-6-chloropyridazin-4-yl)phenoxy)pentanoate

To a stirred solution of compound 4-(3-amino-6-chloropyridazin-4-yl)phenol (0.25 g, 1.13 mmol) in acetone (10 mL) were added methyl 5-bromopentanoate (0.25 mL, 1.69 mmol), K₂CO₃ (0.47 g, 3.39 mmol) at RT and the reaction mixture was heated at 90° C. in a sealed for 16 h. Once the reaction was completed (monitored by TLC), the reaction mixture was concentrated and diluted with EtOAc. The combined organic layer was washed with water, brine, dried over anhydrous sodium sulphate and concentrated under vacuum to give the residue which was purified by preparative HPLC to afford the title compound as pale-yellow solid (0.3 g, 79%). ¹H NMR (400 MHz, DMSO-d₆): δ 7.48 (d, J=8.4 Hz, 1H), 7.31 (s, 1H), 7.05 (d, J=8.4 Hz, 2H), 6.31 (bs, 2H), 4.03 (t, J=6 Hz, 2H), 3.59 (s, 3H), 2.39 (t, J=6.8 Hz, 2H), 1.76-1.69 (m, 4H); LC-MS: m/z 335.9 (M+H).

### Step-ii: Synthesis of methyl 5-(4-(3-amino-6-(2-hydroxyphenyl)pyridazin-4-yl)phenoxy)pentanoate

To a stirred solution of methyl 5-(4-(3-amino-6-chloropyridazin-4-yl)phenoxy)pentanoate (0.3 g, 0.895 mmol), (2-Hydroxyphenyl)boronic acid (0.19 g, 1.34 mmol) in 1,4-dioxane (6 mL) and water (1.5 mL) was added Na₂CO₃ (0.28 g, 2.68 mmol) and degassed with nitrogen for 5 min. followed by Pd(dppf)Cl₂·DCM (0.04 g, 0.04 mmol) was added and the reaction mixture was heated in a microwave for 1 h at 130° C. Once the reaction was completed (monitored by TLC), the reaction mixture was diluted with EtOAc. The combined organic layer was washed with water and brine, dried over anhydrous sodium sulphate and concentrated under vacuum to give the residue which was purified by combiflash column chromatography using 40-50% ethyl acetate in hexane as eluent to afford the title compound as off-white solid (0.05 g, 14%). LC-MS: m/z 394.1 (M+H).

### Step-iii: Synthesis of 5-(4-(3-amino-6-(2-hydroxyphenyl)pyridazin-4-yl)phenoxy)pentanoic acid

To a stirred solution of compound, methyl 5-(4-(3-amino-6-(2-hydroxyphenyl)pyridazin-4-yl)phenoxy)pentanoate (0.05 g, 0.127 mmol) in methanol:THF:H₂O (1 mL:1 mL:1 mL) mixture was added LiOH·H₂O (0.01 g, 0.25 mmol) at 0° C. The reaction mixture was stirred for 16 h at RT. The reaction mixture was then evaporated under reduced pressure and the resultant residue was diluted with methanol and acidified to pH 6 using Amberlite® IT120 and filtered and filtrate was concentrated under vacuum to afford the title compound as a sticky solid (0.04 g, 81%). LC-MS: m/z 380.05 (M+H).

### Step-iv: Synthesis of (2S,4R)-1-((S)-2-(5-(4-(3-amino-6-(2-hydroxyphenyl)pyridazin-4-yl)phenoxy)pentanamido)-3,3-dimethylbutanoyl)-4-hydroxy-N—((S)-1-(4-(thiazol-5-yl)phenyl)ethyl)pyrrolidine-2-carboxamide

To a solution of 5-(4-(3-amino-6-(2-hydroxyphenyl)pyridazin-4-yl)phenoxy)pentanoic acid (0.04 g, 0.101 mmol) and (2S,4R)-1-((S)-2-amino-3,3-dimethylbutanoyl)-4-hydroxy-N—((S)-1-(4-(4-methylthiazol-5-yl)phenyl)ethyl) pyrrolidine-2-carboxamide hydrochloride (0.06 g, 0.121 eq.) in DMF (2 mL) at 0° C. was added HATU (0.06 g, 0.151 mmol) followed by the dropwise addition of DIPEA (0.05 mL, 0.303 mmol) and the reaction mixture was stirred for 2 h at RT. The reaction mixture was poured into ice cold water and extracted with ethyl acetate. The combined organic layer was washed with water and brine, dried over anhydrous sodium sulphate and concentrated under vacuum to get the crude product which was purified by combiflash column chromatography using 5-5% methanol in DCM as eluent to afford the title compound white solid (0.02 g, 24%). ¹H NMR (400 MHz, DMSO-d₆): δ 13.71 (s, 1H), 8.99 (s, 1H), 8.40 (d, J=7.8 Hz, 1H), 7.97 (s, 1H), 7.95-7.90 (m, 1H), 7.89 (d, J=9.3 Hz, 1H), 7.58 (d, J=8.8 Hz, 2H), 7.45-7.37 (m, 5H), 7.25 (t, J=7.6 Hz, 1H), 7.10 (d, J=8.8 Hz, 2H), 6.93-6.87 (m, 2H), 6.44 (bs, 2H), 5.12 (d, J=3.4 Hz, 1H), 4.91 (d, J=7.4 Hz, 1H), 4.54 (d, J=9.3 Hz, 1H), 4.42 (t, J=8.0 Hz, 1H), 4.28 (s, 1H), 4.05 (d, J=6.4 Hz, 2H), 3.61 (bs, 2H), 2.45 (s, 3H), 2.23-2.22 (m, 1H), 2.08-1.99 (m, 1H), 1.78-1.65 (m, 5H), 1.37 (d, J=6.8 Hz, 3H), 0.95 (s, 9H); LCMS: m/z 806.3 (M+H).

### Example-29: (2S,4R)-1-(2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 59)

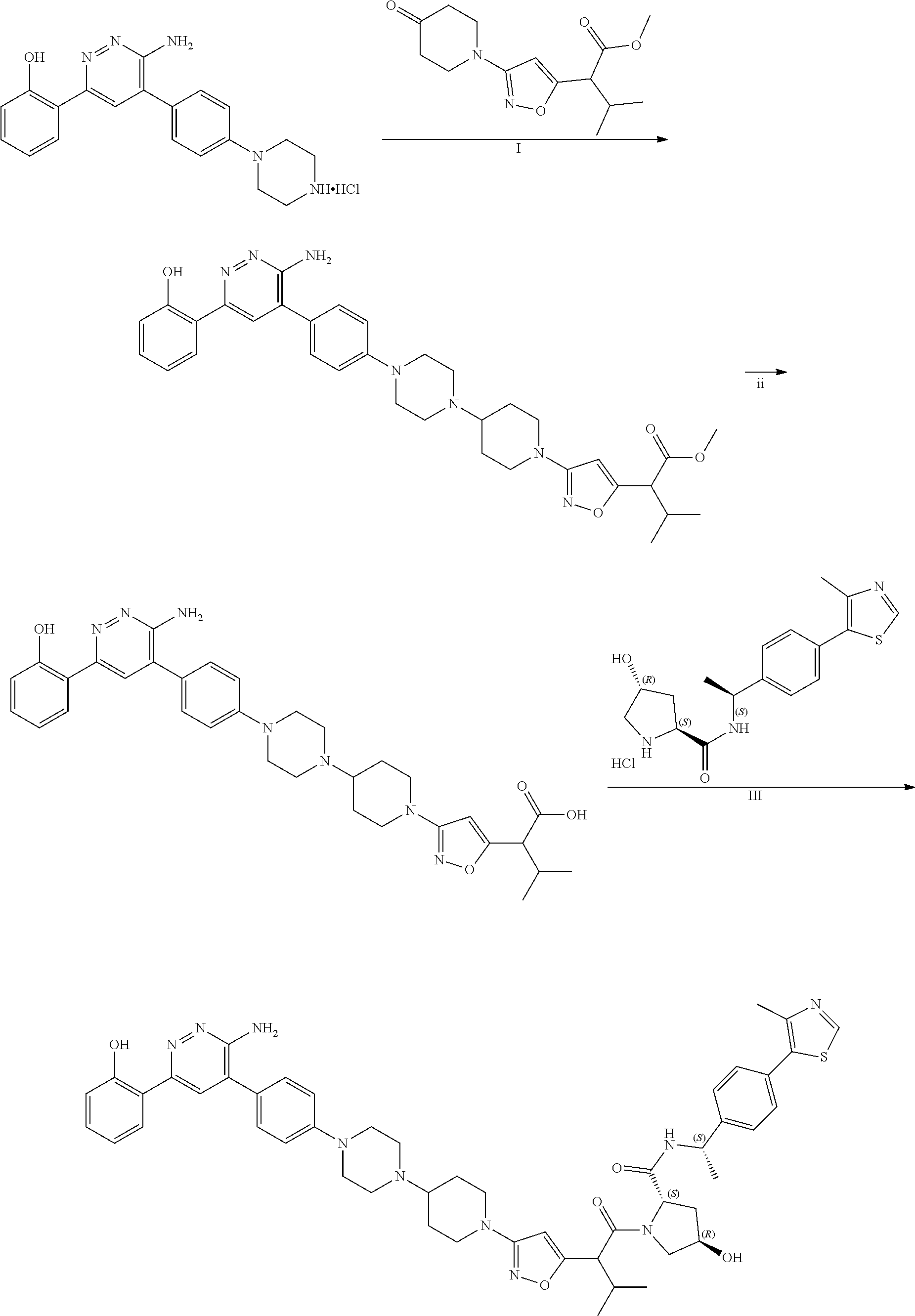

### Step-i: Synthesis of methyl 2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoate

To a stirred solution of 2-(6-amino-5-(4-(piperazin-1-yl)phenyl)pyridazin-3-yl)phenol hydrochloride (0.01 g, 0.357 mmol) and methyl 3-methyl-2-(3-(4-oxopiperidin-1-yl)isoxazol-5-yl)butanoate (0.15 g, 0.428 mmol) in 6 mL THF:DMSO (2:1) mixture were added KOAc (0.105 g, 1.07 mmol) and acetic acid (0.2 mL) and molecular sieves (4 Å) and the reaction mixture was stirred at 70° C. for 16 h. Then the reaction mixture was cooled to 0° C. and sodium cyano borohydride (0.23 g, 1.07 mmol) was added and the reaction mixture was stirred at RT for 6 h. The reaction was monitored by TLC. After completion of the reaction, the reaction mixture was poured into ice water to the obtained solid was filtered and washed with water to get the crude. The crude product was purified by combi flash column chromatography using 5% MeOH in DCM as eluent to afford the title compound as a light-yellow solid (0.20 g, 91.5%). LCMS: m/z 612.4 (M+H).

### Step-ii: Synthesis of 2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoic acid

To a stirred solution of methyl 2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoate (0.2 g, 0.327 mmol) in THF:H₂O (1 mL:1 mL) mixture was added LiOH·H₂O (0.04 g, 1.63 mmol) at 0° C. The reaction mixture was stirred for 2 h at RT. The reaction mixture was then evaporated under reduced pressure and the resultant residue was diluted with water (3 mL) and acidified to pH 6 using 1 N HCl solution at 0° C. and stirred for 30 mins. The solid so formed was filtered and dried under vacuum to afford the title compound as a light-yellow solid (0.2 g, crude); LCMS: m/z 598.4 (M+H).

### Step-iii: Synthesis of (2SAR)-1-(2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(4-(4-(4-(3-amino-6-(2-hydroxyphenyl)pyridazin-4-yl)phenyl)piperazin-1-yl)piperidin-1-yl)isoxazol-5-yl)-3-methylbutanoic acid (0.2 g, 0.335 mmol) and (2S,4R)-4-hydroxy-N—((S)-1-(4-(4-methylthiazol-5-yl)phenyl)ethyl)pyrrolidine-2-carboxamide hydrochloride (0.136 g, 0.369 mmol) in DMF (5 mL) at 0° C. was added HATU (0.19 g, 0.5 mmol) followed by the dropwise addition of DIPEA (0.216 mL, 1.67 mmol) and the reaction mixture was stirred for 2 h at RT. Then the reaction mixture was poured into ice cold water and stirred for 30 mins. Solid so formed was filtered and dried under vacuum to get the crude product. Crude product was first purified by combi flash column chromatography using 3% methanol in DCM followed by prep HPLC purification to afford the title compound off white solid (0.010 g, 44%). LC-MS: 911.6 (M+H).

### Example-30: (2S,4R)-1-(2-(4-(4-(4-(3-amino-6-(2-hydroxyphenyl)pyridazin-4-yl)phenyl)piperazin-1-yl)piperidin-1-yl)-3,3-dimethylbutanoyl)-4-hydroxy-N—((S)-1-(4-(4-methylthiazol-5-yl)phenyl)ethyl)pyrrolidine-2-carboxamide (compound 60)

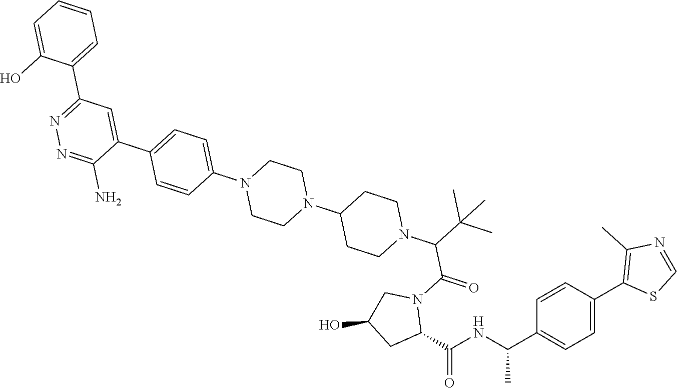 [SMILES: Cc1ncsc1-c1ccc([C@H](C)NC(=O)[C@@H]2C[C@@H](O)CN2C(=O)C(N2CCC(N3CCN(c4ccc(-c5cc(-c6ccccc6O)nnc5N)cc4)CC3)CC2)C(C)(C)C)cc1]

Compound-60 was prepared by the procedure similar to the one described in Example-29 with appropriate variations in reactants, quantities of reagents, protections and deprotections, solvents and reaction conditions. LC-MS: m/z 858.6 (M+H).

Although the present application has been illustrated by certain of the preceding examples, it is not to be construed as being limited thereby; but rather, the present application encompasses the generic area as hereinbefore disclosed. Various modifications and embodiments can be made without departing from the spirit and scope thereof. For example, the following compounds which can be prepared by following similar procedure as described above with suitable modification known to the one ordinary skilled in the art are also included in the scope of the present application:

| Compound | Structure |
|---|---|
| 61 | 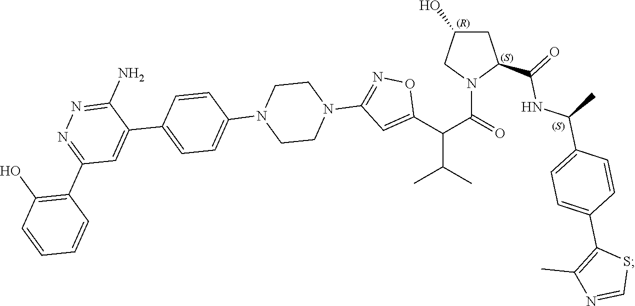 |
| 62 | 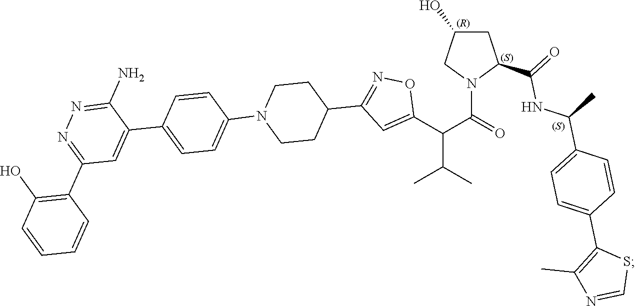 |
| 63 | 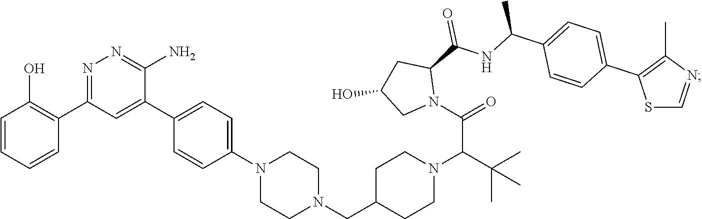 [SMILES: Cc1ncsc1-c1ccc([C@H](C)NC(=O)[C@@H]2C[C@@H](O)CN2C(=O)C(N2CCC(CN3CCN(c4ccc(-c5cc(-c6ccccc6O)nnc5N)cc4)CC3)CC2)C(C)(C)C)cc1] |
| 64 | 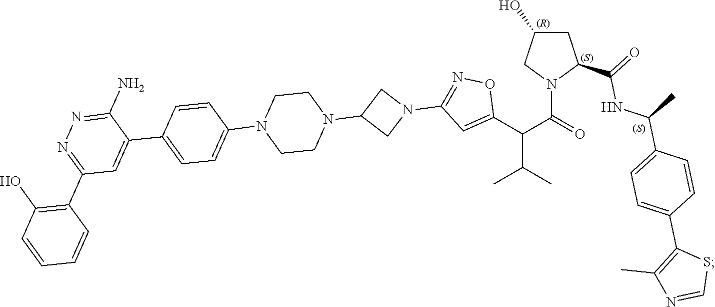 |
| 65 | 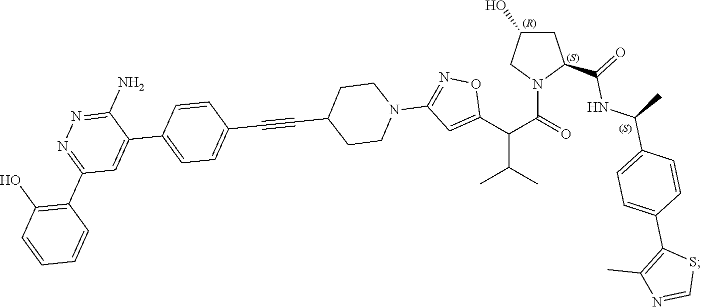 |
| 66 | 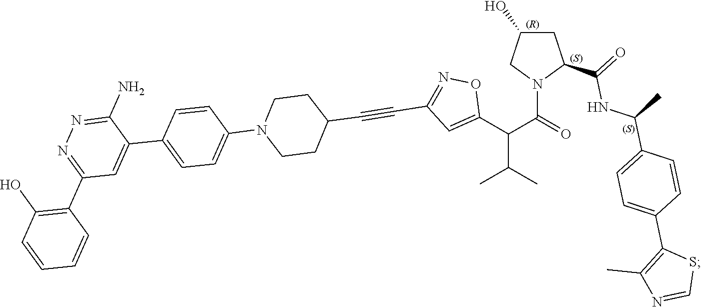 |
| 67 | 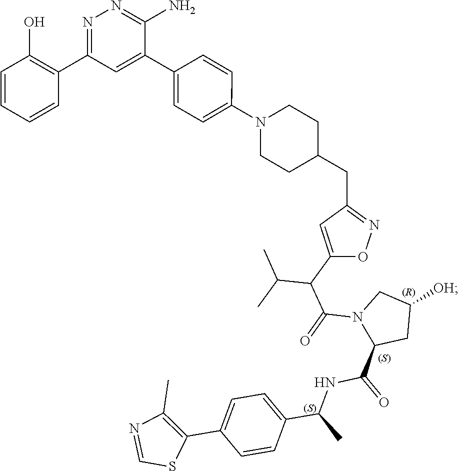 |
| 68 | 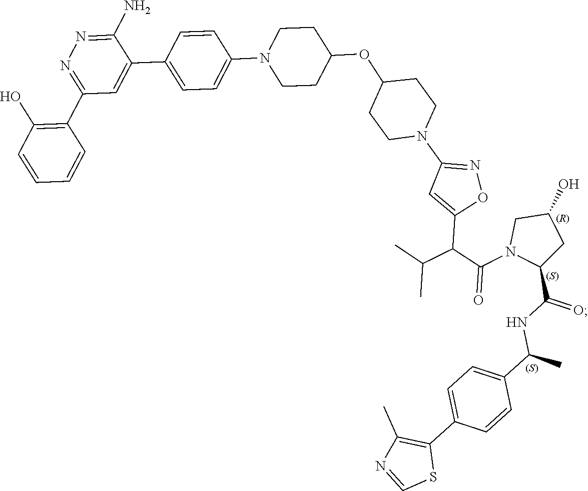 |
| 69 | 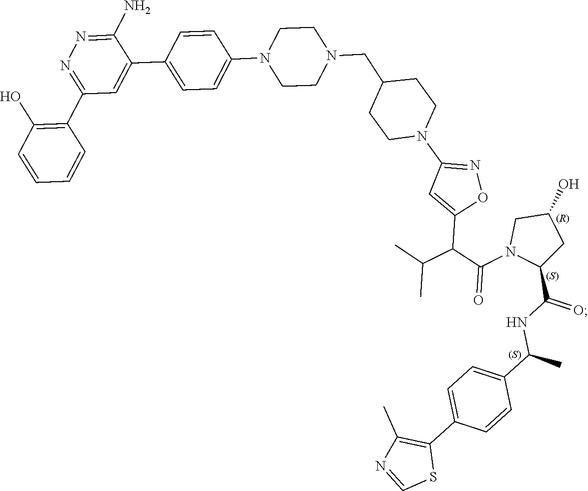 |
| | and |
| 70 | 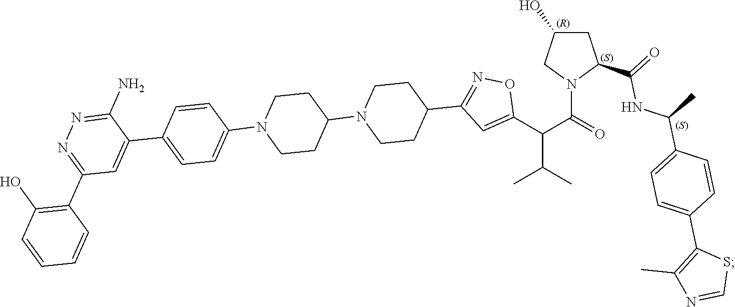 |

or a pharmaceutically acceptable salt or a stereoisomer or a tautomer thereof.

### Example-P1: Determination of Anti Proliferative Activity of Compounds in Cell Lines VCaP by Cell Titer Glo©(Promega) Assay

VCaP (ATCC CRL-2876) cells were seeded in 96 well plate flat black clear bottom plates (Corning, Cat. No 3904) using complete DMEM Medium. Next day, compounds listed in the present invention were added to cells from 10 mM stocks made in DMSO (Sigma Cat no. D2650). Each concentration of compound was tested in triplicate with DMSO concentration at a final percentage not exceeding 0.3 in the cells. After the incubation of VCaP cells with compound for 8 days assay was terminated using 50 μl of CellTiter Glo© reagent (Promega, Cat. no G7572). CellTiter-Glo® Luminescent reagent determines the number of viable cells based on quantitation of ATP present which is an indicator of cell number and metabolic activity. Luminescence readings were taken in Victor-3 instrument. Percent inhibition of proliferation was calculated using formula, % inhibition=100−(luminescence value of test/luminescence value of DMSO control)*100. DMSO control (0%)=Cells in complete media with DMSO; blank=Media alone containing DMSO. EC₅₀ was calculated using graph pad prism software.

Selected compounds of the present invention were screened in the above-mentioned assay procedures for determination of EC₅₀ (VCaP) values and the results are summarized into groups A, B and C in below table. Herein the group “A” refers to EC₅₀ values lower than l0 nM, group “B” refers to EC₅₀ values between l0 nM-100 nM (both inclusive) and group “C” refers to EC₅₀ values greater than 100 nM.

| Group | Compounds of the present invention |
|---|---|
| A | 12, 19, 24, 25, 29, 35, 36, 37, 40, |
| | 42, 43, 44, 45, 53 and 59. |
| B | 1, 6, 11, 28, 30, 31, 38, 41, |
| | 48, 51 and 58. |
| C | 21, 32, 34, 39, 50, 52 and 60 |
| | (20 % inhibition at 1 μM). |

### Example-P2: Determination of SMARCA2 and SMARCA4 Degradation in VCaP Cells by Western Blot

VCaP (ATCC CRL-2876) was plated in 6 well plates using complete Dulbecco's Modified Eagle's Medium. On the third day, compounds of present invention were added to cells from 10 mM stocks made in DMSO (Sigma Cat no. D2650). Each compound was tested at 100 nM concentration with DMSO not exceeding final percentage of 0.3 in the cells. Cells were incubated with the compound for 16 hours followed by harvesting with 1×RIPA lysis buffer (Thermo Fischer, catalogue number #89900) containing protease inhibitor cocktail (Sigma catalogue number #P-8340). Equal amount of protein was loaded on SDS PAGE gel for electrophoresis. Western blot was carried out for detection of either SMARCA2 (Cell signalling technologies, catalogue number #11966) or SMARCA4 antibody (Cell signalling technologies, catalogue number #52251). Beta-Tubulin antibody (Cell signalling technologies, catalogue number #86298) was used as loading control. Percentage of SMARCA2 or SMARCA4 degradation was calculated by using formula:

$$\% Degradation = 100 - ( normalized band intensity in treated sample / normalized band intensity in DMSO sample ) * 100.$$

The results are given below.

| | Percent degradation | |
|---|---|---|
| Com- | (at 100 nM) | |
| pound | SMARCA2 | SMARCA4 |
| 4 | 98 | 11 |
| 5 | 57 | 62 |
| 6 | 100 | 85 |
| 8 | 0 | 18 |
| 9 | 30 | 62 |
| 11 | 98 | 51 |
| 12 | 98 | 54 |
| 13 | 27 | 38 |
| 15 | 8 | 16 |
| 17 | 7 | 35 |
| 18 | 98 | 93 |
| 19 | 96 | 96 |
| 20 | 98 | 97 |
| 21 | 97 | 71 |
| 22 | 2 | 7 |
| 23 | 82 | 49 |
| 24 | 95 | 96 |
| 25 | 97 | 92 |
| 27 | 59 | 47 |
| 28 | 96 | 67 |
| 29 | 95 | 95 |
| 30 | 92 | 63 |
| 31 | 99 | 90 |
| 32 | 90 | 0 |
| 33 | 42 | 0 |
| 34 | 91 | 0 |
| 35 | 97 | 88 |
| 36 | 99 | 86 |
| 37 | 100 | 97 |
| 38 | 100 | 91 |
| 39 | 78 | 0 |
| 40 | 97 | 45 |
| 41 | 92 | 52 |
| 42 | 99 | 94 |
| 43 | 97 | 86 |
| 44 | 98 | 89 |
| 45 | 97 | 92 |
| 46 | 98 | 96 |
| 47 | 99 | 95 |
| 48 | 97 | 20 |
| 49 | 66 | 0 |
| 50 | 85 | 0 |
| 51 | 95 | 24 |
| 52 | 97 | 70 |
| 53 | 98 | 89 |
| 54 | 99 | 97 |
| 55 | 99 | 71 |
| 58 | 99 | 90 |

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents and the specification, along with such variations.

We claim:

1. A compound of formula (I): 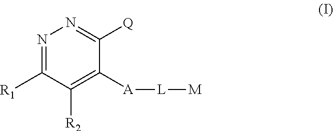
or a pharmaceutically acceptable salt or a stereoisomer or a tautomer thereof;
wherein,
A represents 5- to 6-membered heteroarylenyl or 6-membered arylenyl; wherein the arylenyl and heteroarylenyl are unsubstituted or substituted with 1, 2 or 3 Rₐ;
Rₐ, at each occurrence, independently represents hydroxy, hydroxyalkyl, halogen, alkoxy, alkyl, haloalkyl or cyano;
R₁ is halogen, alkyl, haloalkyl, alkoxy, hydroxy, hydroxyalkyl, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano;
R₂ is hydrogen, hydroxy, hydroxyalkyl, halogen, alkoxy, alkyl, amino, aminoalkyl, haloalkyl or cyano;
Q is amino, aminoalkyl, hydroxy, hydroxyalkyl, halogen, alkoxy, alkyl, haloalkyl or cyano;
L is a bond, —N(Rₓ)—(CRₓR_{y})p-O—, —O—(CRₓR_{y})p-, —O—(CRₓR_{y})p-O—, —C≡C—(CRₓR_{y})p-, -heterocycloalkylenyl-heterocycloalkylenyl-, -heterocycloalkylenyl-O—, -heterocycloalkylenyl-C≡C—, —C≡C-heterocycloalkylenyl-, —O-heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p₁-heterocycloalkylenyl- or -heterocycloalkylenyl-O-heterocycloalkylenyl-; wherein the cycloalkylenyl, heteroarylenyl and heterocycloalkylenyl are unsubstituted or substituted with 1, 2 or 3 R_{d}; and the left side of L group is attached to A and right side of L group is attached to M;
R_{d}, at each occurrence, is independently selected from hydroxy, halogen, alkyl and alkoxy; or any two R_{d} groups attached with the same C atom together form an oxo group;
Rₓ and R_{y}, at each occurrence, are independently selected from hydrogen and alkyl;
M is selected from M-1 and M-2: 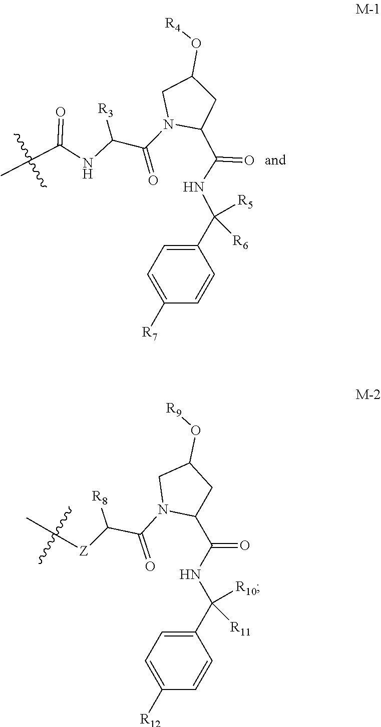
wherein
Z is 5- to 6-membered heteroarylenyl or 5- to 6-membered heterocycloalkylenyl; wherein the heteroarylenyl and heterocycloalkylenyl groups are unsubstituted or substituted with oxo, hydroxy, halogen, alkyl or alkoxy;
R₃ and R₈ independently represents alkyl, haloalkyl or hydroxyalkyl;
R₄ and R₉ independently represents hydrogen, alkyl, haloalkyl or hydroxyalkyl;
R₅, R₆, R₁₀ and R₁₁ are independently selected from hydrogen, alkyl, halogen, heteroalkyl, haloalkyl, hydroxyalkyl and acyl;
R₇ and R₁₂ independently represents thiazolyl which is unsubstituted or substituted with alkyl, hydroxy, amino or haloalkyl;
‘p’ is an integer selected from 0, 1, 2, 3, 4, 5 and 6; and
‘p₁’ is an integer selected from 1, 2, 3 and 4.

2. The compound of claim 1, wherein R₁ is halogen, alkyl, haloalkyl, alkoxy, 6- to 10-membered aryl or 5- to 10-membered heteroaryl; wherein the aryl and heteroaryl groups are unsubstituted or substituted with 1, 2 or 3 substituent(s) independently selected from oxo, hydroxy, alkoxy, halogen, alkyl, haloalkyl, amino and cyano.

3. The compound of any one of claims 1 to 2, wherein R₁ is methoxy, chloro, trifluoromethyl, phenyl or pyridinyl; wherein the phenyl and pyridinyl are unsubstituted or substituted with 1, 2 or 3 substituent(s) independently selected from oxo, hydroxy, methoxy, fluoro, chloro, difluoromethyl and trifluoromethyl.

4. The compound of any one of claims 1 to 3, wherein R₂ is hydrogen.

5. The compound of any one of claims 1 to 4, wherein A represents phenylenyl which is unsubstituted or substituted with 1 or 2 Rₐ.

6. The compound of any one of claims 1 to 5, wherein A represents 5- to 6-membered heteroarylenyl which is unsubstituted or substituted with 1 or 2 Rₐ.

7. The compound of any one of claims 1 to 6, wherein A represents phenylenyl, furanylenyl, thienylenyl, pyrrolylenyl, pyrazolylenyl, imidazolylenyl, oxazolylenyl, isoxazolylenyl, thiazolylenyl, isothiazolylenyl, 1H-tetrazolylenyl, oxadiazolylenyl, triazolylenyl, pyridylenyl, pyrimidinylenyl, pyrazinylenyl, pyridazinylenyl, 1,2,3-triazinylenyl, 1,2,4-triazinylenyl or 1,3,5-triazinylenyl; wherein each group is unsubstituted or substituted with 1 or 2 substituent(s) independently selected from hydroxy, hydroxyalkyl, halogen, alkoxy, alkyl, haloalkyl and cyano.

8. The compound of any one of claims 1 to 7, wherein Q represents amino or alkoxy.

9. The compound of any one of claims 1 to 8, wherein L is a bond.

10. The compound of any one of claims 1 to 9, wherein L is —N(Rₓ)—(CRₓR_{y})p-O—, —O—(CRₓR_{y})p-, —O—(CRₓR_{y})p-O— or —C≡C—(CRₓR_{y})p-.

11. The compound of claim 10, wherein L is —N(CH₃)—CH₂CH₂—O—, —N(CH₃)—CH₂CH₂CH₂-0-, —N(CH₃)—CH₂CH₂CH₂CH₂—O—, —NH—CH₂CH₂—O—, —NH—CH₂CH₂CH₂—O—, —O—CH₂—, —O—CH₂CH₂—, —O—CH₂CH₂CH₂—, —O—CH₂CH₂—O—, —O—CH₂CH₂CH₂CH₂—, —C≡C—CH₂—, —C≡C—CH₂CH₂— or —C≡C—CH₂CH₂CH₂—.

12. The compound of any one of claims 1 to 11, wherein L is -heterocycloalkylenyl-heterocycloalkylenyl-, -heterocycloalkylenyl-O—, -heterocycloalkylenyl-C≡C—, —C≡C-heterocycloalkylenyl-, —O-heterocycloalkylenyl-(CRₓR_{y})p-, —N(Rₓ)-heterocycloalkylenyl-(CRₓR_{y})p-, -cycloalkylenyl-(CRₓR_{y})p-, -heteroarylenyl-(CRₓR_{y})p-, -heterocycloalkylenyl-(CRₓR_{y})p-, -heterocycloalkylenyl-(CRₓR_{y})p₁-heterocycloalkylenyl- or -heterocycloalkylenyl-O-heterocycloalkylenyl-.

13. The compound of claim 12, wherein the heterocycloalkylenyl is azetidinylenyl, pyrrolidinylenyl, piperidinylenyl, piperazinylenyl, tetrahydropyranyl, tetrahydropyridazinylenyl, morpholinylenyl, thiomorpholinylenyl, 1,4-dioxanylenyl, dioxidothiomorpholinylenyl, oxapiperazinylenyl, oxapiperidinylenyl, tetrahydropyranylenyl, dihydropyranylenyl or dihydropyrimidinylenyl; wherein each group is unsubstituted or substituted with 1 or 2 R_{d}.

14. The compound of claim 12, wherein the cycloalkylenyl is cyclopropylenyl, cyclobutylenyl, cyclopentylenyl, cyclohexylenyl or cycloheptylenyl.

15. The compound of any one of claims 9 to 14, wherein L is —N(CH₃)—CH₂CH₂—O—, —N(CH₃)—CH₂CH₂CH₂—O—, —N(CH₃)—CH₂CH₂CH₂CH₂—O—, —NH—CH₂CH₂—O—, —NH—CH₂CH₂CH₂—O—, —O—CH₂—, —O—CH₂CH₂—, —O—CH₂CH₂CH₂—, —O—CH₂CH₂CH₂CH₂—, —O—CH₂CH₂—O—, —C≡C—CH₂—, —C≡C—CH₂CH₂—, —C≡C—CH₂CH₂CH₂—, 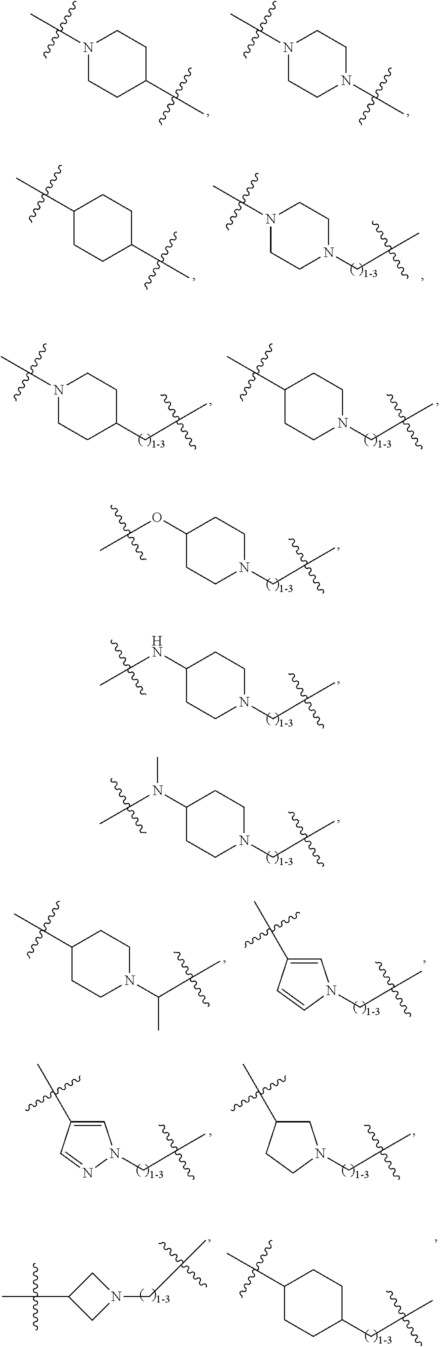 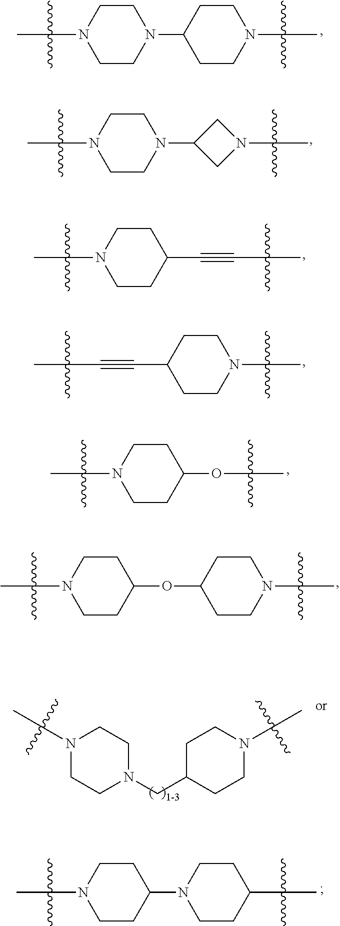
wherein each ring is unsubstituted or substituted with 1 or 2 R_{d}.

16. The compound of claim 1, wherein M is represented by the formula M-1; 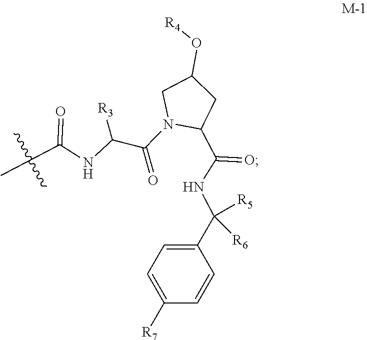
wherein
R₃ represents alkyl, haloalkyl or hydroxyalkyl;
R₄ represents hydrogen, alkyl, haloalkyl or hydroxyalkyl;
R₅ and R₆ are independently selected from hydrogen, alkyl, halogen, haloalkyl and hydroxyalkyl; and
R₇ represents thiazolyl which is unsubstituted or substituted with alkyl or haloalkyl.

17. The compound of claim 16, wherein M-1 is represented by formula M-1A or M-1B:

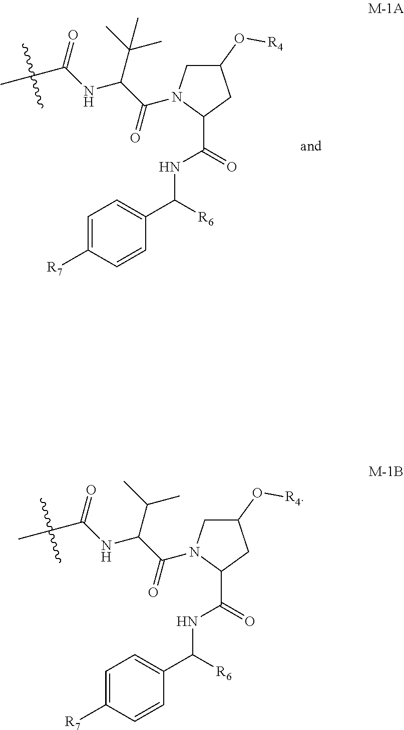

18. The compound of anyone claims 16 to 17, wherein M-1 is selected from: 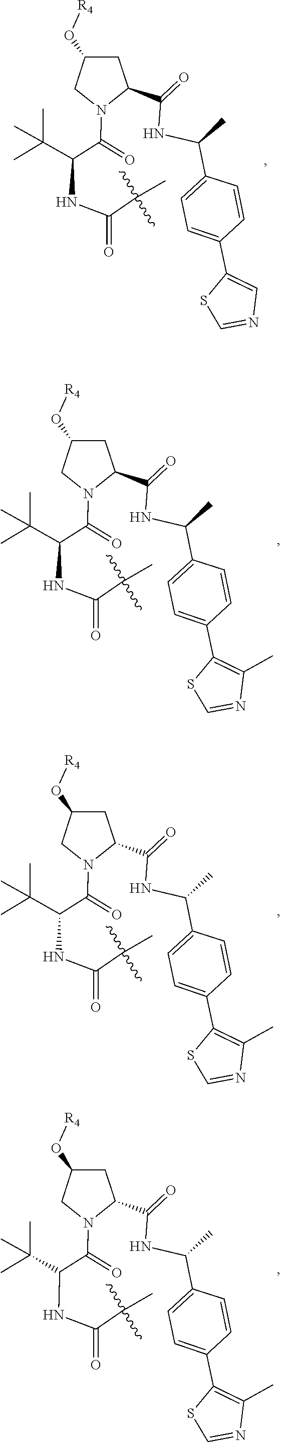 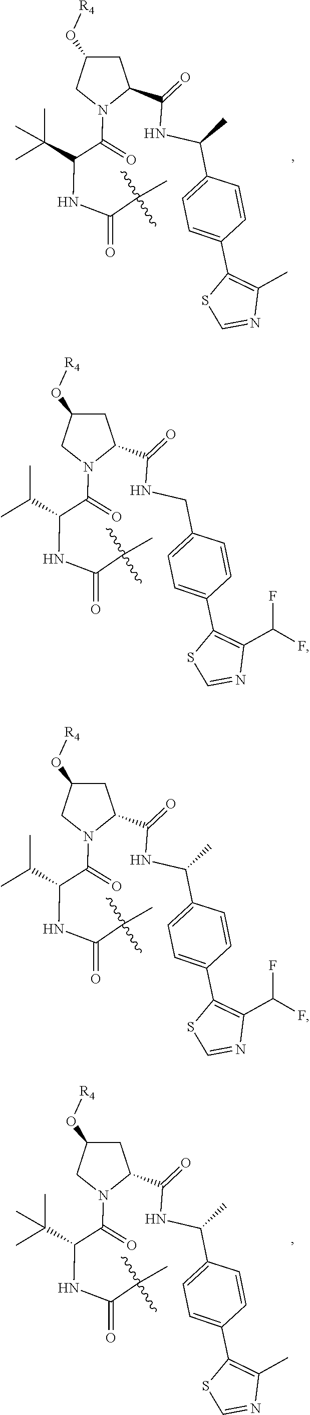 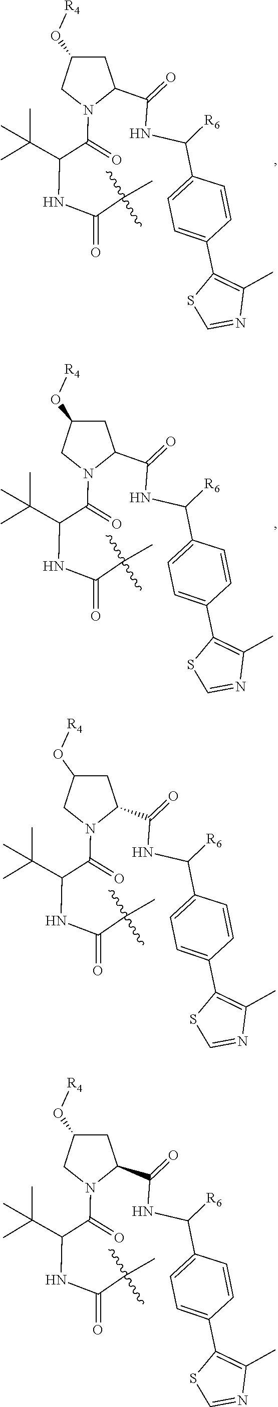 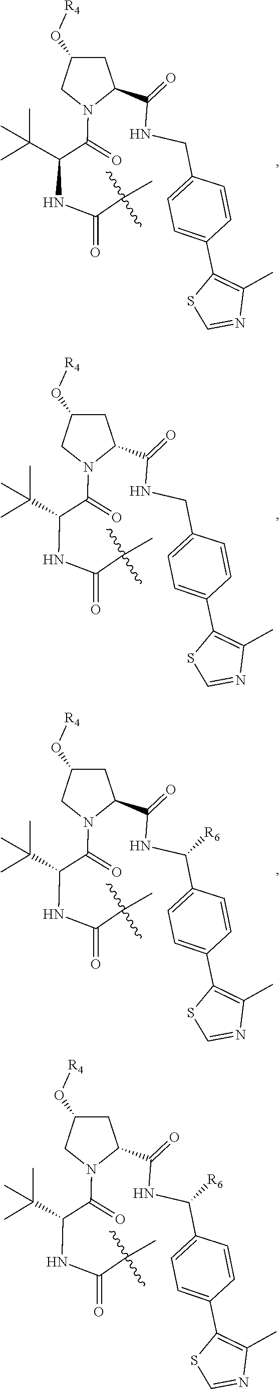 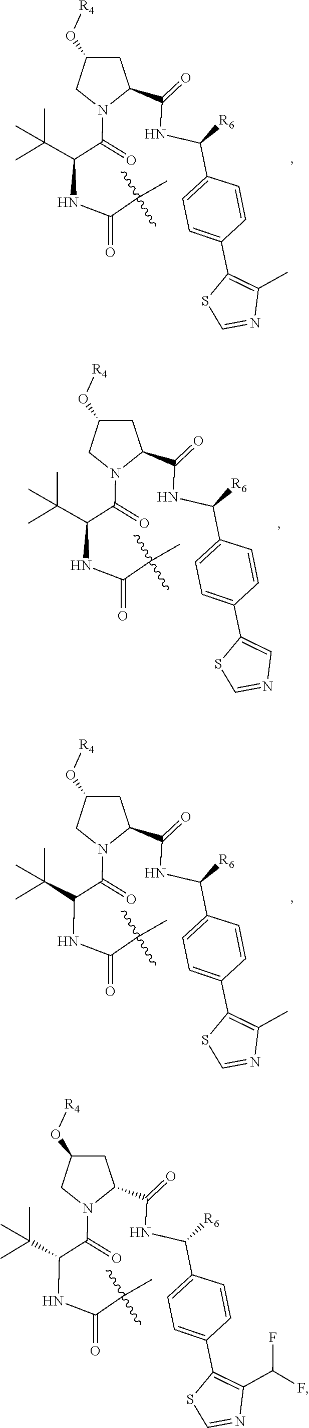 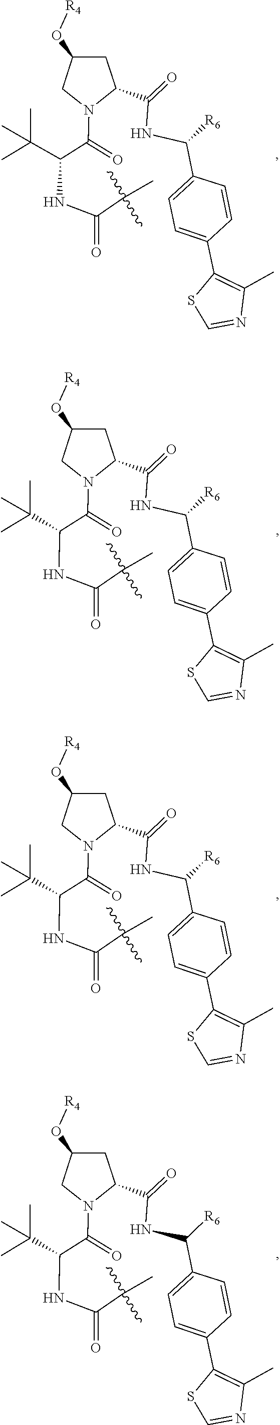 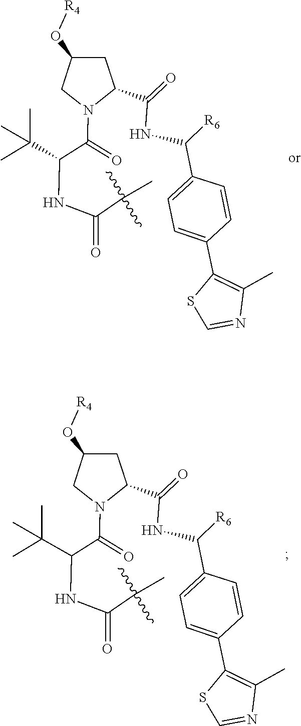
wherein, R₄ represents hydrogen; and R₆ represents alkyl.

19. The compound of claim 1, wherein M is reperesented by formula M-2; 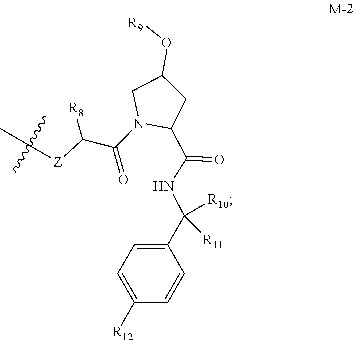
wherein,
Z is oxazolylenyl, pyrazolylenyl, isoxazolylenyl or piperidinylenyl;
R₈ represents alkyl, heteroalkyl, haloalkyl or hydroxyalkyl;
R₉ represents hydrogen, alkyl, haloalkyl or hydroxyalkyl;
R₁₀ and R₁₁ are independently selected from hydrogen, alkyl, halogen, heteroalkyl, haloalkyl and hydroxyalkyl; and
R₁₂ represents thiazolyl which is unsubstituted or substituted with alkyl or haloalkyl.

20. The compound of claim 19, wherein M-2 is represented by formula M-2A or M-2B:

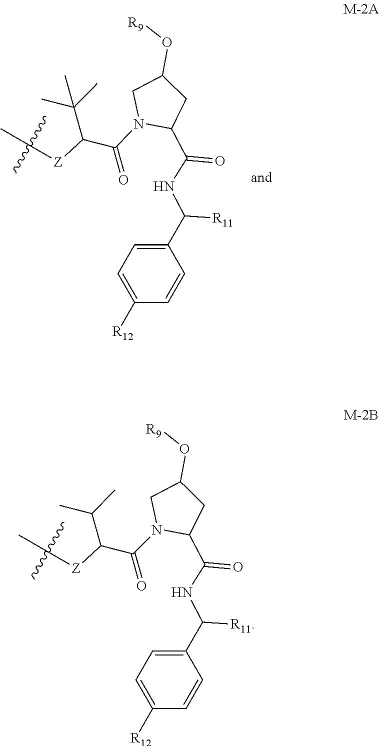

21. The compound of any one of claims 19-20, wherein M-2 is represented by formula: 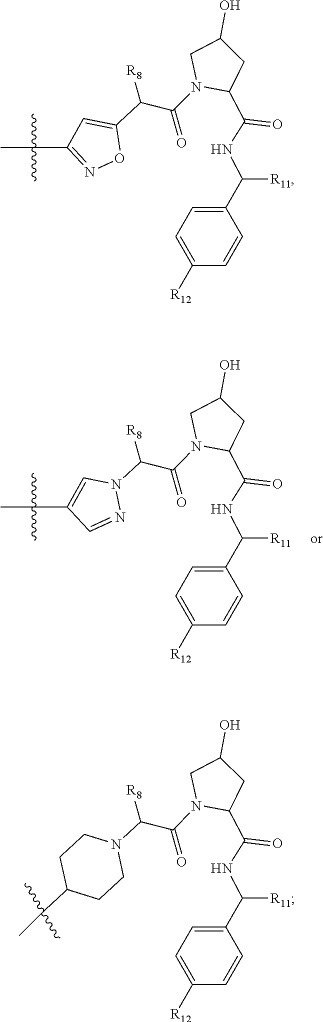
wherein, R₈ represents alkyl; R₁₁ represents hydrogen or alkyl; and
R₁₂ represents thiazolyl which is unsubstituted or substituted with alkyl or haloalkyl.

22. The compound of any one of claims 19-21, wherein M-2 is

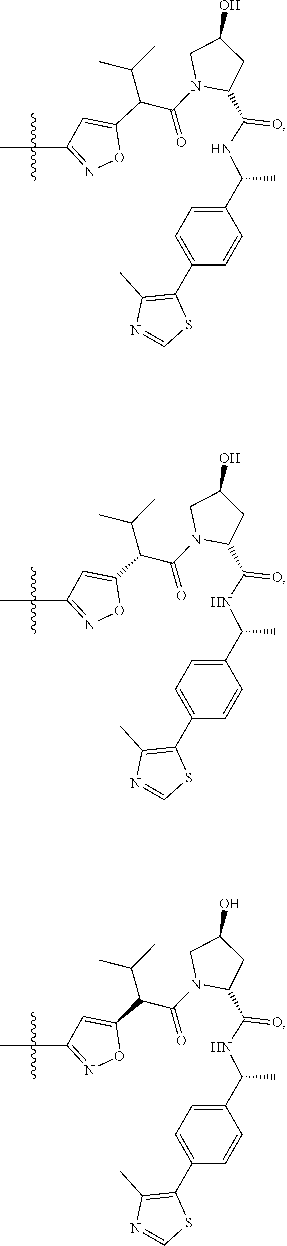

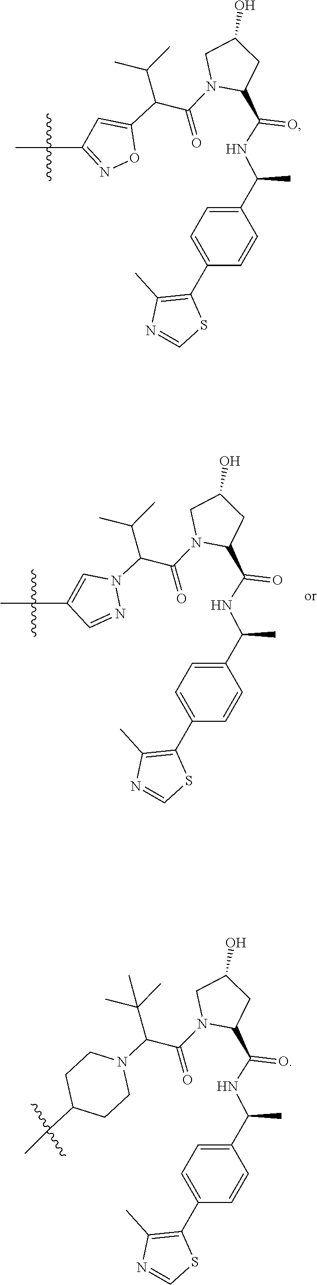

23. The compound of claim 1, represented by compound of formula (IA):

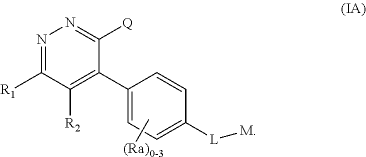

24. The compound of claim 23, wherein Q is selected from amino and alkoxy.

25. The compound of claim 23 or 24, wherein R₁ is halogen, alkyl, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen and haloalkyl.

26. The compound of any one of claim 23 to 25, wherein R₂ is hydrogen.

27. The compound of any one of claim 23 to 26, wherein Rₐ, at each occurrence, independently represents halogen.

28. The compound of any one of claims 23 to 27, wherein
L is a bond, —N(CH₃)—CH₂CH₂—O—, —NH—CH₂CH₂—O—, —O—CH₂—, —O—CH₂—CH₂—O—, —O—CH₂CH₂CH₂CH₂—, —C≡C—CH₂—, —C≡C—CH₂CH₂—, —C≡C—CH₂CH₂CH₂—, 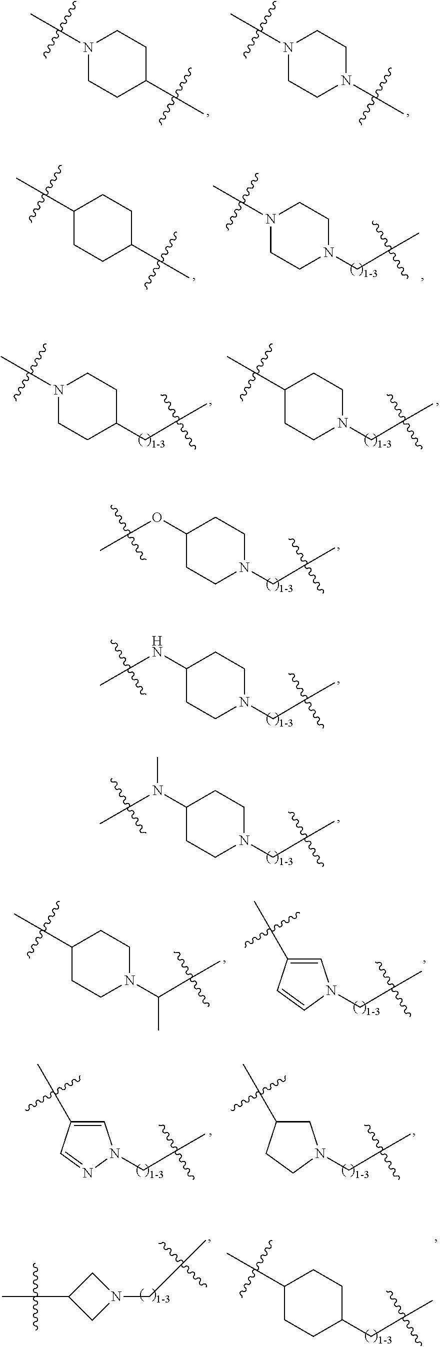 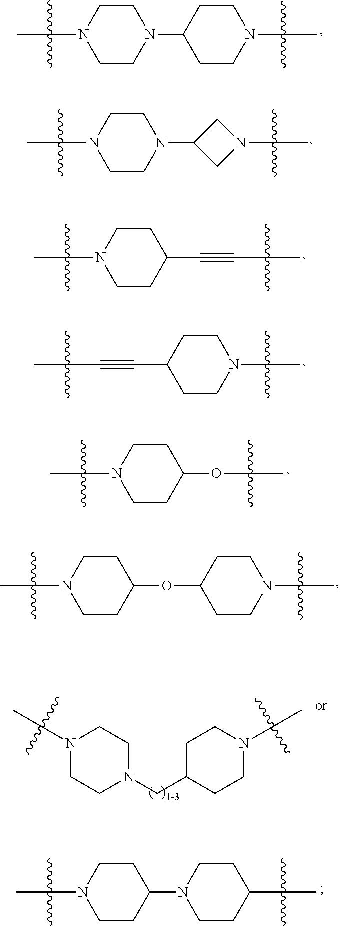
wherein each ring is unsubstituted or substituted with 1 or 2 R_{d}.

29. The compound of any one of claims 23 to 28, wherein
M is selected from M-1 and M-2;
Z is oxazolylenyl, pyrazolylenyl, isoxazolylenyl or piperidinylenyl;
R₃ and R₈ independently represents alkyl;
R₄ and R₉ independently represents hydrogen or alkyl;
R₅, R₆, R₁₀ and R₁₁ are independently selected from hydrogen and alkyl; and
R₇ and R₁₂ independently represents thiazolyl which is unsubstituted or substituted with alkyl or haloalkyl.

30. The compound of any one of claims 23 to 29, wherein
Rₐ, at each occurrence, independently represents halogen;
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from oxo, hydroxy, alkoxy, halogen and haloalkyl;
R₂ is hydrogen;
Q is amino or alkoxy;
L is a bond, —O—CH₂—, —O—CH₂CH₂CH₂CH₂—, —O—CH₂CH₂—O—, —NH—CH₂CH₂—O—, —N(CH₃)—CH₂CH₂—O—, —C≡C—CH₂—, —C≡C—CH₂CH₂—, —C≡C—CH₂CH₂CH₂—, 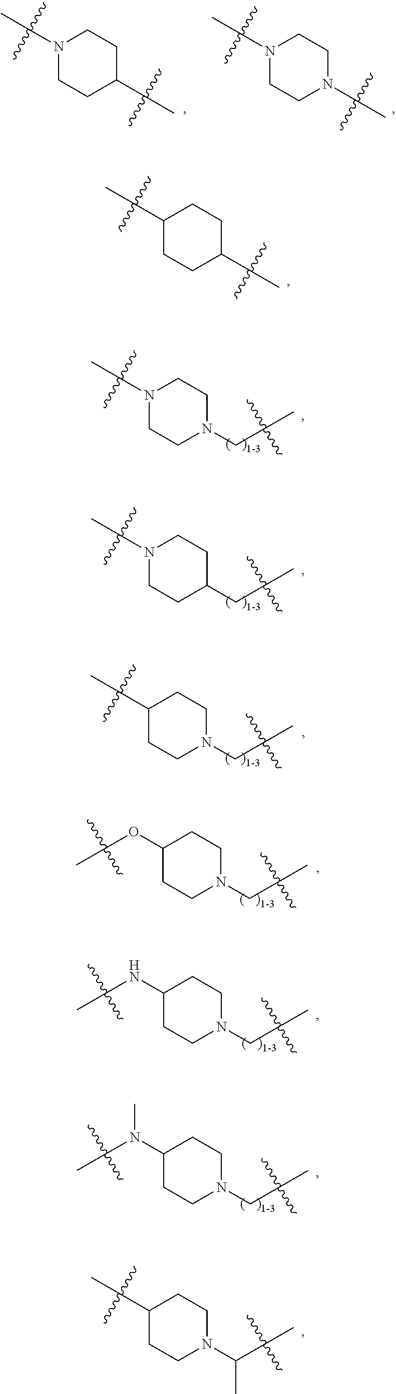 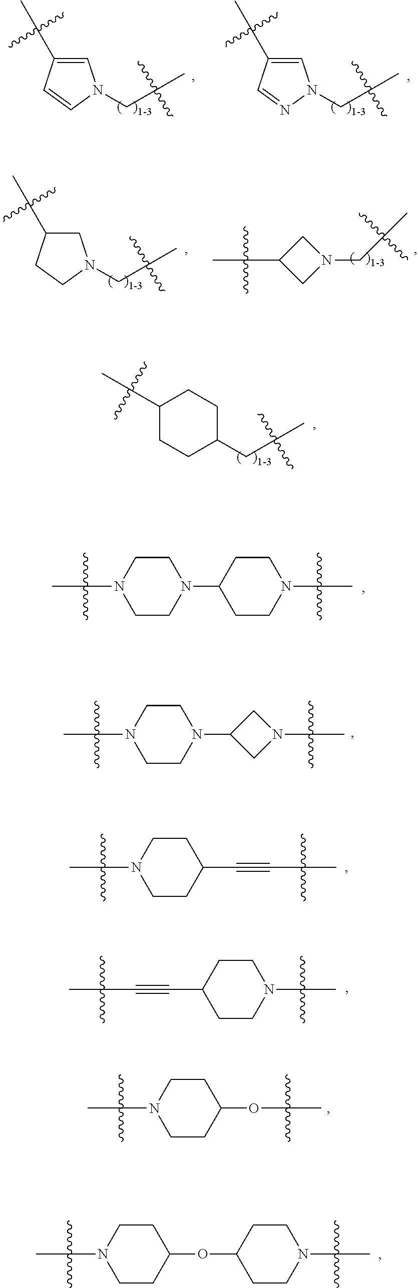 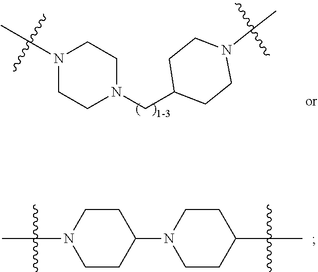
wherein each ring is unsubstituted or substituted with 1 or 2 R_{d};
R_{d}, at each occurrence, is independently selected from alkyl, hydroxy and halogen; or
any two R_{d} groups attached with the same C atom together form an oxo group;
M is selected from M-1 and M-2;
Z is oxazolylenyl, pyrazolylenyl, isoxazolylenyl or piperidinylenyl;
R₃ and R₈ independently represents alkyl;
R₄ and R₉ independently represents hydrogen;
R₅, R₆, R₁₀ and R₁₁ are independently selected from hydrogen and alkyl; and
R₇ and R₁₂ independently represents thiazolyl which is unsubstituted or substituted with alkyl or haloalkyl.

31. The compound of claim 1, represented by compound of formula (IB):

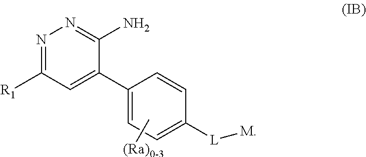

32. The compound of claim 31, wherein Rₐ, at each occurrence, independently represents halogen.

33. The compound of any one of claims 31 to 32, wherein
L is a bond, —O—CH₂—, —O—CH₂CH₂CH₂CH₂—, —O—CH₂CH₂—O—, —NH—CH₂CH₂—O—, —N(CH₃)—CH₂CH₂—O—, —C≡C—CH₂—, —C≡C—CH₂CH₂—, —C≡C—CH₂CH₂CH₂—, 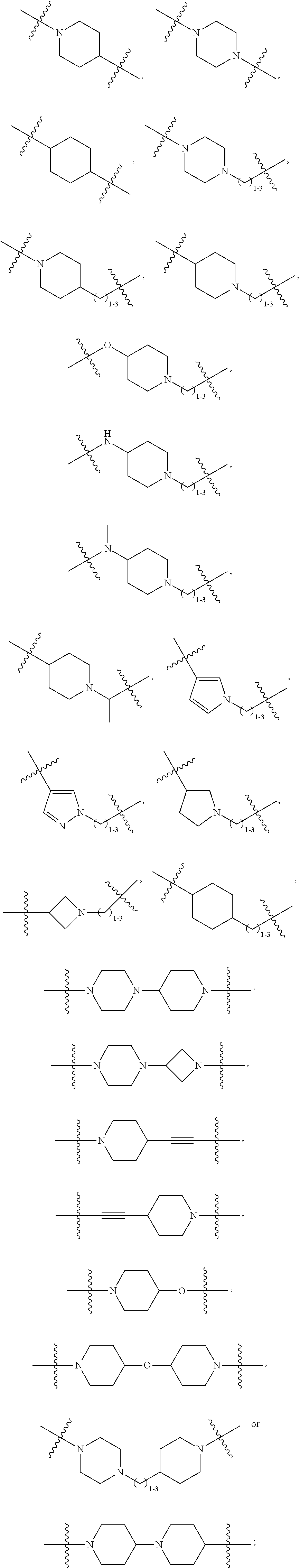
wherein each ring is unsubstituted or substituted with 1 or 2 R_{d}.

34. The compound of any one of claim 31 to 33, wherein M is selected from M-1 and M-2; wherein
Z is oxazolylenyl, pyrazolylenyl, isoxazolylenyl or piperidinylenyl;
R₃ and R₈ independently represents alkyl;
R₄ and R₉ independently represents hydrogen or alkyl;
R₅, R₆, R₁₀ and R₁₁ are independently selected from hydrogen and alkyl; and
R₇ and R₁₂ independently represents thiazolyl which is unsubstituted or substituted with alkyl or haloalkyl.

35. The compound of any one of claims 31 to 34, wherein
Rₐ at each occurrence, independently represents halogen;
R₁ is halogen, haloalkyl, alkoxy, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
L is a bond, —O—CH₂—, —O—CH₂CH₂CH₂CH₂—, —O—CH₂CH₂—O—, —NH—CH₂CH₂—O—, —N(CH₃)—CH₂CH₂—O—, —C≡C—CH₂—, —C≡C—CH₂CH₂—, —C≡C—CH₂CH₂CH₂—, 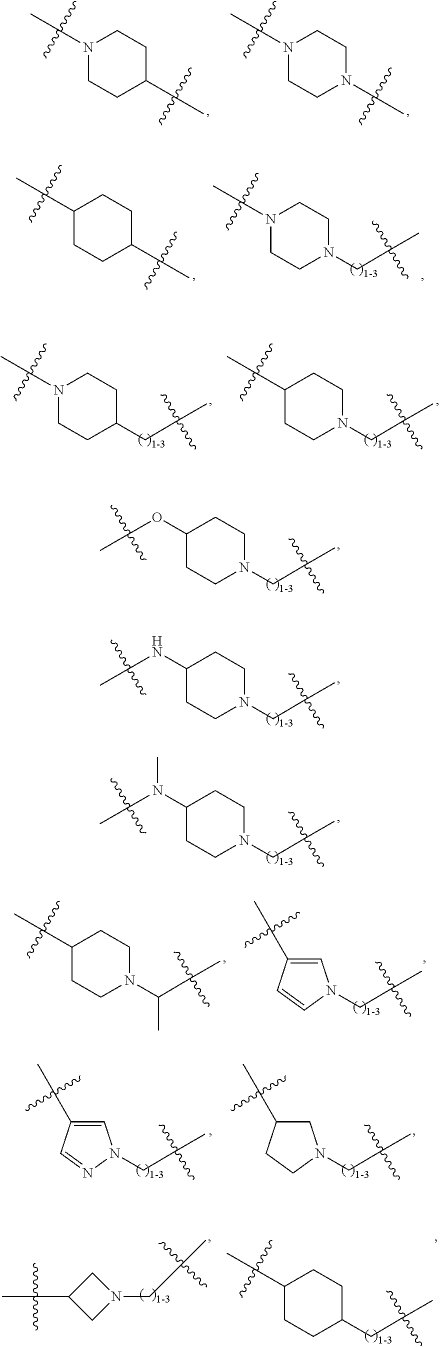 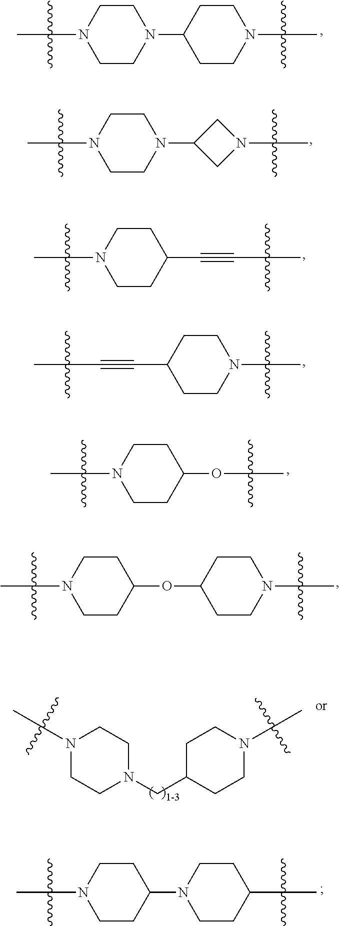
wherein each ring is unsubstituted or substituted with 1 or 2 R_{d};
R_{d}, at each occurrence, is independently selected from alkyl, hydroxy and halogen; or
any two R_{d} groups attached with the same C atom together form an oxo group;
M is selected from M-1 and M-2;
Z is oxazolylenyl, pyrazolylenyl, isoxazolylenyl or piperidinylenyl;
R₃ and R₈ independently represents alkyl;
R₄ and R₉ independently represents hydrogen;
R₅, R₆, R₁₀ and R₁₁ are independently selected from hydrogen and alkyl; and
R₇ and R₁₂ independently represents thiazolyl which is unsubstituted or substituted with alkyl or haloalkyl.

36. The compound of claim 1, represented by compound of formula (IC):

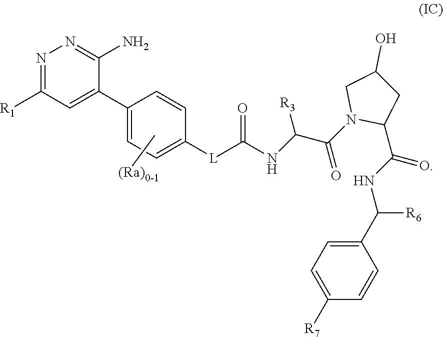

37. The compound of claim 36, wherein Rₐ represents halogen.

38. The compound of claim 36 or 37, wherein L is a bond, —O—CH₂—, —O—CH₂CH₂CH₂CH₂—, —O—CH₂CH₂—O—, —NH—CH₂CH₂—O—, —N(CH₃)—CH₂CH₂—O—, —C≡C—CH₂—, —C≡C—CH₂CH₂—, —C≡C—CH₂CH₂CH₂—, 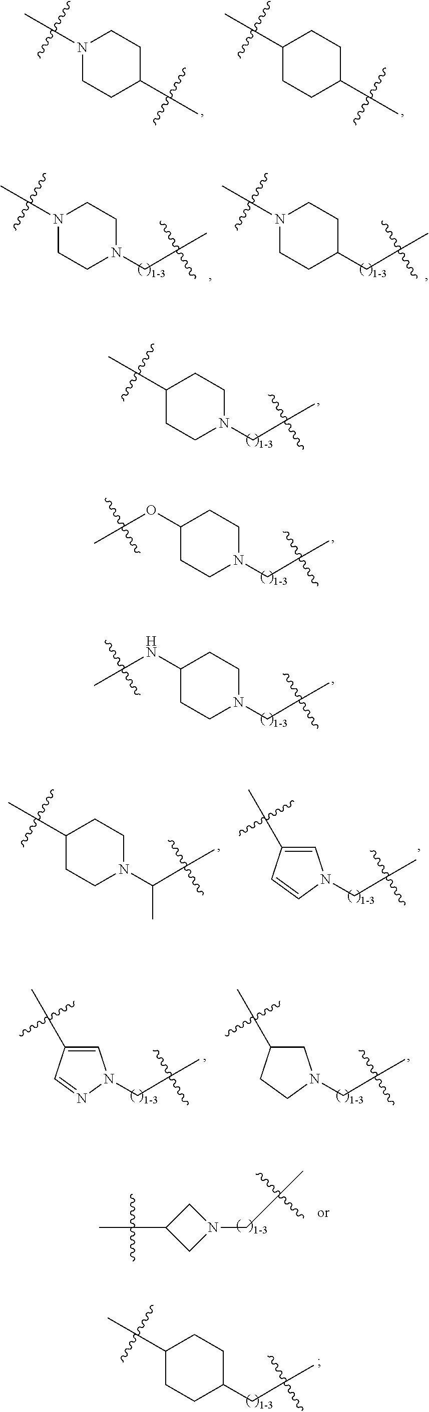
wherein each ring is unsubstituted or substituted with 1 or 2 R_{d}.

39. The compound of claim 36, wherein
R₃ represents alkyl;
R₆ is selected from hydrogen and alkyl; and
R₇ represents thiazolyl which is unsubstituted or substituted with alkyl or haloalkyl.

40. The compound of any one of claims 36 to 39, wherein
R₁ is halogen, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
Rₐ represents alkyl;
L is a bond, —O—CH₂—, —O—CH₂CH₂CH₂CH₂—, —O—CH₂CH₂—O—, —NH—CH₂CH₂—O—, —N(CH₃)—CH₂CH₂—O—, —C≡C—CH₂—, —C≡C—CH₂CH₂—, —C≡C—CH₂CH₂CH₂—, 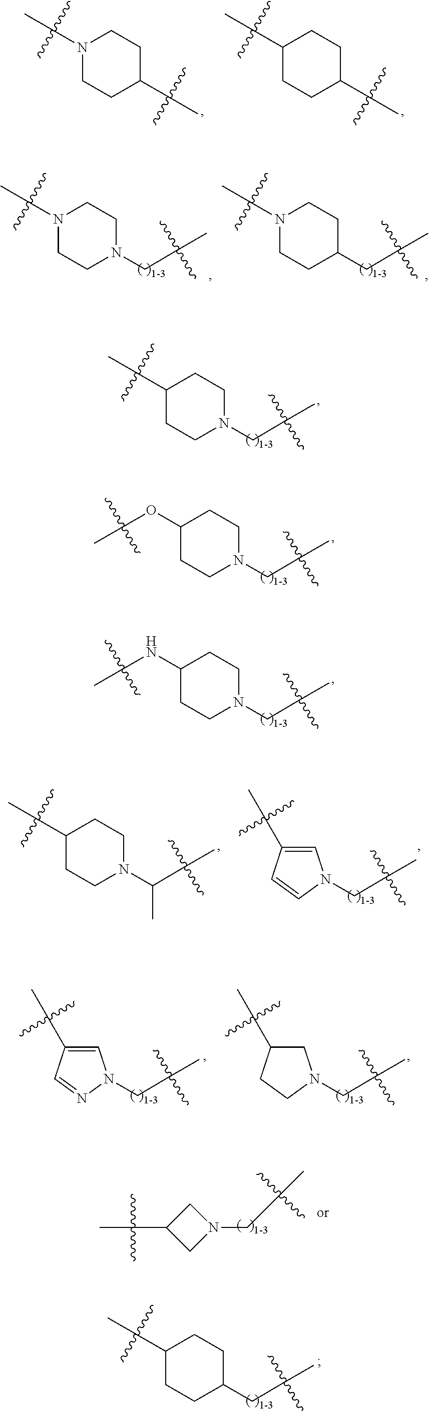
wherein each ring is unsubstituted or substituted with 1 or 2 R_{d};
R_{d}, at each occurrence, selected from alkyl, hydroxy and halogen; or any two R_{d} groups attached with the same C atom together form an oxo group;
R₃ represents alkyl;
R₆ is selected from hydrogen and alkyl; and
R₇ represents thiazolyl which is unsubstituted or substituted with alkyl or haloalkyl.

41. The compound of claim 1, represented by compound of formula (ID):

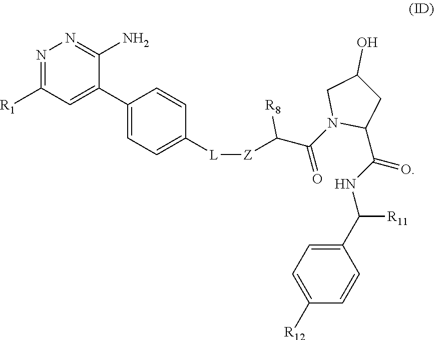

42. The compound of claim 41, wherein
L is a bond, —O—CH₂CH₂—O—, —NH—CH₂CH₂—O—, —N(CH₃)—CH₂CH₂—O—, 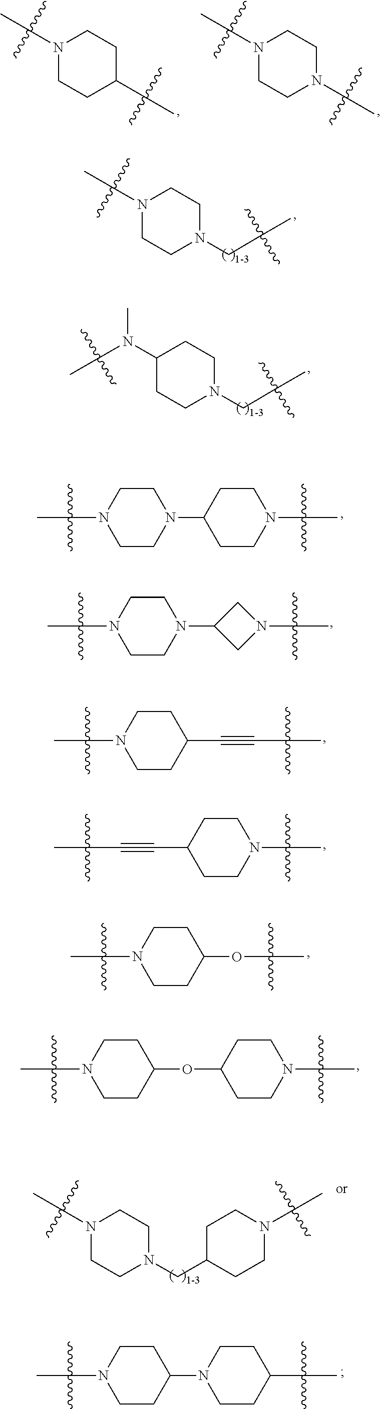
wherein each ring is unsubstituted or substituted with 1 or 2 R_{d};
R_{d}, at each occurrence, is independently selected from alkyl, hydroxy and halogen; or
any two R_{d} groups attached with the same C atom together form an oxo group.

43. The compound of claim 41 or 42, wherein
Z is oxazolylenyl, pyrazolylenyl, isoxazolylenyl or piperidinylenyl;
R₈ represents alkyl;
R₁₁ is selected from hydrogen and alkyl; and
R₁₂ represents thiazolyl which is unsubstituted or substituted with alkyl or haloalkyl.

44. The compound of any one of claims 41 to 43, wherein
R₁ is halogen, 6- to 10-membered aryl or 5- to 10-membered heteroaryl; wherein the aryl and heteroaryl groups are unsubstituted or substituted with 1 or 2 substituent(s) independently selected from hydroxy, alkoxy, halogen and haloalkyl;
L is a bond, —O—CH₂CH₂—O—, —NH—CH₂CH₂—O—, —N(CH₃)—CH₂CH₂—O—, 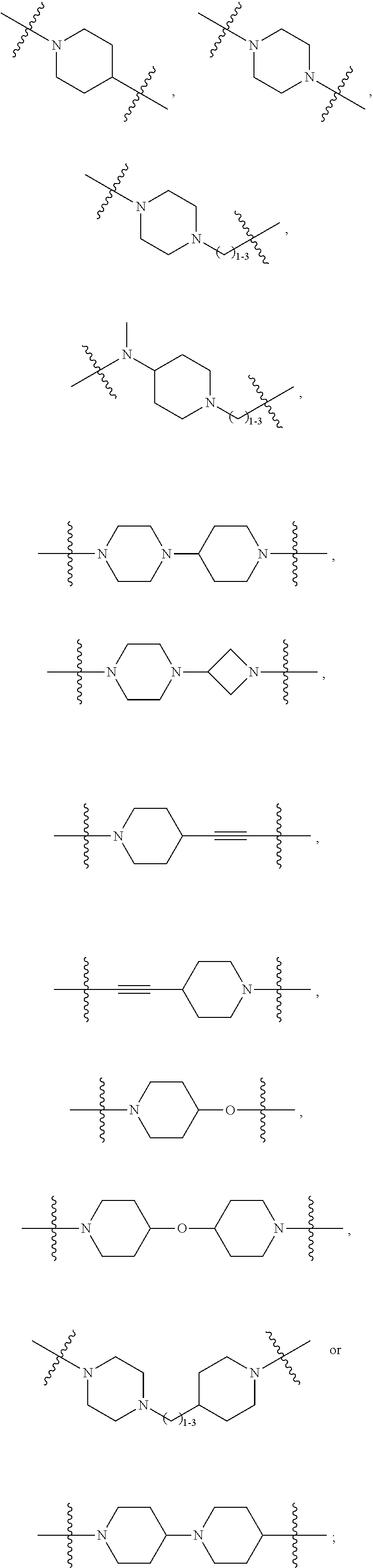
wherein each ring is unsubstituted or substituted with 1 or 2 R_{d};
R_{d}, at each occurrence, is independently selected from alkyl, hydroxy and halogen; or
any two R_{d} groups attached with the same C atom together form an oxo group;
Z is oxazolylenyl, pyrazolylenyl, isoxazolylenyl or piperidinylenyl;
R₈ represents alkyl;
R₁₁ is selected from hydrogen and alkyl; and
R₁₂ represents thiazolyl which is unsubstituted or substituted with alkyl or haloalkyl.

45. The compound of any one of claims 1 to 44, is selected from:

| Compound | Structure |
|---|---|
| 1 | 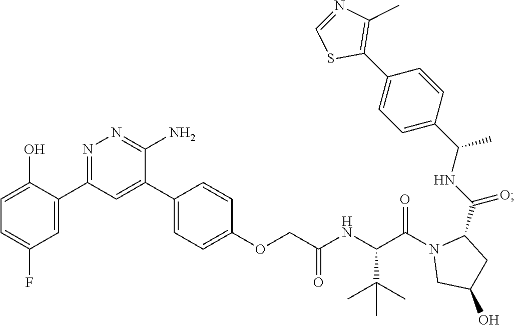 [SMILES: Cc1ncsc1-c1ccc([C@H](C)NC(=O)[C@@H]2C[C@@H](O)CN2C(=O)[C@@H](NC(=O)COc2ccc(-c3cc(-c4cc(F)ccc4O)nnc3N)cc2)C(C)(C)C)cc1] |
| 2 | 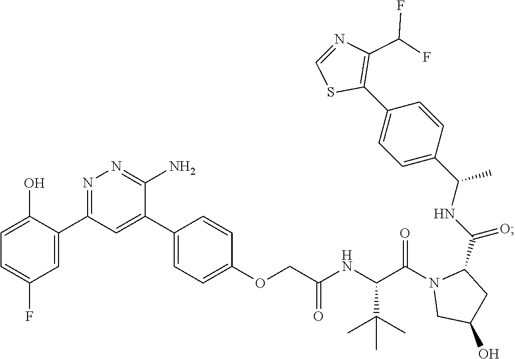 [SMILES: C[C@H](NC(=O)[C@@H]1C[C@@H](O)CN1C(=O)[C@@H](NC(=O)COc1ccc(-c2cc(-c3cc(F)ccc3O)nnc2N)cc1)C(C)(C)C)c1ccc(-c2scnc2C(F)F)cc1] |
| 3 | 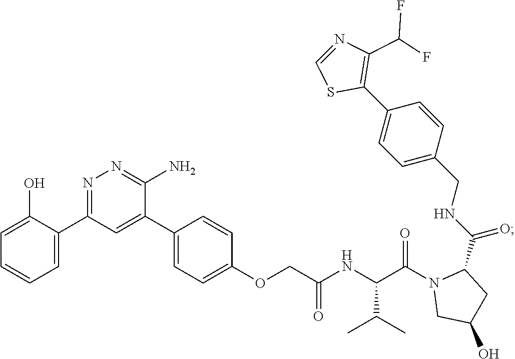 [SMILES: CC(C)[C@H](NC(=O)COc1ccc(-c2cc(-c3ccccc3O)nnc2N)cc1)C(=O)N1C[C@H](O)C[C@H]1C(=O)NCc1ccc(-c2scnc2C(F)F)cc1] |
| 4 | 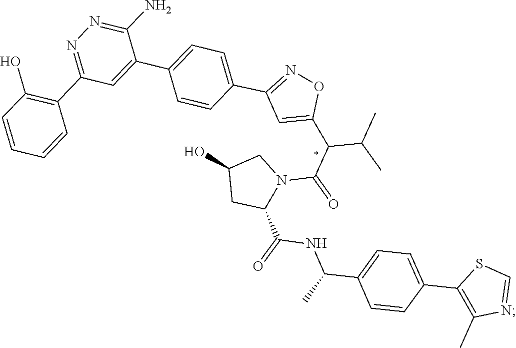 [SMILES: Cc1ncsc1-c1ccc([C@H](C)NC(=O)[C@@H]2C[C@@H](O)CN2C(=O)C(c2cc(-c3ccc(-c4cc(-c5ccccc5O)nnc4N)cc3)no2)C(C)C)cc1] |
| 5 | Isomer-1 of compound 4; |
| 6 | Isomer-2 of compound 4; |
| 7 | 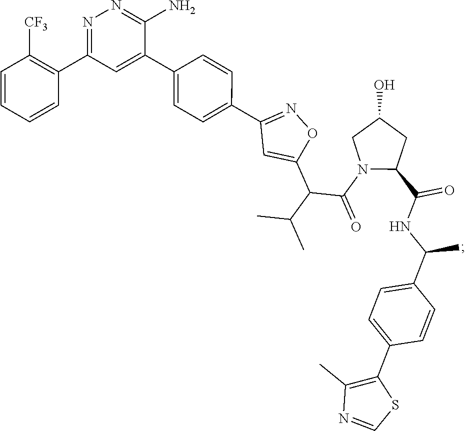 [SMILES: Cc1ncsc1-c1ccc([C@H](C)NC(=O)[C@@H]2C[C@@H](O)CN2C(=O)C(c2cc(-c3ccc(-c4cc(-c5ccccc5C(F)(F)F)nnc4N)cc3)no2)C(C)C)cc1] |
| 8 | 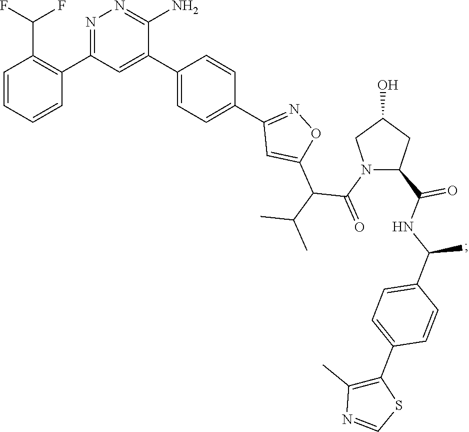 [SMILES: Cc1ncsc1-c1ccc([C@H](C)NC(=O)[C@@H]2C[C@@H](O)CN2C(=O)C(c2cc(-c3ccc(-c4cc(-c5ccccc5C(F)F)nnc4N)cc3)no2)C(C)C)cc1] |
| 9 | 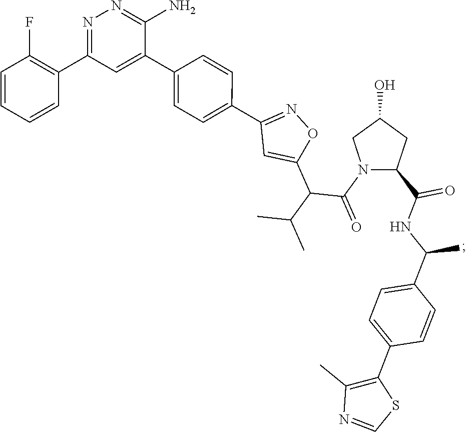 [SMILES: Cc1ncsc1-c1ccc([C@H](C)NC(=O)[C@@H]2C[C@@H](O)CN2C(=O)C(c2cc(-c3ccc(-c4cc(-c5ccccc5F)nnc4N)cc3)no2)C(C)C)cc1] |
| 10 | 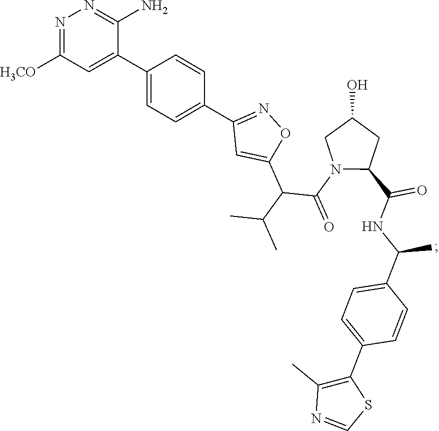 [SMILES: COc1cc(-c2ccc(-c3cc(C(C(=O)N4C[C@H](O)C[C@H]4C(=O)N[C@@H](C)c4ccc(-c5scnc5C)cc4)C(C)C)on3)cc2)c(N)nn1] |
| 11 | 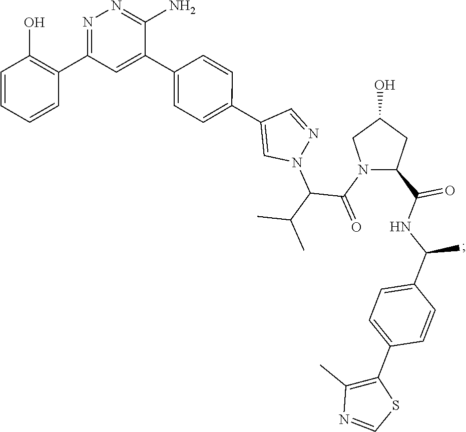 [SMILES: Cc1ncsc1-c1ccc([C@H](C)NC(=O)[C@@H]2C[C@@H](O)CN2C(=O)C(C(C)C)n2cc(-c3ccc(-c4cc(-c5ccccc5O)nnc4N)cc3)cn2)cc1] |
| 12 | 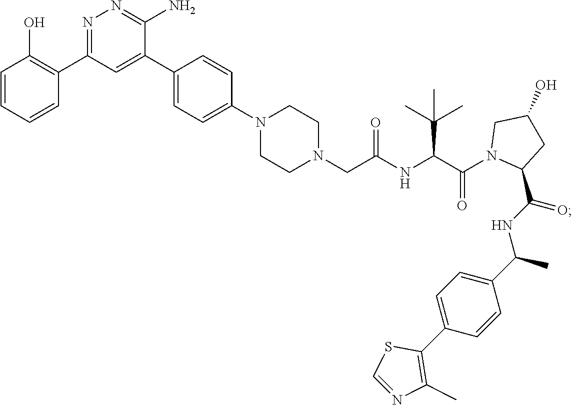 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)cc3)CC2)C(C)(C)C)cc1] |
| 13 | 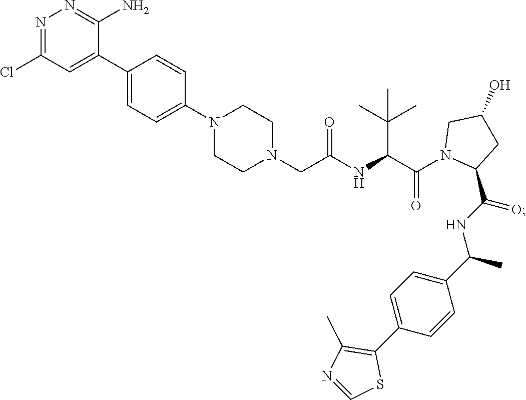 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(Cl)nnc4N)cc3)CC2)C(C)(C)C)cc1] |
| 14 | 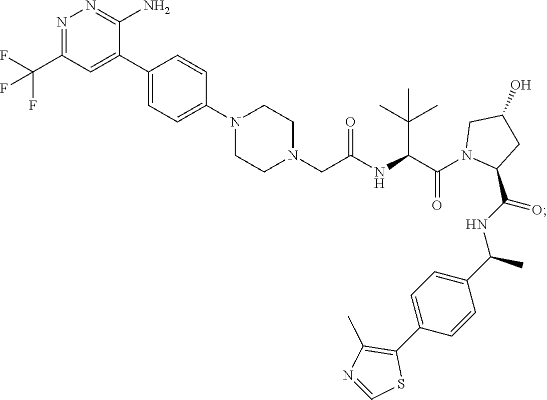 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(C(F)(F)F)nnc4N)cc3)CC2)C(C)(C)C)cc1] |
| 15 | 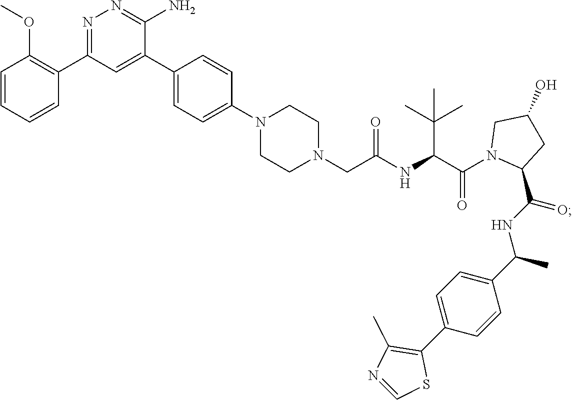 [SMILES: COc1ccccc1-c1cc(-c2ccc(N3CCN(CC(=O)N[C@H](C(=O)N4C[C@H](O)C[C@H]4C(=O)N[C@@H](C)c4ccc(-c5scnc5C)cc4)C(C)(C)C)CC3)cc2)c(N)nn1] |
| 16 | 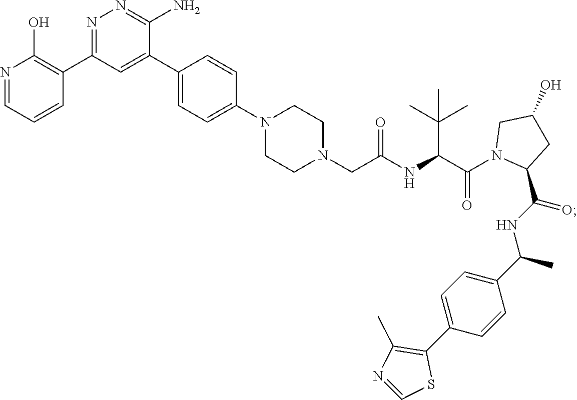 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5cccnc5O)nnc4N)cc3)CC2)C(C)(C)C)cc1] |
| 17 | 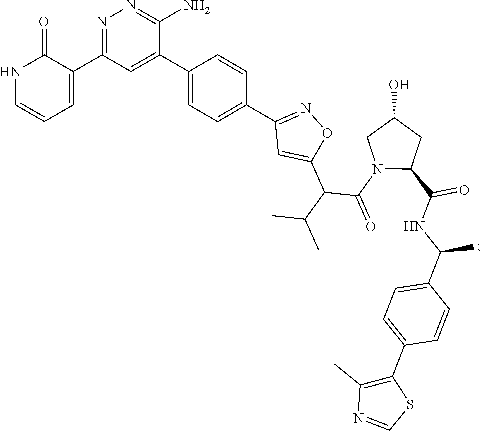 [SMILES: Cc1ncsc1-c1ccc([C@H](C)NC(=O)[C@@H]2C[C@@H](O)CN2C(=O)C(c2cc(-c3ccc(-c4cc(-c5ccc[nH]c5=O)nnc4N)cc3)no2)C(C)C)cc1] |
| 18 | 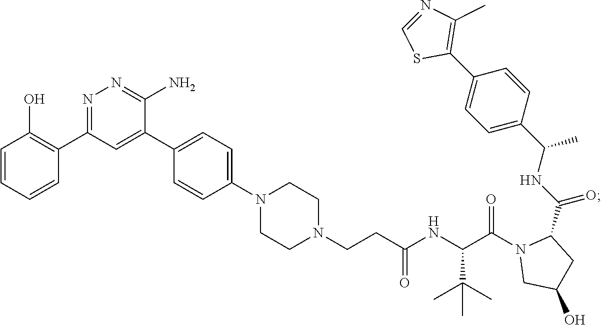 [SMILES: Cc1ncsc1-c1ccc([C@H](C)NC(=O)[C@@H]2C[C@@H](O)CN2C(=O)[C@@H](NC(=O)CCN2CCN(c3ccc(-c4cc(-c5ccccc5O)nnc4N)cc3)CC2)C(C)(C)C)cc1] |
| 19 | 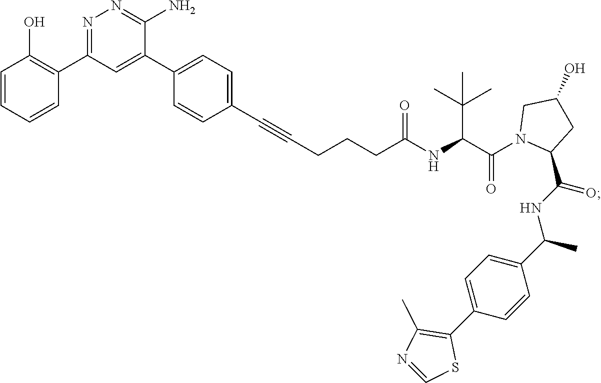 [SMILES: Cc1ncsc1-c1ccc([C@H](C)NC(=O)[C@@H]2C[C@@H](O)CN2C(=O)[C@@H](NC(=O)CCCC#Cc2ccc(-c3cc(-c4ccccc4O)nnc3N)cc2)C(C)(C)C)cc1] |
| 20 | 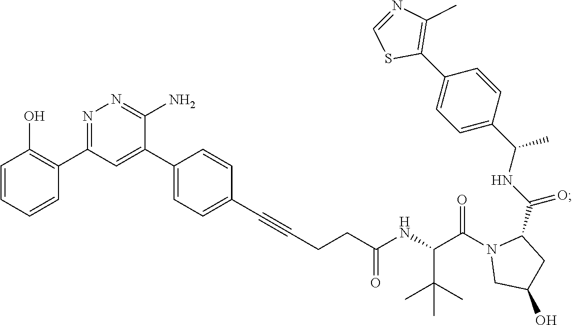 [SMILES: Cc1ncsc1-c1ccc([C@H](C)NC(=O)[C@@H]2C[C@@H](O)CN2C(=O)[C@@H](NC(=O)CCC#Cc2ccc(-c3cc(-c4ccccc4O)nnc3N)cc2)C(C)(C)C)cc1] |
| 21 | 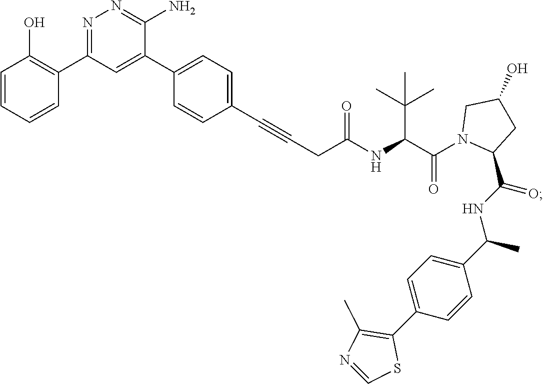 [SMILES: Cc1ncsc1-c1ccc([C@H](C)NC(=O)[C@@H]2C[C@@H](O)CN2C(=O)[C@@H](NC(=O)CC#Cc2ccc(-c3cc(-c4ccccc4O)nnc3N)cc2)C(C)(C)C)cc1] |
| 22 | 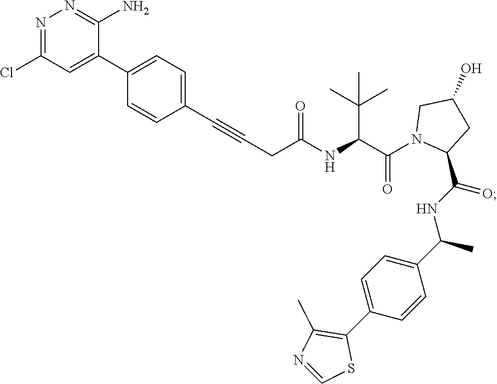 [SMILES: Cc1ncsc1-c1ccc([C@H](C)NC(=O)[C@@H]2C[C@@H](O)CN2C(=O)[C@@H](NC(=O)CC#Cc2ccc(-c3cc(Cl)nnc3N)cc2)C(C)(C)C)cc1] |
| 23 | 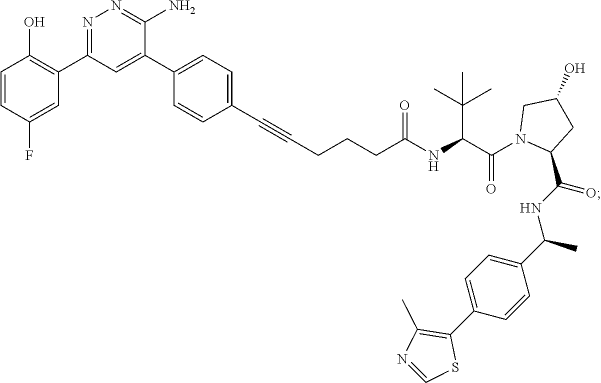 [SMILES: Cc1ncsc1-c1ccc([C@H](C)NC(=O)[C@@H]2C[C@@H](O)CN2C(=O)[C@@H](NC(=O)CCCC#Cc2ccc(-c3cc(-c4cc(F)ccc4O)nnc3N)cc2)C(C)(C)C)cc1] |
| 24 | 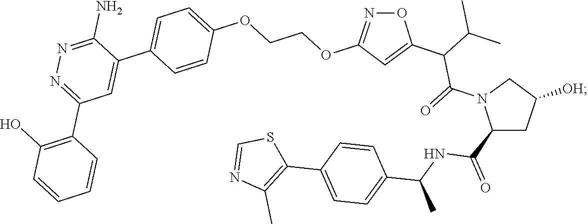 [SMILES: Cc1ncsc1-c1ccc([C@H](C)NC(=O)[C@@H]2C[C@@H](O)CN2C(=O)C(c2cc(OCCOc3ccc(-c4cc(-c5ccccc5O)nnc4N)cc3)no2)C(C)C)cc1] |
| 25 | 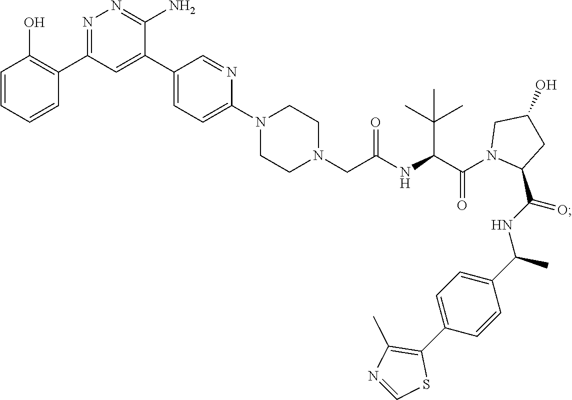 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)cn3)CC2)C(C)(C)C)cc1] |
| 26 | 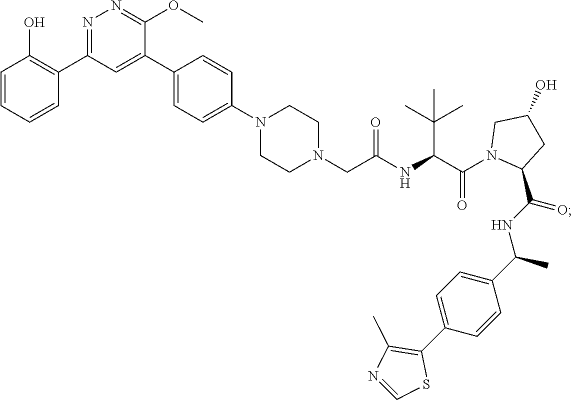 [SMILES: COc1nnc(-c2ccccc2O)cc1-c1ccc(N2CCN(CC(=O)N[C@H](C(=O)N3C[C@H](O)C[C@H]3C(=O)N[C@@H](C)c3ccc(-c4scnc4C)cc3)C(C)(C)C)CC2)cc1] |
| 27 | 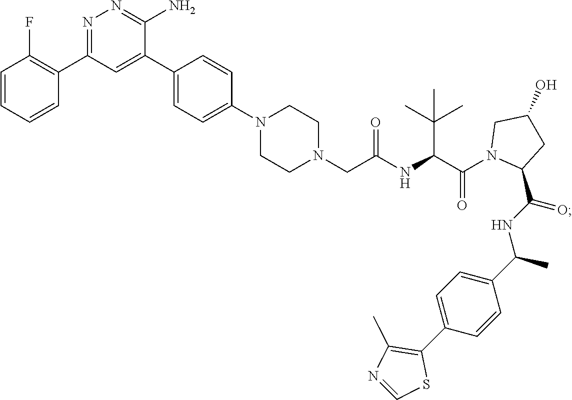 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5F)nnc4N)cc3)CC2)C(C)(C)C)cc1] |
| 28 | 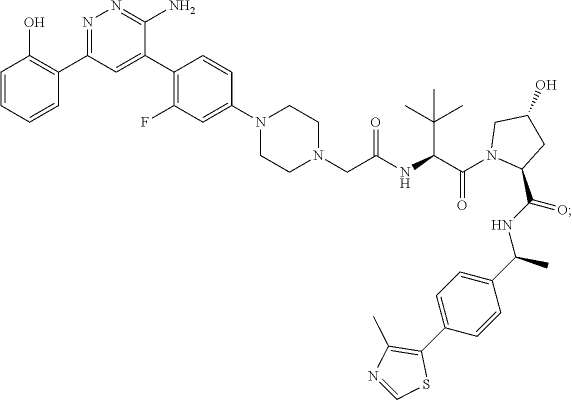 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)c(F)c3)CC2)C(C)(C)C)cc1] |
| 29 | 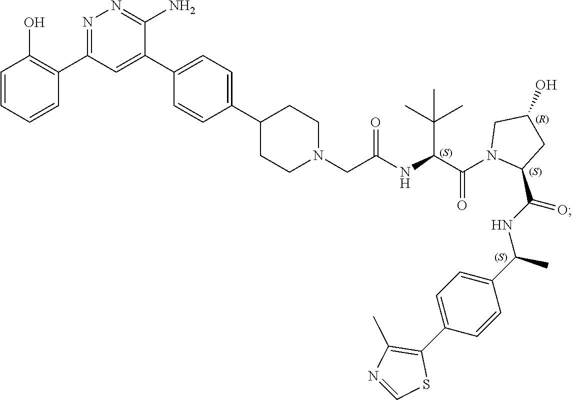 |
| 30 | 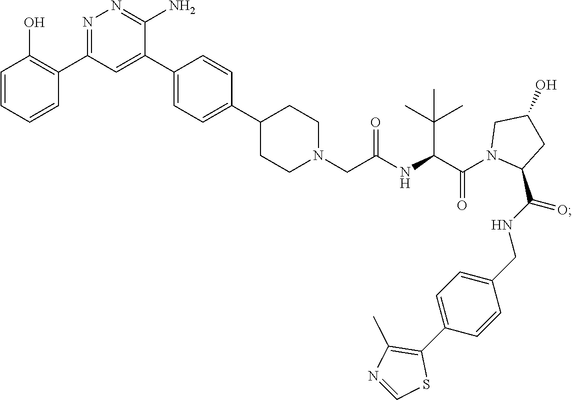 [SMILES: Cc1ncsc1-c1ccc(CNC(=O)[C@@H]2C[C@@H](O)CN2C(=O)[C@@H](NC(=O)CN2CCC(c3ccc(-c4cc(-c5ccccc5O)nnc4N)cc3)CC2)C(C)(C)C)cc1] |
| 31 | 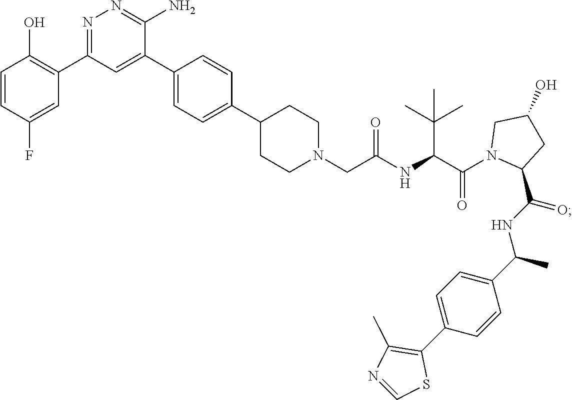 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5cc(F)ccc5O)nnc4N)cc3)CC2)C(C)(C)C)cc1] |
| 32 | 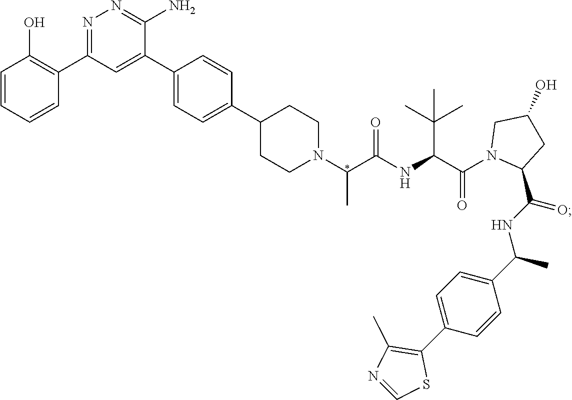 [SMILES: Cc1ncsc1-c1ccc([C@H](C)NC(=O)[C@@H]2C[C@@H](O)CN2C(=O)[C@@H](NC(=O)C(C)N2CCC(c3ccc(-c4cc(-c5ccccc5O)nnc4N)cc3)CC2)C(C)(C)C)cc1] |
| 33 | Isomer-1 of compound 32; |
| 34 | Isomer-2 of compound 32; |
| 35 | 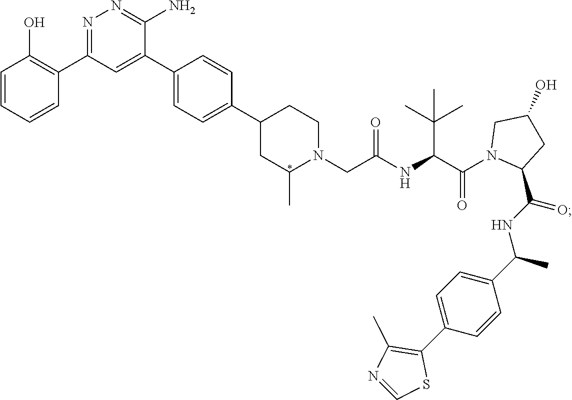 [SMILES: Cc1ncsc1-c1ccc([C@H](C)NC(=O)[C@@H]2C[C@@H](O)CN2C(=O)[C@@H](NC(=O)CN2CCC(c3ccc(-c4cc(-c5ccccc5O)nnc4N)cc3)CC2C)C(C)(C)C)cc1] |
| 36 | Isomer-1 of compound 35; |
| 37 | Isomer-2 of compound 35; |
| 38 | 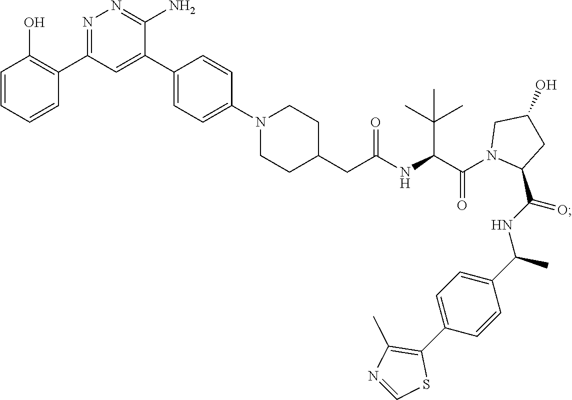 [SMILES: Cc1ncsc1-c1ccc([C@H](C)NC(=O)[C@@H]2C[C@@H](O)CN2C(=O)[C@@H](NC(=O)CC2CCN(c3ccc(-c4cc(-c5ccccc5O)nnc4N)cc3)CC2)C(C)(C)C)cc1] |
| 39 | 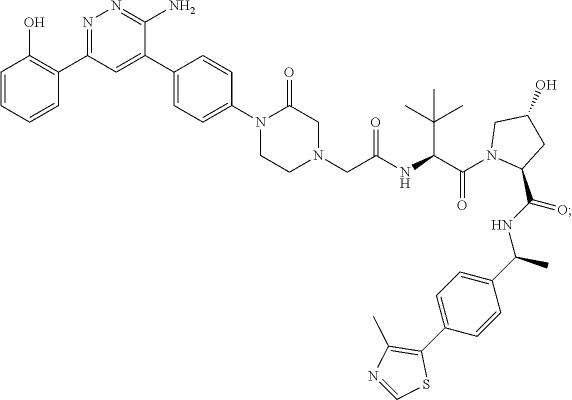 [SMILES: Cc1ncsc1-c1ccc([C@H](C)NC(=O)[C@@H]2C[C@@H](O)CN2C(=O)[C@@H](NC(=O)CN2CCN(c3ccc(-c4cc(-c5ccccc5O)nnc4N)cc3)C(=O)C2)C(C)(C)C)cc1] |
| 40 | 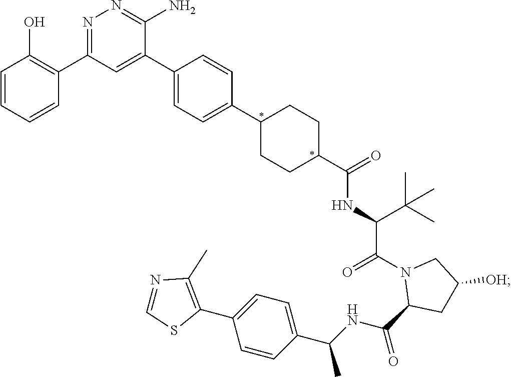 [SMILES: Cc1ncsc1-c1ccc([C@H](C)NC(=O)[C@@H]2C[C@@H](O)CN2C(=O)[C@@H](NC(=O)C2CCC(c3ccc(-c4cc(-c5ccccc5O)nnc4N)cc3)CC2)C(C)(C)C)cc1] |
| 41 | Isomer-1 of compound 40; |
| 42 | Isomer-2 of compound 40; |
| 43 | 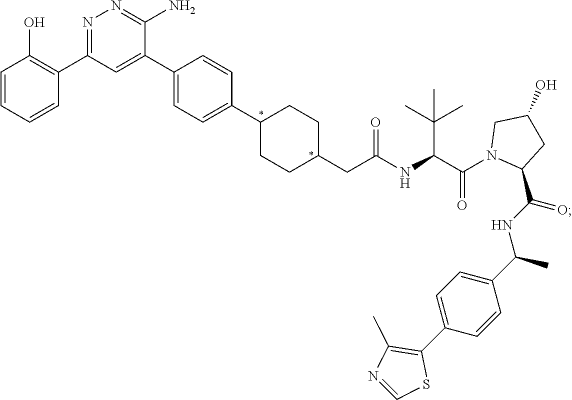 [SMILES: Cc1ncsc1-c1ccc([C@H](C)NC(=O)[C@@H]2C[C@@H](O)CN2C(=O)[C@@H](NC(=O)CC2CCC(c3ccc(-c4cc(-c5ccccc5O)nnc4N)cc3)CC2)C(C)(C)C)cc1] |
| 44 | Isomer-1 of compound 43; |
| 45 | Isomer-2 of compound 43; |
| 46 | 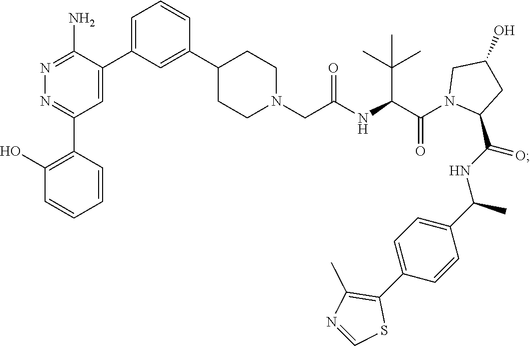 [SMILES: Cc1ncsc1-c1ccc([C@H](C)NC(=O)[C@@H]2C[C@@H](O)CN2C(=O)[C@@H](NC(=O)CN2CCC(c3cccc(-c4cc(-c5ccccc5O)nnc4N)c3)CC2)C(C)(C)C)cc1] |
| 47 | 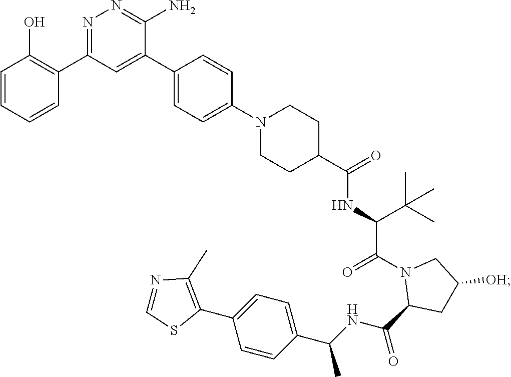 [SMILES: Cc1ncsc1-c1ccc([C@H](C)NC(=O)[C@@H]2C[C@@H](O)CN2C(=O)[C@@H](NC(=O)C2CCN(c3ccc(-c4cc(-c5ccccc5O)nnc4N)cc3)CC2)C(C)(C)C)cc1] |
| 48 | 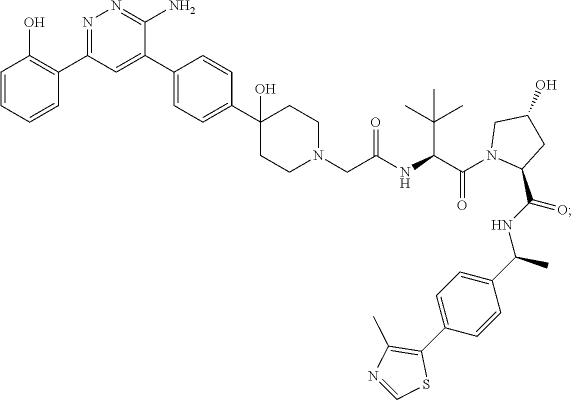 [SMILES: Cc1ncsc1-c1ccc([C@H](C)NC(=O)[C@@H]2C[C@@H](O)CN2C(=O)[C@@H](NC(=O)CN2CCC(O)(c3ccc(-c4cc(-c5ccccc5O)nnc4N)cc3)CC2)C(C)(C)C)cc1] |
| 49 | 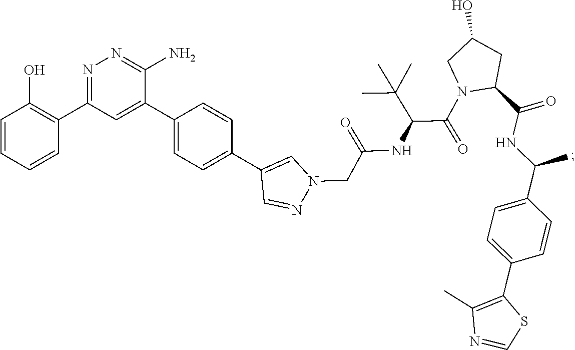 [SMILES: Cc1ncsc1-c1ccc([C@H](C)NC(=O)[C@@H]2C[C@@H](O)CN2C(=O)[C@@H](NC(=O)Cn2cc(-c3ccc(-c4cc(-c5ccccc5O)nnc4N)cc3)cn2)C(C)(C)C)cc1] |
| 50 | 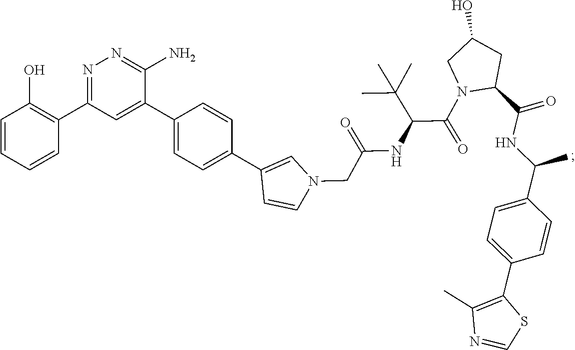 [SMILES: Cc1ncsc1-c1ccc([C@H](C)NC(=O)[C@@H]2C[C@@H](O)CN2C(=O)[C@@H](NC(=O)Cn2ccc(-c3ccc(-c4cc(-c5ccccc5O)nnc4N)cc3)c2)C(C)(C)C)cc1] |
| 51 | 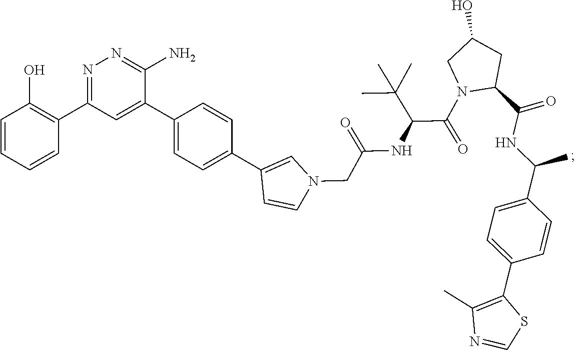 [SMILES: Cc1ncsc1-c1ccc([C@H](C)NC(=O)[C@@H]2C[C@@H](O)CN2C(=O)[C@@H](NC(=O)Cn2ccc(-c3ccc(-c4cc(-c5ccccc5O)nnc4N)cc3)c2)C(C)(C)C)cc1] |
| 52 | 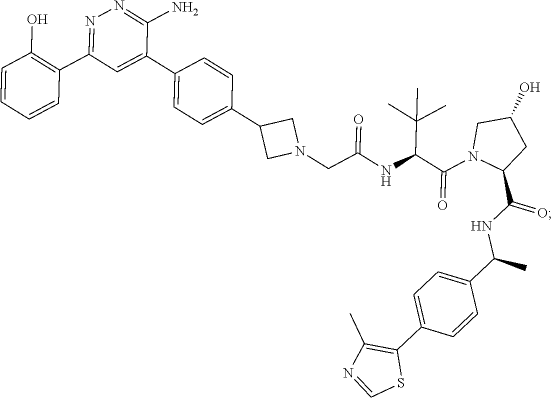 [SMILES: Cc1ncsc1-c1ccc([C@H](C)NC(=O)[C@@H]2C[C@@H](O)CN2C(=O)[C@@H](NC(=O)CN2CC(c3ccc(-c4cc(-c5ccccc5O)nnc4N)cc3)C2)C(C)(C)C)cc1] |
| 53 | 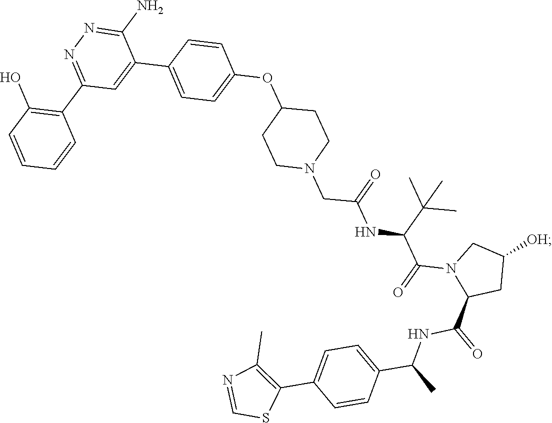 [SMILES: Cc1ncsc1-c1ccc([C@H](C)NC(=O)[C@@H]2C[C@@H](O)CN2C(=O)[C@@H](NC(=O)CN2CCC(Oc3ccc(-c4cc(-c5ccccc5O)nnc4N)cc3)CC2)C(C)(C)C)cc1] |
| 54 | 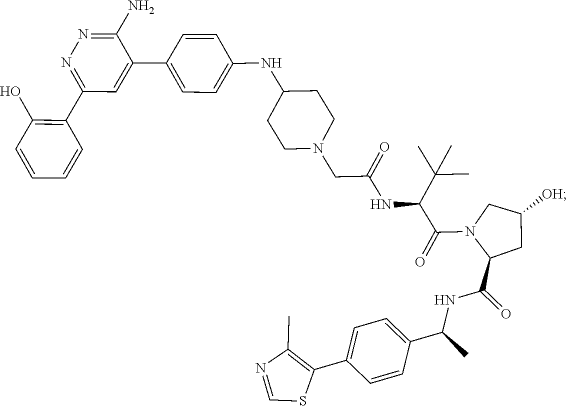 [SMILES: Cc1ncsc1-c1ccc([C@H](C)NC(=O)[C@@H]2C[C@@H](O)CN2C(=O)[C@@H](NC(=O)CN2CCC(Nc3ccc(-c4cc(-c5ccccc5O)nnc4N)cc3)CC2)C(C)(C)C)cc1] |
| 55 | 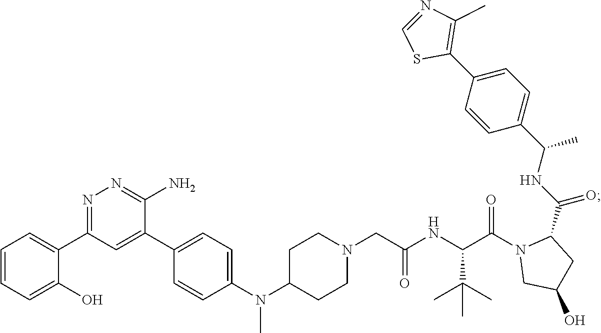 [SMILES: Cc1ncsc1-c1ccc([C@H](C)NC(=O)[C@@H]2C[C@@H](O)CN2C(=O)[C@@H](NC(=O)CN2CCC(N(C)c3ccc(-c4cc(-c5ccccc5O)nnc4N)cc3)CC2)C(C)(C)C)cc1] |
| 56 | 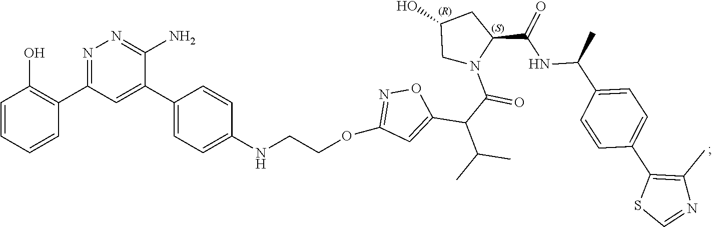 |
| 57 | 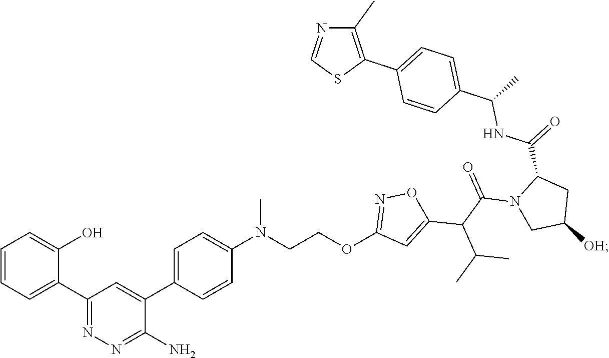 [SMILES: Cc1ncsc1-c1ccc([C@H](C)NC(=O)[C@@H]2C[C@@H](O)CN2C(=O)C(c2cc(OCCN(C)c3ccc(-c4cc(-c5ccccc5O)nnc4N)cc3)no2)C(C)C)cc1] |
| 58 | 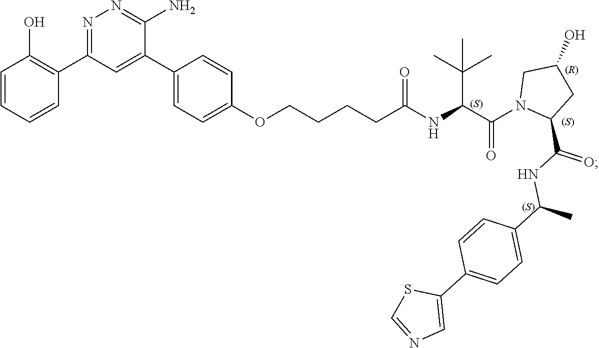 |
| 59 | 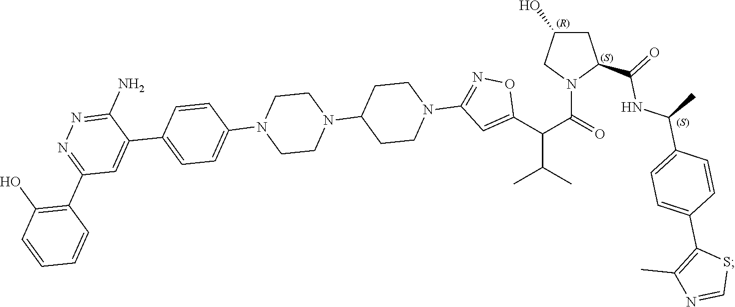 |
| 60 | 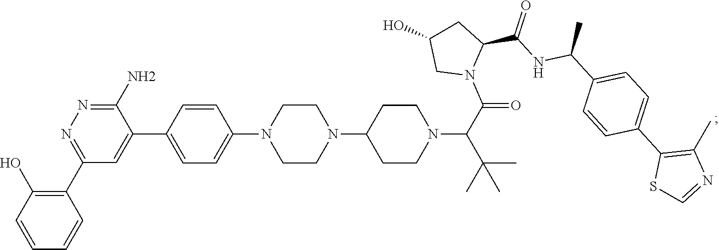 [SMILES: Cc1ncsc1-c1ccc([C@H](C)NC(=O)[C@@H]2C[C@@H](O)CN2C(=O)C(N2CCC(N3CCN(c4ccc(-c5cc(-c6ccccc6O)nnc5N)cc4)CC3)CC2)C(C)(C)C)cc1] |
| 61 | 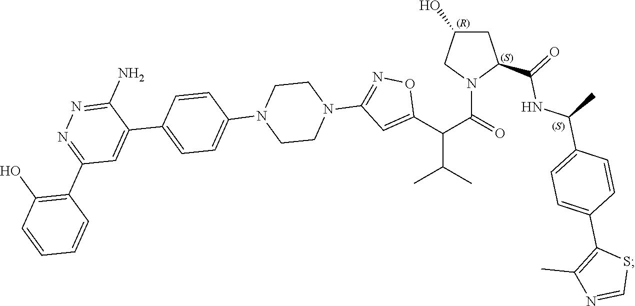 |
| 62 | 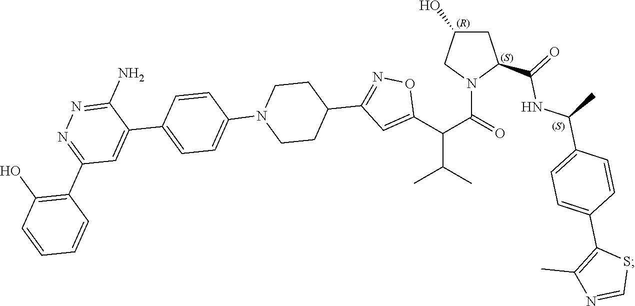 |
| 63 | 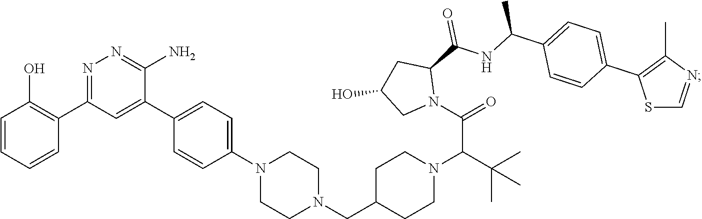 [SMILES: Cc1ncsc1-c1ccc([C@H](C)NC(=O)[C@@H]2C[C@@H](O)CN2C(=O)C(N2CCC(CN3CCN(c4ccc(-c5cc(-c6ccccc6O)nnc5N)cc4)CC3)CC2)C(C)(C)C)cc1] |
| 64 | 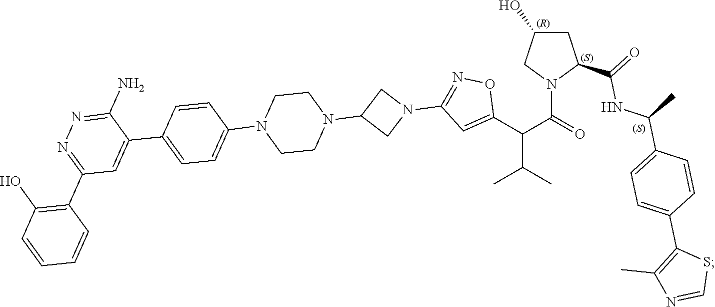 |
| 65 | 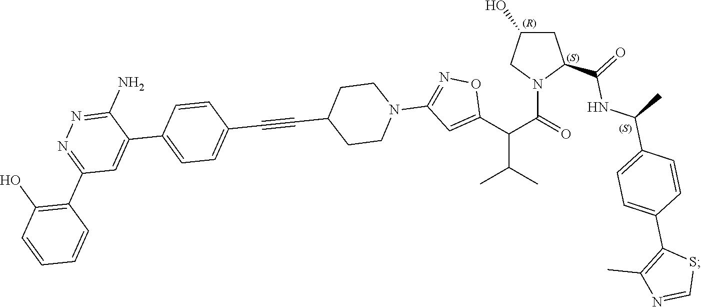 |
| 66 | 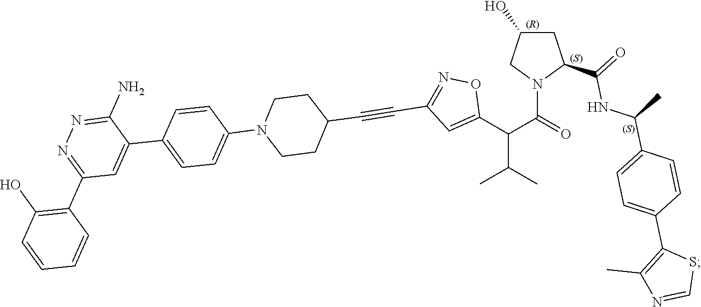 |
| 67 | 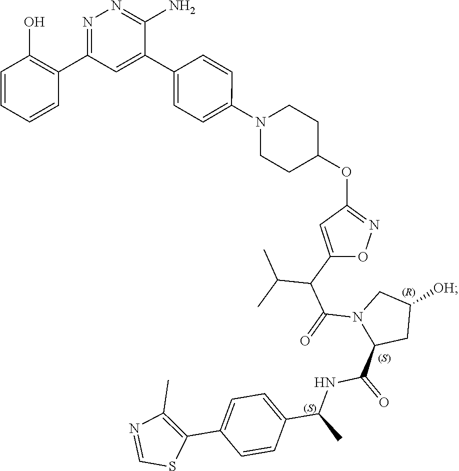 |
| 68 | 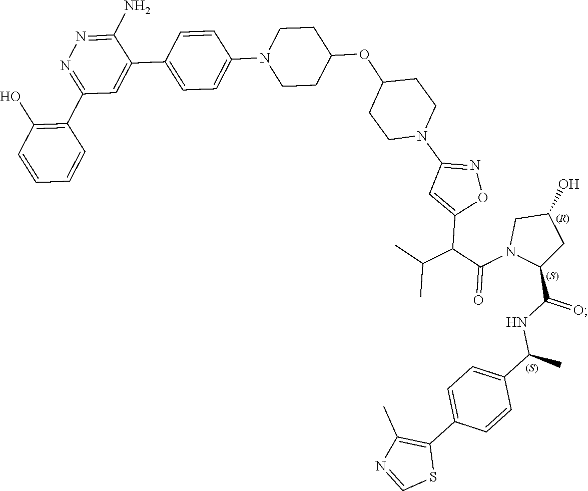 |
| 69 | 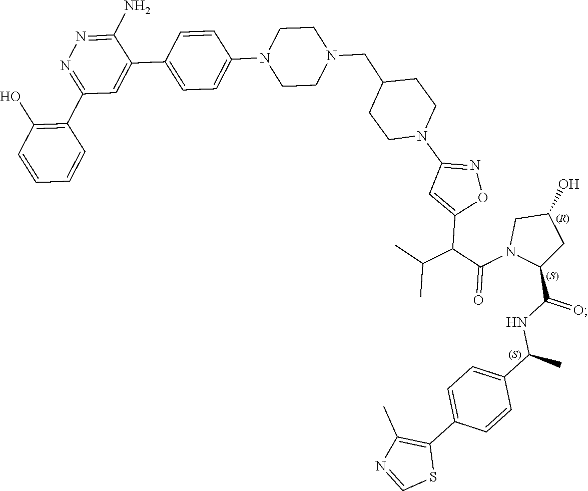 |
| | and |
| 70 | 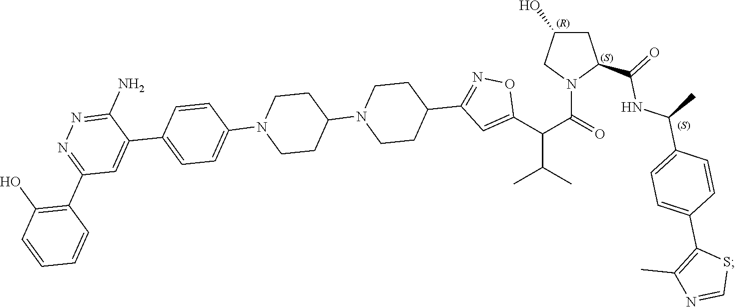 |

or a pharmaceutically acceptable salt or a stereoisomer or a tautomer thereof.

46. A pharmaceutical composition comprising the compound of any one of claims 1 to 45 or a pharmaceutically acceptable salt or a stereoisomer or a tautomer thereof and a pharmaceutically acceptable carrier or an excipient.

47. The pharmaceutical composition of claim 46, for use in degrading a target protein in a subject, wherein the target protein is SMARCA2 and/or SMARCA4.

48. The pharmaceutical composition for use of claim 47, wherein the subject is afflicted with a disease or disorder dependent upon at least one of SMARCA2 and SMARCA4.

49. The pharmaceutical composition for use of claim 47 or 48, wherein the disease or disorder is cancer selected from hematologic cancers, lung cancer (i.e. non-small cell lung cancer), acoustic neuroma, acute leukemia, acute lymphocytic leukemia, acute myelocytic leukemia (adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

50. A compound according to any one of claims 1 to 45, or a pharmaceutically acceptable salt or a stereoisomer or a tautomer thereof for use as a medicament.

51. A method for degrading a target protein in a subject comprising administering to the subject in need thereof a therapeutically effective amount of the compound according to any one of claims 1 to 45 or a pharmaceutically acceptable salt or a stereoisomer or a tautomer thereof.

52. The method of claim 51, wherein the target protein is SMARCA2 and/or SMARCA4.

53. A method for treating a disease or disorder dependent upon at least one of SMARCA2 and SMARCA4 in a subject comprising administering to the subject in need thereof a therapeutically effective amount of a compound according to any one of claims 1 to 45 or a pharmaceutically acceptable salt or a stereoisomer or a tautomer thereof.

54. The method of claim 53, wherein the disease or disorder dependent upon SMARCA2 and/or SMARCA4 is cancer selected from hematologic cancers, lung cancer (i.e. non-small cell lung cancer), acoustic neuroma, acute leukemia, acute lymphocytic leukemia, acute myelocytic leukemia (adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

55. A compound according to any one of claims 1 to 45 for use in the treatment of a disease or disorder dependent upon SMARCA2 and/or SMARCA4.

56. The compound for use according to claim 55, wherein the disease or disorder dependent upon SMARCA2 and/or SMARCA4 is cancer.

57. Use of a compound or a pharmaceutical acceptable salt or a stereoisomer or a tautomer thereof according to any one of claims 1 to 45, in the manufacture of a medicament for the treatment of a disease or disorder dependent upon SMARCA2 and/or SMARCA4; wherein the disease or disorder is cancer.
